(19) United States
(12) Patent Application Publication
(10) Pub. No.: US 2026/0242466 A1
(43) Pub. Date: Aug. 20, 2026

(54) ANTIBODY TARGETING CD112R OR ANTIGEN-BINDING FRAGMENT THEREOF AND USE THEREOF

(71) Applicant: HANGZHOU ZHONGMEIHUADONG PHARMACEUTICAL CO., LTD., Zhejiang (CN)

(72) Inventors: Qingyu SHU, Zhejiang (CN); Chen LIN, Zhejiang (CN); Xuefei FANG, Zhejiang (CN); Min CHEN, Zhejiang (CN)

(21) Appl. No.: 19/143,944

(22) PCT Filed: Dec. 4, 2023
(86) PCT No.: PCT/CN2023/136100

§ 371 Date: Jun. 27, 2025

(30) Foreign Application Priority Data
Dec. 30, 2022 (CN) 202211743931.1
May 8, 2023 (CN) 202310508581.9

(51) Int. Cl. C07K 16/28; A61P 35/00; A61P 37/04

(52) U.S. Cl. CPC C07K 16/2803; A61P 35/00; A61P 37/04; C07K 2317/24; C07K 2317/52; C07K 2317/565; C07K 2317/76; C07K 2317/92

(57) ABSTRACT

The present application relates to an anti-CD112R antibody or an antigen-binding fragment thereof, and a preparation method therefor and the use thereof. The present invention further relates to the therapeutic and diagnostic uses of the antibodies and antibody fragments.

### TECHNICAL FIELD

The present application relates to an antibody or an antigen-binding fragment thereof that specifically recognizes CD112R, and a preparation method and use thereof.

### BACKGROUND ART

CD112R, also known as Poliovirus Receptor Related Immunoglobulin Domain-containing Protein, Q6DKI7 or C7orfl5, is a transmembrane domain protein of 326 amino acids in length, having a signal peptide (spanning amino acids 1 to 40), an ectodomain (spanning amino acids 41 to 171), a transmembrane domain (spanning amino acids 172 to 190), and a cytoplasmic domain (spanning amino acids 191 to 326). CD112R binds to Poliovirus Receptor Related Protein 2 (PVLR2, also known as nectin2, CD112 or herpesvirus entry mediator B (HVEB), human plasma membrane glycoprotein), i.e., the binding partner of CD112R.

Administration of anti-CD112R immunotherapy provides an opportunity to increase, enhance and maintain an immune response. CD112R is an inhibitory receptor mainly expressed by T cells and NK cells, and competes with the activating receptor CD226 for binding to CD112. The interaction between CD112 and CD112R has a higher affinity than the interaction with CD226, thereby effectively regulating CD226-mediated cell activation. Anti CD112R antibodies blocking the interaction with CD112 directly limit downstream of CD112R inhibitory signaling, while promote greater immune cell activation by increasing the interaction between CD226 with CD112.

Treatment with anti-CD112R antibodies provides an opportunity to down-regulate inhibitory signals that may occur when CD112R-expressing immune cells bind to CD112 on tumor cells and/or myeloid cells within tumor microenvironments, and may enhance, increase and maintain anti-tumor immune responses.

Although the prior art has disclosed anti-CD112R antibodies, there is still an urgent need to develop high quality anti-CD112R antibodies.

### SUMMARY

In a first aspect, the present disclosure provides an antibody targeting human CD112R, which has the following advantages:
(1) binding human/cynomolgus monkey CD112R protein with high affinity;
(2) inhibiting or blocking the binding of human CD112R protein to human CD112 or cells expressing human CD112;
(3) After blocking the binding of CD112R protein to CD112, CD8 T cells was stimulated to kill tumor cells.

In one embodiment, the invention provides an anti-CD112R antibody that specifically binds to CD112R and an antigen-binding fragment thereof, comprising:
1) three heavy chain CDRs (HCDR1, HCDR2, HCDR3) comprised in the heavy chain variable region as set forth in SEQ ID NO: 430 and three light chain CDRs (LCDR1, LCDR2, LCDR3) comprised in the light chain variable region as set forth in SEQ ID NO: 431;
2) three heavy chain CDRs (HCDR1, HCDR2, HCDR3) comprised in the heavy chain variable region as set forth in SEQ ID NO: 432 and three light chain CDRs (LCDR1, LCDR2, LCDR3) comprised in the light chain variable region as set forth in SEQ ID NO: 433;
3) three heavy chain CDRs (HCDR1, HCDR2, HCDR3) comprised in the heavy chain variable region as set forth in SEQ ID NO: 434 and three light chain CDRs (LCDR1, LCDR2, LCDR3) comprised in the light chain variable region as set forth in SEQ ID NO: 435;
4) three heavy chain CDRs (HCDR1, HCDR2, HCDR3) comprised in the heavy chain variable region as set forth in SEQ ID NO: 436 and three light chain CDRs (LCDR1, LCDR2, LCDR3) comprised in the light chain variable region as set forth in SEQ ID NO: 437;
5) three heavy chain CDRs (HCDR1, HCDR2, HCDR3) comprised in the heavy chain variable region as set forth in SEQ ID NO: 438 and three light chain CDRs (LCDR1, LCDR2, LCDR3) comprised in the light chain variable region as set forth in SEQ ID NO: 439;
6) three heavy chain CDRs (HCDR1, HCDR2, HCDR3) comprised in the heavy chain variable region as set forth in SEQ ID NO: 438 and three light chain CDRs (LCDR1, LCDR2, LCDR3) comprised in the light chain variable region as set forth in SEQ ID NO: 440;
7) three heavy chain CDRs (HCDR1, HCDR2, HCDR3) comprised in the heavy chain variable region as set forth in SEQ ID NO: 441 and three light chain CDRs (LCDR1, LCDR2, LCDR3) comprised in the light chain variable region as set forth in SEQ ID NO: 442;
8) three heavy chain CDRs (HCDR1, HCDR2, HCDR3) comprised in the heavy chain variable region as set forth in SEQ ID NO: 443 and three light chain CDRs (LCDR1, LCDR2, LCDR3) comprised in the light chain variable region as set forth in SEQ ID NO: 444;
9) three heavy chain CDRs (HCDR1, HCDR2, HCDR3) comprised in the heavy chain variable region as set forth in SEQ ID NO: 445 and three light chain CDRs (LCDR1, LCDR2, LCDR3) comprised in the light chain variable region as set forth in SEQ ID NO: 446;
10) three heavy chain CDRs (HCDR1, HCDR2, HCDR3) comprised in the heavy chain variable region as set forth in SEQ ID NO: 447 and three light chain CDRs (LCDR1, LCDR2, LCDR3) comprised in the light chain variable region as set forth in SEQ ID NO: 448;
11) three heavy chain CDRs (HCDR1, HCDR2, HCDR3) comprised in the heavy chain variable region as set forth in SEQ ID NO: 449 and three light chain CDRs (LCDR1, LCDR2, LCDR3) comprised in the light chain variable region as set forth in SEQ ID NO: 450;
12) three heavy chain CDRs (HCDR1, HCDR2, HCDR3) comprised in the heavy chain variable region as set forth in SEQ ID NO: 451 and three light chain CDRs (LCDR1, LCDR2, LCDR3) comprised in the light chain variable region as set forth in SEQ ID NO: 452;
13) three heavy chain CDRs (HCDR1, HCDR2, HCDR3) comprised in the heavy chain variable region as set forth in SEQ ID NO: 453 and three light chain CDRs (LCDR1, LCDR2, LCDR3) comprised in the light chain variable region as set forth in SEQ ID NO: 454;
14) three heavy chain CDRs (HCDR1, HCDR2, HCDR3) comprised in the heavy chain variable region as set forth in SEQ ID NO: 455 and three light chain CDRs (LCDR1, LCDR2, LCDR3) comprised in the light chain variable region as set forth in SEQ ID NO: 456;
15) three heavy chain CDRs (HCDR1, HCDR2, HCDR3) comprised in the heavy chain variable region as set forth in SEQ ID NO: 457 and three light chain CDRs (LCDR1, LCDR2, LCDR3) comprised in the light chain variable region as set forth in SEQ ID NO: 458;
16) three heavy chain CDRs (HCDR1, HCDR2, HCDR3) comprised in the heavy chain variable region as set forth in SEQ ID NO: 459 and three light chain CDRs (LCDR1, LCDR2, LCDR3) comprised in the light chain variable region as set forth in SEQ ID NO: 460;
17) three heavy chain CDRs (HCDR1, HCDR2, HCDR3) comprised in the heavy chain variable region as set forth in SEQ ID NO: 461 and three light chain CDRs (LCDR1, LCDR2, LCDR3) comprised in the light chain variable region as set forth in SEQ ID NO: 462;
18) three heavy chain CDRs (HCDR1, HCDR2, HCDR3) comprised in the heavy chain variable region as set forth in SEQ ID NO: 463 and three light chain CDRs (LCDR1, LCDR2, LCDR3) comprised in the light chain variable region as set forth in SEQ ID NO: 464;
19) three heavy chain CDRs (HCDR1, HCDR2, HCDR3) comprised in the heavy chain variable region as set forth in SEQ ID NO: 465 and three light chain CDRs (LCDR1, LCDR2, LCDR3) comprised in the light chain variable region as set forth in SEQ ID NO: 466;
20) three heavy chain CDRs (HCDR1, HCDR2, HCDR3) comprised in the heavy chain variable region as set forth in SEQ ID NO: 467 and three light chain CDRs (LCDR1, LCDR2, LCDR3) comprised in the light chain variable region as set forth in SEQ ID NO: 468;
21) three heavy chain CDRs (HCDR1, HCDR2, HCDR3) comprised in the heavy chain variable region as set forth in SEQ ID NO: 469 and three light chain CDRs (LCDR1, LCDR2, LCDR3) comprised in the light chain variable region as set forth in SEQ ID NO: 470;
22) three heavy chain CDRs (HCDR1, HCDR2, HCDR3) comprised in the heavy chain variable region as set forth in SEQ ID NO: 471 and three light chain CDRs (LCDR1, LCDR2, LCDR3) comprised in the light chain variable region as set forth in SEQ ID NO: 472;
23) three heavy chain CDRs (HCDR1, HCDR2, HCDR3) comprised in the heavy chain variable region as set forth in SEQ ID NO: 473 and three light chain CDRs (LCDR1, LCDR2, LCDR3) comprised in the light chain variable region as set forth in SEQ ID NO: 474;
24) three heavy chain CDRs (HCDR1, HCDR2, HCDR3) comprised in the heavy chain variable region as set forth in SEQ ID NO: 475 and three light chain CDRs (LCDR1, LCDR2, LCDR3) comprised in the light chain variable region as set forth in SEQ ID NO: 476;
25) three heavy chain CDRs (HCDR1, HCDR2, HCDR3) comprised in the heavy chain variable region as set forth in SEQ ID NO: 477 and three light chain CDRs (LCDR1, LCDR2, LCDR3) comprised in the light chain variable region as set forth in SEQ ID NO: 478;
26) three heavy chain CDRs (HCDR1, HCDR2, HCDR3) comprised in the heavy chain variable region as set forth in SEQ ID NO: 479 and three light chain CDRs (LCDR1, LCDR2, LCDR3) comprised in the light chain variable region as set forth in SEQ ID NO: 480;
27) three heavy chain CDRs (HCDR1, HCDR2, HCDR3) comprised in the heavy chain variable region as set forth in SEQ ID NO: 481 and three light chain CDRs (LCDR1, LCDR2, LCDR3) comprised in the light chain variable region as set forth in SEQ ID NO: 482;
28) three heavy chain CDRs (HCDR1, HCDR2, HCDR3) comprised in the heavy chain variable region as set forth in SEQ ID NO: 483 and three light chain CDRs (LCDR1, LCDR2, LCDR3) comprised in the light chain variable region as set forth in SEQ ID NO: 484;
29) three heavy chain CDRs (HCDR1, HCDR2, HCDR3) comprised in the heavy chain variable region as set forth in SEQ ID NO: 485 and three light chain CDRs (LCDR1, LCDR2, LCDR3) comprised in the light chain variable region as set forth in SEQ ID NO: 486;
30) three heavy chain CDRs (HCDR1, HCDR2, HCDR3) comprised in the heavy chain variable region as set forth in SEQ ID NO: 487 and three light chain CDRs (LCDR1, LCDR2, LCDR3) comprised in the light chain variable region as set forth in SEQ ID NO: 456;
31) three heavy chain CDRs (HCDR1, HCDR2, HCDR3) comprised in the heavy chain variable region as set forth in SEQ ID NO: 488 and three light chain CDRs (LCDR1, LCDR2, LCDR3) comprised in the light chain variable region as set forth in SEQ ID NO: 489;
32) three heavy chain CDRs (HCDR1, HCDR2, HCDR3) comprised in the heavy chain variable region as set forth in SEQ ID NO: 490 and three light chain CDRs (LCDR1, LCDR2, LCDR3) comprised in the light chain variable region as set forth in SEQ ID NO: 491;
33) three heavy chain CDRs (HCDR1, HCDR2, HCDR3) comprised in the heavy chain variable region as set forth in SEQ ID NO: 492 and three light chain CDRs (LCDR1, LCDR2, LCDR3) comprised in the light chain variable region as set forth in SEQ ID NO: 489;
34) three heavy chain CDRs (HCDR1, HCDR2, HCDR3) comprised in the heavy chain variable region as set forth in SEQ ID NO: 493 and three light chain CDRs (LCDR1, LCDR2, LCDR3) comprised in the light chain variable region as set forth in SEQ ID NO: 494;
35) three heavy chain CDRs (HCDR1, HCDR2, HCDR3) comprised in the heavy chain variable region as set forth in SEQ ID NO: 493 and three light chain CDRs (LCDR1, LCDR2, LCDR3) comprised in the light chain variable region as set forth in SEQ ID NO: 495;
36) three heavy chain CDRs (HCDR1, HCDR2, HCDR3) comprised in the heavy chain variable region as set forth in SEQ ID NO: 496 and three light chain CDRs (LCDR1, LCDR2, LCDR3) comprised in the light chain variable region as set forth in SEQ ID NO: 497;
37) three heavy chain CDRs (HCDR1, HCDR2, HCDR3) comprised in the heavy chain variable region as set forth in SEQ ID NO: 498 and three light chain CDRs (LCDR1, LCDR2, LCDR3) comprised in the light chain variable region as set forth in SEQ ID NO: 497;
38) three heavy chain CDRs (HCDR1, HCDR2, HCDR3) comprised in the heavy chain variable region as set forth in SEQ ID NO: 499 and three light chain CDRs (LCDR1, LCDR2, LCDR3) comprised in the light chain variable region as set forth in SEQ ID NO: 500;
39) three heavy chain CDRs (HCDR1, HCDR2, HCDR3) comprised in the heavy chain variable region as set forth in SEQ ID NO: 501 and three light chain CDRs (LCDR1, LCDR2, LCDR3) comprised in the light chain variable region as set forth in SEQ ID NO: 503;
40) three heavy chain CDRs (HCDR1, HCDR2, HCDR3) comprised in the heavy chain variable region as set forth in SEQ ID NO: 502 and three light chain CDRs (LCDR1, LCDR2, LCDR3) comprised in the light chain variable region as set forth in SEQ ID NO: 503;
41) three heavy chain CDRs (HCDR1, HCDR2, HCDR3) comprised in the heavy chain variable region as set forth in SEQ ID NO: 504 and three light chain CDRs (LCDR1, LCDR2, LCDR3) comprised in the light chain variable region as set forth in SEQ ID NO: 505;
42) three heavy chain CDRs (HCDR1, HCDR2, HCDR3) comprised in the heavy chain variable region as set forth in SEQ ID NO: 499 and three light chain CDRs (LCDR1, LCDR2, LCDR3) comprised in the light chain variable region as set forth in SEQ ID NO: 506;
43) three heavy chain CDRs (HCDR1, HCDR2, HCDR3) comprised in the heavy chain variable region as set forth in SEQ ID NO: 507 and three light chain CDRs (LCDR1, LCDR2, LCDR3) comprised in the light chain variable region as set forth in SEQ ID NO: 503;
44) three heavy chain CDRs (HCDR1, HCDR2, HCDR3) comprised in the heavy chain variable region as set forth in SEQ ID NO: 508 and three light chain CDRs (LCDR1, LCDR2, LCDR3) comprised in the light chain variable region as set forth in SEQ ID NO: 509;
45) three heavy chain CDRs (HCDR1, HCDR2, HCDR3) comprised in the heavy chain variable region as set forth in SEQ ID NO: 510 and three light chain CDRs (LCDR1, LCDR2, LCDR3) comprised in the light chain variable region as set forth in SEQ ID NO: 511;
46) three heavy chain CDRs (HCDR1, HCDR2, HCDR3) comprised in the heavy chain variable region as set forth in SEQ ID NO: 512 and three light chain CDRs (LCDR1, LCDR2, LCDR3) comprised in the light chain variable region as set forth in SEQ ID NO: 513;
47) three heavy chain CDRs (HCDR1, HCDR2, HCDR3) comprised in the heavy chain variable region as set forth in SEQ ID NO: 514 and three light chain CDRs (LCDR1, LCDR2, LCDR3) comprised in the light chain variable region as set forth in SEQ ID NO: 515;
48) three heavy chain CDRs (HCDR1, HCDR2, HCDR3) comprised in the heavy chain variable region as set forth in SEQ ID NO: 501 and three light chain CDRs (LCDR1, LCDR2, LCDR3) comprised in the light chain variable region as set forth in SEQ ID NO: 516;
49) three heavy chain CDRs (HCDR1, HCDR2, HCDR3) comprised in the heavy chain variable region as set forth in SEQ ID NO: 517 and three light chain CDRs (LCDR1, LCDR2, LCDR3) comprised in the light chain variable region as set forth in SEQ ID NO: 518;
50) three heavy chain CDRs (HCDR1, HCDR2, HCDR3) comprised in the heavy chain variable region as set forth in SEQ ID NO: 519 and three light chain CDRs (LCDR1, LCDR2, LCDR3) comprised in the light chain variable region as set forth in SEQ ID NO: 520;
51) three heavy chain CDRs (HCDR1, HCDR2, HCDR3) comprised in the heavy chain variable region as set forth in SEQ ID NO: 521 and three light chain CDRs (LCDR1, LCDR2, LCDR3) comprised in the light chain variable region as set forth in SEQ ID NO: 522;
52) three heavy chain CDRs (HCDR1, HCDR2, HCDR3) comprised in the heavy chain variable region as set forth in SEQ ID NO: 523 and three light chain CDRs (LCDR1, LCDR2, LCDR3) comprised in the light chain variable region as set forth in SEQ ID NO: 524;
53) three heavy chain CDRs (HCDR1, HCDR2, HCDR3) comprised in the heavy chain variable region as set forth in SEQ ID NO: 525 and three light chain CDRs (LCDR1, LCDR2, LCDR3) comprised in the light chain variable region as set forth in SEQ ID NO: 486;
54) three heavy chain CDRs (HCDR1, HCDR2, HCDR3) comprised in the heavy chain variable region as set forth in SEQ ID NO: 526 and three light chain CDRs (LCDR1, LCDR2, LCDR3) comprised in the light chain variable region as set forth in SEQ ID NO: 527;
55) three heavy chain CDRs (HCDR1, HCDR2, HCDR3) comprised in the heavy chain variable region as set forth in SEQ ID NO: 528 and three light chain CDRs (LCDR1, LCDR2, LCDR3) comprised in the light chain variable region as set forth in SEQ ID NO: 529;
56) three heavy chain CDRs (HCDR1, HCDR2, HCDR3) comprised in the heavy chain variable region as set forth in SEQ ID NO: 530 and three light chain CDRs (LCDR1, LCDR2, LCDR3) comprised in the light chain variable region as set forth in SEQ ID NO: 531;
57) three heavy chain CDRs (HCDR1, HCDR2, HCDR3) comprised in the heavy chain variable region as set forth in SEQ ID NO: 530 and three light chain CDRs (LCDR1, LCDR2, LCDR3) comprised in the light chain variable region as set forth in SEQ ID NO: 533;
58) three heavy chain CDRs (HCDR1, HCDR2, HCDR3) comprised in the heavy chain variable region as set forth in SEQ ID NO: 532 and three light chain CDRs (LCDR1, LCDR2, LCDR3) comprised in the light chain variable region as set forth in SEQ ID NO: 531;
59) three heavy chain CDRs (HCDR1, HCDR2, HCDR3) comprised in the heavy chain variable region as set forth in SEQ ID NO: 532 and three light chain CDRs (LCDR1, LCDR2, LCDR3) comprised in the light chain variable region as set forth in SEQ ID NO: 533;
60) three heavy chain CDRs (HCDR1, HCDR2, HCDR3) comprised in the heavy chain variable region as set forth in SEQ ID NO: 534 and three light chain CDRs (LCDR1, LCDR2, LCDR3) comprised in the light chain variable region as set forth in SEQ ID NO: 535;
61) three heavy chain CDRs (HCDR1, HCDR2, HCDR3) comprised in the heavy chain variable region as set forth in SEQ ID NO: 536 and three light chain CDRs (LCDR1, LCDR2, LCDR3) comprised in the light chain variable region as set forth in SEQ ID NO: 537;
62) three heavy chain CDRs (HCDR1, HCDR2, HCDR3) comprised in the heavy chain variable region as set forth in SEQ ID NO: 538 and three light chain CDRs (LCDR1, LCDR2, LCDR3) comprised in the light chain variable region as set forth in SEQ ID NO: 539;
63) three heavy chain CDRs (HCDR1, HCDR2, HCDR3) comprised in the heavy chain variable region as set forth in SEQ ID NO: 540 and three light chain CDRs (LCDR1, LCDR2, LCDR3) comprised in the light chain variable region as set forth in SEQ ID NO: 541;
64) three heavy chain CDRs (HCDR1, HCDR2, HCDR3) comprised in the heavy chain variable region as set forth in SEQ ID NO: 540 and three light chain CDRs (LCDR1, LCDR2, LCDR3) comprised in the light chain variable region as set forth in SEQ ID NO: 542;
65) three heavy chain CDRs (HCDR1, HCDR2, HCDR3) comprised in the heavy chain variable region as set forth in SEQ ID NO: 543 and three light chain CDRs (LCDR1, LCDR2, LCDR3) comprised in the light chain variable region as set forth in SEQ ID NO: 544;
66) three heavy chain CDRs (HCDR1, HCDR2, HCDR3) comprised in the heavy chain variable region as set forth in SEQ ID NO: 545 and three light chain CDRs (LCDR1, LCDR2, LCDR3) comprised in the light chain variable region as set forth in SEQ ID NO: 546;
67) three heavy chain CDRs (HCDR1, HCDR2, HCDR3) comprised in the heavy chain variable region as set forth in SEQ ID NO: 547 and three light chain CDRs (LCDR1, LCDR2, LCDR3) comprised in the light chain variable region as set forth in SEQ ID NO: 548;
68) three heavy chain CDRs (HCDR1, HCDR2, HCDR3) comprised in the heavy chain variable region as set forth in SEQ ID NO: 549 and three light chain CDRs (LCDR1, LCDR2, LCDR3) comprised in the light chain variable region as set forth in SEQ ID NO: 550;
69) three heavy chain CDRs (HCDR1, HCDR2, HCDR3) comprised in the heavy chain variable region as set forth in SEQ ID NO: 551 and three light chain CDRs (LCDR1, LCDR2, LCDR3) comprised in the light chain variable region as set forth in SEQ ID NO: 552;
70) three heavy chain CDRs (HCDR1, HCDR2, HCDR3) comprised in the heavy chain variable region as set forth in SEQ ID NO: 553 and three light chain CDRs (LCDR1, LCDR2, LCDR3) comprised in the light chain variable region as set forth in SEQ ID NO: 554;
71) three heavy chain CDRs (HCDR1, HCDR2, HCDR3) comprised in the heavy chain variable region as set forth in SEQ ID NO: 555 and three light chain CDRs (LCDR1, LCDR2, LCDR3) comprised in the light chain variable region as set forth in SEQ ID NO: 556;
72) three heavy chain CDRs (HCDR1, HCDR2, HCDR3) comprised in the heavy chain variable region as set forth in SEQ ID NO: 557 and three light chain CDRs (LCDR1, LCDR2, LCDR3) comprised in the light chain variable region as set forth in SEQ ID NO: 558;
73) three heavy chain CDRs (HCDR1, HCDR2, HCDR3) comprised in the heavy chain variable region as set forth in SEQ ID NO: 559 and three light chain CDRs (LCDR1, LCDR2, LCDR3) comprised in the light chain variable region as set forth in SEQ ID NO: 560;
74) three heavy chain CDRs (HCDR1, HCDR2, HCDR3) comprised in the heavy chain variable region as set forth in SEQ ID NO: 561 and three light chain CDRs (LCDR1, LCDR2, LCDR3) comprised in the light chain variable region as set forth in SEQ ID NO: 554;
75) three heavy chain CDRs (HCDR1, HCDR2, HCDR3) comprised in the heavy chain variable region as set forth in SEQ ID NO: 562 and three light chain CDRs (LCDR1, LCDR2, LCDR3) comprised in the light chain variable region as set forth in SEQ ID NO: 563;
76) three heavy chain CDRs (HCDR1, HCDR2, HCDR3) comprised in the heavy chain variable region as set forth in SEQ ID NO: 564 and three light chain CDRs (LCDR1, LCDR2, LCDR3) comprised in the light chain variable region as set forth in SEQ ID NO: 565;
77) three heavy chain CDRs (HCDR1, HCDR2, HCDR3) comprised in the heavy chain variable region as set forth in SEQ ID NO: 566 and three light chain CDRs (LCDR1, LCDR2, LCDR3) comprised in the light chain variable region as set forth in SEQ ID NO: 567;
78) three heavy chain CDRs (HCDR1, HCDR2, HCDR3) comprised in the heavy chain variable region as set forth in SEQ ID NO: 568 and three light chain CDRs (LCDR1, LCDR2, LCDR3) comprised in the light chain variable region as set forth in SEQ ID NO: 569;
79) three heavy chain CDRs (HCDR1, HCDR2, HCDR3) comprised in the heavy chain variable region as set forth in SEQ ID NO: 570 and three light chain CDRs (LCDR1, LCDR2, LCDR3) comprised in the light chain variable region as set forth in SEQ ID NO: 571;
80) three heavy chain CDRs (HCDR1, HCDR2, HCDR3) comprised in the heavy chain variable region as set forth in SEQ ID NO: 572 and three light chain CDRs (LCDR1, LCDR2, LCDR3) comprised in the light chain variable region as set forth in SEQ ID NO: 573;
81) three heavy chain CDRs (HCDR1, HCDR2, HCDR3) comprised in the heavy chain variable region as set forth in SEQ ID NO: 574 and three light chain CDRs (LCDR1, LCDR2, LCDR3) comprised in the light chain variable region as set forth in SEQ ID NO: 575;
82) three heavy chain CDRs (HCDR1, HCDR2, HCDR3) comprised in the heavy chain variable region as set forth in SEQ ID NO: 576 and three light chain CDRs (LCDR1, LCDR2, LCDR3) comprised in the light chain variable region as set forth in SEQ ID NO: 578;
83) three heavy chain CDRs (HCDR1, HCDR2, HCDR3) comprised in the heavy chain variable region as set forth in SEQ ID NO: 577 and three light chain CDRs (LCDR1, LCDR2, LCDR3) comprised in the light chain variable region as set forth in SEQ ID NO: 578;
84) three heavy chain CDRs (HCDR1, HCDR2, HCDR3) comprised in the heavy chain variable region as set forth in SEQ ID NO: 579 and three light chain CDRs (LCDR1, LCDR2, LCDR3) comprised in the light chain variable region as set forth in SEQ ID NO: 580;
85) three heavy chain CDRs (HCDR1, HCDR2, HCDR3) comprised in the heavy chain variable region as set forth in SEQ ID NO: 581 and three light chain CDRs (LCDR1, LCDR2, LCDR3) comprised in the light chain variable region as set forth in SEQ ID NO: 582;
86) three heavy chain CDRs (HCDR1, HCDR2, HCDR3) comprised in the heavy chain variable region as set forth in SEQ ID NO: 583 and three light chain CDRs (LCDR1, LCDR2, LCDR3) comprised in the light chain variable region as set forth in SEQ ID NO: 584;
87) three heavy chain CDRs (HCDR1, HCDR2, HCDR3) comprised in the heavy chain variable region as set forth in SEQ ID NO: 585 and three light chain CDRs (LCDR1, LCDR2, LCDR3) comprised in the light chain variable region as set forth in SEQ ID NO: 586;
88) three heavy chain CDRs (HCDR1, HCDR2, HCDR3) comprised in the heavy chain variable region as set forth in SEQ ID NO: 587 and three light chain CDRs (LCDR1, LCDR2, LCDR3) comprised in the light chain variable region as set forth in SEQ ID NO: 588;
89) three heavy chain CDRs (HCDR1, HCDR2, HCDR3) comprised in the heavy chain variable region as set forth in SEQ ID NO: 587 and three light chain CDRs (LCDR1, LCDR2, LCDR3) comprised in the light chain variable region as set forth in SEQ ID NO: 589;
90) three heavy chain CDRs (HCDR1, HCDR2, HCDR3) comprised in the heavy chain variable region as set forth in SEQ ID NO: 590 and three light chain CDRs (LCDR1, LCDR2, LCDR3) comprised in the light chain variable region as set forth in SEQ ID NO: 591;
91) three heavy chain CDRs (HCDR1, HCDR2, HCDR3) comprised in the heavy chain variable region as set forth in SEQ ID NO: 592 and three light chain CDRs (LCDR1, LCDR2, LCDR3) comprised in the light chain variable region as set forth in SEQ ID NO: 593;
92) three heavy chain CDRs (HCDR1, HCDR2, HCDR3) comprised in the heavy chain variable region as set forth in SEQ ID NO: 592 and three light chain CDRs (LCDR1, LCDR2, LCDR3) comprised in the light chain variable region as set forth in SEQ ID NO: 594;
93) three heavy chain CDRs (HCDR1, HCDR2, HCDR3) comprised in the heavy chain variable region as set forth in SEQ ID NO: 595 and three light chain CDRs (LCDR1, LCDR2, LCDR3) comprised in the light chain variable region as set forth in SEQ ID NO: 596;
94) three heavy chain CDRs (HCDR1, HCDR2, HCDR3) comprised in the heavy chain variable region as set forth in SEQ ID NO: 595 and three light chain CDRs (LCDR1, LCDR2, LCDR3) comprised in the light chain variable region as set forth in SEQ ID NO: 594;
95) three heavy chain CDRs (HCDR1, HCDR2, HCDR3) comprised in the heavy chain variable region as set forth in SEQ ID NO: 597 and three light chain CDRs (LCDR1, LCDR2, LCDR3) comprised in the light chain variable region as set forth in SEQ ID NO: 598;
96) three heavy chain CDRs (HCDR1, HCDR2, HCDR3) comprised in the heavy chain variable region as set forth in SEQ ID NO: 597 and three light chain CDRs (LCDR1, LCDR2, LCDR3) comprised in the light chain variable region as set forth in SEQ ID NO: 594;
97) three heavy chain CDRs (HCDR1, HCDR2, HCDR3) comprised in the heavy chain variable region as set forth in SEQ ID NO: 599 and three light chain CDRs (LCDR1, LCDR2, LCDR3) comprised in the light chain variable region as set forth in SEQ ID NO: 600;
98) three heavy chain CDRs (HCDR1, HCDR2, HCDR3) comprised in the heavy chain variable region as set forth in SEQ ID NO: 601 and three light chain CDRs (LCDR1, LCDR2, LCDR3) comprised in the light chain variable region as set forth in SEQ ID NO: 602;
99) three heavy chain CDRs (HCDR1, HCDR2, HCDR3) comprised in the heavy chain variable region as set forth in SEQ ID NO: 603 and three light chain CDRs (LCDR1, LCDR2, LCDR3) comprised in the light chain variable region as set forth in SEQ ID NO: 604;
100) three heavy chain CDRs (HCDR1, HCDR2, HCDR3) comprised in the heavy chain variable region as set forth in SEQ ID NO: 605 and three light chain CDRs (LCDR1, LCDR2, LCDR3) comprised in the light chain variable region as set forth in SEQ ID NO: 606;
101) three heavy chain CDRs (HCDR1, HCDR2, HCDR3) comprised in the heavy chain variable region as set forth in SEQ ID NO: 607 and three light chain CDRs (LCDR1, LCDR2, LCDR3) comprised in the light chain variable region as set forth in SEQ ID NO: 608;
102) three heavy chain CDRs (HCDR1, HCDR2, HCDR3) comprised in the heavy chain variable region as set forth in SEQ ID NO: 609 and three light chain CDRs (LCDR1, LCDR2, LCDR3) comprised in the light chain variable region as set forth in SEQ ID NO: 610;
103) three heavy chain CDRs (HCDR1, HCDR2, HCDR3) comprised in the heavy chain variable region as set forth in SEQ ID NO: 611 and three light chain CDRs (LCDR1, LCDR2, LCDR3) comprised in the light chain variable region as set forth in SEQ ID NO: 612;
104) three heavy chain CDRs (HCDR1, HCDR2, HCDR3) comprised in the heavy chain variable region as set forth in SEQ ID NO: 613 and three light chain CDRs (LCDR1, LCDR2, LCDR3) comprised in the light chain variable region as set forth in SEQ ID NO: 614;
105) three heavy chain CDRs (HCDR1, HCDR2, HCDR3) comprised in the heavy chain variable region as set forth in SEQ ID NO: 615 and three light chain CDRs (LCDR1, LCDR2, LCDR3) comprised in the light chain variable region as set forth in SEQ ID NO: 616;
106) three heavy chain CDRs (HCDR1, HCDR2, HCDR3) comprised in the heavy chain variable region as set forth in SEQ ID NO: 617 and three light chain CDRs (LCDR1, LCDR2, LCDR3) comprised in the light chain variable region as set forth in SEQ ID NO: 618;
107) three heavy chain CDRs (HCDR1, HCDR2, HCDR3) comprised in the heavy chain variable region as set forth in SEQ ID NO: 619 and three light chain CDRs (LCDR1, LCDR2, LCDR3) comprised in the light chain variable region as set forth in SEQ ID NO: 620;
108) three heavy chain CDRs (HCDR1, HCDR2, HCDR3) comprised in the heavy chain variable region as set forth in SEQ ID NO: 621 and three light chain CDRs (LCDR1, LCDR2, LCDR3) comprised in the light chain variable region as set forth in SEQ ID NO: 622;
109) three heavy chain CDRs (HCDR1, HCDR2, HCDR3) comprised in the heavy chain variable region as set forth in SEQ ID NO: 623 and three light chain CDRs (LCDR1, LCDR2, LCDR3) comprised in the light chain variable region as set forth in SEQ ID NO: 624;
110) three heavy chain CDRs (HCDR1, HCDR2, HCDR3) comprised in the heavy chain variable region as set forth in SEQ ID NO: 625 and three light chain CDRs (LCDR1, LCDR2, LCDR3) comprised in the light chain variable region as set forth in SEQ ID NO: 626;
111) three heavy chain CDRs (HCDR1, HCDR2, HCDR3) comprised in the heavy chain variable region as set forth in SEQ ID NO: 627 and three light chain CDRs (LCDR1, LCDR2, LCDR3) comprised in the light chain variable region as set forth in SEQ ID NO: 628;
112) three heavy chain CDRs (HCDR1, HCDR2, HCDR3) comprised in the heavy chain variable region as set forth in SEQ ID NO: 629 and three light chain CDRs (LCDR1, LCDR2, LCDR3) comprised in the light chain variable region as set forth in SEQ ID NO: 630;
113) three heavy chain CDRs (HCDR1, HCDR2, HCDR3) comprised in the heavy chain variable region as set forth in SEQ ID NO: 631 and three light chain CDRs (LCDR1, LCDR2, LCDR3) comprised in the light chain variable region as set forth in SEQ ID NO: 632;
114) three heavy chain CDRs (HCDR1, HCDR2, HCDR3) comprised in the heavy chain variable region as set forth in SEQ ID NO: 617 and three light chain CDRs (LCDR1, LCDR2, LCDR3) comprised in the light chain variable region as set forth in SEQ ID NO: 633;
115) three heavy chain CDRs (HCDR1, HCDR2, HCDR3) comprised in the heavy chain variable region as set forth in SEQ ID NO: 634 and three light chain CDRs (LCDR1, LCDR2, LCDR3) comprised in the light chain variable region as set forth in SEQ ID NO: 635;
116) three heavy chain CDRs (HCDR1, HCDR2, HCDR3) comprised in the heavy chain variable region as set forth in SEQ ID NO: 636 and three light chain CDRs (LCDR1, LCDR2, LCDR3) comprised in the light chain variable region as set forth in SEQ ID NO: 637;
117) three heavy chain CDRs (HCDR1, HCDR2, HCDR3) comprised in the heavy chain variable region as set forth in SEQ ID NO: 638 and three light chain CDRs (LCDR1, LCDR2, LCDR3) comprised in the light chain variable region as set forth in SEQ ID NO: 639;
118) three heavy chain CDRs (HCDR1, HCDR2, HCDR3) comprised in the heavy chain variable region as set forth in SEQ ID NO: 640 and three light chain CDRs (LCDR1, LCDR2, LCDR3) comprised in the light chain variable region as set forth in SEQ ID NO: 641;
119) three heavy chain CDRs (HCDR1, HCDR2, HCDR3) comprised in the heavy chain variable region as set forth in SEQ ID NO: 642 and three light chain CDRs (LCDR1, LCDR2, LCDR3) comprised in the light chain variable region as set forth in SEQ ID NO: 643;
120) three heavy chain CDRs (HCDR1, HCDR2, HCDR3) comprised in the heavy chain variable region as set forth in SEQ ID NO: 644 and three light chain CDRs (LCDR1, LCDR2, LCDR3) comprised in the light chain variable region as set forth in SEQ ID NO: 645;
121) three heavy chain CDRs (HCDR1, HCDR2, HCDR3) comprised in the heavy chain variable region as set forth in SEQ ID NO: 646 and three light chain CDRs (LCDR1, LCDR2, LCDR3) comprised in the light chain variable region as set forth in SEQ ID NO: 647;
122) three heavy chain CDRs (HCDR1, HCDR2, HCDR3) comprised in the heavy chain variable region as set forth in SEQ ID NO: 648 and three light chain CDRs (LCDR1, LCDR2, LCDR3) comprised in the light chain variable region as set forth in SEQ ID NO: 649;
123) three heavy chain CDRs (HCDR1, HCDR2, HCDR3) comprised in the heavy chain variable region as set forth in SEQ ID NO: 650 and three light chain CDRs (LCDR1, LCDR2, LCDR3) comprised in the light chain variable region as set forth in SEQ ID NO: 651;
124) three heavy chain CDRs (HCDR1, HCDR2, HCDR3) comprised in the heavy chain variable region as set forth in SEQ ID NO: 652 and three light chain CDRs (LCDR1, LCDR2, LCDR3) comprised in the light chain variable region as set forth in SEQ ID NO: 653;
125) three heavy chain CDRs (HCDR1, HCDR2, HCDR3) comprised in the heavy chain variable region as set forth in SEQ ID NO: 654 and three light chain CDRs (LCDR1, LCDR2, LCDR3) comprised in the light chain variable region as set forth in SEQ ID NO: 655;
126) 3 heavy chain CDRs (HCDR1, HCDR2, HCDR3) included in the heavy chain variable region shown in SEQ ID NO: 656 and three light chain CDRs (LCDR1, LCDR2, LCDR3) included in the light chain variable region shown in SEQ ID NO: 653.

In one embodiment, the invention provides an anti-CD112R antibody that specifically bind to CD112R and an antigen-binding fragment thereof, comprising:
1) HCDR1, HCDR2, HCDR3 comprising the sequence as set forth in SEQ ID NOs: 1, 64 and 162, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NOs: 239, 307 and 357, or a sequence containing an amino acid substitution (e.g., conservative substitution), deletion or insertion of one or more and no more than 3 amino acids relative to the sequence;
2) HCDR1, HCDR2, HCDR3 comprising the sequence as set forth in SEQ ID NOs: 2, 65 and 162, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NOs: 239, 308, and 358, or a sequence containing an amino acid substitution (e.g., conservative substitution), deletion or insertion of one or more and no more than 3 amino acids relative to the sequence;
3) HCDR1, HCDR2, HCDR3 comprising the sequence as set forth in SEQ ID NO: 3, 66, 163, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 240, 309, 359, or a sequence containing an amino acid substitution (e.g., conservative substitution), deletion or insertion of one or more and no more than 3 amino acids relative to the sequence;
4) HCDR1, HCDR2, HCDR3 comprising the sequence as set forth in SEQ ID NO: 4, 67, 164, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 310, 360, or a sequence containing an amino acid substitution (e.g., conservative substitution), deletion or insertion of one or more and no more than 3 amino acids relative to the sequence;
5) HCDR1, HCDR2, HCDR3 comprising the sequence as set forth in SEQ ID NO: 5, 68, 165, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 361, or a sequence containing an amino acid substitution (e.g., conservative substitution), deletion or insertion of one or more and no more than 3 amino acids relative to the sequence;
6) HCDR1, HCDR2, HCDR3 comprising the sequence as set forth in SEQ ID NO: 5, 68, 165, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 242, 312, 362, or a sequence containing an amino acid substitution (e.g., conservative substitution), deletion or insertion of one or more and no more than 3 amino acids relative to the sequence;
7) HCDR1, HCDR2, HCDR3 comprising the sequence as set forth in SEQ ID NO: 6, 69, 166, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 243, 313, 363, or a sequence containing an amino acid substitution (e.g., conservative substitution), deletion or insertion of one or more and no more than 3 amino acids relative to the sequence;
8) HCDR1, HCDR2, HCDR3 comprising the sequence as set forth in SEQ ID NO: 7, 70, 167, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 244, 314, 364, or a sequence containing an amino acid substitution (e.g., conservative substitution), deletion or insertion of one or more and no more than 3 amino acids relative to the sequence;
9) HCDR1, HCDR2, HCDR3 comprising the sequence as set forth in SEQ ID NO: 8, 71, 168, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 245, 313, 365, or a sequence containing an amino acid substitution (e.g., conservative substitution), deletion or insertion of one or more and no more than 3 amino acids relative to the sequence;
10) HCDR1, HCDR2, HCDR3 comprising the sequence as set forth in SEQ ID NO: 3, 72, 163, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 359, or a sequence containing an amino acid substitution (e.g., conservative substitution), deletion or insertion of one or more and no more than 3 amino acids relative to the sequence;
11) HCDR1, HCDR2, HCDR3 comprising the sequence as set forth in SEQ ID NO: 9, 73, 168, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 245, 313, 363, or a sequence containing an amino acid substitution (e.g., conservative substitution), deletion or insertion of one or more and no more than 3 amino acids relative to the sequence;
12) HCDR1, HCDR2, HCDR3 comprising the sequence as set forth in SEQ ID NO: 3, 74, 163, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 246, 311, 359, or a sequence containing an amino acid substitution (e.g., conservative substitution), deletion or insertion of one or more and no more than 3 amino acids relative to the sequence;
13) HCDR1, HCDR2, HCDR3 comprising the sequence as set forth in SEQ ID NO: 10, 75, 169, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 315, 366, or a sequence containing an amino acid substitution (e.g., conservative substitution), deletion or insertion of one or more and no more than 3 amino acids relative to the sequence;
14) HCDR1, HCDR2, HCDR3 comprising the sequence as set forth in SEQ ID NO: 10, 75, 170, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 315, 366, or a sequence containing an amino acid substitution (e.g., conservative substitution), deletion or insertion of one or more and no more than 3 amino acids relative to the sequence;
15) HCDR1, HCDR2, HCDR3 comprising the sequence as set forth in SEQ ID NO: 9, 76, 168, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 243, 313, 363, or a sequence containing an amino acid substitution (e.g., conservative substitution), deletion or insertion of one or more and no more than 3 amino acids relative to the sequence;
16) HCDR1, HCDR2, HCDR3 comprising the sequence as set forth in SEQ ID NO: 9, 77, 168, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 248, 313, 363, or a sequence containing an amino acid substitution (e.g., conservative substitution), deletion or insertion of one or more and no more than 3 amino acids relative to the sequence;
17) HCDR1, HCDR2, HCDR3 comprising the sequence as set forth in SEQ ID NO: 8, 77, 168, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 243, 313, 365, or a sequence containing an amino acid substitution (e.g., conservative substitution), deletion or insertion of one or more and no more than 3 amino acids relative to the sequence;
18) HCDR1, HCDR2, HCDR3 comprising the sequence as set forth in SEQ ID NO: 7, 78, 167, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 244, 314, 364, or a sequence containing an amino acid substitution (e.g., conservative substitution), deletion or insertion of one or more and no more than 3 amino acids relative to the sequence;
19) HCDR1, HCDR2, HCDR3 comprising the sequence as set forth in SEQ ID NO: 11, 79, 171, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 249, 316, 367, or a sequence containing an amino acid substitution (e.g., conservative substitution), deletion or insertion of one or more and no more than 3 amino acids relative to the sequence;
20) HCDR1, HCDR2, HCDR3 comprising the sequence as set forth in SEQ ID NO: 2, 80, 162, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 317, 368, or a sequence containing an amino acid substitution (e.g., conservative substitution), deletion or insertion of one or more and no more than 3 amino acids relative to the sequence;
21) HCDR1, HCDR2, HCDR3 comprising the sequence as set forth in SEQ ID NO: 12, 81, 172, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 250, 315, 369, or a sequence containing an amino acid substitution (e.g., conservative substitution), deletion or insertion of one or more and no more than 3 amino acids relative to the sequence;
22) HCDR1, HCDR2, HCDR3 comprising the sequence as set forth in SEQ ID NO: 10, 82, 169, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 315, 370, or a sequence containing an amino acid substitution (e.g., conservative substitution), deletion or insertion of one or more and no more than 3 amino acids relative to the sequence;
23) HCDR1, HCDR2, HCDR3 comprising the sequence as set forth in SEQ ID NO: 2, 83, 173, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 242, 312, 371, or a sequence containing an amino acid substitution (e.g., conservative substitution), deletion or insertion of one or more and no more than 3 amino acids relative to the sequence;
24) HCDR1, HCDR2, HCDR3 comprising the sequence as set forth in SEQ ID NO: 13, 84, 173, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 242, 312, 372, or a sequence containing an amino acid substitution (e.g., conservative substitution), deletion or insertion of one or more and no more than 3 amino acids relative to the sequence;
25) HCDR1, HCDR2, HCDR3 comprising the sequence as set forth in SEQ ID NO: 2, 85, 174, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 251, 318, 373, or a sequence containing an amino acid substitution (e.g., conservative substitution), deletion or insertion of one or more and no more than 3 amino acids relative to the sequence;
26) HCDR1, HCDR2, HCDR3 comprising the sequence as set forth in SEQ ID NO: 14, 86, 175, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 252, 319, 374, or a sequence containing an amino acid substitution (e.g., conservative substitution), deletion or insertion of one or more and no more than 3 amino acids relative to the sequence;
27) HCDR1, HCDR2, HCDR3 comprising the sequence as set forth in SEQ ID NO: 15, 87, 176, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 253, 320, 375, or a sequence containing an amino acid substitution (e.g., conservative substitution), deletion or insertion of one or more and no more than 3 amino acids relative to the sequence;
28) HCDR1, HCDR2, HCDR3 comprising the sequence as set forth in SEQ ID NO: 16, 88, 177, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 254, 321, 376, or a sequence containing an amino acid substitution (e.g., conservative substitution), deletion or insertion of one or more and no more than 3 amino acids relative to the sequence;
29) HCDR1, HCDR2, HCDR3 comprising the sequence as set forth in SEQ ID NO: 2, 89, 178, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 377, or a sequence containing an amino acid substitution (e.g., conservative substitution), deletion or insertion of one or more and no more than 3 amino acids relative to the sequence;
30) HCDR1, HCDR2, HCDR3 comprising the sequence as set forth in SEQ ID NO: 17, 75, 169, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 315, 366, or a sequence containing an amino acid substitution (e.g., conservative substitution), deletion or insertion of one or more and no more than 3 amino acids relative to the sequence;
31) HCDR1, HCDR2, HCDR3 comprising the sequence as set forth in SEQ ID NO: 18, 85, 162, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 322, 378, or a sequence containing an amino acid substitution (e.g., conservative substitution), deletion or insertion of one or more and no more than 3 amino acids relative to the sequence;
32) HCDR1, HCDR2, HCDR3 comprising the sequence as set forth in SEQ ID NO: 2, 90, 162, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 255, 308, 368, or a sequence containing an amino acid substitution (e.g., conservative substitution), deletion or insertion of one or more and no more than 3 amino acids relative to the sequence;
33) HCDR1, HCDR2, HCDR3 comprising the sequence as set forth in SEQ ID NO: 19, 91, 179, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 322, 378, or a sequence containing an amino acid substitution (e.g., conservative substitution), deletion or insertion of one or more and no more than 3 amino acids relative to the sequence;
34) HCDR1, HCDR2, HCDR3 comprising the sequence as set forth in SEQ ID NO: 2, 85, 180, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 256, 323, 379, or a sequence containing an amino acid substitution (e.g., conservative substitution), deletion or insertion of one or more and no more than 3 amino acids relative to the sequence;
35) HCDR1, HCDR2, HCDR3 comprising the sequence as set forth in SEQ ID NO: 2, 85, 180, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 308, 380, or a sequence containing an amino acid substitution (e.g., conservative substitution), deletion or insertion of one or more and no more than 3 amino acids relative to the sequence;
36) HCDR1, HCDR2, HCDR3 comprising the sequence as set forth in SEQ ID NO: 20, 92, 181, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 257, 324, 381, or a sequence containing an amino acid substitution (e.g., conservative substitution), deletion or insertion of one or more and no more than 3 amino acids relative to the sequence;
37) HCDR1, HCDR2, HCDR3 comprising the sequence as set forth in SEQ ID NO: 21, 93, 182, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 258, 325, 382, or a sequence containing an amino acid substitution (e.g., conservative substitution), deletion or insertion of one or more and no more than 3 amino acids relative to the sequence;
38) HCDR1, HCDR2, HCDR3 comprising the sequence as set forth in SEQ ID NO: 22, 94, 183, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 259, 323, 383, or a sequence containing an amino acid substitution (e.g., conservative substitution), deletion or insertion of one or more and no more than 3 amino acids relative to the sequence;
39) HCDR1, HCDR2, HCDR3 comprising the sequence as set forth in SEQ ID NO: 23, 94, 183, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 259, 323, 383, or a sequence containing an amino acid substitution (e.g., conservative substitution), deletion or insertion of one or more and no more than 3 amino acids relative to the sequence;
40) HCDR1, HCDR2, HCDR3 comprising the sequence as set forth in SEQ ID NO: 24, 95, 184, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 326, 384, or a sequence containing an amino acid substitution (e.g., conservative substitution), deletion or insertion of one or more and no more than 3 amino acids relative to the sequence;
41) HCDR1, HCDR2, HCDR3 comprising the sequence as set forth in SEQ ID NO: 21, 93, 182, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 326, 385, or a sequence containing an amino acid substitution (e.g., conservative substitution), deletion or insertion of one or more and no more than 3 amino acids relative to the sequence;
42) HCDR1, HCDR2, HCDR3 comprising the sequence as set forth in SEQ ID NO: 22, 94, 183, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 259, 323, 383, or a sequence containing an amino acid substitution (e.g., conservative substitution), deletion or insertion of one or more and no more than 3 amino acids relative to the sequence;
43) HCDR1, HCDR2, HCDR3 comprising the sequence as set forth in SEQ ID NO: 25, 96, 185, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 260, 327, 386, or a sequence containing an amino acid substitution (e.g., conservative substitution), deletion or insertion of one or more and no more than 3 amino acids relative to the sequence;
44) HCDR1, HCDR2, HCDR3 comprising the sequence as set forth in SEQ ID NO: 26, 97, 186, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 261, 328, 387, or a sequence containing an amino acid substitution (e.g., conservative substitution), deletion or insertion of one or more and no more than 3 amino acids relative to the sequence;
45) HCDR1, HCDR2, HCDR3 comprising the sequence as set forth in SEQ ID NO: 27, 98, 187, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 262, 328, 388, or a sequence containing an amino acid substitution (e.g., conservative substitution), deletion or insertion of one or more and no more than 3 amino acids relative to the sequence;
46) HCDR1, HCDR2, HCDR3 comprising the sequence as set forth in SEQ ID NO: 28, 99, 188, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 263, 328, 388, or a sequence containing an amino acid substitution (e.g., conservative substitution), deletion or insertion of one or more and no more than 3 amino acids relative to the sequence;
47) HCDR1, HCDR2, HCDR3 comprising the sequence as set forth in SEQ ID NO: 22, 94, 183, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 308, 389, or a sequence containing an amino acid substitution (e.g., conservative substitution), deletion or insertion of one or more and no more than 3 amino acids relative to the sequence;
48) HCDR1, HCDR2, HCDR3 comprising the sequence as set forth in SEQ ID NO: 5, 100, 189, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 264, 329, 390, or a sequence containing an amino acid substitution (e.g., conservative substitution), deletion or insertion of one or more and no more than 3 amino acids relative to the sequence;
49) HCDR1, HCDR2, HCDR3 comprising the sequence as set forth in SEQ ID NO: 24, 101, 190, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 326, 385, or a sequence containing an amino acid substitution (e.g., conservative substitution), deletion or insertion of one or more and no more than 3 amino acids relative to the sequence;
50) HCDR1, HCDR2, HCDR3 comprising the sequence as set forth in SEQ ID NO: 28, 102, 191, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 265, 328, 391, or a sequence containing an amino acid substitution (e.g., conservative substitution), deletion or insertion of one or more and no more than 3 amino acids relative to the sequence;
51) HCDR1, HCDR2, HCDR3 comprising the sequence as set forth in SEQ ID NO: 28, 103, 192, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 266, 328, 388, or a sequence containing an amino acid substitution (e.g., conservative substitution), deletion or insertion of one or more and no more than 3 amino acids relative to the sequence;
52) HCDR1, HCDR2, HCDR3 comprising the sequence as set forth in SEQ ID NO: 29, 104, 163, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 377, or a sequence containing an amino acid substitution (e.g., conservative substitution), deletion or insertion of one or more and no more than 3 amino acids relative to the sequence;
53) HCDR1, HCDR2, HCDR3 comprising the sequence as set forth in SEQ ID NO: 30, 105, 193, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 267, 330, 392, or a sequence containing an amino acid substitution (e.g., conservative substitution), deletion or insertion of one or more and no more than 3 amino acids relative to the sequence;
54) HCDR1, HCDR2, HCDR3 comprising the sequence as set forth in SEQ ID NO: 5, 106, 194, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 268, 331, 393, or a sequence containing an amino acid substitution (e.g., conservative substitution), deletion or insertion of one or more and no more than 3 amino acids relative to the sequence;
55) HCDR1, HCDR2, HCDR3 comprising the sequence as set forth in SEQ ID NO: 31, 107, 195, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 256, 318, 392, or a sequence containing an amino acid substitution (e.g., conservative substitution), deletion or insertion of one or more and no more than 3 amino acids relative to the sequence;
56) HCDR1, HCDR2, HCDR3 comprising the sequence as set forth in SEQ ID NO: 31, 107, 195, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 269, 332, 394, or a sequence containing an amino acid substitution (e.g., conservative substitution), deletion or insertion of one or more and no more than 3 amino acids relative to the sequence;
57) HCDR1, HCDR2, HCDR3 comprising the sequence as set forth in SEQ ID NO: 32, 108, 196, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 256, 318, 392, or a sequence containing an amino acid substitution (e.g., conservative substitution), deletion or insertion of one or more and no more than 3 amino acids relative to the sequence;
58) HCDR1, HCDR2, HCDR3 comprising the sequence as set forth in SEQ ID NO: 32, 108, 196, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 269, 332, 394, or a sequence containing an amino acid substitution (e.g., conservative substitution), deletion or insertion of one or more and no more than 3 amino acids relative to the sequence;
59) HCDR1, HCDR2, HCDR3 comprising the sequence as set forth in SEQ ID NO: 29, 109, 197, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 395, or a sequence containing an amino acid substitution (e.g., conservative substitution), deletion or insertion of one or more and no more than 3 amino acids relative to the sequence;
60) HCDR1, HCDR2, HCDR3 comprising the sequence as set forth in SEQ ID NO: 33, 110, 198, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 270, 333, 396, or a sequence containing an amino acid substitution (e.g., conservative substitution), deletion or insertion of one or more and no more than 3 amino acids relative to the sequence;
61) HCDR1, HCDR2, HCDR3 comprising the sequence as set forth in SEQ ID NO: 5, 111, 199, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 256, 318, 397, or a sequence containing an amino acid substitution (e.g., conservative substitution), deletion or insertion of one or more and no more than 3 amino acids relative to the sequence;
62) HCDR1, HCDR2, HCDR3 comprising the sequence as set forth in SEQ ID NO: 34, 112, 200, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 269, 334, 398, or a sequence containing an amino acid substitution (e.g., conservative substitution), deletion or insertion of one or more and no more than 3 amino acids relative to the sequence;
63) HCDR1, HCDR2, HCDR3 comprising the sequence as set forth in SEQ ID NO: 34, 112, 200, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 271, 335, 399, or a sequence containing an amino acid substitution (e.g., conservative substitution), deletion or insertion of one or more and no more than 3 amino acids relative to the sequence;
64) HCDR1, HCDR2, HCDR3 comprising the sequence as set forth in SEQ ID NO: 35, 113, 201, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 400, or a sequence containing an amino acid substitution (e.g., conservative substitution), deletion or insertion of one or more and no more than 3 amino acids relative to the sequence;
65) HCDR1, HCDR2, HCDR3 comprising the sequence as set forth in SEQ ID NO: 36, 114, 202, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 272, 336, 401, or a sequence containing an amino acid substitution (e.g., conservative substitution), deletion or insertion of one or more and no more than 3 amino acids relative to the sequence;
66) HCDR1, HCDR2, HCDR3 comprising the sequence as set forth in SEQ ID NO: 29, 115, 203, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 402, or a sequence containing an amino acid substitution (e.g., conservative substitution), deletion or insertion of one or more and no more than 3 amino acids relative to the sequence;
67) HCDR1, HCDR2, HCDR3 comprising the sequence as set forth in SEQ ID NO: 37, 116, 204, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 273, 337, 403, or a sequence containing an amino acid substitution (e.g., conservative substitution), deletion or insertion of one or more and no more than 3 amino acids relative to the sequence;
68) HCDR1, HCDR2, HCDR3 comprising the sequence as set forth in SEQ ID NO: 5, 117, 205, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 274, 338, 404, or a sequence containing an amino acid substitution (e.g., conservative substitution), deletion or insertion of one or more and no more than 3 amino acids relative to the sequence;
69) HCDR1, HCDR2, HCDR3 comprising the sequence as set forth in SEQ ID NO: 38, 110, 198, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 270, 333, 405, or a sequence containing an amino acid substitution (e.g., conservative substitution), deletion or insertion of one or more and no more than 3 amino acids relative to the sequence;
70) HCDR1, HCDR2, HCDR3 comprising the sequence as set forth in SEQ ID NO: 39, 118, 204, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 275, 337, 406, or a sequence containing an amino acid substitution (e.g., conservative substitution), deletion or insertion of one or more and no more than 3 amino acids relative to the sequence;
71) HCDR1, HCDR2, HCDR3 comprising the sequence as set forth in SEQ ID NO: 5, 119, 206, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 276, 338, 407, or a sequence containing an amino acid substitution (e.g., conservative substitution), deletion or insertion of one or more and no more than 3 amino acids relative to the sequence;
72) HCDR1, HCDR2, HCDR3 comprising the sequence as set forth in SEQ ID NO: 29, 109, 197, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 395, or a sequence containing an amino acid substitution (e.g., conservative substitution), deletion or insertion of one or more and no more than 3 amino acids relative to the sequence;
73) HCDR1, HCDR2, HCDR3 comprising the sequence as set forth in SEQ ID NO: 38, 110, 198, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 270, 333, 405, or a sequence containing an amino acid substitution (e.g., conservative substitution), deletion or insertion of one or more and no more than 3 amino acids relative to the sequence;
74) HCDR1, HCDR2, HCDR3 comprising the sequence as set forth in SEQ ID NO: 5, 120, 205, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 277, 338, 407, or a sequence containing an amino acid substitution (e.g., conservative substitution), deletion or insertion of one or more and no more than 3 amino acids relative to the sequence;
75) HCDR1, HCDR2, HCDR3 comprising the sequence as set forth in SEQ ID NO: 5, 121, 207, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 278, 338, 404, or a sequence containing an amino acid substitution (e.g., conservative substitution), deletion or insertion of one or more and no more than 3 amino acids relative to the sequence;
76) HCDR1, HCDR2, HCDR3 comprising the sequence as set forth in SEQ ID NO: 35, 122, 201, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 279, 311, 408, or a sequence containing an amino acid substitution (e.g., conservative substitution), deletion or insertion of one or more and no more than 3 amino acids relative to the sequence;
77) HCDR1, HCDR2, HCDR3 comprising the sequence as set forth in SEQ ID NO: 35, 123, 208, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 280, 311, 400, or a sequence containing an amino acid substitution (e.g., conservative substitution), deletion or insertion of one or more and no more than 3 amino acids relative to the sequence;
78) HCDR1, HCDR2, HCDR3 comprising the sequence as set forth in SEQ ID NO: 5, 124, 209, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 281, 338, 407, or a sequence containing an amino acid substitution (e.g., conservative substitution), deletion or insertion of one or more and no more than 3 amino acids relative to the sequence;
79) HCDR1, HCDR2, HCDR3 comprising the sequence as set forth in SEQ ID NO: 40, 125, 210, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 282, 339, 409, or a sequence containing an amino acid substitution (e.g., conservative substitution), deletion or insertion of one or more and no more than 3 amino acids relative to the sequence;
80) HCDR1, HCDR2, HCDR3 comprising the sequence as set forth in SEQ ID NO: 41, 126, 211, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 245, 333, 410, or a sequence containing an amino acid substitution (e.g., conservative substitution), deletion or insertion of one or more and no more than 3 amino acids relative to the sequence;
81) HCDR1, HCDR2, HCDR3 comprising the sequence as set forth in SEQ ID NO: 10, 127, 212, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 308, 411, or a sequence containing an amino acid substitution (e.g., conservative substitution), deletion or insertion of one or more and no more than 3 amino acids relative to the sequence;
82) HCDR1, HCDR2, HCDR3 comprising the sequence as set forth in SEQ ID NO: 40, 125, 213, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 308, 411, or a sequence containing an amino acid substitution (e.g., conservative substitution), deletion or insertion of one or more and no more than 3 amino acids relative to the sequence;
83) HCDR1, HCDR2, HCDR3 comprising the sequence as set forth in SEQ ID NO: 42, 128, 214, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 283, 324, 412, or a sequence containing an amino acid substitution (e.g., conservative substitution), deletion or insertion of one or more and no more than 3 amino acids relative to the sequence;
84) HCDR1, HCDR2, HCDR3 comprising the sequence as set forth in SEQ ID NO: 43, 129, 215, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 284, 339, 409, or a sequence containing an amino acid substitution (e.g., conservative substitution), deletion or insertion of one or more and no more than 3 amino acids relative to the sequence;
85) HCDR1, HCDR2, HCDR3 comprising the sequence as set forth in SEQ ID NO: 42, 130, 216, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 285, 340, 409, or a sequence containing an amino acid substitution (e.g., conservative substitution), deletion or insertion of one or more and no more than 3 amino acids relative to the sequence;
86) HCDR1, HCDR2, HCDR3 comprising the sequence as set forth in SEQ ID NO: 44, 131, 217, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 286, 341, 413, or a sequence containing an amino acid substitution (e.g., conservative substitution), deletion or insertion of one or more and no more than 3 amino acids relative to the sequence;
87) HCDR1, HCDR2, HCDR3 comprising the sequence as set forth in SEQ ID NO: 44, 132, 218, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 282, 341, 414, or a sequence containing an amino acid substitution (e.g., conservative substitution), deletion or insertion of one or more and no more than 3 amino acids relative to the sequence;
88) HCDR1, HCDR2, HCDR3 comprising the sequence as set forth in SEQ ID NO: 44, 132, 218, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 287, 342, 415, or a sequence containing an amino acid substitution (e.g., conservative substitution), deletion or insertion of one or more and no more than 3 amino acids relative to the sequence;
89) HCDR1, HCDR2, HCDR3 comprising the sequence as set forth in SEQ ID NO: 45, 133, 219, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 288, 343, 416, or a sequence containing an amino acid substitution (e.g., conservative substitution), deletion or insertion of one or more and no more than 3 amino acids relative to the sequence;
90) HCDR1, HCDR2, HCDR3 comprising the sequence as set forth in SEQ ID NO: 46, 134, 220, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 289, 324, 417, or a sequence containing an amino acid substitution (e.g., conservative substitution), deletion or insertion of one or more and no more than 3 amino acids relative to the sequence;
91) HCDR1, HCDR2, HCDR3 comprising the sequence as set forth in SEQ ID NO: 46, 134, 220, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 290, 344, 418, or a sequence containing an amino acid substitution (e.g., conservative substitution), deletion or insertion of one or more and no more than 3 amino acids relative to the sequence;
92) HCDR1, HCDR2, HCDR3 comprising the sequence as set forth in SEQ ID NO: 47, 135, 221, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 291, 345, 407, or a sequence containing an amino acid substitution (e.g., conservative substitution), deletion or insertion of one or more and no more than 3 amino acids relative to the sequence;
93) HCDR1, HCDR2, HCDR3 comprising the sequence as set forth in SEQ ID NO: 47, 135, 221, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 290, 344, 418, or a sequence containing an amino acid substitution (e.g., conservative substitution), deletion or insertion of one or more and no more than 3 amino acids relative to the sequence;
94) HCDR1, HCDR2, HCDR3 comprising the sequence as set forth in SEQ ID NO: 48, 136, 222, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 292, 346, 409, or a sequence containing an amino acid substitution (e.g., conservative substitution), deletion or insertion of one or more and no more than 3 amino acids relative to the sequence;
95) HCDR1, HCDR2, HCDR3 comprising the sequence as set forth in SEQ ID NO: 48, 136, 222, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 290, 344, 418, or a sequence containing an amino acid substitution (e.g., conservative substitution), deletion or insertion of one or more and no more than 3 amino acids relative to the sequence;
96) HCDR1, HCDR2, HCDR3 comprising the sequence as set forth in SEQ ID NO: 49, 137, 163, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 240, 347, 419, or a sequence containing an amino acid substitution (e.g., conservative substitution), deletion or insertion of one or more and no more than 3 amino acids relative to the sequence;
97) HCDR1, HCDR2, HCDR3 comprising the sequence as set forth in SEQ ID NO: 45, 138, 223, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 288, 343, 416, or a sequence containing an amino acid substitution (e.g., conservative substitution), deletion or insertion of one or more and no more than 3 amino acids relative to the sequence;
98) HCDR1, HCDR2, HCDR3 comprising the sequence as set forth in SEQ ID NO: 50, 139, 224, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 249, 348, 420, or a sequence containing an amino acid substitution (e.g., conservative substitution), deletion or insertion of one or more and no more than 3 amino acids relative to the sequence;
99) HCDR1, HCDR2, HCDR3 comprising the sequence as set forth in SEQ ID NO: 51, 140, 225, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 256, 349, 421, or a sequence containing an amino acid substitution (e.g., conservative substitution), deletion or insertion of one or more and no more than 3 amino acids relative to the sequence;
100) HCDR1, HCDR2, HCDR3 comprising the sequence as set forth in SEQ ID NO: 47, 141, 221, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 293, 345, 407, or a sequence containing an amino acid substitution (e.g., conservative substitution), deletion or insertion of one or more and no more than 3 amino acids relative to the sequence;
101) HCDR1, HCDR2, HCDR3 comprising the sequence as set forth in SEQ ID NO: 52, 142, 226, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 294, 350, 422, or a sequence containing an amino acid substitution (e.g., conservative substitution), deletion or insertion of one or more and no more than 3 amino acids relative to the sequence;
102) HCDR1, HCDR2, HCDR3 comprising the sequence as set forth in SEQ ID NO: 53, 143, 178, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 295, 311, 423, or a sequence containing an amino acid substitution (e.g., conservative substitution), deletion or insertion of one or more and no more than 3 amino acids relative to the sequence;
103) HCDR1, HCDR2, HCDR3 comprising the sequence as set forth in SEQ ID NO: 53, 144, 178, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 361, or a sequence containing an amino acid substitution (e.g., conservative substitution), deletion or insertion of one or more and no more than 3 amino acids relative to the sequence;
104) HCDR1, HCDR2, HCDR3 comprising the sequence as set forth in SEQ ID NO: 53, 145, 227, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 361, or a sequence containing an amino acid substitution (e.g., conservative substitution), deletion or insertion of one or more and no more than 3 amino acids relative to the sequence;
105) HCDR1, HCDR2, HCDR3 comprising the sequence as set forth in SEQ ID NO: 54, 146, 228, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 296, 328, 391, or a sequence containing an amino acid substitution (e.g., conservative substitution), deletion or insertion of one or more and no more than 3 amino acids relative to the sequence;
106) HCDR1, HCDR2, HCDR3 comprising the sequence as set forth in SEQ ID NO: 55, 147, 228, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 266, 328, 391, or a sequence containing an amino acid substitution (e.g., conservative substitution), deletion or insertion of one or more and no more than 3 amino acids relative to the sequence;
107) HCDR1, HCDR2, HCDR3 comprising the sequence as set forth in SEQ ID NO: 50, 148, 229, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 297, 351, 424, or a sequence containing an amino acid substitution (e.g., conservative substitution), deletion or insertion of one or more and no more than 3 amino acids relative to the sequence;
108) HCDR1, HCDR2, HCDR3 comprising the sequence as set forth in SEQ ID NO: 56, 149, 230, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 298, 323, 425, or a sequence containing an amino acid substitution (e.g., conservative substitution), deletion or insertion of one or more and no more than 3 amino acids relative to the sequence;
109) HCDR1, HCDR2, HCDR3 comprising the sequence as set forth in SEQ ID NO: 57, 150, 231, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 299, 338, 407, or a sequence containing an amino acid substitution (e.g., conservative substitution), deletion or insertion of one or more and no more than 3 amino acids relative to the sequence;
110) HCDR1, HCDR2, HCDR3 comprising the sequence as set forth in SEQ ID NO: 58, 151, 232, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 300, 352, 426, or a sequence containing an amino acid substitution (e.g., conservative substitution), deletion or insertion of one or more and no more than 3 amino acids relative to the sequence;
111) HCDR1, HCDR2, HCDR3 comprising the sequence as set forth in SEQ ID NO: 59, 152, 233, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 301, 328, 388, or a sequence containing an amino acid substitution (e.g., conservative substitution), deletion or insertion of one or more and no more than 3 amino acids relative to the sequence;
112) HCDR1, HCDR2, HCDR3 comprising the sequence as set forth in SEQ ID NO: 60, 153, 234, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 239, 308, 360, or a sequence containing an amino acid substitution (e.g., conservative substitution), deletion or insertion of one or more and no more than 3 amino acids relative to the sequence;
113) HCDR1, HCDR2, HCDR3 comprising the sequence as set forth in SEQ ID NO: 54, 146, 228, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 302, 328, 391, or a sequence containing an amino acid substitution (e.g., conservative substitution), deletion or insertion of one or more and no more than 3 amino acids relative to the sequence;
114) HCDR1, HCDR2, HCDR3 comprising the sequence as set forth in SEQ ID NO: 61, 154, 235, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 241, 311, 361, or a sequence containing an amino acid substitution (e.g., conservative substitution), deletion or insertion of one or more and no more than 3 amino acids relative to the sequence;
115) HCDR1, HCDR2, HCDR3 comprising the sequence as set forth in SEQ ID NO: 40, 125, 210, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 282, 353, 409, or a sequence containing an amino acid substitution (e.g., conservative substitution), deletion or insertion of one or more and no more than 3 amino acids relative to the sequence;
116) HCDR1, HCDR2, HCDR3 comprising the sequence as set forth in SEQ ID NO: 44, 155, 236, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 303, 324, 409, or a sequence containing an amino acid substitution (e.g., conservative substitution), deletion or insertion of one or more and no more than 3 amino acids relative to the sequence;
117) HCDR1, HCDR2, HCDR3 comprising the sequence as set forth in SEQ ID NO: 44, 156, 217, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 282, 341, 414, or a sequence containing an amino acid substitution (e.g., conservative substitution), deletion or insertion of one or more and no more than 3 amino acids relative to the sequence;
118) HCDR1, HCDR2, HCDR3 comprising the sequence as set forth in SEQ ID NO: 62, 157, 237, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 304, 332, 427, or a sequence containing an amino acid substitution (e.g., conservative substitution), deletion or insertion of one or more and no more than 3 amino acids relative to the sequence;
119) HCDR1, HCDR2, HCDR3 comprising the sequence as set forth in SEQ ID NO: 24, 158, 184, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 247, 326, 428, or a sequence containing an amino acid substitution (e.g., conservative substitution), deletion or insertion of one or more and no more than 3 amino acids relative to the sequence;
120) HCDR1, HCDR2, HCDR3 comprising the sequence as set forth in SEQ ID NO: 3, 159, 189, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 249, 354, 382, or a sequence containing an amino acid substitution (e.g., conservative substitution), deletion or insertion of one or more and no more than 3 amino acids relative to the sequence;
121) HCDR1, HCDR2, HCDR3 comprising the sequence as set forth in SEQ ID NO: 63, 160, 238, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 305, 355, 429, or a sequence containing an amino acid substitution (e.g., conservative substitution), deletion or insertion of one or more and no more than 3 amino acids relative to the sequence; or
122) HCDR1, HCDR2, HCDR3 comprising the sequence as set forth in SEQ ID NO: 42, 161, 214, or a sequence containing an amino acid substitution (e.g., conservative substitution), deletion or insertion of one or more and no more than 3 amino acids relative to the sequence; and LCDR1, LCDR2, LCDR3 comprising the sequence as set forth in SEQ ID NO: 306, 356, 409, or a sequence containing an amino acid substitution (e.g., conservative substitution), deletion or insertion of one or more and no more than 3 amino acids relative to the sequence.

In one embodiment, the invention provides an anti-CD112R antibody that specifically binds CD112R and an antigen-binding fragment thereof, comprising a heavy chain variable region, wherein:
1) the heavy chain variable region comprises the amino acid sequence as set forth in SEQ ID NO: 430, or an amino acid sequence having at least 90%, 91%, 92%, 93%, 94%, 95%, 96%, 97%, 98% or 99% identity with the amino acid sequence of SEQ ID NO: 430, or consists of SEQ ID NO: 430;
2) the heavy chain variable region comprises the amino acid sequence as set forth in SEQ ID NO: 432, or an amino acid sequence having at least 90%, 91%, 92%, 93%, 94%, 95%, 96%, 97%, 98% or 99% identity with the amino acid sequence of SEQ ID NO: 432, or consists of SEQ ID NO: 432;
3) the heavy chain variable region comprises the amino acid sequence as set forth in SEQ ID NO: 434, or an amino acid sequence having at least 90%, 91%, 92%, 93%, 94%, 95%, 96%, 97%, 98% or 99% identity with the amino acid sequence of SEQ ID NO: 434, or consists of SEQ ID NO: 434;
4) the heavy chain variable region comprises the amino acid sequence as set forth in SEQ ID NO: 436, or an amino acid sequence having at least 90%, 91%, 92%, 93%, 94%, 95%, 9%%, 97%, 98% or 99% identity with the amino acid sequence of SEQ ID NO: 436, or consists of SEQ ID NO: 436;
5) the heavy chain variable region comprises the amino acid sequence as set forth in SEQ ID NO: 438, or an amino acid sequence having at least 90%, 91%, 92%, 93%, 94%, 95%, 96%, 97%, 98% or 99% identity with the amino acid sequence of SEQ ID NO: 438, or consists of SEQ ID NO: 438;
6) the heavy chain variable region comprises the amino acid sequence as set forth in SEQ ID NO: 441, or an amino acid sequence having at least 90%, 91%, 92%, 93%, 94%, 95%, 96%, 97%, 98% or 99% identity with the amino acid sequence of SEQ ID NO: 441, or consists of SEQ ID NO: 441;
7) the heavy chain variable region comprises the amino acid sequence as set forth in SEQ ID NO: 443, or an amino acid sequence having at least 90%, 91%, 92%, 93%, 94%, 95%, 96%, 97%, 98% or 99% identity with the amino acid sequence of SEQ ID NO: 443, or consists of SEQ ID NO: 443;
8) the heavy chain variable region comprises the amino acid sequence as set forth in SEQ ID NO: 445, or an amino acid sequence having at least 90%, 91%, 92%, 93%, 94%, 95%, 96%, 97%, 98% or 99% identity with the amino acid sequence of SEQ ID NO: 445, or consists of SEQ ID NO: 445;
9) the heavy chain variable region comprises the amino acid sequence as set forth in SEQ ID NO: 447, or an amino acid sequence having at least 90%, 91%, 92%, 93%, 94%, 95%, 96%, 97%, 98% or 99% identity with the amino acid sequence of SEQ ID NO: 447, or consists of SEQ ID NO: 447;
10) the heavy chain variable region comprises the amino acid sequence as set forth in SEQ ID NO: 449, or an amino acid sequence having at least 90%, 91%, 92%, 93%, 94%, 95%, 96%, 97%, 98% or 99% identity with the amino acid sequence of SEQ ID NO: 449, or consists of SEQ ID NO: 449;
11) the heavy chain variable region comprises the amino acid sequence as set forth in SEQ ID NO: 451, or an amino acid sequence having at least 90%, 91%, 92%, 93%, 94%, 95%, 96%, 97%, 98% or 99% identity with the amino acid sequence of SEQ ID NO: 451, or consists of SEQ ID NO: 451;
12) the heavy chain variable region comprises the amino acid sequence as set forth in SEQ ID NO: 453, or an amino acid sequence having at least 90%, 91%, 92%, 93%, 94%, 95%, 96%, 97%, 98% or 99% identity with the amino acid sequence of SEQ ID NO: 453, or consists of SEQ ID NO: 453;
13) the heavy chain variable region comprises the amino acid sequence as set forth in SEQ ID NO: 455, or an amino acid sequence having at least 90%, 91%, 92%, 93%, 94%, 95%, 96%, 97%, 98% or 99% identity with the amino acid sequence of SEQ ID NO: 455, or consists of SEQ ID NO: 455;
14) the heavy chain variable region comprises the amino acid sequence as set forth in SEQ ID NO: 457, or an amino acid sequence having at least 90%, 91%, 92%, 93%, 94%, 95%, 96%, 97%, 98% or 99% identity with the amino acid sequence of SEQ ID NO: 457, or consists of SEQ ID NO: 457;
15) the heavy chain variable region comprises the amino acid sequence as set forth in SEQ ID NO: 459, or an amino acid sequence having at least 90%, 91%, 92%, 93%, 94%, 95%, 9%%, 97%, 98% or 99% identity with the amino acid sequence of SEQ ID NO: 459, or consists of SEQ ID NO: 459;
16) the heavy chain variable region comprises the amino acid sequence as set forth in SEQ ID NO: 461, or an amino acid sequence having at least 90%, 91%, 92%, 93%, 94%, 95%, 96%, 97%, 98% or 99% identity with the amino acid sequence of SEQ ID NO: 461, or consists of SEQ ID NO: 461;
17) the heavy chain variable region comprises the amino acid sequence as set forth in SEQ ID NO: 463, or an amino acid sequence having at least 90%, 91%, 92%, 93%, 94%, 95%, 96%, 97%, 98% or 99% identity with the amino acid sequence of SEQ ID NO: 463, or consists of SEQ ID NO: 463;
18) the heavy chain variable region comprises the amino acid sequence as set forth in SEQ ID NO: 465, or an amino acid sequence having at least 90%, 91%, 92%, 93%, 94%, 95%, 96%, 97%, 98% or 99% identity with the amino acid sequence of SEQ ID NO: 465, or consists of SEQ ID NO: 465;
19) the heavy chain variable region comprises the amino acid sequence as set forth in SEQ ID NO: 467, or an amino acid sequence having at least 90%, 91%, 92%, 93%, 94%, 95%, 96%, 97%, 98% or 99% identity with the amino acid sequence of SEQ ID NO: 467, or consists of SEQ ID NO: 467;
20) the heavy chain variable region comprises the amino acid sequence as set forth in SEQ ID NO: 469, or an amino acid sequence having at least 90%, 91%, 92%, 93%, 94%, 95%, 96%, 97%, 98% or 99% identity with the amino acid sequence of SEQ ID NO: 469, or consists of SEQ ID NO: 469;
21) the heavy chain variable region comprises the amino acid sequence as set forth in SEQ ID NO: 471, or an amino acid sequence having at least 90%, 91%, 92%, 93%, 94%, 95%, 96%, 97%, 98% or 99% identity with the amino acid sequence of SEQ ID NO: 471, or consists of SEQ ID NO: 471;
22) the heavy chain variable region comprises the amino acid sequence as set forth in SEQ ID NO: 473, or an amino acid sequence having at least 90%, 91%, 92%, 93%, 94%, 95%, 96%, 97%, 98% or 99% identity with the amino acid sequence of SEQ ID NO: 473, or consists of SEQ ID NO: 473;
23) the heavy chain variable region comprises the amino acid sequence as set forth in SEQ ID NO: 475, or an amino acid sequence having at least 90%, 91%, 92%, 93%, 94%, 95%, 96%, 97%, 98% or 99% identity with the amino acid sequence of SEQ ID NO: 475, or consists of SEQ ID NO: 475;
24) the heavy chain variable region comprises the amino acid sequence as set forth in SEQ ID NO: 477, or an amino acid sequence having at least 90%, 91%, 92%, 93%, 94%, 95%, 96%, 97%, 98% or 99% identity with the amino acid sequence of SEQ ID NO: 477, or consists of SEQ ID NO: 477;
25) the heavy chain variable region comprises the amino acid sequence as set forth in SEQ ID NO: 479, or an amino acid sequence having at least 90%, 91%, 92%, 93%, 94%, 95%, 96%, 97%, 98% or 99% identity with the amino acid sequence of SEQ ID NO: 479, or consists of SEQ ID NO: 479;
26) the heavy chain variable region comprises the amino acid sequence as set forth in SEQ ID NO: 481, or an amino acid sequence having at least 90%, 91%, 92%, 93%, 94%, 95%, 9%%, 97%, 98% or 99% identity with the amino acid sequence of SEQ ID NO: 481, or consists of SEQ ID NO: 481;
27) the heavy chain variable region comprises the amino acid sequence as set forth in SEQ ID NO: 483, or an amino acid sequence having at least 90%, 91%, 92%, 93%, 94%, 95%, 96%, 97%, 98% or 99% identity with the amino acid sequence of SEQ ID NO: 483, or consists of SEQ ID NO: 483;
28) the heavy chain variable region comprises the amino acid sequence as set forth in SEQ ID NO: 485, or an amino acid sequence having at least 90%, 91%, 92%, 93%, 94%, 95%, 96%, 97%, 98% or 99% identity with the amino acid sequence of SEQ ID NO: 485, or consists of SEQ ID NO: 485;
29) the heavy chain variable region comprises the amino acid sequence as set forth in SEQ ID NO: 487, or an amino acid sequence having at least 90%, 91%, 92%, 93%, 94%, 95%, 96%, 97%, 98% or 99% identity with the amino acid sequence of SEQ ID NO: 487, or consists of SEQ ID NO: 487;
30) the heavy chain variable region comprises the amino acid sequence as set forth in SEQ ID NO: 488, or an amino acid sequence having at least 90%, 91%, 92%, 93%, 94%, 95%, 96%, 97%, 98% or 99% identity with the amino acid sequence of SEQ ID NO: 488, or consists of SEQ ID NO: 488;
31) the heavy chain variable region comprises the amino acid sequence as set forth in SEQ ID NO: 490, or an amino acid sequence having at least 90%, 91%, 92%, 93%, 94%, 95%, 96%, 97%, 98% or 99% identity with the amino acid sequence of SEQ ID NO: 490, or consists of SEQ ID NO: 490;
32) the heavy chain variable region comprises the amino acid sequence as set forth in SEQ ID NO: 492, or an amino acid sequence having at least 90%, 91%, 92%, 93%, 94%, 95%, 96%, 97%, 98% or 99% identity with the amino acid sequence of SEQ ID NO: 492, or consists of SEQ ID NO: 492;
33) the heavy chain variable region comprises the amino acid sequence as set forth in SEQ ID NO: 493, or an amino acid sequence having at least 90%, 91%, 92%, 93%, 94%, 95%, 96%, 97%, 98% or 99% identity with the amino acid sequence of SEQ ID NO: 493, or consists of SEQ ID NO: 493;
34) the heavy chain variable region comprises the amino acid sequence as set forth in SEQ ID NO: 496, or an amino acid sequence having at least 90%, 91%, 92%, 93%, 94%, 95%, 96%, 97%, 98% or 99% identity with the amino acid sequence of SEQ ID NO: 496, or consists of SEQ ID NO: 496;
35) the heavy chain variable region comprises the amino acid sequence as set forth in SEQ ID NO: 498, or an amino acid sequence having at least 90%, 91%, 92%, 93%, 94%, 95%, 96%, 97%, 98% or 99% identity with the amino acid sequence of SEQ ID NO: 498, or consists of SEQ ID NO: 498;
36) the heavy chain variable region comprises the amino acid sequence as set forth in SEQ ID NO: 499, or an amino acid sequence having at least 90%, 91%, 92%, 93%, 94%, 95%, 96%, 97%, 98% or 99% identity with the amino acid sequence of SEQ ID NO: 499, or consists of SEQ ID NO: 499;
37) the heavy chain variable region comprises the amino acid sequence as set forth in SEQ ID NO: 501, or an amino acid sequence having at least 90%, 91%, 92%, 93%, 94%, 95%, 9%%, 97%, 98% or 99% identity with the amino acid sequence of SEQ ID NO: 501, or consists of SEQ ID NO: 501;
38) the heavy chain variable region comprises the amino acid sequence as set forth in SEQ ID NO: 502, or an amino acid sequence having at least 90%, 91%, 92%, 93%, 94%, 95%, 96%, 97%, 98% or 99% identity with the amino acid sequence of SEQ ID NO: 502, or consists of SEQ ID NO: 502;
39) the heavy chain variable region comprises the amino acid sequence as set forth in SEQ ID NO: 504, or an amino acid sequence having at least 90%, 91%, 92%, 93%, 94%, 95%, 96%, 97%, 98% or 99% identity with the amino acid sequence of SEQ ID NO: 504, or consists of SEQ ID NO: 504;
40) the heavy chain variable region comprises the amino acid sequence as set forth in SEQ ID NO: 507, or an amino acid sequence having at least 90%, 91%, 92%, 93%, 94%, 95%, 96%, 97%, 98% or 99% identity with the amino acid sequence of SEQ ID NO: 507, or consists of SEQ ID NO: 507;
41) the heavy chain variable region comprises the amino acid sequence as set forth in SEQ ID NO: 508, or an amino acid sequence having at least 90%, 91%, 92%, 93%, 94%, 95%, 96%, 97%, 98% or 99% identity with the amino acid sequence of SEQ ID NO: 508, or consists of SEQ ID NO: 508;
42) the heavy chain variable region comprises the amino acid sequence as set forth in SEQ ID NO: 510, or an amino acid sequence having at least 90%, 91%, 92%, 93%, 94%, 95%, 96%, 97%, 98% or 99% identity with the amino acid sequence of SEQ ID NO: 510, or consists of SEQ ID NO: 510;
43) the heavy chain variable region comprises the amino acid sequence as set forth in SEQ ID NO: 512, or an amino acid sequence having at least 90%, 91%, 92%, 93%, 94%, 95%, 96%, 97%, 98% or 99% identity with the amino acid sequence of SEQ ID NO: 512, or consists of SEQ ID NO: 512;
44) the heavy chain variable region comprises the amino acid sequence as set forth in SEQ ID NO: 514, or an amino acid sequence having at least 90%, 91%, 92%, 93%, 94%, 95%, 96%, 97%, 98% or 99% identity with the amino acid sequence of SEQ ID NO: 514, or consists of SEQ ID NO: 514;
45) the heavy chain variable region comprises the amino acid sequence as set forth in SEQ ID NO: 517, or an amino acid sequence having at least 90%, 91%, 92%, 93%, 94%, 95%, 96%, 97%, 98% or 99% identity with the amino acid sequence of SEQ ID NO: 517, or consists of SEQ ID NO: 517;
46) the heavy chain variable region comprises the amino acid sequence as set forth in SEQ ID NO: 519, or an amino acid sequence having at least 90%, 91%, 92%, 93%, 94%, 95%, 96%, 97%, 98% or 99% identity with the amino acid sequence of SEQ ID NO: 519, or consists of SEQ ID NO: 519;
47) the heavy chain variable region comprises the amino acid sequence as set forth in SEQ ID NO: 521, or an amino acid sequence having at least 90%, 91%, 92%, 93%, 94%, 95%, 96%, 97%, 98% or 99% identity with the amino acid sequence of SEQ ID NO: 521, or consists of SEQ ID NO: 521;
48) the heavy chain variable region comprises the amino acid sequence as set forth in SEQ ID NO: 523, or an amino acid sequence having at least 90%, 91%, 92%, 93%, 94%, 95%, 9%%, 97%, 98% or 99% identity with the amino acid sequence of SEQ ID NO: 523, or consists of SEQ ID NO: 523;
49) the heavy chain variable region comprises the amino acid sequence as set forth in SEQ ID NO: 525, or an amino acid sequence having at least 90%, 91%, 92%, 93%, 94%, 95%, 96%, 97%, 98% or 99% identity with the amino acid sequence of SEQ ID NO: 525, or consists of SEQ ID NO: 525;
50) the heavy chain variable region comprises the amino acid sequence as set forth in SEQ ID NO: 526, or an amino acid sequence having at least 90%, 91%, 92%, 93%, 94%, 95%, 96%, 97%, 98% or 99% identity with the amino acid sequence of SEQ ID NO: 526, or consists of SEQ ID NO: 526;
51) the heavy chain variable region comprises the amino acid sequence as set forth in SEQ ID NO: 528, or an amino acid sequence having at least 90%, 91%, 92%, 93%, 94%, 95%, 96%, 97%, 98% or 99% identity with the amino acid sequence of SEQ ID NO: 528, or consists of SEQ ID NO: 528;
52) the heavy chain variable region comprises the amino acid sequence as set forth in SEQ ID NO: 530, or an amino acid sequence having at least 90%, 91%, 92%, 93%, 94%, 95%, 96%, 97%, 98% or 99% identity with the amino acid sequence of SEQ ID NO: 530, or consists of SEQ ID NO: 530;
53) the heavy chain variable region comprises the amino acid sequence as set forth in SEQ ID NO: 532, or an amino acid sequence having at least 90%, 91%, 92%, 93%, 94%, 95%, 96%, 97%, 98% or 99% identity with the amino acid sequence of SEQ ID NO: 532, or consists of SEQ ID NO: 532;
54) the heavy chain variable region comprises the amino acid sequence as set forth in SEQ ID NO: 534, or an amino acid sequence having at least 90%, 91%, 92%, 93%, 94%, 95%, 96%, 97%, 98% or 99% identity with the amino acid sequence of SEQ ID NO: 534, or consists of SEQ ID NO: 534;
55) the heavy chain variable region comprises the amino acid sequence as set forth in SEQ ID NO: 536, or an amino acid sequence having at least 90%, 91%, 92%, 93%, 94%, 95%, 96%, 97%, 98% or 99% identity with the amino acid sequence of SEQ ID NO: 536, or consists of SEQ ID NO: 536;
56) the heavy chain variable region comprises the amino acid sequence as set forth in SEQ ID NO: 538, or an amino acid sequence having at least 90%, 91%, 92%, 93%, 94%, 95%, 96%, 97%, 98% or 99% identity with the amino acid sequence of SEQ ID NO: 538, or consists of SEQ ID NO: 538;
57) the heavy chain variable region comprises the amino acid sequence as set forth in SEQ ID NO: 540, or an amino acid sequence having at least 90%, 91%, 92%, 93%, 94%, 95%, 96%, 97%, 98% or 99% identity with the amino acid sequence of SEQ ID NO: 540, or consists of SEQ ID NO: 540;
58) the heavy chain variable region comprises the amino acid sequence as set forth in SEQ ID NO: 543, or an amino acid sequence having at least 90%, 91%, 92%, 93%, 94%, 95%, 96%, 97%, 98% or 99% identity with the amino acid sequence of SEQ ID NO: 543, or consists of SEQ ID NO: 543;
59) the heavy chain variable region comprises the amino acid sequence as set forth in SEQ ID NO: 545, or an amino acid sequence having at least 90%, 91%, 92%, 93%, 94%, 95%, 9%%, 97%, 98% or 99% identity with the amino acid sequence of SEQ ID NO: 545, or consists of SEQ ID NO: 545;
60) the heavy chain variable region comprises the amino acid sequence as set forth in SEQ ID NO: 547, or an amino acid sequence having at least 90%, 91%, 92%, 93%, 94%, 95%, 96%, 97%, 98% or 99% identity with the amino acid sequence of SEQ ID NO: 547, or consists of SEQ ID NO: 547;
61) the heavy chain variable region comprises the amino acid sequence as set forth in SEQ ID NO: 549, or an amino acid sequence having at least 90%, 91%, 92%, 93%, 94%, 95%, 96%, 97%, 98% or 99% identity with the amino acid sequence of SEQ ID NO: 549, or consists of SEQ ID NO: 549;
62) the heavy chain variable region comprises the amino acid sequence as set forth in SEQ ID NO: 551, or an amino acid sequence having at least 90%, 91%, 92%, 93%, 94%, 95%, 96%, 97%, 98% or 99% identity with the amino acid sequence of SEQ ID NO: 551, or consists of SEQ ID NO: 551;
63) the heavy chain variable region comprises the amino acid sequence as set forth in SEQ ID NO: 553, or an amino acid sequence having at least 90%, 91%, 92%, 93%, 94%, 95%, 96%, 97%, 98% or 99% identity with the amino acid sequence of SEQ ID NO: 553, or consists of SEQ ID NO: 553;
64) the heavy chain variable region comprises the amino acid sequence as set forth in SEQ ID NO: 555, or an amino acid sequence having at least 90%, 91%, 92%, 93%, 94%, 95%, 96%, 97%, 98% or 99% identity with the amino acid sequence of SEQ ID NO: 555, or consists of SEQ ID NO: 555;
65) the heavy chain variable region comprises the amino acid sequence as set forth in SEQ ID NO: 557, or an amino acid sequence having at least 90%, 91%, 92%, 93%, 94%, 95%, 96%, 97%, 98% or 99% identity with the amino acid sequence of SEQ ID NO: 557, or consists of SEQ ID NO: 557;
66) the heavy chain variable region comprises the amino acid sequence as set forth in SEQ ID NO: 559, or an amino acid sequence having at least 90%, 91%, 92%, 93%, 94%, 95%, 96%, 97%, 98% or 99% identity with the amino acid sequence of SEQ ID NO: 559, or consists of SEQ ID NO: 559;
67) the heavy chain variable region comprises the amino acid sequence as set forth in SEQ ID NO: 561, or an amino acid sequence having at least 90%, 91%, 92%, 93%, 94%, 95%, 96%, 97%, 98% or 99% identity with the amino acid sequence of SEQ ID NO: 561, or consists of SEQ ID NO: 561;
68) the heavy chain variable region comprises the amino acid sequence as set forth in SEQ ID NO: 562, or an amino acid sequence having at least 90%, 91%, 92%, 93%, 94%, 95%, 96%, 97%, 98% or 99% identity with the amino acid sequence of SEQ ID NO: 562, or consists of SEQ ID NO: 562;
69) the heavy chain variable region comprises the amino acid sequence as set forth in SEQ ID NO: 564, or an amino acid sequence having at least 90%, 91%, 92%, 93%, 94%, 95%, 96%, 97%, 98% or 99% identity with the amino acid sequence of SEQ ID NO: 564, or consists of SEQ ID NO: 564;
70) the heavy chain variable region comprises the amino acid sequence as set forth in SEQ ID NO: 566, or an amino acid sequence having at least 90%, 91%, 92%, 93%, 94%, 95%, 9%%, 97%, 98% or 99% identity with the amino acid sequence of SEQ ID NO: 566, or consists of SEQ ID NO: 566;
71) the heavy chain variable region comprises the amino acid sequence as set forth in SEQ ID NO: 568, or an amino acid sequence having at least 90%, 91%, 92%, 93%, 94%, 95%, 96%, 97%, 98% or 99% identity with the amino acid sequence of SEQ ID NO: 568, or consists of SEQ ID NO: 568;
72) the heavy chain variable region comprises the amino acid sequence as set forth in SEQ ID NO: 570, or an amino acid sequence having at least 90%, 91%, 92%, 93%, 94%, 95%, 96%, 97%, 98% or 99% identity with the amino acid sequence of SEQ ID NO: 570, or consists of SEQ ID NO: 570;
73) the heavy chain variable region comprises the amino acid sequence as set forth in SEQ ID NO: 572, or an amino acid sequence having at least 90%, 91%, 92%, 93%, 94%, 95%, 96%, 97%, 98% or 99% identity with the amino acid sequence of SEQ ID NO: 572, or consists of SEQ ID NO: 572;
74) the heavy chain variable region comprises the amino acid sequence as set forth in SEQ ID NO: 574, or an amino acid sequence having at least 90%, 91%, 92%, 93%, 94%, 95%, 96%, 97%, 98% or 99% identity with the amino acid sequence of SEQ ID NO: 574, or consists of SEQ ID NO: 574;
75) the heavy chain variable region comprises the amino acid sequence as set forth in SEQ ID NO: 576, or an amino acid sequence having at least 90%, 91%, 92%, 93%, 94%, 95%, 96%, 97%, 98% or 99% identity with the amino acid sequence of SEQ ID NO: 576, or consists of SEQ ID NO: 576;
76) the heavy chain variable region comprises the amino acid sequence as set forth in SEQ ID NO: 577, or an amino acid sequence having at least 90%, 91%, 92%, 93%, 94%, 95%, 96%, 97%, 98% or 99% identity with the amino acid sequence of SEQ ID NO: 577, or consists of SEQ ID NO: 577;
77) the heavy chain variable region comprises the amino acid sequence as set forth in SEQ ID NO: 579, or an amino acid sequence having at least 90%, 91%, 92%, 93%, 94%, 95%, 96%, 97%, 98% or 99% identity with the amino acid sequence of SEQ ID NO: 579, or consists of SEQ ID NO: 579;
78) the heavy chain variable region comprises the amino acid sequence as set forth in SEQ ID NO: 581, or an amino acid sequence having at least 90%, 91%, 92%, 93%, 94%, 95%, 96%, 97%, 98% or 99% identity with the amino acid sequence of SEQ ID NO: 581, or consists of SEQ ID NO: 581;
79) the heavy chain variable region comprises the amino acid sequence as set forth in SEQ ID NO: 583, or an amino acid sequence having at least 90%, 91%, 92%, 93%, 94%, 95%, 96%, 97%, 98% or 99% identity with the amino acid sequence of SEQ ID NO: 583, or consists of SEQ ID NO: 583;
80) the heavy chain variable region comprises the amino acid sequence as set forth in SEQ ID NO: 585, or an amino acid sequence having at least 90%, 91%, 92%, 93%, 94%, 95%, 96%, 97%, 98% or 99% identity with the amino acid sequence of SEQ ID NO: 585, or consists of SEQ ID NO: 585;
81) the heavy chain variable region comprises the amino acid sequence as set forth in SEQ ID NO: 587, or an amino acid sequence having at least 90%, 91%, 92%, 93%, 94%, 95%, 9%%, 97%, 98% or 99% identity with the amino acid sequence of SEQ ID NO: 587, or consists of SEQ ID NO: 587;
82) the heavy chain variable region comprises the amino acid sequence as set forth in SEQ ID NO: 590, or an amino acid sequence having at least 90%, 91%, 92%, 93%, 94%, 95%, 96%, 97%, 98% or 99% identity with the amino acid sequence of SEQ ID NO: 590, or consists of SEQ ID NO: 590;
83) the heavy chain variable region comprises the amino acid sequence as set forth in SEQ ID NO: 592, or an amino acid sequence having at least 90%, 91%, 92%, 93%, 94%, 95%, 96%, 97%, 98% or 99% identity with the amino acid sequence of SEQ ID NO: 592, or consists of SEQ ID NO: 592;
84) the heavy chain variable region comprises the amino acid sequence as set forth in SEQ ID NO: 595, or an amino acid sequence having at least 90%, 91%, 92%, 93%, 94%, 95%, 96%, 97%, 98% or 99% identity with the amino acid sequence of SEQ ID NO: 595, or consists of SEQ ID NO: 595;
85) the heavy chain variable region comprises the amino acid sequence as set forth in SEQ ID NO: 597, or an amino acid sequence having at least 90%, 91%, 92%, 93%, 94%, 95%, 96%, 97%, 98% or 99% identity with the amino acid sequence of SEQ ID NO: 597, or consists of SEQ ID NO: 597;
86) the heavy chain variable region comprises the amino acid sequence as set forth in SEQ ID NO: 599, or an amino acid sequence having at least 90%, 91%, 92%, 93%, 94%, 95%, 96%, 97%, 98% or 99% identity with the amino acid sequence of SEQ ID NO: 599, or consists of SEQ ID NO: 599;
87) the heavy chain variable region comprises the amino acid sequence as set forth in SEQ ID NO: 601, or an amino acid sequence having at least 90%, 91%, 92%, 93%, 94%, 95%, 96%, 97%, 98% or 99% identity with the amino acid sequence of SEQ ID NO: 601, or consists of SEQ ID NO: 601;
88) the heavy chain variable region comprises the amino acid sequence as set forth in SEQ ID NO: 603, or an amino acid sequence having at least 90%, 91%, 92%, 93%, 94%, 95%, 96%, 97%, 98% or 99% identity with the amino acid sequence of SEQ ID NO: 603, or consists of SEQ ID NO: 603;
89) the heavy chain variable region comprises the amino acid sequence as set forth in SEQ ID NO: 605, or an amino acid sequence having at least 90%, 91%, 92%, 93%, 94%, 95%, 96%, 97%, 98% or 99% identity with the amino acid sequence of SEQ ID NO: 605, or consists of SEQ ID NO: 605;
90) the heavy chain variable region comprises the amino acid sequence as set forth in SEQ ID NO: 607, or an amino acid sequence having at least 90%, 91%, 92%, 93%, 94%, 95%, 96%, 97%, 98% or 99% identity with the amino acid sequence of SEQ ID NO: 607, or consists of SEQ ID NO: 607;
91) the heavy chain variable region comprises the amino acid sequence as set forth in SEQ ID NO: 609, or an amino acid sequence having at least 90%, 91%, 92%, 93%, 94%, 95%, 96%, 97%, 98% or 99% identity with the amino acid sequence of SEQ ID NO: 609, or consists of SEQ ID NO: 609;
92) the heavy chain variable region comprises the amino acid sequence as set forth in SEQ ID NO: 611, or an amino acid sequence having at least 90%, 91%, 92%, 93%, 94%, 95%, 9%%, 97%, 98% or 99% identity with the amino acid sequence of SEQ ID NO: 611, or consists of SEQ ID NO: 611;
93) the heavy chain variable region comprises the amino acid sequence as set forth in SEQ ID NO: 613, or an amino acid sequence having at least 90%, 91%, 92%, 93%, 94%, 95%, 96%, 97%, 98% or 99% identity with the amino acid sequence of SEQ ID NO: 613, or consists of SEQ ID NO: 613;
94) the heavy chain variable region comprises the amino acid sequence as set forth in SEQ ID NO: 615, or an amino acid sequence having at least 90%, 91%, 92%, 93%, 94%, 95%, 96%, 97%, 98% or 99% identity with the amino acid sequence of SEQ ID NO: 615, or consists of SEQ ID NO: 615;
95) the heavy chain variable region comprises the amino acid sequence as set forth in SEQ ID NO: 617, or an amino acid sequence having at least 90%, 91%, 92%, 93%, 94%, 95%, 96%, 97%, 98% or 99% identity with the amino acid sequence of SEQ ID NO: 617, or consists of SEQ ID NO: 617;
96) the heavy chain variable region comprises the amino acid sequence as set forth in SEQ ID NO: 619, or an amino acid sequence having at least 90%, 91%, 92%, 93%, 94%, 95%, 96%, 97%, 98% or 99% identity with the amino acid sequence of SEQ ID NO: 619, or consists of SEQ ID NO: 619;
97) the heavy chain variable region comprises the amino acid sequence as set forth in SEQ ID NO: 621, or an amino acid sequence having at least 90%, 91%, 92%, 93%, 94%, 95%, 96%, 97%, 98% or 99% identity with the amino acid sequence of SEQ ID NO: 621, or consists of SEQ ID NO: 621;
98) the heavy chain variable region comprises the amino acid sequence as set forth in SEQ ID NO: 623, or an amino acid sequence having at least 90%, 91%, 92%, 93%, 94%, 95%, 96%, 97%, 98% or 99% identity with the amino acid sequence of SEQ ID NO: 623, or consists of SEQ ID NO: 623;
99) the heavy chain variable region comprises the amino acid sequence as set forth in SEQ ID NO: 625, or an amino acid sequence having at least 90%, 91%, 92%, 93%, 94%, 95%, 96%, 97%, 98% or 99% identity with the amino acid sequence of SEQ ID NO: 625, or consists of SEQ ID NO: 625;
100) the heavy chain variable region comprises the amino acid sequence as set forth in SEQ ID NO: 627, or an amino acid sequence having at least 90%, 91%, 92%, 93%, 94%, 95%, 96%, 97%, 98% or 99% identity with the amino acid sequence of SEQ ID NO: 627, or consists of SEQ ID NO: 627;
101) the heavy chain variable region comprises the amino acid sequence as set forth in SEQ ID NO: 629, or an amino acid sequence having at least 90%, 91%, 92%, 93%, 94%, 95%, 96%, 97%, 98% or 99% identity with the amino acid sequence of SEQ ID NO: 629, or consists of SEQ ID NO: 629;
102) the heavy chain variable region comprises the amino acid sequence as set forth in SEQ ID NO: 631, or an amino acid sequence having at least 90%, 91%, 92%, 93%, 94%, 95%, 96%, 97%, 98% or 99% identity with the amino acid sequence of SEQ ID NO: 631, or consists of SEQ ID NO: 631;
103) the heavy chain variable region comprises the amino acid sequence as set forth in SEQ ID NO: 634, or an amino acid sequence having at least 90%, 91%, 92%, 93%, 94%, 95% 96%, 97%, 98% or 99% identity with the amino acid sequence of SEQ ID NO: 634, or consists of SEQ ID NO: 634;
104) the heavy chain variable region comprises the amino acid sequence as set forth in SEQ ID NO: 636, or an amino acid sequence having at least 90%, 91%, 92%, 93%, 94% 95%, 96%, 97%, 98% or 99% identity with the amino acid sequence of SEQ ID NO: 636, or consists of SEQ ID NO: 636;
105) the heavy chain variable region comprises the amino acid sequence as set forth in SEQ ID NO: 638, or an amino acid sequence having at least 90%, 91%, 92%, 93%, 94% 95%, 96%, 97%, 98% or 99% identity with the amino acid sequence of SEQ ID NO: 638, or consists of SEQ ID NO: 638;
106) the heavy chain variable region comprises the amino acid sequence as set forth in SEQ ID NO: 640, or an amino acid sequence having at least 90%, 91%, 92%, 93%, 94%, 95%, 96%, 97%, 98% or 99% identity with the amino acid sequence of SEQ ID NO: 640, or consists of SEQ ID NO: 640;
107) the heavy chain variable region comprises the amino acid sequence as set forth in SEQ ID NO: 642, or an amino acid sequence having at least 90%, 91%, 92%, 93%, 94%, 95%, 96%, 97%, 98% or 99% identity with the amino acid sequence of SEQ ID NO: 642, or consists of SEQ ID NO: 642;
108) the heavy chain variable region comprises the amino acid sequence as set forth in SEQ ID NO: 644, or an amino acid sequence having at least 90%, 91%, 92%, 93%, 94% 95%, 96%, 97%, 98% or 99% identity with the amino acid sequence of SEQ ID NO: 644, or consists of SEQ ID NO: 644;
109) the heavy chain variable region comprises the amino acid sequence as set forth in SEQ ID NO: 646, or an amino acid sequence having at least 90%, 91%, 92%, 93% 94%, 95%, 96%, 97%, 98% or 99% identity with the amino acid sequence of SEQ ID NO: 646, or consists of SEQ ID NO: 646;
110) the heavy chain variable region comprises the amino acid sequence as set forth in SEQ ID NO: 648, or an amino acid sequence having at least 90%, 91%, 92%, 93%, 94%, 95%, 96%, 97%, 98% or 99% identity with the amino acid sequence of SEQ ID NO: 648, or consists of SEQ ID NO: 648;
111) the heavy chain variable region comprises the amino acid sequence as set forth in SEQ ID NO: 650, or an amino acid sequence having at least 90%, 91%, 92%, 93%, 94%, 95%, 96%, 97%, 98% or 99% identity with the amino acid sequence of SEQ ID NO: 650, or consists of SEQ ID NO: 650;
112) the heavy chain variable region comprises the amino acid sequence as set forth in SEQ ID NO: 652, or an amino acid sequence having at least 90%, 91%, 92%, 93%, 94%, 95%, 96%, 97%, 98% or 99% identity with the amino acid sequence of SEQ ID NO: 652, or consists of SEQ ID NO: 652;
113) the heavy chain variable region comprises the amino acid sequence as set forth in SEQ ID NO: 654, or an amino acid sequence having at least 90%, 91%, 92%, 93%, 94%, 95%, 96%, 97%, 98% or 99% identity with the amino acid sequence of SEQ ID NO: 654, or consists of SEQ ID NO: 654; or
114) The heavy chain variable region comprises the amino acid sequence as set forth in SEQ ID NO: 656, or an amino acid sequence having at least 90%, 91%, 92%, 93%, 94%, 95%, 96%, 97%, 98% or 99% identity to the amino acid sequence of SEQ ID NO: 656, or consists of SEQ ID NO: 656.

In one embodiment, the invention provides an anti-CD112R antibody that specifically binds CD112R and an antigen-binding fragment thereof, comprising a light chain variable region, wherein:
1) the light chain variable region comprises the amino acid sequence as set forth in SEQ ID NO: 431, or an amino acid sequence having at least 90%, 91%, 92%, 93%, 94%, 95%, 96%, 97%, 98% or 99% identity with the amino acid sequence of SEQ ID NO: 431, or consists of SEQ ID NO: 431;
2) the light chain variable region comprises the amino acid sequence as set forth in SEQ ID NO: 433, or an amino acid sequence having at least 90%, 91%, 92%, 93%, 94%, 95%, 96%, 97%, 98% or 99% identity with the amino acid sequence of SEQ ID NO; 433, or consists of SEQ ID NO: 433;
3) the light chain variable region comprises the amino acid sequence as set forth in SEQ ID NO: 435, or an amino acid sequence having at least 90%, 91%, 92%, 93%, 94%, 95%, 96%, 97%, 98% or 99% identity with the amino acid sequence of SEQ ID NO: 435, or consists of SEQ ID NO: 435;
4) the light chain variable region comprises the amino acid sequence as set forth in SEQ ID NO: 437, or an amino acid sequence having at least 90%, 91%, 92%, 93%, 94%, 95%, 96%, 97%, 98% or 99% identity with the amino acid sequence of SEQ ID NO: 437, or consists of SEQ ID NO: 437;
5) the light chain variable region comprises the amino acid sequence as set forth in SEQ ID NO: 439, or an amino acid sequence having at least 90%, 91%, 92%, 93%, 94%, 95%, 96%, 97%, 98% or 99% identity with the amino acid sequence of SEQ ID NO: 439, or consists of SEQ ID NO: 439;
6) the light chain variable region comprises the amino acid sequence as set forth in SEQ ID NO: 440, or an amino acid sequence having at least 90%, 91%, 92%, 93%, 94%, 95%, 96%, 97%, 98% or 99% identity with the amino acid sequence of SEQ ID NO: 440, or consists of SEQ ID NO: 440;
7) the light chain variable region comprises the amino acid sequence as set forth in SEQ ID NO: 442, or an amino acid sequence having at least 90%, 91%, 92%, 93%, 94%, 95%, 9%%, 97%, 98% or 99% identity with the amino acid sequence of SEQ ID NO: 442, or consists of SEQ ID NO: 442;
8) the light chain variable region comprises the amino acid sequence as set forth in SEQ ID NO: 444, or an amino acid sequence having at least 90%, 91%, 92%, 93%, 94%, 95%, 96%, 97%, 98% or 99% identity with the amino acid sequence of SEQ ID NO: 444, or consists of SEQ ID NO: 444;
9) the light chain variable region comprises the amino acid sequence as set forth in SEQ ID NO: 446, or an amino acid sequence having at least 90%, 91%, 92%, 93%, 94%, 95%, 96%, 97%, 98% or 99% identity with the amino acid sequence of SEQ ID NO: 446, or consists of SEQ ID NO: 446;
10) the light chain variable region comprises the amino acid sequence as set forth in SEQ ID NO: 448, or an amino acid sequence having at least 90%, 91%, 92%, 93%, 94%, 95%, 96%, 97%, 98% or 99% identity with the amino acid sequence of SEQ ID NO: 448, or consists of SEQ ID NO: 448;
11) the light chain variable region comprises the amino acid sequence as set forth in SEQ ID NO: 450, or an amino acid sequence having at least 90%, 91%, 92%, 93%, 94%, 95%, 96%, 97%, 98% or 99% identity with the amino acid sequence of SEQ ID NO: 450, or consists of SEQ ID NO: 450;
12) the light chain variable region comprises the amino acid sequence as set forth in SEQ ID NO: 452, or an amino acid sequence having at least 90%, 91%, 92%, 93%, 94%, 95%, 96%, 97%, 98% or 99% identity with the amino acid sequence of SEQ ID NO: 452, or consists of SEQ ID NO: 452;
13) the light chain variable region comprises the amino acid sequence as set forth in SEQ ID NO: 454, or an amino acid sequence having at least 90%, 91%, 92%, 93%, 94%, 95%, 96%, 97%, 98% or 99% identity with the amino acid sequence of SEQ ID NO: 454, or consists of SEQ ID NO: 454;
14) the light chain variable region comprises the amino acid sequence as set forth in SEQ ID NO: 456, or an amino acid sequence having at least 90%, 91%, 92%, 93%, 94%, 95%, 96%, 97%, 98% or 99% identity with the amino acid sequence of SEQ ID NO: 456, or consists of SEQ ID NO: 456;
15) the light chain variable region comprises the amino acid sequence as set forth in SEQ ID NO: 458, or an amino acid sequence having at least 90%, 91%, 92%, 93%, 94%, 95%, 96%, 97%, 98% or 99% identity with the amino acid sequence of SEQ ID NO: 458, or consists of SEQ ID NO: 458;
16) the light chain variable region comprises the amino acid sequence as set forth in SEQ ID NO: 460, or an amino acid sequence having at least 90%, 91%, 92%, 93%, 94%, 95%, 96%, 97%, 98% or 99% identity with the amino acid sequence of SEQ ID NO: 460, or consists of SEQ ID NO: 460;
17) the light chain variable region comprises the amino acid sequence as set forth in SEQ ID NO: 462, or an amino acid sequence having at least 90%, 91%, 92%, 93%, 94%, 95%, 96%, 97%, 98% or 99% identity with the amino acid sequence of SEQ ID NO: 462, or consists of SEQ ID NO: 462;
18) the light chain variable region comprises the amino acid sequence as set forth in SEQ ID NO: 464, or an amino acid sequence having at least 90%, 91%, 92%, 93%, 94%, 95%, 96%, 97%, 98% or 99% identity with the amino acid sequence of SEQ ID NO: 464, or consists of SEQ ID NO: 464;
19) the light chain variable region comprises the amino acid sequence as set forth in SEQ ID NO: 466, or an amino acid sequence having at least 90%, 91%, 92%, 93%, 94%, 95%, 96%, 97%, 98% or 99% identity with the amino acid sequence of SEQ ID NO: 466, or consists of SEQ ID NO: 466;
20) the light chain variable region comprises the amino acid sequence as set forth in SEQ ID NO: 468, or an amino acid sequence having at least 90%, 91%, 92%, 93%, 94%, 95%, 96%, 97%, 98% or 99% identity with the amino acid sequence of SEQ ID NO: 468, or consists of SEQ ID NO: 468;
21) the light chain variable region comprises the amino acid sequence as set forth in SEQ ID NO: 470, or an amino acid sequence having at least 90%, 91%, 92%, 93%, 94%, 95%, 96%, 97%, 98% or 99% identity with the amino acid sequence of SEQ ID NO: 470, or consists of SEQ ID NO: 470;
22) the light chain variable region comprises the amino acid sequence as set forth in SEQ ID NO: 472, or an amino acid sequence having at least 90%, 91%, 92%, 93%, 94%, 95%, 96%, 97%, 98% or 99% identity with the amino acid sequence of SEQ ID NO: 472, or consists of SEQ ID NO: 472;
23) the light chain variable region comprises the amino acid sequence as set forth in SEQ ID NO: 474, or an amino acid sequence having at least 90%, 91%, 92%, 93%, 94%, 95%, 96%, 97%, 98% or 99% identity with the amino acid sequence of SEQ ID NO: 474, or consists of SEQ ID NO: 474;
24) the light chain variable region comprises the amino acid sequence as set forth in SEQ ID NO: 476, or an amino acid sequence having at least 90%, 91%, 92%, 93%, 94%, 95%, 96%, 97%, 98% or 99% identity with the amino acid sequence of SEQ ID NO: 476, or consists of SEQ ID NO: 476;
25) the light chain variable region comprises the amino acid sequence as set forth in SEQ ID NO: 478, or an amino acid sequence having at least 90%, 91%, 92%, 93%, 94%, 95%, 96%, 97%, 98% or 99% identity with the amino acid sequence of SEQ ID NO: 478, or consists of SEQ ID NO: 478;
26) the light chain variable region comprises the amino acid sequence as set forth in SEQ ID NO: 480, or an amino acid sequence having at least 90%, 91%, 92%, 93%, 94%, 95%, 96%, 97%, 98% or 99% identity with the amino acid sequence of SEQ ID NO: 480, or consists of SEQ ID NO: 480;
27) the light chain variable region comprises the amino acid sequence as set forth in SEQ ID NO: 482, or an amino acid sequence having at least 90%, 91%, 92%, 93%, 94%, 95%, 96%, 97%, 98% or 99% identity with the amino acid sequence of SEQ ID NO: 482, or consists of SEQ ID NO: 482;
28) the light chain variable region comprises the amino acid sequence as set forth in SEQ ID NO: 484, or an amino acid sequence having at least 90%, 91%, 92%, 93%, 94%, 95%, 96%, 97%, 98% or 99% identity with the amino acid sequence of SEQ ID NO: 484, or consists of SEQ ID NO: 484;
29) the light chain variable region comprises the amino acid sequence as set forth in SEQ ID NO: 486, or an amino acid sequence having at least 90%, 91%, 92%, 93%, 94%, 95%, 96%, 97%, 98% or 99% identity with the amino acid sequence of SEQ ID NO: 486, or consists of SEQ ID NO: 486;
30) the light chain variable region comprises the amino acid sequence as set forth in SEQ ID NO: 489, or an amino acid sequence having at least 90%, 91%, 92%, 93%, 94%, 95%, 96%, 97%, 98% or 99% identity with the amino acid sequence of SEQ ID NO: 489, or consists of SEQ ID NO: 489;
31) the light chain variable region comprises the amino acid sequence as set forth in SEQ ID NO: 491, or an amino acid sequence having at least 90%, 91%, 92%, 93%, 94%, 95%, 96%, 97%, 98% or 99% identity with the amino acid sequence of SEQ ID NO: 491, or consists of SEQ ID NO: 491;
32) the light chain variable region comprises the amino acid sequence as set forth in SEQ ID NO: 494, or an amino acid sequence having at least 90%, 91%, 92%, 93%, 94%, 95%, 96%, 97%, 98% or 99% identity with the amino acid sequence of SEQ ID NO: 494, or consists of SEQ ID NO: 494;
33) the light chain variable region comprises the amino acid sequence as set forth in SEQ ID NO: 495, or an amino acid sequence having at least 90%, 91%, 92%, 93%, 94%, 95%, 96%, 97%, 98% or 99% identity with the amino acid sequence of SEQ ID NO: 495, or consists of SEQ ID NO: 495;
34) the light chain variable region comprises the amino acid sequence as set forth in SEQ ID NO: 497, or an amino acid sequence having at least 90%, 91%, 92%, 93%, 94%, 95%, 96%, 97%, 98% or 99% identity with the amino acid sequence of SEQ ID NO: 497, or consists of SEQ ID NO: 497;
35) the light chain variable region comprises the amino acid sequence as set forth in SEQ ID NO: 500, or an amino acid sequence having at least 90%, 91%, 92%, 93%, 94%, 95%, 96%, 97%, 98% or 99% identity with the amino acid sequence of SEQ ID NO: 500, or consists of SEQ ID NO: 500;
36) the light chain variable region comprises the amino acid sequence as set forth in SEQ ID NO: 503, or an amino acid sequence having at least 90%, 91%, 92%, 93%, 94%, 95%, 96%, 97%, 98% or 99% identity with the amino acid sequence of SEQ ID NO: 503, or consists of SEQ ID NO: 503;
37) the light chain variable region comprises the amino acid sequence as set forth in SEQ ID NO: 505, or an amino acid sequence having at least 90%, 91%, 92%, 93%, 94%, 95%, 96%, 97%, 98% or 99% identity with the amino acid sequence of SEQ ID NO: 505, or consists of SEQ ID NO: 505;
38) the light chain variable region comprises the amino acid sequence as set forth in SEQ ID NO: 506, or an amino acid sequence having at least 90%, 91%, 92%, 93%, 94%, 95%, 96%, 97%, 98% or 99% identity with the amino acid sequence of SEQ ID NO: 506, or consists of SEQ ID NO: 506;
39) the light chain variable region comprises the amino acid sequence as set forth in SEQ ID NO: 509, or an amino acid sequence having at least 90%, 91%, 92%, 93%, 94%, 95%, 96%, 97%, 98% or 99% identity with the amino acid sequence of SEQ ID NO: 509, or consists of SEQ ID NO: 509;
40) the light chain variable region comprises the amino acid sequence as set forth in SEQ ID NO: 511, or an amino acid sequence having at least 90%, 91%, 92%, 93%, 94%, 95%, 96%, 97%, 98% or 99% identity with the amino acid sequence of SEQ ID NO: 511, or consists of SEQ ID NO: 511;
41) the light chain variable region comprises the amino acid sequence as set forth in SEQ ID NO: 513, or an amino acid sequence having at least 90%, 91%, 92%, 93%, 94%, 95%, 96%, 97%, 98% or 99% identity with the amino acid sequence of SEQ ID NO: 513, or consists of SEQ ID NO: 513;
42) the light chain variable region comprises the amino acid sequence as set forth in SEQ ID NO: 515, or an amino acid sequence having at least 90%, 91%, 92%, 93%, 94%, 95%, 96%, 97%, 98% or 99% identity with the amino acid sequence of SEQ ID NO: 515, or consists of SEQ ID NO: 515;
43) the light chain variable region comprises the amino acid sequence as set forth in SEQ ID NO: 516, or an amino acid sequence having at least 90%, 91%, 92%, 93%, 94%, 95%, 96%, 97%, 98% or 99% identity with the amino acid sequence of SEQ ID NO: 516, or consists of SEQ ID NO: 516;
44) the light chain variable region comprises the amino acid sequence as set forth in SEQ ID NO: 518, or an amino acid sequence having at least 90%, 91%, 92%, 93%, 94%, 95%, 96%, 97%, 98% or 99% identity with the amino acid sequence of SEQ ID NO: 518, or consists of SEQ ID NO: 518;
45) the light chain variable region comprises the amino acid sequence as set forth in SEQ ID NO: 520, or an amino acid sequence having at least 90%, 91%, 92%, 93%, 94%, 95%, 96%, 97%, 98% or 99% identity with the amino acid sequence of SEQ ID NO: 520, or consists of SEQ ID NO: 520;
46) the light chain variable region comprises the amino acid sequence as set forth in SEQ ID NO: 522, or an amino acid sequence having at least 90%, 91%, 92%, 93%, 94%, 95%, 96%, 97%, 98% or 99% identity with the amino acid sequence of SEQ ID NO: 522, or consists of SEQ ID NO: 522;
47) the light chain variable region comprises the amino acid sequence as set forth in SEQ ID NO: 524, or an amino acid sequence having at least 90%, 91%, 92%, 93%, 94%, 95%, 96%, 97%, 98% or 99% identity with the amino acid sequence of SEQ ID NO: 524, or consists of SEQ ID NO: 524;
48) the light chain variable region comprises the amino acid sequence as set forth in SEQ ID NO: 527, or an amino acid sequence having at least 90%, 91%, 92%, 93%, 94%, 95%, 96%, 97%, 98% or 99% identity with the amino acid sequence of SEQ ID NO: 527, or consists of SEQ ID NO: 527;
49) the light chain variable region comprises the amino acid sequence as set forth in SEQ ID NO: 529, or an amino acid sequence having at least 90%, 91%, 92%, 93%, 94%, 95%, 96%, 97%, 98% or 99% identity with the amino acid sequence of SEQ ID NO: 529, or consists of SEQ ID NO: 529;
50) the light chain variable region comprises the amino acid sequence as set forth in SEQ ID NO: 531, or an amino acid sequence having at least 90%, 91%, 92%, 93%, 94%, 95%, 96%, 97%, 98% or 99% identity with the amino acid sequence of SEQ ID NO: 531, or consists of SEQ ID NO: 531;
51) the light chain variable region comprises the amino acid sequence as set forth in SEQ ID NO: 533, or an amino acid sequence having at least 90%, 91%, 92%, 93%, 94%, 95%, 9%%, 97%, 98% or 99% identity with the amino acid sequence of SEQ ID NO: 533, or consists of SEQ ID NO: 533;
52) the light chain variable region comprises the amino acid sequence as set forth in SEQ ID NO: 535, or an amino acid sequence having at least 90%, 91%, 92%, 93%, 94%, 95%, 96%, 97%, 98% or 99% identity with the amino acid sequence of SEQ ID NO: 535, or consists of SEQ ID NO: 535;
53) the light chain variable region comprises the amino acid sequence as set forth in SEQ ID NO: 537, or an amino acid sequence having at least 90%, 91%, 92%, 93%, 94%, 95%, 96%, 97%, 98% or 99% identity with the amino acid sequence of SEQ ID NO: 537, or consists of SEQ ID NO: 537;
54) the light chain variable region comprises the amino acid sequence as set forth in SEQ ID NO: 539, or an amino acid sequence having at least 90%, 91%, 92%, 93%, 94%, 95%, 96%, 97%, 98% or 99% identity with the amino acid sequence of SEQ ID NO: 539, or consists of SEQ ID NO: 539;
55) the light chain variable region comprises the amino acid sequence as set forth in SEQ ID NO: 541, or an amino acid sequence having at least 90%, 91%, 92%, 93%, 94%, 95%, 96%, 97%, 98% or 99% identity with the amino acid sequence of SEQ ID NO: 541, or consists of SEQ ID NO: 541;
56) the light chain variable region comprises the amino acid sequence as set forth in SEQ ID NO: 542, or an amino acid sequence having at least 90%, 91%, 92%, 93%, 94%, 95%, 96%, 97%, 98% or 99% identity with the amino acid sequence of SEQ ID NO: 542, or consists of SEQ ID NO: 542;
57) the light chain variable region comprises the amino acid sequence as set forth in SEQ ID NO: 544, or an amino acid sequence having at least 90%, 91%, 92%, 93%, 94%, 95%, 96%, 97%, 98% or 99% identity with the amino acid sequence of SEQ ID NO: 544, or consists of SEQ ID NO: 544;
58) the light chain variable region comprises the amino acid sequence as set forth in SEQ ID NO: 546, or an amino acid sequence having at least 90%, 91%, 92%, 93%, 94%, 95%, 96%, 97%, 98% or 99% identity with the amino acid sequence of SEQ ID NO: 546, or consists of SEQ ID NO: 546;
59) the light chain variable region comprises the amino acid sequence as set forth in SEQ ID NO: 548, or an amino acid sequence having at least 90%, 91%, 92%, 93%, 94%, 95%, 96%, 97%, 98% or 99% identity with the amino acid sequence of SEQ ID NO: 548, or consists of SEQ ID NO: 548;
60) the light chain variable region comprises the amino acid sequence as set forth in SEQ ID NO: 550, or an amino acid sequence having at least 90%, 91%, 92%, 93%, 94%, 95%, 96%, 97%, 98% or 99% identity with the amino acid sequence of SEQ ID NO: 550, or consists of SEQ ID NO: 550;
61) the light chain variable region comprises the amino acid sequence as set forth in SEQ ID NO: 552, or an amino acid sequence having at least 90%, 91%, 92%, 93%, 94%, 95%, 96%, 97%, 98% or 99% identity with the amino acid sequence of SEQ ID NO: 552, or consists of SEQ ID NO: 552;
62) the light chain variable region comprises the amino acid sequence as set forth in SEQ ID NO: 554, or an amino acid sequence having at least 90%, 91%, 92%, 93%, 94%, 95%, 9%%, 97%, 98% or 99% identity with the amino acid sequence of SEQ ID NO: 554, or consists of SEQ ID NO: 554;
63) the light chain variable region comprises the amino acid sequence as set forth in SEQ ID NO: 556, or an amino acid sequence having at least 90%, 91%, 92%, 93%, 94%, 95%, 96%, 97%, 98% or 99% identity with the amino acid sequence of SEQ ID NO: 556, or consists of SEQ ID NO: 556;
64) the light chain variable region comprises the amino acid sequence as set forth in SEQ ID NO: 558, or an amino acid sequence having at least 90%, 91%, 92%, 93%, 94%, 95%, 96%, 97%, 98% or 99% identity with the amino acid sequence of SEQ ID NO: 558, or consists of SEQ ID NO: 558;
65) the light chain variable region comprises the amino acid sequence as set forth in SEQ ID NO: 560, or an amino acid sequence having at least 90%, 91%, 92%, 93%, 94%, 95%, 96%, 97%, 98% or 99% identity with the amino acid sequence of SEQ ID NO: 560, or consists of SEQ ID NO: 560;
66) the light chain variable region comprises the amino acid sequence as set forth in SEQ ID NO: 563, or an amino acid sequence having at least 90%, 91%, 92%, 93%, 94%, 95%, 96%, 97%, 98% or 99% identity with the amino acid sequence of SEQ ID NO: 563, or consists of SEQ ID NO: 563;
67) the light chain variable region comprises the amino acid sequence as set forth in SEQ ID NO: 565, or an amino acid sequence having at least 90%, 91%, 92%, 93%, 94%, 95%, 96%, 97%, 98% or 99% identity with the amino acid sequence of SEQ ID NO: 565, or consists of SEQ ID NO: 565;
68) the light chain variable region comprises the amino acid sequence as set forth in SEQ ID NO: 567, or an amino acid sequence having at least 90%, 91%, 92%, 93%, 94%, 95%, 96%, 97%, 98% or 99% identity with the amino acid sequence of SEQ ID NO: 567, or consists of SEQ ID NO: 567;
69) the light chain variable region comprises the amino acid sequence as set forth in SEQ ID NO: 569, or an amino acid sequence having at least 90%, 91%, 92%, 93%, 94%, 95%, 96%, 97%, 98% or 99% identity with the amino acid sequence of SEQ ID NO: 569, or consists of SEQ ID NO: 569;
70) the light chain variable region comprises the amino acid sequence as set forth in SEQ ID NO: 571, or an amino acid sequence having at least 90%, 91%, 92%, 93%, 94%, 95%, 96%, 97%, 98% or 99% identity with the amino acid sequence of SEQ ID NO: 571, or consists of SEQ ID NO: 571;
71) the light chain variable region comprises the amino acid sequence as set forth in SEQ ID NO: 573, or an amino acid sequence having at least 90%, 91%, 92%, 93%, 94%, 95%, 96%, 97%, 98% or 99% identity with the amino acid sequence of SEQ ID NO: 573, or consists of SEQ ID NO: 573;
72) the light chain variable region comprises the amino acid sequence as set forth in SEQ ID NO: 575, or an amino acid sequence having at least 90%, 91%, 92%, 93%, 94%, 95%, 96%, 97%, 98% or 99% identity with the amino acid sequence of SEQ ID NO: 575, or consists of SEQ ID NO: 575;
73) the light chain variable region comprises the amino acid sequence as set forth in SEQ ID NO: 578, or an amino acid sequence having at least 90%, 91%, 92%, 93%, 94%, 95%, 9%%, 97%, 98% or 99% identity with the amino acid sequence of SEQ ID NO: 578, or consists of SEQ ID NO: 578;
74) the light chain variable region comprises the amino acid sequence as set forth in SEQ ID NO: 580, or an amino acid sequence having at least 90%, 91%, 92%, 93%, 94%, 95%, 96%, 97%, 98% or 99% identity with the amino acid sequence of SEQ ID NO: 580, or consists of SEQ ID NO: 580;
75) the light chain variable region comprises the amino acid sequence as set forth in SEQ ID NO: 582, or an amino acid sequence having at least 90%, 91%, 92%, 93%, 94%, 95%, 96%, 97%, 98% or 99% identity with the amino acid sequence of SEQ ID NO: 582, or consists of SEQ ID NO: 582;
76) the light chain variable region comprises the amino acid sequence as set forth in SEQ ID NO: 584, or an amino acid sequence having at least 90%, 91%, 92%, 93%, 94%, 95%, 96%, 97%, 98% or 99% identity with the amino acid sequence of SEQ ID NO: 584, or consists of SEQ ID NO: 584;
77) the light chain variable region comprises the amino acid sequence as set forth in SEQ ID NO: 586, or an amino acid sequence having at least 90%, 91%, 92%, 93%, 94%, 95%, 96%, 97%, 98% or 99% identity with the amino acid sequence of SEQ ID NO: 586, or consists of SEQ ID NO: 586;
78) the light chain variable region comprises the amino acid sequence as set forth in SEQ ID NO: 588, or an amino acid sequence having at least 90%, 91%, 92%, 93%, 94%, 95%, 96%, 97%, 98% or 99% identity with the amino acid sequence of SEQ ID NO: 588, or consists of SEQ ID NO: 588;
79) the light chain variable region comprises the amino acid sequence as set forth in SEQ ID NO: 589, or an amino acid sequence having at least 90%, 91%, 92%, 93%, 94%, 95%, 96%, 97%, 98% or 99% identity with the amino acid sequence of SEQ ID NO: 589, or consists of SEQ ID NO: 589;
80) the light chain variable region comprises the amino acid sequence as set forth in SEQ ID NO: 591, or an amino acid sequence having at least 90%, 91%, 92%, 93%, 94%, 95%, 96%, 97%, 98% or 99% identity with the amino acid sequence of SEQ ID NO: 591, or consists of SEQ ID NO: 591;
81) the light chain variable region comprises the amino acid sequence as set forth in SEQ ID NO: 593, or an amino acid sequence having at least 90%, 91%, 92%, 93%, 94%, 95%, 96%, 97%, 98% or 99% identity with the amino acid sequence of SEQ ID NO: 593, or consists of SEQ ID NO: 593;
82) the light chain variable region comprises the amino acid sequence as set forth in SEQ ID NO: 594, or an amino acid sequence having at least 90%, 91%, 92%, 93%, 94%, 95%, 96%, 97%, 98% or 99% identity with the amino acid sequence of SEQ ID NO: 594, or consists of SEQ ID NO: 594;
83) the light chain variable region comprises the amino acid sequence as set forth in SEQ ID NO: 596, or an amino acid sequence having at least 90%, 91%, 92%, 93%, 94%, 95%, 96%, 97%, 98% or 99% identity with the amino acid sequence of SEQ ID NO: 596, or consists of SEQ ID NO: 596;
84) the light chain variable region comprises the amino acid sequence as set forth in SEQ ID NO: 598, or an amino acid sequence having at least 90%, 91%, 92%, 93%, 94%, 95%, 96%, 97%, 98% or 99% identity with the amino acid sequence of SEQ ID NO: 598, or consists of SEQ ID NO: 598;
85) the light chain variable region comprises the amino acid sequence as set forth in SEQ ID NO: 600, or an amino acid sequence having at least 90%, 91%, 92%, 93%, 94%, 95%, 96%, 97%, 98% or 99% identity with the amino acid sequence of SEQ ID NO: 600, or consists of SEQ ID NO: 600;
86) the light chain variable region comprises the amino acid sequence as set forth in SEQ ID NO: 602, or an amino acid sequence having at least 90%, 91%, 92%, 93%, 94%, 95%, 96%, 97%, 98% or 99% identity with the amino acid sequence of SEQ ID NO: 602, or consists of SEQ ID NO: 602;
87) the light chain variable region comprises the amino acid sequence as set forth in SEQ ID NO: 604, or an amino acid sequence having at least 90%, 91%, 92%, 93%, 94%, 95%, 96%, 97%, 98% or 99% identity with the amino acid sequence of SEQ ID NO: 604, or consists of SEQ ID NO: 604;
88) the light chain variable region comprises the amino acid sequence as set forth in SEQ ID NO: 606, or an amino acid sequence having at least 90%, 91%, 92%, 93%, 94%, 95%, 96%, 97%, 98% or 99% identity with the amino acid sequence of SEQ ID NO: 606, or consists of SEQ ID NO: 606;
89) the light chain variable region comprises the amino acid sequence as set forth in SEQ ID NO: 608, or an amino acid sequence having at least 90%, 91%, 92%, 93%, 94%, 95%, 96%, 97%, 98% or 99% identity with the amino acid sequence of SEQ ID NO: 608, or consists of SEQ ID NO: 608;
90) the light chain variable region comprises the amino acid sequence as set forth in SEQ ID NO: 610, or an amino acid sequence having at least 90%, 91%, 92%, 93%, 94%, 95%, 96%, 97%, 98% or 99% identity with the amino acid sequence of SEQ ID NO: 610, or consists of SEQ ID NO: 610;
91) the light chain variable region comprises the amino acid sequence as set forth in SEQ ID NO: 612, or an amino acid sequence having at least 90%, 91%, 92%, 93%, 94%, 95%, 96%, 97%, 98% or 99% identity with the amino acid sequence of SEQ ID NO: 612, or consists of SEQ ID NO: 612;
92) the light chain variable region comprises the amino acid sequence as set forth in SEQ ID NO: 614, or an amino acid sequence having at least 90%, 91%, 92%, 93%, 94%, 95%, 96%, 97%, 98% or 99% identity with the amino acid sequence of SEQ ID NO: 614, or consists of SEQ ID NO: 614;
93) the light chain variable region comprises the amino acid sequence as set forth in SEQ ID NO: 616, or an amino acid sequence having at least 90%, 91%, 92%, 93%, 94%, 95%, 96%, 97%, 98% or 99% identity with the amino acid sequence of SEQ ID NO: 616, or consists of SEQ ID NO: 616;
94) the light chain variable region comprises the amino acid sequence as set forth in SEQ ID NO: 618, or an amino acid sequence having at least 90%, 91%, 92%, 93%, 94%, 95%, 96%, 97%, 98% or 99% identity with the amino acid sequence of SEQ ID NO: 618, or consists of SEQ ID NO: 618;
95) the light chain variable region comprises the amino acid sequence as set forth in SEQ ID NO: 620, or an amino acid sequence having at least 90%, 91%, 92%, 93%, 94%, 95%, 9%%, 97%, 98% or 99% identity with the amino acid sequence of SEQ ID NO: 620, or consists of SEQ ID NO: 620;
96) the light chain variable region comprises the amino acid sequence as set forth in SEQ ID NO: 622, or an amino acid sequence having at least 90%, 91%, 92%, 93%, 94%, 95%, 96%, 97%, 98% or 99% identity with the amino acid sequence of SEQ ID NO: 622, or consists of SEQ ID NO: 622;
97) the light chain variable region comprises the amino acid sequence as set forth in SEQ ID NO: 624, or an amino acid sequence having at least 90%, 91%, 92%, 93%, 94%, 95%, 96%, 97%, 98% or 99% identity with the amino acid sequence of SEQ ID NO: 624, or consists of SEQ ID NO: 624;
98) the light chain variable region comprises the amino acid sequence as set forth in SEQ ID NO: 626, or an amino acid sequence having at least 90%, 91%, 92%, 93%, 94%, 95%, 96%, 97%, 98% or 99% identity with the amino acid sequence of SEQ ID NO: 626, or consists of SEQ ID NO: 626;
99) the light chain variable region comprises the amino acid sequence as set forth in SEQ ID NO: 628, or an amino acid sequence having at least 90%, 91%, 92%, 93%, 94%, 95%, 96%, 97%, 98% or 99% identity with the amino acid sequence of SEQ ID NO: 628, or consists of SEQ ID NO: 628;
100) the light chain variable region comprises the amino acid sequence as set forth in SEQ ID NO: 630, or an amino acid sequence having at least 90%, 91%, 92%, 93%, 94%, 95%, 96%, 97%, 98% or 99% identity with the amino acid sequence of SEQ ID NO: 630, or consists of SEQ ID NO: 630;
101) the light chain variable region comprises the amino acid sequence as set forth in SEQ ID NO: 632, or an amino acid sequence having at least 90%, 91%, 92%, 93%, 94%, 95%, 96%, 97%, 98% or 99% identity with the amino acid sequence of SEQ ID NO: 632, or consists of SEQ ID NO: 632;
102) the light chain variable region comprises the amino acid sequence as set forth in SEQ ID NO: 633, or an amino acid sequence having at least 90%, 91%, 92%, 93%, 94%, 95%, 96%, 97%, 98% or 99% identity with the amino acid sequence of SEQ ID NO: 633, or consists of SEQ ID NO: 633;
103) the light chain variable region comprises the amino acid sequence as set forth in SEQ ID NO: 635, or an amino acid sequence having at least 90%, 91%, 92%, 93%, 94%, 95%, 96%, 97%, 98% or 99% identity with the amino acid sequence of SEQ ID NO: 635, or consists of SEQ ID NO: 635;
104) the light chain variable region comprises the amino acid sequence as set forth in SEQ ID NO: 637, or an amino acid sequence having at least 90%, 91%, 92%, 93%, 94%, 95%, 96%, 97%, 98% or 99% identity with the amino acid sequence of SEQ ID NO: 637, or consists of SEQ ID NO: 637;
105) the light chain variable region comprises the amino acid sequence as set forth in SEQ ID NO: 639, or an amino acid sequence having at least 90%, 91%, 92%, 93%, 94%, 95%, 96%, 97%, 98% or 99% identity with the amino acid sequence of SEQ ID NO: 639, or consists of SEQ ID NO: 639;
106) the light chain variable region comprises the amino acid sequence as set forth in SEQ ID NO: 641, or an amino acid sequence having at least 90%, 91%, 92%, 93%, 94%, 95%, 9%%, 97%, 98% or 99% identity with the amino acid sequence of SEQ ID NO: 641, or consists of SEQ ID NO: 641;
107) the light chain variable region comprises the amino acid sequence as set forth in SEQ ID NO: 643, or an amino acid sequence having at least 90%, 91%, 92%, 93%, 94%, 95%, 96%, 97%, 98% or 99% identity with the amino acid sequence of SEQ ID NO: 643, or consists of SEQ ID NO: 643;
108) the light chain variable region comprises the amino acid sequence as set forth in SEQ ID NO: 645, or an amino acid sequence having at least 90%, 91%, 92%, 93%, 94%, 95%, 96%, 97%, 98% or 99% identity with the amino acid sequence of SEQ ID NO: 645, or consists of SEQ ID NO: 645;
109) the light chain variable region comprises the amino acid sequence as set forth in SEQ ID NO: 647, or an amino acid sequence having at least 90%, 91%, 92%, 93%, 94%, 95%, 96%, 97%, 98% or 99% identity with the amino acid sequence of SEQ ID NO: 647, or consists of SEQ ID NO: 647;
110) the light chain variable region comprises the amino acid sequence as set forth in SEQ ID NO: 649, or an amino acid sequence having at least 90%, 91%, 92%, 93%, 94%, 95%, 96%, 97%, 98% or 99% identity with the amino acid sequence of SEQ ID NO: 649, or consists of SEQ ID NO: 649;
11l) the light chain variable region comprises the amino acid sequence as set forth in SEQ ID NO: 651, or an amino acid sequence having at least 90%, 91%, 92%, 93%, 94%, 95%, 96%, 97%, 98% or 99% identity with the amino acid sequence of SEQ ID NO: 651, or consists of SEQ ID NO: 651;
112) the light chain variable region comprises the amino acid sequence as set forth in SEQ ID NO: 653, or an amino acid sequence having at least 90%, 91%, 92%, 93%, 94%, 95%, %%, 97% 98% or 99% identity to the amino acid sequence of SEQ ID NO: 653, or consists of SEQ ID NO: 653; or
113) The light chain variable region comprises the amino acid sequence as set forth in SEQ ID NO: 655, or an amino acid sequence having at least 90%, 91%, 92%, 93%, 94% 95%, 96% 97%, 98% or 99% identity to the amino acid sequence of SEQ ID NO: 655, or consists of SEQ ID NO: 655.

In another embodiment, the invention provides an anti-CD112R antibody that specifically binds CD112R and an antigen-binding fragment thereof, comprising a heavy chain variable region and a light chain variable region, wherein:
1) the heavy chain variable region comprises the amino acid sequence as set forth in SEQ ID NO: 430, or an amino acid sequence having at least 90%, 91%, 92%, 93%, 94%, 95%, 9%%, 97%, 98% or 99% identity with the amino acid sequence of SEQ ID NO: 430, or consists of SEQ ID NO: 430, and the light chain variable region comprises the amino acid sequence as set forth in SEQ ID NO: 431, or an amino acid sequence having at least 90%, 91%, 92%, 93%, 94%, 95%, 96%, 97%, 98% or 99% identity with the amino acid sequence of SEQ ID NO: 431, or consists of SEQ ID NO: 431;
2) the heavy chain variable region comprises the amino acid sequence as set forth in SEQ ID NO: 432, or an amino acid sequence having at least 90%, 91%, 92%, 93%, 94%, 95%, 96%, 97%, 98% or 99% identity with the amino acid sequence of SEQ ID NO: 432, or consists of SEQ ID NO: 432, and the light chain variable region comprises the amino acid sequence as set forth in SEQ ID NO: 433, or an amino acid sequence having at least 90%, 91%, 92%, 93%, 94%, 95%, 96%, 97%, 98% or 99% identity with the amino acid sequence of SEQ ID NO: 433, or consists of SEQ ID NO: 433;
3) the heavy chain variable region comprises the amino acid sequence as set forth in SEQ ID NO: 434, or an amino acid sequence having at least 90%, 91%, 92%, 93%, 94%, 95%, 96%, 97%, 98% or 99% identity with the amino acid sequence of SEQ ID NO: 434, or consists of SEQ ID NO: 434, and the light chain variable region comprises the amino acid sequence as set forth in SEQ ID NO: 435, or an amino acid sequence having at least 90%, 91%, 92%, 93%, 94%, 95%, 96%, 97%, 98% or 99% identity with the amino acid sequence of SEQ ID NO: 435, or consists of SEQ ID NO: 435;
4) the heavy chain variable region comprises the amino acid sequence as set forth in SEQ ID NO: 436, or an amino acid sequence having at least 90%, 91%, 92%, 93%, 94%, 95%, 96%, 97%, 98% or 99% identity with the amino acid sequence of SEQ ID NO: 436, or consists of SEQ ID NO: 436, and the light chain variable region comprises the amino acid sequence as set forth in SEQ ID NO: 437, or an amino acid sequence having at least 90%, 91%, 92%, 93%, 94%, 95%, 96%, 97%, 98% or 99% identity with the amino acid sequence of SEQ ID NO: 437, or consists of SEQ ID NO: 437;
5) the heavy chain variable region comprises the amino acid sequence as set forth in SEQ ID NO: 438, or an amino acid sequence having at least 90%, 91%, 92%, 93%, 94%, 95%, 96%, 97%, 98% or 99% identity with the amino acid sequence of SEQ ID NO: 438, or consists of SEQ ID NO: 438, and the light chain variable region comprises the amino acid sequence as set forth in SEQ ID NO: 439, or an amino acid sequence having at least 90%, 91%, 92%, 93%, 94%, 95%, 96%, 97%, 98% or 99% identity with the amino acid sequence of SEQ ID NO: 439, or consists of SEQ ID NO: 439;
6) the heavy chain variable region comprises the amino acid sequence as set forth in SEQ ID NO: 438, or an amino acid sequence having at least 90%, 91%, 92%, 93%, 94%, 95%, 9%%, 97%, 98% or 99% identity with the amino acid sequence of SEQ ID NO: 438, or consists of SEQ ID NO: 438, and the light chain variable region comprises the amino acid sequence as set forth in SEQ ID NO: 440, or an amino acid sequence having at least 90%, 91%, 92%, 93%, 94%, 95%, 96%, 97%, 98% or 99% identity with the amino acid sequence of SEQ ID NO: 440, or consists of SEQ ID NO: 440;
7) the heavy chain variable region comprises the amino acid sequence as set forth in SEQ ID NO: 441, or an amino acid sequence having at least 90%, 91%, 92%, 93%, 94%, 95%, 96%, 97%, 98% or 99% identity with the amino acid sequence of SEQ ID NO: 441, or consists of SEQ ID NO: 441, and the light chain variable region comprises the amino acid sequence as set forth in SEQ ID NO: 442, or an amino acid sequence having at least 90%, 91%, 92%, 93%, 94%, 95%, 96%, 97%, 98% or 99% identity with the amino acid sequence of SEQ ID NO: 442, or consists of SEQ ID NO: 442;
8) the heavy chain variable region comprises the amino acid sequence as set forth in SEQ ID NO: 443, or an amino acid sequence having at least 90%, 91%, 92%, 93%, 94%, 95%, 96%, 97%, 98% or 99% identity with the amino acid sequence of SEQ ID NO: 443, or consists of SEQ ID NO: 443, and the light chain variable region comprises the amino acid sequence as set forth in SEQ ID NO: 444, or an amino acid sequence having at least 90%, 91%, 92%, 93%, 94%, 95%, 96%, 97%, 98% or 99% identity with the amino acid sequence of SEQ ID NO: 444, or consists of SEQ ID NO: 444;
9) the heavy chain variable region comprises the amino acid sequence as set forth in SEQ ID NO: 445, or an amino acid sequence having at least 90%, 91%, 92%, 93%, 94%, 95%, 96%, 97%, 98% or 99% identity with the amino acid sequence of SEQ ID NO: 445, or consists of SEQ ID NO: 445, and the light chain variable region comprises the amino acid sequence as set forth in SEQ ID NO: 446, or an amino acid sequence having at least 90%, 91%, 92%, 93%, 94%, 95%, 9%, 97%, 98% or 99% identity with the amino acid sequence of SEQ ID NO: 446, or consists of SEQ ID NO: 446;
10) the heavy chain variable region comprises the amino acid sequence as set forth in SEQ ID NO: 447, or an amino acid sequence having at least 90%, 91%, 92%, 93%, 94%, 95%, 96%, 97%, 98% or 99% identity with the amino acid sequence of SEQ ID NO: 447, or consists of SEQ ID NO: 447, and the light chain variable region comprises the amino acid sequence as set forth in SEQ ID NO: 448, or an amino acid sequence having at least 90%, 91%, 92%, 93%, 94%, 95%, 96%, 97%, 98% or 99% identity with the amino acid sequence of SEQ ID NO: 448, or consists of SEQ ID NO: 448;
11) the heavy chain variable region comprises the amino acid sequence as set forth in SEQ ID NO: 449, or an amino acid sequence having at least 90%, 91%, 92%, 93%, 94%, 95%, 96%, 97%, 98% or 99% identity with the amino acid sequence of SEQ ID NO: 449, or consists of SEQ ID NO: 449, and the light chain variable region comprises the amino acid sequence as set forth in SEQ ID NO: 450, or an amino acid sequence having at least 90%, 91%, 92%, 93%, 94%, 95%, 96%, 97%, 98% or 99% identity with the amino acid sequence of SEQ ID NO: 450, or consists of SEQ ID NO: 450;
12) the heavy chain variable region comprises the amino acid sequence as set forth in SEQ ID NO: 451, or an amino acid sequence having at least 90%, 91%, 92%, 93%, 94%, 95%, 96%, 97%, 98% or 99% identity with the amino acid sequence of SEQ ID NO: 451, or consists of SEQ ID NO: 451, and the light chain variable region comprises the amino acid sequence as set forth in SEQ ID NO: 452, or an amino acid sequence having at least 90%, 91%, 92%, 93%, 94%, 95%, 96%, 97%, 98% or 99% identity with the amino acid sequence of SEQ ID NO: 452, or consists of SEQ ID NO: 452;
13) the heavy chain variable region comprises the amino acid sequence as set forth in SEQ ID NO: 453, or an amino acid sequence having at least 90%, 91%, 92%, 93%, 94%, 95%, 96%, 97%, 98% or 99% identity with the amino acid sequence of SEQ ID NO: 453, or consists of SEQ ID NO: 453, and the light chain variable region comprises the amino acid sequence as set forth in SEQ ID NO: 454, or an amino acid sequence having at least 90%, 91%, 92%, 93%, 94%, 95%, 96%, 97%, 98% or 99% identity with the amino acid sequence of SEQ ID NO: 454, or consists of SEQ ID NO: 454;
14) the heavy chain variable region comprises the amino acid sequence as set forth in SEQ ID NO: 455, or an amino acid sequence having at least 90%, 91%, 92%, 93%, 94%, 95%, 96%, 97%, 98% or 99% identity with the amino acid sequence of SEQ ID NO: 455, or consists of SEQ ID NO: 455, and the light chain variable region comprises the amino acid sequence as set forth in SEQ ID NO: 456, or an amino acid sequence having at least 90%, 91%, 92%, 93%, 94%, 95%, 96%, 97%, 98% or 99% identity with the amino acid sequence of SEQ ID NO: 456, or consists of SEQ ID NO: 456;
15) the heavy chain variable region comprises the amino acid sequence as set forth in SEQ ID NO: 457, or an amino acid sequence having at least 90%, 91%, 92%, 93%, 94%, 95%, 96%, 97%, 98% or 99% identity with the amino acid sequence of SEQ ID NO: 457, or consists of SEQ ID NO: 457, and the light chain variable region comprises the amino acid sequence as set forth in SEQ ID NO: 458, or an amino acid sequence having at least 90%, 91%, 92%, 93%, 94%, 95%, 96%, 97%, 98% or 99% identity with the amino acid sequence of SEQ ID NO: 458, or consists of SEQ ID NO: 458;
16) the heavy chain variable region comprises the amino acid sequence as set forth in SEQ ID NO: 459, or an amino acid sequence having at least 90%, 91%, 92%, 93%, 94%, 95%, 96%, 97%, 98% or 99% identity with the amino acid sequence of SEQ ID NO: 459, or consists of SEQ ID NO: 459, and the light chain variable region comprises the amino acid sequence as set forth in SEQ ID NO: 460, or an amino acid sequence having at least 90%, 91%, 92%, 93%, 94%, 95%, 96%, 97%, 98% or 99% identity with the amino acid sequence of SEQ ID NO: 460, or consists of SEQ ID NO: 460;
17) the heavy chain variable region comprises the amino acid sequence as set forth in SEQ ID NO: 461, or an amino acid sequence having at least 90%, 91%, 92%, 93%, 94%, 95%, 96%, 97%, 98% or 99% identity with the amino acid sequence of SEQ ID NO: 461, or consists of SEQ ID NO: 461, and the light chain variable region comprises the amino acid sequence as set forth in SEQ ID NO: 462, or an amino acid sequence having at least 90%, 91%, 92%, 93%, 94%, 95%, 96%, 97%, 98% or 99% identity with the amino acid sequence of SEQ ID NO: 462, or consists of SEQ ID NO: 462;
18) the heavy chain variable region comprises the amino acid sequence as set forth in SEQ ID NO: 463, or an amino acid sequence having at least 90%, 91%, 92%, 93%, 94%, 95%, 9%%, 97%, 98% or 99% identity with the amino acid sequence of SEQ ID NO: 463, or consists of SEQ ID NO: 463, and the light chain variable region comprises the amino acid sequence as set forth in SEQ ID NO: 464, or an amino acid sequence having at least 90%, 91%, 92%, 93%, 94%, 95%, 96%, 97%, 98% or 99% identity with the amino acid sequence of SEQ ID NO: 464, or consists of SEQ ID NO: 464;
19) the heavy chain variable region comprises the amino acid sequence as set forth in SEQ ID NO: 465, or an amino acid sequence having at least 90%, 91%, 92%, 93%, 94%, 95%, 96%, 97%, 98% or 99% identity with the amino acid sequence of SEQ ID NO: 465, or consists of SEQ ID NO: 465, and the light chain variable region comprises the amino acid sequence as set forth in SEQ ID NO: 466, or an amino acid sequence having at least 90%, 91%, 92%, 93%, 94%, 95%, 96%, 97%, 98% or 99% identity with the amino acid sequence of SEQ ID NO: 466, or consists of SEQ ID NO: 466;
20) the heavy chain variable region comprises the amino acid sequence as set forth in SEQ ID NO: 467, or an amino acid sequence having at least 90%, 91%, 92%, 93%, 94%, 95%, 96%, 97%, 98% or 99% identity with the amino acid sequence of SEQ ID NO: 467, or consists of SEQ ID NO: 467, and the light chain variable region comprises the amino acid sequence as set forth in SEQ ID NO: 468, or an amino acid sequence having at least 90%, 91%, 92%, 93%, 94%, 95%, 96%, 97%, 98% or 99% identity with the amino acid sequence of SEQ ID NO: 468, or consists of SEQ ID NO: 468;
21) the heavy chain variable region comprises the amino acid sequence as set forth in SEQ ID NO: 469, or an amino acid sequence having at least 90%, 91%, 92%, 93%, 94%, 95%, 96%, 97%, 98% or 99% identity with the amino acid sequence of SEQ ID NO: 469, or consists of SEQ ID NO: 469, and the light chain variable region comprises the amino acid sequence as set forth in SEQ ID NO: 470, or an amino acid sequence having at least 90%, 91%, 92%, 93%, 94%, 95%, 96%, 97%, 98% or 99% identity with the amino acid sequence of SEQ ID NO: 470, or consists of SEQ ID NO: 470;
22) the heavy chain variable region comprises the amino acid sequence as set forth in SEQ ID NO: 471, or an amino acid sequence having at least 90%, 91%, 92%, 93%, 94%, 95%, 96%, 97%, 98% or 99% identity with the amino acid sequence of SEQ ID NO: 471, or consists of SEQ ID NO: 471, and the light chain variable region comprises the amino acid sequence as set forth in SEQ ID NO: 472, or an amino acid sequence having at least 90%, 91%, 92%, 93%, 94%, 95%, 96%, 97%, 98% or 99% identity with the amino acid sequence of SEQ ID NO: 472, or consists of SEQ ID NO: 472;
23) the heavy chain variable region comprises the amino acid sequence as set forth in SEQ ID NO: 473, or an amino acid sequence having at least 90%, 91%, 92%, 93%, 94%, 95%, 9%%, 97%, 98% or 99% identity with the amino acid sequence of SEQ ID NO: 473, or consists of SEQ ID NO: 473, and the light chain variable region comprises the amino acid sequence as set forth in SEQ ID NO: 474, or an amino acid sequence having at least 90%, 91%, 92%, 93%, 94%, 95%, 96%, 97%, 98% or 99% identity with the amino acid sequence of SEQ ID NO: 474, or consists of SEQ ID NO: 474;
24) the heavy chain variable region comprises the amino acid sequence as set forth in SEQ ID NO: 475, or an amino acid sequence having at least 90%, 91%, 92%, 93%, 94%, 95%, 96%, 97%, 98% or 99% identity with the amino acid sequence of SEQ ID NO: 475, or consists of SEQ ID NO: 475, and the light chain variable region comprises the amino acid sequence as set forth in SEQ ID NO: 476, or an amino acid sequence having at least 90%, 91%, 92%, 93%, 94%, 95%, 96%, 97%, 98% or 99% identity with the amino acid sequence of SEQ ID NO: 476, or consists of SEQ ID NO: 476;
25) the heavy chain variable region comprises the amino acid sequence as set forth in SEQ ID NO: 477, or an amino acid sequence having at least 90%, 91%, 92%, 93%, 94%, 95%, 96%, 97%, 98% or 99% identity with the amino acid sequence of SEQ ID NO: 477, or consists of SEQ ID NO: 477, and the light chain variable region comprises the amino acid sequence as set forth in SEQ ID NO: 478, or an amino acid sequence having at least 90%, 91%, 92%, 93%, 94%, 95%, 96%, 97%, 98% or 99% identity with the amino acid sequence of SEQ ID NO: 478, or consists of SEQ ID NO: 478;
26) the heavy chain variable region comprises the amino acid sequence as set forth in SEQ ID NO: 479, or an amino acid sequence having at least 90%, 91%, 92%, 93%, 94%, 95%, 96%, 97%, 98% or 99% identity to the amino acid sequence of SEQ ID NO: 479, or consists of SEQ ID NO: 479, and the light chain variable region comprises the amino acid sequence as set forth in SEQ ID NO: 480, or an amino acid sequence having at least 90%, 91%, 92%, 93%, 94%, 95%, 96%, 97%, 98% or 99% identity to the amino acid sequence of SEQ ID NO: 480, or consists of SEQ ID NO: 480; or
27) The heavy chain variable region comprises the amino acid sequence as set forth in SEQ ID NO: 481, or an amino acid sequence having at least 90%, 91%, 92%, 93%, 94%, 95%, 96%, 97%, 98% or 99% identity to the amino acid sequence of SEQ ID NO: 481, or consists of SEQ ID NO: 481, and the light chain variable region comprises the amino acid sequence as set forth in SEQ ID NO: 482, or an amino acid sequence having at least 90%, 91%, 92%, 93%, 94%, 95%, 96%, 97%, 98% or 99% identity to the amino acid sequence of SEQ ID NO: 482, or consists of SEQ ID NO: 482.
28) the heavy chain variable region comprises the amino acid sequence as set forth in SEQ ID NO: 483, or an amino acid sequence having at least 90%, 91%, 92%, 93%, 94%, 95%, 96%, 97%, 98% or 99% identity with the amino acid sequence of SEQ ID NO: 483, or consists of SEQ ID NO: 483, and the light chain variable region comprises the amino acid sequence as set forth in SEQ ID NO: 484, or an amino acid sequence having at least 90%, 91%, 92%, 93%, 94%, 95%, 96%, 97%, 98% or 99% identity with the amino acid sequence of SEQ ID NO: 484, or consists of SEQ ID NO: 484;
29) the heavy chain variable region comprises the amino acid sequence as set forth in SEQ ID NO: 485, or an amino acid sequence having at least 90%, 91%, 92%, 93%, 94%, 95%, 96%, 97%, 98% or 99% identity with the amino acid sequence of SEQ ID NO: 485, or consists of SEQ ID NO: 485, and the light chain variable region comprises the amino acid sequence as set forth in SEQ ID NO: 486, or an amino acid sequence having at least 90%, 91%, 92%, 93%, 94%, 95%, 96%, 97%, 98% or 99% identity with the amino acid sequence of SEQ ID NO: 486, or consists of SEQ ID NO: 486;
30) the heavy chain variable region comprises the amino acid sequence as set forth in SEQ ID NO: 487, or an amino acid sequence having at least 90%, 91%, 92%, 93%, 94%, 95%, 96%, 97%, 98% or 99% identity with the amino acid sequence of SEQ ID NO: 487, or consists of SEQ ID NO: 487, and the light chain variable region comprises the amino acid sequence as set forth in SEQ ID NO: 456, or an amino acid sequence having at least 90%, 91%, 92%, 93%, 94%, 95%, 96%, 97%, 98% or 99% identity with the amino acid sequence of SEQ ID NO: 456, or consists of SEQ ID NO: 456;
31) the heavy chain variable region comprises the amino acid sequence as set forth in SEQ ID NO: 488, or an amino acid sequence having at least 90%, 91%, 92%, 93%, 94%, 95%, 96%, 97%, 98% or 99% identity with the amino acid sequence of SEQ ID NO: 488, or consists of SEQ ID NO: 488, and the light chain variable region comprises the amino acid sequence as set forth in SEQ ID NO: 489, or an amino acid sequence having at least 90%, 91%, 92%, 93%, 94%, 95%, 96%, 97%, 98% or 99% identity with the amino acid sequence of SEQ ID NO: 489, or consists of SEQ ID NO: 489;
32) the heavy chain variable region comprises the amino acid sequence as set forth in SEQ ID NO: 490, or an amino acid sequence having at least 90%, 91%, 92%, 93%, 94%, 95%, 96%, 97%, 98% or 99% identity with the amino acid sequence of SEQ ID NO: 490, or consists of SEQ ID NO: 490, and the light chain variable region comprises the amino acid sequence as set forth in SEQ ID NO: 491, or an amino acid sequence having at least 90%, 91%, 92%, 93%, 94%, 95%, 96%, 97%, 98% or 99% identity with the amino acid sequence of SEQ ID NO: 491, or consists of SEQ ID NO: 491;
33) the heavy chain variable region comprises the amino acid sequence as set forth in SEQ ID NO: 492, or an amino acid sequence having at least 90%, 91%, 92%, 93%, 94%, 95%, 96%, 97%, 98% or 99% identity with the amino acid sequence of SEQ ID NO: 492, or consists of SEQ ID NO: 492, and the light chain variable region comprises the amino acid sequence as set forth in SEQ ID NO: 489, or an amino acid sequence having at least 90%, 91%, 92%, 93%, 94%, 95%, 96%, 97%, 98% or 99% identity with the amino acid sequence of SEQ ID NO: 489, or consists of SEQ ID NO: 489;
34) the heavy chain variable region comprises the amino acid sequence as set forth in SEQ ID NO: 493, or an amino acid sequence having at least 90%, 91%, 92%, 93%, 94%, 95%, 96%, 97%, 98% or 99% identity with the amino acid sequence of SEQ ID NO: 493, or consists of SEQ ID NO: 493, and the light chain variable region comprises the amino acid sequence as set forth in SEQ ID NO: 494, or an amino acid sequence having at least 90%, 91%, 92%, 93%, 94%, 95%, 96%, 97%, 98% or 99% identity with the amino acid sequence of SEQ ID NO: 494, or consists of SEQ ID NO: 494;
35) the heavy chain variable region comprises the amino acid sequence as set forth in SEQ ID NO: 493, or an amino acid sequence having at least 90%, 91%, 92%, 93%, 94%, 95%, 96%, 97%, 98% or 99% identity with the amino acid sequence of SEQ ID NO: 493, or consists of SEQ ID NO: 493, and the light chain variable region comprises the amino acid sequence as set forth in SEQ ID NO: 495, or an amino acid sequence having at least 90%, 91%, 92%, 93%, 94%, 95%, 96%, 97%, 98% or 99% identity with the amino acid sequence of SEQ ID NO: 495, or consists of SEQ ID NO: 495;
36) the heavy chain variable region comprises the amino acid sequence as set forth in SEQ ID NO: 496, or an amino acid sequence having at least 90%, 91%, 92%, 93%, 94%, 95%, 96%, 97%, 98% or 99% identity with the amino acid sequence of SEQ ID NO: 496, or consists of SEQ ID NO: 496, and the light chain variable region comprises the amino acid sequence as set forth in SEQ ID NO: 497, or an amino acid sequence having at least 90%, 91%, 92%, 93%, 94%, 95%, 96%, 97%, 98% or 99% identity with the amino acid sequence of SEQ ID NO: 497, or consists of SEQ ID NO: 497;
37) the heavy chain variable region comprises the amino acid sequence as set forth in SEQ ID NO: 498, or an amino acid sequence having at least 90%, 91%, 92%, 93%, 94%, 95%, 96%, 97%, 98% or 99% identity with the amino acid sequence of SEQ ID NO: 498, or consists of SEQ ID NO: 498, and the light chain variable region comprises the amino acid sequence as set forth in SEQ ID NO: 497, or an amino acid sequence having at least 90%, 91%, 92%, 93%, 94%, 95%, 96%, 97%, 98% or 99% identity with the amino acid sequence of SEQ ID NO: 497, or consists of SEQ ID NO: 497;
38) the heavy chain variable region comprises the amino acid sequence as set forth in SEQ ID NO: 499, or an amino acid sequence having at least 90%, 91%, 92%, 93%, 94%, 95%, 96%, 97%, 98% or 99% identity with the amino acid sequence of SEQ ID NO: 499, or consists of SEQ ID NO: 499, and the light chain variable region comprises the amino acid sequence as set forth in SEQ ID NO: 500, or an amino acid sequence having at least 90%, 91%, 92%, 93%, 94%, 95%, 96%, 97%, 98% or 99% identity with the amino acid sequence of SEQ ID NO: 500, or consists of SEQ ID NO: 500;
39) the heavy chain variable region comprises the amino acid sequence as set forth in SEQ ID NO: 501, or an amino acid sequence having at least 90%, 91%, 92%, 93%, 94%, 95%, 96%, 97%, 98% or 99% identity with the amino acid sequence of SEQ ID NO: 501, or consists of SEQ ID NO: 501, and the light chain variable region comprises the amino acid sequence as set forth in SEQ ID NO: 503, or an amino acid sequence having at least 90%, 91%, 92%, 93%, 94%, 95%, 96%, 97%, 98% or 99% identity with the amino acid sequence of SEQ ID NO: 503, or consists of SEQ ID NO: 503;
40) the heavy chain variable region comprises the amino acid sequence as set forth in SEQ ID NO: 502, or an amino acid sequence having at least 90%, 91%, 92%, 93%, 94%, 95%, 96%, 97%, 98% or 99% identity with the amino acid sequence of SEQ ID NO: 502, or consists of SEQ ID NO: 502, and the light chain variable region comprises the amino acid sequence as set forth in SEQ ID NO: 503, or an amino acid sequence having at least 90%, 91%, 92%, 93%, 94%, 95%, 96%, 97%, 98% or 99% identity with the amino acid sequence of SEQ ID NO: 503, or consists of SEQ ID NO: 503;
41) the heavy chain variable region comprises the amino acid sequence as set forth in SEQ ID NO: 504, or an amino acid sequence having at least 90%, 91%, 92%, 93%, 94%, 95%, 96%, 97%, 98% or 99% identity with the amino acid sequence of SEQ ID NO: 504, or consists of SEQ ID NO: 504, and the light chain variable region comprises the amino acid sequence as set forth in SEQ ID NO: 505, or an amino acid sequence having at least 90%, 91%, 92%, 93%, 94%, 95%, 96%, 97%, 98% or 99% identity with the amino acid sequence of SEQ ID NO: 505, or consists of SEQ ID NO: 505;
42) the heavy chain variable region comprises the amino acid sequence as set forth in SEQ ID NO: 499, or an amino acid sequence having at least 90%, 91%, 92%, 93%, 94%, 95%, 96%, 97%, 98% or 99% identity with the amino acid sequence of SEQ ID NO: 499, or consists of SEQ ID NO: 499, and the light chain variable region comprises the amino acid sequence as set forth in SEQ ID NO: 506, or an amino acid sequence having at least 90%, 91%, 92%, 93%, 94%, 95%, 96%, 97%, 98% or 99% identity with the amino acid sequence of SEQ ID NO: 506, or consists of SEQ ID NO: 506;
43) the heavy chain variable region comprises the amino acid sequence as set forth in SEQ ID NO: 507, or an amino acid sequence having at least 90%, 91%, 92%, 93%, 94%, 95%, 9%%, 97%, 98% or 99% identity with the amino acid sequence of SEQ ID NO: 507, or consists of SEQ ID NO: 507, and the light chain variable region comprises the amino acid sequence as set forth in SEQ ID NO: 503, or an amino acid sequence having at least 90%, 91%, 92%, 93%, 94%, 95%, 96%, 97%, 98% or 99% identity with the amino acid sequence of SEQ ID NO: 503, or consists of SEQ ID NO: 503;
44) the heavy chain variable region comprises the amino acid sequence as set forth in SEQ ID NO: 508, or an amino acid sequence having at least 90%, 91%, 92%, 93%, 94%, 95%, 96%, 97%, 98% or 99% identity with the amino acid sequence of SEQ ID NO: 508, or consists of SEQ ID NO: 508, and the light chain variable region comprises the amino acid sequence as set forth in SEQ ID NO: 509, or an amino acid sequence having at least 90%, 91%, 92%, 93%, 94%, 95%, 96%, 97%, 98% or 99% identity with the amino acid sequence of SEQ ID NO: 509, or consists of SEQ ID NO: 509;
45) the heavy chain variable region comprises the amino acid sequence as set forth in SEQ ID NO: 510, or an amino acid sequence having at least 90%, 91%, 92%, 93%, 94%, 95%, 96%, 97%, 98% or 99% identity with the amino acid sequence of SEQ ID NO: 510, or consists of SEQ ID NO: 510, and the light chain variable region comprises the amino acid sequence as set forth in SEQ ID NO: 511, or an amino acid sequence having at least 90%, 91%, 92%, 93%, 94%, 95%, 96%, 97%, 98% or 99% identity with the amino acid sequence of SEQ ID NO: 511, or consists of SEQ ID NO: 511;
46) the heavy chain variable region comprises the amino acid sequence as set forth in SEQ ID NO: 512, or an amino acid sequence having at least 90%, 91%, 92%, 93%, 94%, 95%, 96%, 97%, 98% or 99% identity to the amino acid sequence of SEQ ID NO: 512, or consists of SEQ ID NO: 512, and the light chain variable region comprises the amino acid sequence as set forth in SEQ ID NO: 513, or an amino acid sequence having at least 90%, 91%, 92%, 93%, 94%, 95%, 96%, 97%, 98% or 99% identity to the amino acid sequence of SEQ ID NO: 513, or consists of SEQ ID NO: 513; or
47) The heavy chain variable region comprises the amino acid sequence as set forth in SEQ ID NO: 514, or an amino acid sequence having at least 90%, 91%, 92%, 93%, 94%, 95%, 96%, 97%, 98% or 99% identity to the amino acid sequence of SEQ ID NO: 514, or consists of SEQ ID NO: 514, and the light chain variable region comprises the amino acid sequence as set forth in SEQ ID NO: 515, or an amino acid sequence having at least 90%, 91%, 92%, 93%, 94%, 95%, 96%, 97%, 98% or 99% identity to the amino acid sequence of SEQ ID NO: 515, or consists of SEQ ID NO: 515.
48) the heavy chain variable region comprises the amino acid sequence as set forth in SEQ ID NO: 501, or an amino acid sequence having at least 90%, 91%, 92%, 93%, 94%, 95%, 96%, 97%, 98% or 99% identity with the amino acid sequence of SEQ ID NO: 501, or consists of SEQ ID NO: 501, and the light chain variable region comprises the amino acid sequence as set forth in SEQ ID NO: 516, or an amino acid sequence having at least 90%, 91%, 92%, 93%, 94%, 95%, 96%, 97%, 98% or 99% identity with the amino acid sequence of SEQ ID NO: 516, or consists of SEQ ID NO: 516;
49) the heavy chain variable region comprises the amino acid sequence as set forth in SEQ ID NO: 517, or an amino acid sequence having at least 90%, 91%, 92%, 93%, 94%, 95%, 96%, 97%, 98% or 99% identity with the amino acid sequence of SEQ ID NO: 517, or consists of SEQ ID NO: 517, and the light chain variable region comprises the amino acid sequence as set forth in SEQ ID NO: 518, or an amino acid sequence having at least 90%, 91%, 92%, 93%, 94%, 95%, 96%, 97%, 98% or 99% identity with the amino acid sequence of SEQ ID NO: 518, or consists of SEQ ID NO: 518;
50) the heavy chain variable region comprises the amino acid sequence as set forth in SEQ ID NO: 519, or an amino acid sequence having at least 90%, 91%, 92%, 93%, 94%, 95%, 96%, 97%, 98% or 99% identity with the amino acid sequence of SEQ ID NO: 519, or consists of SEQ ID NO: 519, and the light chain variable region comprises the amino acid sequence as set forth in SEQ ID NO: 520, or an amino acid sequence having at least 90%, 91%, 92%, 93%, 94%, 95%, 96%, 97%, 98% or 99% identity with the amino acid sequence of SEQ ID NO: 520, or consists of SEQ ID NO: 520;
51) the heavy chain variable region comprises the amino acid sequence as set forth in SEQ ID NO: 521, or an amino acid sequence having at least 90%, 91%, 92%, 93%, 94%, 95%, 96%, 97%, 98% or 99% identity with the amino acid sequence of SEQ ID NO: 521, or consists of SEQ ID NO: 521, and the light chain variable region comprises the amino acid sequence as set forth in SEQ ID NO: 522, or an amino acid sequence having at least 90%, 91%, 92%, 93%, 94%, 95%, 96%, 97%, 98% or 99% identity with the amino acid sequence of SEQ ID NO: 522, or consists of SEQ ID NO: 522;
52) the heavy chain variable region comprises the amino acid sequence as set forth in SEQ ID NO: 523, or an amino acid sequence having at least 90%, 91%, 92%, 93%, 94%, 95%, 96%, 97%, 98% or 99% identity with the amino acid sequence of SEQ ID NO: 523, or consists of SEQ ID NO: 523, and the light chain variable region comprises the amino acid sequence as set forth in SEQ ID NO: 524, or an amino acid sequence having at least 90%, 91%, 92%, 93%, 94%, 95%, 96%, 97%, 98% or 99% identity with the amino acid sequence of SEQ ID NO: 524, or consists of SEQ ID NO: 524;
53) the heavy chain variable region comprises the amino acid sequence as set forth in SEQ ID NO: 525, or an amino acid sequence having at least 90%, 91%, 92%, 93%, 94%, 95%, 96%, 97%, 98% or 99% identity with the amino acid sequence of SEQ ID NO: 525, or consists of SEQ ID NO: 525, and the light chain variable region comprises the amino acid sequence as set forth in SEQ ID NO: 486, or an amino acid sequence having at least 90%, 91%, 92%, 93%, 94%, 95%, 96%, 97%, 98% or 99% identity with the amino acid sequence of SEQ ID NO: 486, or consists of SEQ ID NO: 486;
54) the heavy chain variable region comprises the amino acid sequence as set forth in SEQ ID NO: 526, or an amino acid sequence having at least 90%, 91%, 92%, 93%, 94%, 95%, 96%, 97%, 98% or 99% identity with the amino acid sequence of SEQ ID NO: 526, or consists of SEQ ID NO: 526, and the light chain variable region comprises the amino acid sequence as set forth in SEQ ID NO: 527, or an amino acid sequence having at least 90%, 91%, 92%, 93%, 94%, 95%, 96%, 97%, 98% or 99% identity with the amino acid sequence of SEQ ID NO: 527, or consists of SEQ ID NO: 527;
55) the heavy chain variable region comprises the amino acid sequence as set forth in SEQ ID NO: 528, or an amino acid sequence having at least 90%, 91%, 92%, 93%, 94%, 95%, 96%, 97%, 98% or 99% identity with the amino acid sequence of SEQ ID NO: 528, or consists of SEQ ID NO: 528, and the light chain variable region comprises the amino acid sequence as set forth in SEQ ID NO: 529, or an amino acid sequence having at least 90%, 91%, 92%, 93%, 94%, 95%, 96%, 97%, 98% or 99% identity with the amino acid sequence of SEQ ID NO: 529, or consists of SEQ ID NO: 529;
56) the heavy chain variable region comprises the amino acid sequence as set forth in SEQ ID NO: 530, or an amino acid sequence having at least 90%, 91%, 92%, 93%, 94%, 95%, 9%%, 97%, 98% or 99% identity with the amino acid sequence of SEQ ID NO: 530, or consists of SEQ ID NO: 530, and the light chain variable region comprises the amino acid sequence as set forth in SEQ ID NO: 531, or an amino acid sequence having at least 90%, 91%, 92%, 93%, 94%, 95%, 96%, 97%, 98% or 99% identity with the amino acid sequence of SEQ ID NO: 531, or consists of SEQ ID NO: 531;
57) the heavy chain variable region comprises the amino acid sequence as set forth in SEQ ID NO: 530, or an amino acid sequence having at least 90%, 91%, 92%, 93%, 94%, 95%, 96%, 97%, 98% or 99% identity with the amino acid sequence of SEQ ID NO: 530, or consists of SEQ ID NO: 530, and the light chain variable region comprises the amino acid sequence as set forth in SEQ ID NO: 533, or an amino acid sequence having at least 90%, 91%, 92%, 93%, 94%, 95%, 96%, 97%, 98% or 99% identity with the amino acid sequence of SEQ ID NO: 533, or consists of SEQ ID NO: 533;
58) the heavy chain variable region comprises the amino acid sequence as set forth in SEQ ID NO: 532, or an amino acid sequence having at least 90%, 91%, 92%, 93%, 94%, 95%, 96%, 97%, 98% or 99% identity with the amino acid sequence of SEQ ID NO: 532, or consists of SEQ ID NO: 532, and the light chain variable region comprises the amino acid sequence as set forth in SEQ ID NO: 531, or an amino acid sequence having at least 90%, 91%, 92%, 93%, 94%, 95%, 96%, 97%, 98% or 99% identity with the amino acid sequence of SEQ ID NO: 531, or consists of SEQ ID NO: 531;
59) the heavy chain variable region comprises the amino acid sequence as set forth in SEQ ID NO: 532, or an amino acid sequence having at least 90%, 91%, 92%, 93%, 94%, 95%, 96%, 97%, 98% or 99% identity with the amino acid sequence of SEQ ID NO: 532, or consists of SEQ ID NO: 532, and the light chain variable region comprises the amino acid sequence as set forth in SEQ ID NO: 533, or an amino acid sequence having at least 90%, 91%, 92%, 93%, 94%, 95%, 96%, 97%, 98% or 99% identity with the amino acid sequence of SEQ ID NO: 533, or consists of SEQ ID NO: 533;
60) the heavy chain variable region comprises the amino acid sequence as set forth in SEQ ID NO: 534, or an amino acid sequence having at least 90%, 91%, 92%, 93%, 94%, 95%, 96%, 97%, 98% or 99% identity with the amino acid sequence of SEQ ID NO: 534, or consists of SEQ ID NO: 534, and the light chain variable region comprises the amino acid sequence as set forth in SEQ ID NO: 535, or an amino acid sequence having at least 90%, 91%, 92%, 93%, 94%, 95%, 96%, 97%, 98% or 99% identity with the amino acid sequence of SEQ ID NO: 535, or consists of SEQ ID NO: 535;
61) the heavy chain variable region comprises the amino acid sequence as set forth in SEQ ID NO: 536, or an amino acid sequence having at least 90%, 91%, 92%, 93%, 94%, 95%, 96%, 97%, 98% or 99% identity with the amino acid sequence of SEQ ID NO: 536, or consists of SEQ ID NO: 536, and the light chain variable region comprises the amino acid sequence as set forth in SEQ ID NO: 537, or an amino acid sequence having at least 90%, 91%, 92%, 93%, 94%, 95%, 96%, 97%, 98% or 99% identity with the amino acid sequence of SEQ ID NO: 537, or consists of SEQ ID NO: 537;
62) the heavy chain variable region comprises the amino acid sequence as set forth in SEQ ID NO: 538, or an amino acid sequence having at least 90%, 91%, 92%, 93%, 94%, 95%, 96%, 97%, 98% or 99% identity with the amino acid sequence of SEQ ID NO: 538, or consists of SEQ ID NO: 538, and the light chain variable region comprises the amino acid sequence as set forth in SEQ ID NO: 539, or an amino acid sequence having at least 90%, 91%, 92%, 93%, 94%, 95%, 96%, 97%, 98% or 99% identity with the amino acid sequence of SEQ ID NO: 539, or consists of SEQ ID NO: 539;
63) the heavy chain variable region comprises the amino acid sequence as set forth in SEQ ID NO: 540, or an amino acid sequence having at least 90%, 91%, 92%, 93%, 94%, 95%, 96%, 97%, 98% or 99% identity with the amino acid sequence of SEQ ID NO: 540, or consists of SEQ ID NO: 540, and the light chain variable region comprises the amino acid sequence as set forth in SEQ ID NO: 541, or an amino acid sequence having at least 90%, 91%, 92%, 93%, 94%, 95%, 96%, 97%, 98% or 99% identity with the amino acid sequence of SEQ ID NO: 541, or consists of SEQ ID NO: 541;
64) the heavy chain variable region comprises the amino acid sequence as set forth in SEQ ID NO: 540, or an amino acid sequence having at least 90%, 91%, 92%, 93%, 94%, 95%, 96%, 97%, 98% or 99% identity with the amino acid sequence of SEQ ID NO: 540, or consists of SEQ ID NO: 540, and the light chain variable region comprises the amino acid sequence as set forth in SEQ ID NO: 542, or an amino acid sequence having at least 90%, 91%, 92%, 93%, 94%, 95%, 96%, 97%, 98% or 99% identity with the amino acid sequence of SEQ ID NO: 542, or consists of SEQ ID NO: 542;
65) the heavy chain variable region comprises the amino acid sequence as set forth in SEQ ID NO: 543, or an amino acid sequence having at least 90%, 91%, 92%, 93%, 94%, 95%, 96%, 97%, 98% or 99% identity with the amino acid sequence of SEQ ID NO: 543, or consists of SEQ ID NO: 543, and the light chain variable region comprises the amino acid sequence as set forth in SEQ ID NO: 544, or an amino acid sequence having at least 90%, 91%, 92%, 93%, 94%, 95%, 96%, 97%, 98% or 99% identity with the amino acid sequence of SEQ ID NO: 544, or consists of SEQ ID NO: 544;
66) the heavy chain variable region comprises the amino acid sequence as set forth in SEQ ID NO: 545, or an amino acid sequence having at least 90%, 91%, 92%, 93%, 94%, 95% %%, 97%, 98% or 99% identity to the amino acid sequence of SEQ ID NO: 545, or consists of SEQ ID NO: 545, and the light chain variable region comprises the amino acid sequence as set forth in SEQ ID NO: 546, or an amino acid sequence having at least 90%, 91%, 92%, 93%, 94%, 95%, 96%, 97%, 98% or 99% identity to the amino acid sequence of SEQ ID NO: 546, or consists of SEQ ID NO: 546; or
67) The heavy chain variable region comprises the amino acid sequence as set forth in SEQ ID NO: 547, or an amino acid sequence having at least 90%, 91%, 92%, 93%, 94%, 95%, 96%, 97%, 98% or 99% identity to the amino acid sequence of SEQ ID NO: 547, or consists of SEQ ID NO: 547, and the light chain variable region comprises the amino acid sequence as set forth in SEQ ID NO: 548, or an amino acid sequence having at least 90%, 91%, 92%, 93%, 94%, 95%, 96%, 97%, 98% or 99% identity to the amino acid sequence of SEQ ID NO: 548, or consists of SEQ ID NO: 548.
68) the heavy chain variable region comprises the amino acid sequence as set forth in SEQ ID NO: 549, or an amino acid sequence having at least 90%, 91%, 92%, 93%, 94%, 95%, 96%, 97%, 98% or 99% identity with the amino acid sequence of SEQ ID NO: 549, or consists of SEQ ID NO: 549, and the light chain variable region comprises the amino acid sequence as set forth in SEQ ID NO: 550, or an amino acid sequence having at least 90%, 91%, 92%, 93%, 94%, 95%, 96%, 97%, 98% or 99% identity with the amino acid sequence of SEQ ID NO: 550, or consists of SEQ ID NO: 550;
69) the heavy chain variable region comprises the amino acid sequence as set forth in SEQ ID NO: 551, or an amino acid sequence having at least 90%, 91%, 92%, 93%, 94%, 95%, 96%, 97%, 98% or 99% identity with the amino acid sequence of SEQ ID NO: 551, or consists of SEQ ID NO: 551, and the light chain variable region comprises the amino acid sequence as set forth in SEQ ID NO: 552, or an amino acid sequence having at least 90%, 91%, 92%, 93%, 94%, 95%, 96%, 97%, 98% or 99% identity with the amino acid sequence of SEQ ID NO: 552, or consists of SEQ ID NO: 552;
70) the heavy chain variable region comprises the amino acid sequence as set forth in SEQ ID NO: 553, or an amino acid sequence having at least 90%, 91%, 92%, 93%, 94%, 95%, 96%, 97%, 98% or 99% identity with the amino acid sequence of SEQ ID NO: 553, or consists of SEQ ID NO: 553, and the light chain variable region comprises the amino acid sequence as set forth in SEQ ID NO: 554, or an amino acid sequence having at least 90%, 91%, 92%, 93%, 94%, 95%, 96%, 97%, 98% or 99% identity with the amino acid sequence of SEQ ID NO: 554, or consists of SEQ ID NO: 554;
71) the heavy chain variable region comprises the amino acid sequence as set forth in SEQ ID NO: 555, or an amino acid sequence having at least 90%, 91%, 92%, 93%, 94%, 95%, 96%, 97%, 98% or 99% identity with the amino acid sequence of SEQ ID NO: 555, or consists of SEQ ID NO: 555, and the light chain variable region comprises the amino acid sequence as set forth in SEQ ID NO: 556, or an amino acid sequence having at least 90%, 91%, 92%, 93%, 94%, 95%, 96%, 97%, 98% or 99% identity with the amino acid sequence of SEQ ID NO: 556, or consists of SEQ ID NO: 556;
72) the heavy chain variable region comprises the amino acid sequence as set forth in SEQ ID NO: 557, or an amino acid sequence having at least 90%, 91%, 92%, 93%, 94%, 95%, 96%, 97%, 98% or 99% identity with the amino acid sequence of SEQ ID NO: 557, or consists of SEQ ID NO: 557, and the light chain variable region comprises the amino acid sequence as set forth in SEQ ID NO: 558, or an amino acid sequence having at least 90%, 91%, 92%, 93%, 94%, 95%, 96%, 97%, 98% or 99% identity with the amino acid sequence of SEQ ID NO: 558, or consists of SEQ ID NO: 558;
73) the heavy chain variable region comprises the amino acid sequence as set forth in SEQ ID NO: 559, or an amino acid sequence having at least 90%, 91%, 92%, 93%, 94%, 95%, 96%, 97%, 98% or 99% identity with the amino acid sequence of SEQ ID NO: 559, or consists of SEQ ID NO: 559, and the light chain variable region comprises the amino acid sequence as set forth in SEQ ID NO: 560, or an amino acid sequence having at least 90%, 91%, 92%, 93%, 94%, 95%, 96%, 97%, 98% or 99% identity with the amino acid sequence of SEQ ID NO: 560, or consists of SEQ ID NO: 560;
74) the heavy chain variable region comprises the amino acid sequence as set forth in SEQ ID NO: 561, or an amino acid sequence having at least 90%, 91%, 92%, 93%, 94%, 95%, 96%, 97%, 98% or 99% identity with the amino acid sequence of SEQ ID NO: 561, or consists of SEQ ID NO: 561, and the light chain variable region comprises the amino acid sequence as set forth in SEQ ID NO: 554, or an amino acid sequence having at least 90%, 91%, 92%, 93%, 94%, 95%, 96%, 97%, 98% or 99% identity with the amino acid sequence of SEQ ID NO: 554, or consists of SEQ ID NO: 554;
75) the heavy chain variable region comprises the amino acid sequence as set forth in SEQ ID NO: 562, or an amino acid sequence having at least 90%, 91%, 92%, 93%, 94%, 95%, 96%, 97%, 98% or 99% identity with the amino acid sequence of SEQ ID NO: 562, or consists of SEQ ID NO: 562, and the light chain variable region comprises the amino acid sequence as set forth in SEQ ID NO: 563, or an amino acid sequence having at least 90%, 91%, 92%, 93%, 94%, 95%, 96%, 97%, 98% or 99% identity with the amino acid sequence of SEQ ID NO: 563, or consists of SEQ ID NO: 563;
76) the heavy chain variable region comprises the amino acid sequence as set forth in SEQ ID NO: 564, or an amino acid sequence having at least 90%, 91%, 92%, 93%, 94%, 95%, 96%, 97%, 98% or 99% identity with the amino acid sequence of SEQ ID NO: 564, or consists of SEQ ID NO: 564, and the light chain variable region comprises the amino acid sequence as set forth in SEQ ID NO: 565, or an amino acid sequence having at least 90%, 91%, 92%, 93%, 94%, 95%, 96%, 97%, 98% or 99% identity with the amino acid sequence of SEQ ID NO: 565, or consists of SEQ ID NO: 565;
77) the heavy chain variable region comprises the amino acid sequence as set forth in SEQ ID NO: 566, or an amino acid sequence having at least 90%, 91%, 92%, 93%, 94%, 95%, 96%, 97%, 98% or 99% identity with the amino acid sequence of SEQ ID NO: 566, or consists of SEQ ID NO: 566, and the light chain variable region comprises the amino acid sequence as set forth in SEQ ID NO: 567, or an amino acid sequence having at least 90%, 91%, 92%, 93%, 94%, 95%, 96%, 97%, 98% or 99% identity with the amino acid sequence of SEQ ID NO: 567, or consists of SEQ ID NO: 567;
78) the heavy chain variable region comprises the amino acid sequence as set forth in SEQ ID NO: 568, or an amino acid sequence having at least 90%, 91%, 92%, 93%, 94%, 95%, 96%, 97%, 98% or 99% identity with the amino acid sequence of SEQ ID NO: 568, or consists of SEQ ID NO: 568, and the light chain variable region comprises the amino acid sequence as set forth in SEQ ID NO: 569, or an amino acid sequence having at least 90%, 91%, 92%, 93%, 94%, 95%, 96%, 97%, 98% or 99% identity with the amino acid sequence of SEQ ID NO: 569, or consists of SEQ ID NO: 569;
79) the heavy chain variable region comprises the amino acid sequence as set forth in SEQ ID NO: 570, or an amino acid sequence having at least 90%, 91%, 92%, 93%, 94%, 95%, 96%, 97%, 98% or 99% identity with the amino acid sequence of SEQ ID NO: 570, or consists of SEQ ID NO: 570, and the light chain variable region comprises the amino acid sequence as set forth in SEQ ID NO: 571, or an amino acid sequence having at least 90%, 91%, 92%, 93%, 94%, 95%, 9%, 97%, 98% or 99% identity with the amino acid sequence of SEQ ID NO: 571, or consists of SEQ ID NO: 571;
80) the heavy chain variable region comprises the amino acid sequence as set forth in SEQ ID NO: 572, or an amino acid sequence having at least 90%, 91%, 92%, 93%, 94%, 95%, 96%, 97%, 98% or 99% identity with the amino acid sequence of SEQ ID NO: 572, or consists of SEQ ID NO: 572, and the light chain variable region comprises the amino acid sequence as set forth in SEQ ID NO: 573, or an amino acid sequence having at least 90%, 91%, 92%, 93%, 94%, 95%, 96%, 97%, 98% or 99% identity with the amino acid sequence of SEQ ID NO: 573, or consists of SEQ ID NO: 573;
81) the heavy chain variable region comprises the amino acid sequence as set forth in SEQ ID NO: 574, or an amino acid sequence having at least 90%, 91%, 92%, 93%, 94%, 95%, 9%%, 97%, 98% or 99% identity with the amino acid sequence of SEQ ID NO: 574, or consists of SEQ ID NO: 574, and the light chain variable region comprises the amino acid sequence as set forth in SEQ ID NO: 575, or an amino acid sequence having at least 90%, 91%, 92%, 93%, 94%, 95%, 96%, 97%, 98% or 99% identity with the amino acid sequence of SEQ ID NO: 575, or consists of SEQ ID NO: 575;
82) the heavy chain variable region comprises the amino acid sequence as set forth in SEQ ID NO: 576, or an amino acid sequence having at least 90%, 91%, 92%, 93%, 94%, 95%, 96%, 97%, 98% or 99% identity with the amino acid sequence of SEQ ID NO: 576, or consists of SEQ ID NO: 576, and the light chain variable region comprises the amino acid sequence as set forth in SEQ ID NO: 578, or an amino acid sequence having at least 90%, 91%, 92%, 93%, 94%, 95%, 96%, 97%, 98% or 99% identity with the amino acid sequence of SEQ ID NO: 578, or consists of SEQ ID NO: 578;
83) the heavy chain variable region comprises the amino acid sequence as set forth in SEQ ID NO: 577, or an amino acid sequence having at least 90%, 91%, 92%, 93%, 94%, 95%, 96%, 97%, 98% or 99% identity with the amino acid sequence of SEQ ID NO: 577, or consists of SEQ ID NO: 577, and the light chain variable region comprises the amino acid sequence as set forth in SEQ ID NO: 578, or an amino acid sequence having at least 90%, 91%, 92%, 93%, 94%, 95%, 96%, 97%, 98% or 99% identity with the amino acid sequence of SEQ ID NO: 578, or consists of SEQ ID NO: 578;
84) the heavy chain variable region comprises the amino acid sequence as set forth in SEQ ID NO: 579, or an amino acid sequence having at least 90%, 91%, 92%, 93%, 94%, 95%, 96%, 97%, 98% or 99% identity with the amino acid sequence of SEQ ID NO: 579, or consists of SEQ ID NO: 579, and the light chain variable region comprises the amino acid sequence as set forth in SEQ ID NO: 580, or an amino acid sequence having at least 90%, 91%, 92%, 93%, 94%, 95%, 96%, 97%, 98% or 99% identity with the amino acid sequence of SEQ ID NO: 580, or consists of SEQ ID NO: 580;
85) the heavy chain variable region comprises the amino acid sequence as set forth in SEQ ID NO: 581, or an amino acid sequence having at least 90%, 91%, 92%, 93%, 94%, 95%, 96%, 97%, 98% or 99% identity with the amino acid sequence of SEQ ID NO: 581, or consists of SEQ ID NO: 581, and the light chain variable region comprises the amino acid sequence as set forth in SEQ ID NO: 582, or an amino acid sequence having at least 90%, 91%, 92%, 93%, 94%, 95%, 96%, 97%, 98% or 99% identity with the amino acid sequence of SEQ ID NO: 582, or consists of SEQ ID NO: 582;
86) the heavy chain variable region comprises the amino acid sequence as set forth in SEQ ID NO: 583, or an amino acid sequence having at least 90%, 91%, 92%, 93%, 94%, 95% 96%, 97%, 98% or 99% identity to the amino acid sequence of SEQ ID NO: 583, or consists of SEQ ID NO: 583, and the light chain variable region comprises the amino acid sequence as set forth in SEQ ID NO: 584, or an amino acid sequence having at least 90%, 91%, 92%, 93%, 94%, 95%, 96%, 97%, 98% or 99% identity to the amino acid sequence of SEQ ID NO: 584, or consists of SEQ ID NO: 584; or
87) The heavy chain variable region comprises the amino acid sequence as set forth in SEQ ID NO: 585, or an amino acid sequence having at least 90%, 91%, 92%, 93%, 94%, 95%, 96%, 97%, 98% or 99% identity to the amino acid sequence of SEQ ID NO: 585, or consists of SEQ ID NO: 585, and the light chain variable region comprises the amino acid sequence as set forth in SEQ ID NO: 586, or an amino acid sequence having at least 90%, 91%, 92%, 93%, 94%, 95%, 96%, 97%, 98% or 99% identity to the amino acid sequence of SEQ ID NO: 586, or consists of SEQ ID NO: 586.
88) the heavy chain variable region comprises the amino acid sequence as set forth in SEQ ID NO: 587, or an amino acid sequence having at least 90%, 91%, 92%, 93%, 94%, 95%, 96%, 97%, 98% or 99% identity with the amino acid sequence of SEQ ID NO: 587, or consists of SEQ ID NO: 587, and the light chain variable region comprises the amino acid sequence as set forth in SEQ ID NO: 588, or an amino acid sequence having at least 90%, 91%, 92%, 93%, 94%, 95%, 96%, 97%, 98% or 99% identity with the amino acid sequence of SEQ ID NO: 588, or consists of SEQ ID NO: 588;
89) the heavy chain variable region comprises the amino acid sequence as set forth in SEQ ID NO: 587, or an amino acid sequence having at least 90%, 91%, 92%, 93%, 94%, 95%, 96%, 97%, 98% or 99% identity with the amino acid sequence of SEQ ID NO: 587, or consists of SEQ ID NO: 587, and the light chain variable region comprises the amino acid sequence as set forth in SEQ ID NO: 589, or an amino acid sequence having at least 90%, 91%, 92%, 93%, 94%, 95%, 96%, 97%, 98% or 99% identity with the amino acid sequence of SEQ ID NO: 589, or consists of SEQ ID NO: 589;
90) the heavy chain variable region comprises the amino acid sequence as set forth in SEQ ID NO: 590, or an amino acid sequence having at least 90%, 91%, 92%, 93%, 94%, 95%, 96%, 97%, 98% or 99% identity with the amino acid sequence of SEQ ID NO: 590, or consists of SEQ ID NO: 590, and the light chain variable region comprises the amino acid sequence as set forth in SEQ ID NO: 591, or an amino acid sequence having at least 90%, 91%, 92%, 93%, 94%, 95%, 96%, 97%, 98% or 99% identity with the amino acid sequence of SEQ ID NO: 591, or consists of SEQ ID NO: 591;
91) the heavy chain variable region comprises the amino acid sequence as set forth in SEQ ID NO: 592, or an amino acid sequence having at least 90%, 91%, 92%, 93%, 94%, 95%, 96%, 97%, 98% or 99% identity with the amino acid sequence of SEQ ID NO: 592, or consists of SEQ ID NO: 592, and the light chain variable region comprises the amino acid sequence as set forth in SEQ ID NO: 593, or an amino acid sequence having at least 90%, 91%, 92%, 93%, 94%, 95%, 96%, 97%, 98% or 99% identity with the amino acid sequence of SEQ ID NO: 593, or consists of SEQ ID NO: 593;
92) the heavy chain variable region comprises the amino acid sequence as set forth in SEQ ID NO: 592, or an amino acid sequence having at least 90%, 91%, 92%, 93%, 94%, 95%, 96%, 97%, 98% or 99% identity with the amino acid sequence of SEQ ID NO: 592, or consists of SEQ ID NO: 592, and the light chain variable region comprises the amino acid sequence as set forth in SEQ ID NO: 594, or an amino acid sequence having at least 90%, 91%, 92%, 93%, 94%, 95%, 96%, 97%, 98% or 99% identity with the amino acid sequence of SEQ ID NO: 594, or consists of SEQ ID NO: 594;
93) the heavy chain variable region comprises the amino acid sequence as set forth in SEQ ID NO: 595, or an amino acid sequence having at least 90%, 91%, 92%, 93%, 94%, 95%, 96%, 97%, 98% or 99% identity with the amino acid sequence of SEQ ID NO: 595, or consists of SEQ ID NO: 595, and the light chain variable region comprises the amino acid sequence as set forth in SEQ ID NO: 596, or an amino acid sequence having at least 90%, 91%, 92%, 93%, 94%, 95%, 96%, 97%, 98% or 99% identity with the amino acid sequence of SEQ ID NO: 596, or consists of SEQ ID NO: 596;
94) the heavy chain variable region comprises the amino acid sequence as set forth in SEQ ID NO: 595, or an amino acid sequence having at least 90%, 91%, 92%, 93%, 94%, 95%, 96%, 97%, 98% or 99% identity with the amino acid sequence of SEQ ID NO: 595, or consists of SEQ ID NO: 595, and the light chain variable region comprises the amino acid sequence as set forth in SEQ ID NO: 594, or an amino acid sequence having at least 90%, 91%, 92%, 93%, 94%, 95%, 96%, 97%, 98% or 99% identity with the amino acid sequence of SEQ ID NO: 594, or consists of SEQ ID NO: 594;
95) the heavy chain variable region comprises the amino acid sequence as set forth in SEQ ID NO: 597, or an amino acid sequence having at least 90%, 91%, 92%, 93%, 94%, 95%, 96%, 97%, 98% or 99% identity with the amino acid sequence of SEQ ID NO: 597, or consists of SEQ ID NO: 597, and the light chain variable region comprises the amino acid sequence as set forth in SEQ ID NO: 598, or an amino acid sequence having at least 90%, 91%, 92%, 93%, 94%, 95%, 96%, 97%, 98% or 99% identity with the amino acid sequence of SEQ ID NO: 598, or consists of SEQ ID NO: 598;
96) the heavy chain variable region comprises the amino acid sequence as set forth in SEQ ID NO: 597, or an amino acid sequence having at least 90%, 91%, 92%, 93%, 94%, 95%, 9%%, 97%, 98% or 99% identity with the amino acid sequence of SEQ ID NO: 597, or consists of SEQ ID NO: 597, and the light chain variable region comprises the amino acid sequence as set forth in SEQ ID NO: 594, or an amino acid sequence having at least 90%, 91%, 92%, 93%, 94%, 95%, 96%, 97%, 98% or 99% identity with the amino acid sequence of SEQ ID NO: 594, or consists of SEQ ID NO: 594;
97) the heavy chain variable region comprises the amino acid sequence as set forth in SEQ ID NO: 599, or an amino acid sequence having at least 90%, 91%, 92%, 93%, 94%, 95%, 96%, 97%, 98% or 99% identity with the amino acid sequence of SEQ ID NO: 599, or consists of SEQ ID NO: 599, and the light chain variable region comprises the amino acid sequence as set forth in SEQ ID NO: 600, or an amino acid sequence having at least 90%, 91%, 92%, 93%, 94%, 95%, 96%, 97%, 98% or 99% identity with the amino acid sequence of SEQ ID NO: 600, or consists of SEQ ID NO: 600;
98) the heavy chain variable region comprises the amino acid sequence as set forth in SEQ ID NO: 601, or an amino acid sequence having at least 90%, 91%, 92%, 93%, 94%, 95%, 96%, 97%, 98% or 99% identity with the amino acid sequence of SEQ ID NO: 601, or consists of SEQ ID NO: 601, and the light chain variable region comprises the amino acid sequence as set forth in SEQ ID NO: 602, or an amino acid sequence having at least 90%, 91%, 92%, 93%, 94%, 95%, 96%, 97%, 98% or 99% identity with the amino acid sequence of SEQ ID NO: 602, or consists of SEQ ID NO: 602;
99) the heavy chain variable region comprises the amino acid sequence as set forth in SEQ ID NO: 603, or an amino acid sequence having at least 90%, 91%, 92%, 93%, 94%, 95%, 96%, 97%, 98% or 99% identity with the amino acid sequence of SEQ ID NO: 603, or consists of SEQ ID NO: 603, and the light chain variable region comprises the amino acid sequence as set forth in SEQ ID NO: 604, or an amino acid sequence having at least 90%, 91%, 92%, 93%, 94%, 95%, 96%, 97%, 98% or 99% identity with the amino acid sequence of SEQ ID NO: 604, or consists of SEQ ID NO: 604;
100) the heavy chain variable region comprises the amino acid sequence as set forth in SEQ ID NO: 605, or an amino acid sequence having at least 90%, 91%, 92%, 93% 94%, 95%, 96%, 97%, 98% or 99% identity with the amino acid sequence of SEQ ID NO: 605, or consists of SEQ ID NO: 605, and the light chain variable region comprises the amino acid sequence as set forth in SEQ ID NO: 606, or an amino acid sequence having at least 90%, 91%, 92%, 93%, 94%, 95%, 96%, 97%, 98% or 99% identity with the amino acid sequence of SEQ ID NO: 606, or consists of SEQ ID NO: 606;
101) the heavy chain variable region comprises the amino acid sequence as set forth in SEQ ID NO: 607, or an amino acid sequence having at least 90%, 91%, 92%, 93%, 94%, 95%, 96%, 97%, 98% or 99% identity with the amino acid sequence of SEQ ID NO: 607, or consists of SEQ ID NO: 607, and the light chain variable region comprises the amino acid sequence as set forth in SEQ ID NO: 608, or an amino acid sequence having at least 90/o, 91%, 92%, 93%, 94%, 95%, 96%, 97%, 98% or 99% identity with the amino acid sequence of SEQ ID NO: 608, or consists of SEQ ID NO: 608;
102) the heavy chain variable region comprises the amino acid sequence as set forth in SEQ ID NO: 609, or an amino acid sequence having at least 90%, 91%, 92%, 93%, 94%, 95% 96%, 97%, 98% or 99% identity with the amino acid sequence of SEQ ID NO: 609, or consists of SEQ ID NO: 609, and the light chain variable region comprises the amino acid sequence as set forth in SEQ ID NO: 610, or an amino acid sequence having at least 90%, 91%, 92%, 93%, 94%, 95%, 96%, 97%, 98% or 99% identity with the amino acid sequence of SEQ ID NO: 610, or consists of SEQ ID NO: 610;
103) the heavy chain variable region comprises the amino acid sequence as set forth in SEQ ID NO: 611, or an amino acid sequence having at least 90%, 91%, 92%, 93%, 94% 95%, 96%, 97%, 98% or 99% identity with the amino acid sequence of SEQ ID NO: 611, or consists of SEQ ID NO: 611, and the light chain variable region comprises the amino acid sequence as set forth in SEQ ID NO: 612, or an amino acid sequence having at least 90%, 91%, 92%, 93%, 94%, 95%, 96%, 97%, 98% or 99% identity with the amino acid sequence of SEQ ID NO; 612, or consists of SEQ ID NO: 612;
104) the heavy chain variable region comprises the amino acid sequence as set forth in SEQ ID NO: 613, or an amino acid sequence having at least 90%, 91%, 92%, 93%, 94%, 95%, 96%, 97%, 98% or 99% identity with the amino acid sequence of SEQ ID NO: 613, or consists of SEQ ID NO: 613, and the light chain variable region comprises the amino acid sequence as set forth in SEQ ID NO: 614, or an amino acid sequence having at least 90%, 91%, 92%, 93%, 94%, 95%, 96%, 97%, 98% or 99% identity with the amino acid sequence of SEQ ID NO: 614, or consists of SEQ ID NO: 614;
105) the heavy chain variable region comprises the amino acid sequence as set forth in SEQ ID NO: 615, or an amino acid sequence having at least 90%, 91%, 92%, 93%, 94% 95%, 96%, 97%, 98% or 99% identity with the amino acid sequence of SEQ ID NO: 615, or consists of SEQ ID NO: 615, and the light chain variable region comprises the amino acid sequence as set forth in SEQ ID NO: 616, or an amino acid sequence having at least 90%, 91%, 92%, 93%, 94%, 95%, 96%, 97%, 98% or 99% identity with the amino acid sequence of SEQ ID NO: 616, or consists of SEQ ID NO: 616;
106) the heavy chain variable region comprises the amino acid sequence as set forth in SEQ ID NO: 617, or an amino acid sequence having at least 90%, 91%, 92%, 93%, 94%, 95%, 96%, 97%, 98% or 99% identity to the amino acid sequence of SEQ ID NO: 617, or consists of SEQ ID NO: 617, and the light chain variable region comprises the amino acid sequence as set forth in SEQ ID NO: 618, or an amino acid sequence having at least 90%, 91%, 92%, 93%, 94%, 95%, 96%, 97%, 98% or 99% identity to the amino acid sequence of SEQ ID NO: 618, or consists of SEQ ID NO: 618; or
107) The heavy chain variable region comprises the amino acid sequence as set forth in SEQ ID NO: 619, or an amino acid sequence having at least 90%, 91%, 92%, 93%, 94%, 95%, 96%, 97%, 98% or 99% identity to the amino acid sequence of SEQ ID NO: 619, or consists of SEQ ID NO: 619, and the light chain variable region comprises the amino acid sequence as set forth in SEQ ID NO: 620, or an amino acid sequence having at least 90%, 91%, 92%, 93%, 94%, 95%, 96%, 97%, 98% or 99% identity to the amino acid sequence of SEQ ID NO: 620, or consists of SEQ ID NO: 620.
108) the heavy chain variable region comprises the amino acid sequence as set forth in SEQ ID NO: 621, or an amino acid sequence having at least 90%, 91%, 92%, 93%, 94% 95%, 96%, 97%, 98% or 99% identity with the amino acid sequence of SEQ ID NO: 621, or consists of SEQ ID NO: 621, and the light chain variable region comprises the amino acid sequence as set forth in SEQ ID NO: 622, or an amino acid sequence having at least 90%, 91%, 92%, 93%, 94%, 95%, 96%, 97%, 98% or 99% identity with the amino acid sequence of SEQ ID NO: 622, or consists of SEQ ID NO: 622;
109) the heavy chain variable region comprises the amino acid sequence as set forth in SEQ ID NO: 623, or an amino acid sequence having at least 90%, 91%, 92%, 93%, 94%, 95%, 96%, 97%, 98% or 99% identity with the amino acid sequence of SEQ ID NO: 623, or consists of SEQ ID NO: 623, and the light chain variable region comprises the amino acid sequence as set forth in SEQ ID NO: 624, or an amino acid sequence having at least 90%, 91%, 92%, 93%, 94%, 95%, %%, 97%, 98% or 99% identity with the amino acid sequence of SEQ ID NO: 624, or consists of SEQ ID NO: 624;
110) the heavy chain variable region comprises the amino acid sequence as set forth in SEQ ID NO: 625, or an amino acid sequence having at least 90%, 91%, 92%, 93%, 94%, 95%, 96%, 97%, 98% or 99% identity with the amino acid sequence of SEQ ID NO: 625, or consists of SEQ ID NO: 625, and the light chain variable region comprises the amino acid sequence as set forth in SEQ ID NO: 626, or an amino acid sequence having at least 90%, 91%, 92%, 93%, 94%, 95%, 96%, 97%, 98% or 99% identity with the amino acid sequence of SEQ ID NO: 626, or consists of SEQ ID NO: 626;
111) the heavy chain variable region comprises the amino acid sequence as set forth in SEQ ID NO: 627, or an amino acid sequence having at least 90%, 91%, 92%, 93% 94%, 95%, 96%, 97%, 98% or 99% identity with the amino acid sequence of SEQ ID NO: 627, or consists of SEQ ID NO: 627, and the light chain variable region comprises the amino acid sequence as set forth in SEQ ID NO: 628, or an amino acid sequence having at least 90%, 91%, 92%, 93%, 94%, 95%, 96%, 97%, 98% or 99% identity with the amino acid sequence of SEQ ID NO: 628, or consists of SEQ ID NO: 628;
112) the heavy chain variable region comprises the amino acid sequence as set forth in SEQ ID NO: 629, or an amino acid sequence having at least 90%, 91%, 92%, 93%, 94%, 95%, 96%, 97%, 98% or 99% identity with the amino acid sequence of SEQ ID NO: 629, or consists of SEQ ID NO: 629, and the light chain variable region comprises the amino acid sequence as set forth in SEQ ID NO: 630, or an amino acid sequence having at least 90%, 91%, 92%, 93%, 94%, 95%, 96%, 97%, 98% or 99% identity with the amino acid sequence of SEQ ID NO: 630, or consists of SEQ ID NO: 630;
113) the heavy chain variable region comprises the amino acid sequence as set forth in SEQ ID NO: 631, or an amino acid sequence having at least 90%, 91%, 92%, 93%, 94%, 95%, 96%, 97%, 98% or 99% identity with the amino acid sequence of SEQ ID NO: 631, or consists of SEQ ID NO: 631, and the light chain variable region comprises the amino acid sequence as set forth in SEQ ID NO: 632, or an amino acid sequence having at least 90/o, 91%, 92%, 93%, 94%, 95%, 96%, 97%, 98% or 99% identity with the amino acid sequence of SEQ ID NO: 632, or consists of SEQ ID NO: 632;
114) the heavy chain variable region comprises the amino acid sequence as set forth in SEQ ID NO: 617, or an amino acid sequence having at least 90%, 91%, 92%, 93%, 94% 95%, 96%, 97%, 98% or 99% identity with the amino acid sequence of SEQ ID NO: 617, or consists of SEQ ID NO: 617, and the light chain variable region comprises the amino acid sequence as set forth in SEQ ID NO: 633, or an amino acid sequence having at least 90%, 91%, 92%, 93%, 94%, 95%, 96%, 97%, 98% or 99% identity with the amino acid sequence of SEQ ID NO: 633, or consists of SEQ ID NO: 633;
115) the heavy chain variable region comprises the amino acid sequence as set forth in SEQ ID NO: 634, or an amino acid sequence having at least 90%, 91%, 92%, 93%, 94%, 95%, 96%, 97%, 98% or 99% identity with the amino acid sequence of SEQ ID NO: 634, or consists of SEQ ID NO: 634, and the light chain variable region comprises the amino acid sequence as set forth in SEQ ID NO: 635, or an amino acid sequence having at least 90%, 91%, 92%, 93%, 94%, 95%, 96%, 97%, 98% or 99% identity with the amino acid sequence of SEQ ID NO: 635, or consists of SEQ ID NO: 635;
116) the heavy chain variable region comprises the amino acid sequence as set forth in SEQ ID NO: 636, or an amino acid sequence having at least 90%, 91%, 92%, 93%, 94%, 95%, 96%, 97%, 98% or 99% identity with the amino acid sequence of SEQ ID NO: 636, or consists of SEQ ID NO: 636, and the light chain variable region comprises the amino acid sequence as set forth in SEQ ID NO: 637, or an amino acid sequence having at least 90%, 91%, 92%, 93%, 94%, 95%, 96%, 97%, 98% or 99% identity with the amino acid sequence of SEQ ID NO: 637, or consists of SEQ ID NO: 637;
117) the heavy chain variable region comprises the amino acid sequence as set forth in SEQ ID NO: 638, or an amino acid sequence having at least 90%, 91%, 92%, 93% 94%, 95%, 96%, 97%, 98% or 99% identity with the amino acid sequence of SEQ ID NO: 638, or consists of SEQ ID NO: 638, and the light chain variable region comprises the amino acid sequence as set forth in SEQ ID NO: 639, or an amino acid sequence having at least 90%, 91%, 92%, 93%, 94%, 95%, 96%, 97%, 98% or 99% identity with the amino acid sequence of SEQ ID NO: 639, or consists of SEQ ID NO: 639; 118) the heavy chain variable region comprises the amino acid sequence as set forth in SEQ ID NO: 640, or an amino acid sequence having at least 90%, 91%, 92%, 93%, 94%, 95%, 96%, 97%, 98% or 99% identity with the amino acid sequence of SEQ ID NO: 640, or consists of SEQ ID NO: 640, and the light chain variable region comprises the amino acid sequence as set forth in SEQ ID NO: 641, or an amino acid sequence having at least 90%, 91%, 92%, 93%, 94%, 95%, 96%, 97%, 98% or 99% identity with the amino acid sequence of SEQ ID NO: 641, or consists of SEQ ID NO: 641;
119) the heavy chain variable region comprises the amino acid sequence as set forth in SEQ ID NO: 642, or an amino acid sequence having at least 90%, 91%, 92%, 93%, 94% 95%, 96%, 97%, 98% or 99% identity with the amino acid sequence of SEQ ID NO: 642, or consists of SEQ ID NO: 642, and the light chain variable region comprises the amino acid sequence as set forth in SEQ ID NO: 643, or an amino acid sequence having at least 90%, 91%, 92%, 93%, 94%, 95%, 96%, 97%, 98% or 99% identity with the amino acid sequence of SEQ ID NO: 643, or consists of SEQ ID NO: 643;
120) the heavy chain variable region comprises the amino acid sequence as set forth in SEQ ID NO: 644, or an amino acid sequence having at least 90%, 91%, 92%, 93%, 94%, 95%, 96%, 97%, 98% or 99% identity with the amino acid sequence of SEQ ID NO: 644, or consists of SEQ ID NO: 644, and the light chain variable region comprises the amino acid sequence as set forth in SEQ ID NO: 645, or an amino acid sequence having at least 90%, 91%, 92%, 93%, 94%, 95%, 96%, 97%, 98% or 99% identity with the amino acid sequence of SEQ ID NO: 645, or consists of SEQ ID NO: 645;
121) the heavy chain variable region comprises the amino acid sequence as set forth in SEQ ID NO: 646, or an amino acid sequence having at least 90%, 91%, 92%, 93%, 94%, 95%, 96%, 97%, 98% or 99% identity with the amino acid sequence of SEQ ID NO: 646, or consists of SEQ ID NO: 646, and the light chain variable region comprises the amino acid sequence as set forth in SEQ ID NO: 647, or an amino acid sequence having at least 90%, 91%, 92%, 93%, 94%, 95%, 96%, 97%, 98% or 99% identity with the amino acid sequence of SEQ ID NO: 647, or consists of SEQ ID NO: 647;
122) the heavy chain variable region comprises the amino acid sequence as set forth in SEQ ID NO: 648, or an amino acid sequence having at least 90%, 91%, 92%, 93% 94%, 95%, 96%, 97%, 98% or 99% identity with the amino acid sequence of SEQ ID NO: 648, or consists of SEQ ID NO: 648, and the light chain variable region comprises the amino acid sequence as set forth in SEQ ID NO: 649, or an amino acid sequence having at least 90%, 91%, 92%, 93%, 94%, 95%, 96%, 97%, 98% or 99% identity with the amino acid sequence of SEQ ID NO: 649, or consists of SEQ ID NO: 649;
123) the heavy chain variable region comprises the amino acid sequence as set forth in SEQ ID NO: 650, or an amino acid sequence having at least 90%, 91%, 92%, 93%, 94%, 95%, 96%, 97%, 98% or 99% identity with the amino acid sequence of SEQ ID NO: 650, or consists of SEQ ID NO: 650, and the light chain variable region comprises the amino acid sequence as set forth in SEQ ID NO: 651, or an amino acid sequence having at least 90%, 91%, 92%, 93%, 94%, 95%, 96%, 97%, 98% or 99% identity with the amino acid sequence of SEQ ID NO: 651, or consists of SEQ ID NO: 651;
124) the heavy chain variable region comprises the amino acid sequence as set forth in SEQ ID NO: 652, or an amino acid sequence having at least 90%, 91%, 92%, 93%, 94%, 95%, 96%, 97%, 98% or 99% identity with the amino acid sequence of SEQ ID NO: 652, or consists of SEQ ID NO: 652, and the light chain variable region comprises the amino acid sequence as set forth in SEQ ID NO: 653, or an amino acid sequence having at least 90/o, 91%, 92%, 93%, 94%, 95%, 96%, 97%, 98% or 99% identity with the amino acid sequence of SEQ ID NO: 653, or consists of SEQ ID NO: 653;
125) the heavy chain variable region comprises the amino acid sequence as set forth in SEQ ID NO: 654, or an amino acid sequence having at least 90%, 91%, 92%, 93%, 94% 95%, 96%, 97%, 98% or 99% identity with the amino acid sequence of SEQ ID NO: 654, or consists of SEQ ID NO: 654, and the light chain variable region comprises the amino acid sequence as set forth in SEQ ID NO: 655, or an amino acid sequence having at least 90%, 91%, 92%, 93%, 94%, 95%, 96%, 97%, 98% or 99% identity with the amino acid sequence of SEQ ID NO; 655, or consists of SEQ ID NO: 655;
126) The heavy chain variable region comprises the amino acid sequence as set forth in SEQ ID NO: 656, or an amino acid sequence having at least 90%, 91%, 92%, 93%, 94%, 95%, 96%, 97%, 98% or 99% identity to the amino acid sequence of SEQ ID NO: 656, or consists of SEQ ID NO: 656, and the light chain variable region comprises the amino acid sequence as set forth in SEQ ID NO: 653, or an amino acid sequence having at least 90%, 91%, 92%, 93%, 94%, 95%, 96%, 97%, 98% or 99% identity to the amino acid sequence of SEQ ID NO: 653, or consists of SEQ ID NO: 653.
In some embodiments, the antibody or antigen-binding fragment thereof further comprises a heavy chain and/or light chain constant region sequence from a consensus sequence of human antibody germ lines. The light chain constant region is preferably a human source κ or lambda chain constant region. The heavy chain constant region may be γ, μ, α, δ, or epsilon chain, and in some embodiments, the heavy chain constant region is preferably a constant region sequence of human IgG1, IgG2, IgG3, IgG4. In one embodiment, the light chain constant region comprises, or consists of, the sequence as set forth in SEQ ID NO: 660. In another embodiment, the heavy chain constant region comprises the Fc sequence as set forth in SEQ ID NO: 661. In yet another embodiment, the heavy chain constant region comprises or consists of the sequence as set forth in SEQ ID NO: 659.

It should be understood that sequence variants of these constant region domains may also be used, for example comprising one or more amino acid modifications, wherein the amino acid sites are the (1991) EU index system identity of Kabat et al

In certain embodiments of the antibody of any of the preceding, the antibody is a monoclonal antibody.

In certain embodiments of the antibody of any of the preceding, the antibody is a full-length antibody.

In one embodiment, the anti-CD112R antibody of the invention is an intact antibody, such as IgG1, IgG2, IgG3, IgG4 antibody. In another embodiment, the anti-CD112R antibody of the invention encompasses only an antigen binding portion thereof, such as Fab, Fab′-SH, Fv, scFv, or (Fab′)₂ fragment).

In the second aspect, the invention provides an isolated polynucleotide molecule encoding any antibody or antigen-binding fragment thereof according to the first aspect.

In the third aspect, the invention provides an expression vector comprising the nucleotide molecule according to the second aspect. In one embodiment, the vector is a eukaryotic expression vector.

In the fourth aspect, the invention provides a host cell comprising the vector according to the third aspect or the nucleotide molecule according to the second aspect. In some embodiments, the host cell is prokaryotic, such as E. coli. In other embodiments, the host cell is eukaryotic, such as 293 cells, CHO cells, yeast cells, or plant cells.

In the fifth aspect, the invention provides a pharmaceutical composition comprising (1) the antibody or an antigen-binding fragment thereof according to the first aspect, or the polynucleotide molecule according to the second aspect, or the expression vector according to the third aspect, or the host cell according to the fourth aspect, and (2) a pharmaceutically acceptable carrier or excipient.

In the sixth aspect, the invention provides a method for preparing the anti-CD112R antibody or antigen-binding fragment thereof described herein, comprising expressing the antibody or antigen-binding fragment thereof in a host cell described herein under conditions suitable for expression of the antibody or antigen-binding fragment thereof, and recovering the expressed antibody or antigen-binding fragment thereof from the host cell.

In the seventh aspect, the antibody or antigen-binding fragment thereof or the pharmaceutical composition according to the invention can also be administered in combination with one or more other therapies, e.g., therapeutic modalities and/or other therapeutics, for the uses as described herein, e.g., for the prevention and/or treatment of the associated diseases or conditions mentioned herein.

In the eighth aspect, the invention provides a pharmaceutical combination comprising the antibody or antigen-binding fragment thereof as described herein or the pharmaceutical composition, and one or more additional therapeutics.

In the ninth aspect, the invention provides a method for preventing or treating an CD112R-mediated disease in a subject in need thereof, comprising administering to the subject a prophylactically effective amount or a therapeutically effective amount of the antibody or antigen-binding fragment thereof according to the invention, the polynucleotide molecule as described herein, the expression vector as described herein, the host cell as described herein, the pharmaceutical composition as described herein, or the pharmaceutical combination as described herein, preferably the disease or condition is an CD112R-associated cancer, more preferably the cancer is selected from melanoma, renal cancer, prostate cancer, breast cancer, colon cancer, lung cancer, bone cancer, pancreatic cancer, skin cancer, head and neck cancer, uterine cancer, ovarian cancer and rectal cancer.

In the tenth aspect, the invention provides use of the antibody or antigen-binding fragment thereof described herein, the polynucleotide molecule described herein, the expression vector described herein, the host cell described herein, the pharmaceutical composition described herein, or the pharmaceutical combination described herein in the preparation of a medicament for the treatment and/or prevention of an CD112R-mediated disease or condition, preferably the disease or condition is an CD112R-associated cancer, more preferably the cancer is selected from melanoma, renal cancer, prostate cancer, breast cancer, colon cancer, lung cancer, bone cancer, pancreatic cancer, skin cancer, head and neck cancer, uterine cancer, ovarian cancer, and rectal cancer.

In the eleventh aspect, the invention provides the antibody or antigen-binding fragment thereof as described herein, the polynucleotide molecule as described herein, the expression vector as described herein, the host cell as described herein, the pharmaceutical composition as described herein, or the pharmaceutical combination described herein for the treatment and/or prevention of an CD112R-mediated disease or condition, preferably the disease or condition is cancer, more preferably the cancer is selected from melanoma, renal cancer, prostate cancer, breast cancer, colon cancer, lung cancer, bone cancer, pancreatic cancer, skin cancer, head and neck cancer, uterine cancer, ovarian cancer, and rectal cancer.

In the twelfth aspect, the invention further provides a kit comprising the anti-CD112R antibody or the antigen-binding fragment thereof according to the invention, the polynucleotide molecule according to the invention, the expression vector according to the invention, the host cell according to the invention, the pharmaceutical composition according to the invention, or the pharmaceutical combination according to the invention, or a pharmaceutical composition, and instructions for use. The kit may also comprise a suitable container. In certain embodiments, the kit further comprises a device for administration. Kits generally include a label indicating the intended use and/or method of use of the contents of the kit. The term “label” includes any written or recorded material provided on or with the kit or otherwise provided with the kit.

In another aspect, the invention provides a method for detecting the presence of CD112R in a sample using the antibody or antigen-binding fragment thereof described herein or a detection composition containing the antibody or antigen-binding fragment thereof.

### DETAILED DESCRIPTION OF THE INVENTION

Before describing the invention in detail, it should be understood that the invention is not limited to the specific methods and experimental conditions in this specification, as the methods and conditions may be varied. In addition, the terminology used herein is for the purpose of describing particular embodiments only and is not intended to be limiting.

### I. Definitions

Unless defined otherwise, all technical and scientific terms used herein have the same meanings as commonly understood by one of ordinary skill in the art. For the purposes of the invention, the following terms are defined below.

The term “about” when used in conjunction with a numerical value is intended to encompass a numerical value within a range having a lower limit than the specified number value 10% and an upper limit value greater than the specified number value 10%.

The term “and/or” when used to connect two or more alternatives should be understood to mean any two or more of the alternatives.

As used herein, the terms “comprise” or “comprising” are intended to include the recited element, integer or step, but do not exclude any other elements, integers or steps. Herein, when the term “comprise” or “comprising” is used, the case consisting of the recited elements, integers or steps is also covered, unless otherwise specified. For example, when reference is made to an antibody variable region “comprising” a specific sequence, it is also intended to cover an antibody variable region consisting of the specific sequence.

The terms “CD112R”, “PVR Related Immunoglobulin Domain”, “CD112 receptor”, “Poliovirus Receptor Related Immunoglobulin Domain”, “Nectin2 receptor”, “C7orfl5”, “transmembrane protein PVRIG” are all used interchangeably and include variants, subtypes, species homologs of human CD112R or CD112R of other species and analogs having at least one common epitope of CD112R, unless otherwise specified. The term includes untreated full length CD112R and any form of CD112R due to treatment in cells. The term encompasses both “full-length” unprocessed CD112R and any form of CD112R or any fragment thereof produced by intracellular processing, such as splice variants or allelic variants. The variant or homolog maintains the activity of CD112R activity. e.g., at least 50%, 80%, 90%, 95%, 96%, 97%, 98%, 99%, or 100%. The variant or homolog has at least 90%, 95%, %, 97%, 98%, 99%, or 100% amino acid sequence identity over full sequence or partial sequence (e.g., 50, 100, 150, or 200 contiguous amino acid portions) as compared to a naturally occurring CD112R protein.

The term “antibody” is used herein in its broadest sense and encompasses numerous antibody structures, including but not limited to monoclonal antibodies, polyclonal antibodies, multispecific antibodies (e.g., bispecific antibodies), and antibody fragments, as long as they exhibit the desired antigen binding activity. Intact antibody typically comprises at least two full-length heavy chains and two full-length light chains, but in some cases may comprise fewer chains, for example a naturally occurring antibody in camelids may comprise only heavy chains.

The term “antibody fragment” refers to a molecule that is different from an intact antibody and comprises a portion of the intact antibody and is capable of binding to an antigen to which the intact antibody binds. Examples of antibody fragments include but are not limited to Fv, Fab, Fab′, Fab′-SH, F(ab′)₂; diabodies; linear antibodies; single-chain antibodies (e.g. scFv); single-domain antibodies; bivalent or bispecific antibodies or fragments thereof; camelid antibodies (heavy chain antibodies); and multispecific antibodies formed from antibody fragments.

The term “variable region” or “variable domain” refers to a domain of an antibody heavy or light chain involved in binding of an antibody to an antigen. The variable domains of the heavy and light chains of a natural antibody typically have similar structures, with each domain comprising four conserved framework regions (FRs) and three complementarity determining regions (CDRs) (see, e.g., Kindt et al. Kuby Immunology, 6th ed., W. H. Freeman and Co., page 91 (2007)). A single VH or VL domain is sufficient to confer antigen-binding specificity.

A “complementarity determining region” or “CDR region” or “CDR” is a region of the antibody variable domain that is highly variable in sequence and forms structurally determined loops (“hypervariable loops”) and/or contains antigen-contacting residues (“antigen-contacting sites”). The CDRs are primarily responsible for binding to epitopes in antigen. The CDRs in the variable domain are commonly referred to as CDR1, CDR2 and CDR3, numbered sequentially from the N-terminus. In a given variable region amino acid sequence, the exact amino acid sequence boundaries of each CDR can be determined using any one or a combination of many well-known antibody CDR designation systems, including, for example: Chothia (Chothia et al. (1989) Nature 342:877-883, Al-L azikani et al., “Standard formulations for the canonical structures of immunoglobulins”, Journal of Molecular Biology, 273, 927-948 (1997)) based on three-dimensional structure of antibody and topology of CDR loops, Kabat (Kabat et al., Sequences of Proteins of Immunological Interest, 4th ed., U.S. Department of Health and Human Services, National Institutes of Health (1987)), AbM (University of Bath), Contact (University College London), International ImMunoGeneTics database (IMGT) based on antibody sequence variability, and North CDR definitions based on affinity propagation clustering with a large number of crystal structures.

Unless otherwise specified, in the invention, the term “CDR” or “CDR sequence” encompasses a CDR sequence determined in any of the methods described above.

The CDR may also be determined based on having the same AbM numbering locations as the reference CDR sequence (e.g., any of the CDRs of the present examples). In one embodiment, the positions of CDRs of the antibody of the invention are determined according to AbM numbering scheme.

Unless otherwise specified, in the invention, when reference is made to the position of residues in an antibody variable region and a CDR, including heavy chain variable region residues, it refers to the numbering position according to the AbM numbering system.

By “humanized antibody” is meant a chimeric antibody comprising amino acid residues from non-human CDRs and amino acid residues from human FRs. In some embodiments, all or substantially all of the CDRs (e.g., CDRs) in a humanized antibody correspond to those of a non-human antibody, and all or substantially all of the FRs correspond to those of a human antibody. The humanized antibody optionally may comprise at least a portion of an antibody constant region derived from a human antibody. “Humanized form” of an antibody (e.g., a non-human antibody) refers to an antibody that has been humanized.

As used herein, the term “bind” or “specifically binds” means that the binding effect is selective to the antigen and may be distinguished from unwanted or non-specific interactions. The ability of the antigen-binding site to bind to a specific antigen can be determined by methods such as enzyme-linked immunosorbent assay (ELISA).

The term “half effective concentration (EC₅₀)” refers to the concentration of a drug, antibody or toxin that induces a response at 50% between baseline and maximum after a specified exposure time.

The term “therapeutic” as used herein encompasses any substance that is effective in prevention or treatment of a tumor, such as cancer, including chemotherapeutic agents, cytokines, angiogenesis inhibitors, cytotoxic agents, other antibodies, small molecule drugs, or immunomodulators (e.g., immunosuppressive agents).

The term “treatment” refers to slowing, interrupting, retarding, alleviating, stopping, reducing, or reversing the progression or severity of a pre-existing symptom, disorder, condition, or disease. Desirable therapeutic effects include, but not limited to, preventing the occurrence or recurrence of a disease, reducing symptoms, reducing any direct or indirect pathological consequences of a disease, preventing metastasis, reducing the rate of disease progression, ameliorating or alleviating a disease state, and alleviating or improving prognosis. In some embodiments, the antibody of the invention is used to delay disease development or to slow the progression of a disease.

The term “prevention” includes inhibition of the occurrence or development of a disease or disorder or a symptom of a particular disease or disorder. In some embodiments, a subject having a history of cancer families is a candidate for a prophylactic regimen. Generally, in the context of cancer, the term “prevention” refers to the administration of a drug prior to the onset of signs or symptoms of cancer, particularly in subjects at a risk of having a cancer.

The term “effective amount” refers to the amount or dose of an antibody or conjugate or composition of the invention that produces a desired effect in a patient in need of treatment or prevention following administration to the patient in single or multiple doses. Effective amounts can be readily determined by attending physicians as those skilled in the art by considering a variety of factors such as species of mammals; weight, age and general health; specific diseases involved, degree or severity of disease; response of individual patients; specific antibody administered; mode of administration; bioavailability characteristics of administered formulations; selected dosing regimen; and use of any concomitant therapy.

The term “therapeutically effective amount” refers to an amount effective to achieve a desired therapeutic result at a desired dose and for a desired period of time.

The therapeutically effective amount of an antibody or antibody fragment or conjugate or composition thereof can vary depending on a variety of factors, such as disease state, age, sex, and weight of the subject, and the ability of the antibody or antibody portion to elicit a desired response in the subject. A therapeutically effective amount is also an amount in which any toxic or detrimental effects of an antibody or antibody fragment or conjugate or composition thereof are less than the therapeutically beneficial effects. “Therapeutically effective amount” preferably inhibits measurable parameters (e.g., tumor growth rate, tumor volume, etc.) by at least about 20%, more preferably at least about 40%, even more preferably at least about 50%, 60%, or 70% and still more preferably at least about 80% or 90% relative to untreated subjects. The ability of a compound to inhibit measurable parameters (e.g., cancer) can be evaluated in an animal model system that predicts efficacy in human tumors.

The term “prophylactically effective amount” refers to an amount effective to achieve a desired prophylactic result at a desired dose and for a desired period of time. Typically, the prophylactically effective amount will be less than the therapeutically effective amount, since the prophylactic dose is used in a subject prior to or at an earlier stage of the disease.

The term “pharmaceutical composition” refers to a composition that allows an active ingredient contained therein to be present in a biological active form and does not contain an additional ingredient that has unacceptable toxicity to a subject in which the composition is administered.

### II. Compositions of the Invention

In some embodiments, the invention provides a composition comprising any of the anti-CD112R antibodies described herein and antigen-binding fragments thereof, preferably the composition is a pharmaceutical composition. In one embodiment, the composition further comprises a pharmaceutic adjuvant, such as a pharmaceutically acceptable carrier, a pharmaceutically acceptable excipient, including a buffer, as is known in the art. In one embodiment, the composition (e.g., a pharmaceutical composition) comprises the anti-CD112R antibody and antigen-binding fragment thereof of the invention, as well as a combination of one or more other therapeutics.

As used herein, “pharmaceutically acceptable carrier” includes any and all solvents, dispersion media, isotonic agents and absorption delaying agents and the like that are physiologically compatible.

For the use and use of pharmaceutical adjuvant, see also “Handbook of Pharmaceutical Excipients,” Eighth Edition, R. C. Rowe, P. J. Seskey and S. C. Owen, Pharmaceutical Press, London, Chicago.

The compositions of the invention can be in a variety of forms. These forms include, for example, liquid, semi-solid, and solid dosage forms such as liquid solutions (e.g., injectable solutions and infusion solutions), powders or suspensions, liposome formulations, and suppositories. Preferred forms depend on the intended mode of administration and therapeutic use.

The route of administration of the composition of the invention is via known methods, for example, orally, intravenous injection, intraperitoneal, intracerebral (parenchyma), intracerebroventricular, intramuscular, intraocular, intraarterial, portal or intralesional routes; by sustained release systems or by implanted devices. In certain embodiments, the composition may be administered by bolus injection or by continuous infusion or by an implant device.

The subject may be a mammal, e.g., a primate, preferably a higher primate, e.g., a human (e.g., a subject having a disease described herein or having a risk of having a disease described herein). In one embodiment, a subject has or has a risk of having a disease described herein (e.g., cancer). In certain embodiments, the subject receives or has received other treatments, such as chemotherapy treatment and/or radiotherapy. In some embodiments, the subject has previously received or is receiving immunotherapy.

Drug comprising the antibody described herein can be prepared by mixing the anti-CD112R antibody and antigen-binding fragment thereof of the invention having a desired purity with one or more optional pharmaceutical adjuvants, preferably in the form of lyophilized preparation or aqueous solution.

The pharmaceutical composition or formulation of the invention may further comprise more than one active ingredients required for the particular indication being treated, preferably those having complementary activities that do not adversely affect each other. For example, it may be desirable to also provide other therapeutics, including chemotherapeutic agents, angiogenesis inhibitors, cytokines, cytotoxic agents, other antibodies, small molecule drugs or immunomodulators (e.g., immune checkpoint inhibitors or agonists), and the like. The active ingredient is suitably combined in an amount effective for the intended use.

### III. Preparation of Antibodies of the Invention

In one embodiment, the invention provides a method of preparing an anti-CD112R antibody, comprising culturing a host cell comprising a nucleic acid encoding the anti-CD112R antibody or an expression vector comprising the nucleic acid under conditions suitable for expression of the nucleic acid encoding the anti-CD112R antibody, and optionally isolating the anti-CD112R antibody. In an embodiment, the method further comprises recovering the anti-CD112R antibody from the host cell (or host cell culture medium).

To recombinantly produce the anti-CD112R antibody of the invention, the nucleic acid encoding the anti-CD112R antibody of the invention is firstly isolated and is inserted into a vector for further cloning and/or expression in a host cell. Such nucleic acids are readily isolated and sequenced using conventional protocols, such as by using oligonucleotide probes capable of specifically binding to nucleic acids encoding the anti-CD112R antibody of the invention.

The anti-CD112R antibody of the invention prepared as described herein may be purified by known prior art techniques such as high performance liquid chromatography, ion exchange chromatography, gel electrophoresis, affinity chromatography, size exclusion chromatography, and the like. The actual conditions used to purify a particular protein also depend on factors such as net charge, hydrophobicity, hydrophilicity, etc., and are apparent to those skilled in the art.

### EXAMPLES

The invention is further illustrated by the following examples, however, it is to be understood that the examples are described by way of illustration instead of limitation, and that various modifications may be made by those skilled in the art. The present disclosure will be further described in detail below with reference to specific examples, but these examples are not intended to limit the scope of the invention. The experimental methods for which specific conditions are not indicated in the examples of the present disclosure are generally carried out under conventional conditions, or according to the conditions recommended by the raw materials or commercial manufacturers; and the unspecified reagents are generally conventional reagents commercially available.

Unless expressly indicated to the contrary, examples of the invention will employ methods of conventional chemistry, biochemistry, organic chemistry, molecular biology, microbiology, recombinant DNA technology, genetics, immunology, and cell biology within the skill of the art.

### Example 1: Preparation and Identification of Raw Materials

### 1.1 CD112R Control Antibody Preparation and Identification

CD112R control antibody preparation: the present application used anti-CD112R antibody CHA.7.518 (hereinafter referred to as Tab-1) as a positive control antibody, and gene synthesis by GenScript Biotech Corporation was according to the sequence disclosed in WO %2016/134333, and an expression plasmid was constructed, where the specific VH and VL sequences of Tab-1 were shown in SEQ ID NOs: 657 and 658. Expi293F cells were transiently transfected and cultured for 6 days, the supernatant was collected, and antibody affinity purification was performed using Protein A Magnetic Beads (GenScript Biotech Corporation). Protein A Magnetic Beads were added to the supernatant and incubated at room temperature for 2 hours. After incubation, Protein A Magnetic Beads were equilibrated with Binding Buffer (PBS, pH 7.4) for 10 column volumes. The Protein A Magnetic Beads were added to an elution buffer (0.1M citric acid buffer, pH 3.0) to elute, the eluate was collected, a Neutralizing buffer (0.1M Tris pH 9.0) of 1/10 volume of the eluate were added to adjust pH to be neutral. After elution, Protein A Magnetic Beads were equilibrated with Binding buffer for 10 column volumes. The antibody was eluted and neutralized, placed in a dialysis bag for dialyzing against Dialysis buffer (PBS, pH 7.4) for 2 hours at room temperature, and dialysis buffer was replaced at 2-8° C. for 16 hours. The antibody was transferred from the dialysis bag to a centrifuge tube and the volume was recorded.

CD112R control antibody identification: the activity of the prepared positive control antibody Tab-1 was detected by using the purchased CD112R-His antigen protein (Acro Biosysterm, PVG-H52H4), and the specific method was as follows; CD112R-His (0.5 μg/mL, 100 μL/well) was coated on a 96-well ELISA plate at 37° C. for 1 hour; after washing once, the plate was blocked with 1% BSA prepared in PBS for 1 hour; after washing for 3 times, the control antibody Tab-1 diluted gradiently with PBS was added and incubated at room temperature for 1 hour; after washing the plate, a secondary antibody (Peroxidase-AffiniPure Goat Anti-Mouse IgG, Fcγ Fragment Specific) diluted with PBS (1:6000) was added and incubated at 37° C. for 0.5 hours, the plate was washed for 3 times, and the TMB was added for development for 5-20 min; after stopping development, the data was read at OD450 with a reader. The results showed that the expressed control antibody Tab-1 can bind the CD112R protein, and as the antibody concentration decreased, the OD450 reads decreased, indicating that the control antibody had normal anti-CD112R activity.

### Example 2: Construction and Identification of Cell Lines Expressing Human and Monkey CD112R

Construction of CHOK1 cell lines overexpressing human and monkey CD112R (hereinafter referred to as huCD112R-CHO K1; cynoCD112R-CHO K1): the nucleic acid sequences encoding full-length human (Uniprot ID: Q6DKI7) and monkey CD112R (Uniprot ID: A0A2K5WVV8) were constructed onto lentiviral vectors. Then, the lentiviral plasmid was transfected into HEK293T cells for virus package, cultured for 4 days, and the cell culture supernatant was collected. CHO-K1 cells were transfected with the harvested lentivirus supernatant, and Puromycin was used as screening pressure. After screening for 3 days, the antigen expression in cell pool was screened by flow cytometry. After obtaining the positive cell pool, the positive cell pool was selected by using limited dilution method or flow sorting. After monoclone selection, the culture was continued for about 1 week, and the antigen expression in cell strains was identified.

Flow identification of antigen expression in cell strain: the cell strains in logarithmic growth phase were digested and plated into a 96-well plate, washed with FACS buffer (1×PBS buffer containing 2% of FBS v/v), and incubated with a PBS gradient diluted primary Mouse anti-human PVRIG antibody (GenScript Probio, Cat. No.: C523UGG270-2D) for 30 min; after washing, the prepared fluorescent secondary PE Goat anti-mouse IgG antibody (BioLegend, Cat. No. 405307) was added, and incubated at 4° C. for 30 min; and finally detected by flow cytometry (Beckman, CytoFLEXAOO-1-1102). The results showed that huCD112R-CHO K1 and cynoCD112R-CHO K1 cell lines with high surface expression human and monkey CD112R were obtained.

### Example 3: Animal Immunization and Immune Library Construction

### 3.1 Immune Protocol

Different species of mice (Balb/c, SJL, A/J) were immunized with various immunogens, CD112R-His or CD112R-mFc protein antigen (Acro Biosysterm, PVG-H5259); CD112R expression plasmid-DNA antigen; CD112R overexpressing CHO cells, alone or alternatively. Mice were immunized by subcutaneous/intraperitoneal injection or gene-gun depending on the type of antigen selected, once every two weeks, for a total of 3-5 immunizations. Mouse blood was taken one week after the last immunization for immune titer detection, and finally, the mice was boosted once again with the above antigen.

TABLE 1
Summary of immunization in this example

| | Immunization Summary |
|---|---|
| 1 | CD112R-His protein antigen (Acro Biosysterm, |
| | PVG-H52H4) |
| 2 | CD112R-mFc protein antigen (Acro Biosysterm, |
| | PVG-H5259) |
| 3 | DNA antigen (SEQ ID NO: 662) + cell antigen |
| | (human protein overexpressing cell line constructed |
| | in Example 2) + CD112R-His protein antigen (Acro |
| | Biosysterm, PVG-H52H4) |
| 4 | CD112R-His protein antigen (Acro Biosysterm, |
| | PVG-H52H4) + cell antigen (human protein |
| | overexpressing cell line constructed in Example |
| | 2) + CD112R-His protein antigen (Acro Biosysterm, |
| | PVG-H52H4 |

3.2 Detection of Antibody Titer in Mice Serum after Immunization

The antigen was diluted to 0.5 μg/mL with coating solution (PBS, pH 7.4), mixed well and added into an ELISA plate, 100 μL/well, and placed in a refrigerator at 4° C. overnight for 16 h. The coating solution was discarded, the plate was washed once, and the plate was dried. 150 μL of blocking solution (3% BSA solution) was added to each well and placed in a 37° C. incubator for 1 h. The plate was taken out and the blocking solution was discarded. After gradient dilution of mice serum, 100 μL/well was added to the wells of the plate. Incubation for 1 h at 37° C. incubator. The plate was taken out, the liquid in the plate was discarded, and the plate was washed 3 times. 100 μL of the diluted enzyme-labeled secondary antibody Peroxidase-AffiniPure Goat Anti-Mouse IgG, Fcγ Fragment Specific (min X Hu, Bov, Hrs Sr Prot) was added to each well and incubated at 37° C. for 0.5 hours. The ELISA plate was taken out, the liquid in the ELISA plate was discarded, the plate was washed for 3 times, and 100 μl of color development solution was added after the plate was dried, and reacted at 25° C. for 15 min. 50 μL of 1M HCl was added to terminate the reaction, and the reading at OD450 was performed with a microplate reader. Mice with a titer greater than 512000 (dilution fold) were selected for hybridoma fusion.

### 3.3 Hybridoma Cell Fusion

A suspension of myeloma cells (SP2/0) was prepared and the total number of SP2/0 cells was calculated with a hemocytometer. The spleen cell suspension was prepared, and the total number of cells was counted using the blood cell counting plate. Myeloma cells and splenocytes were mixed at a ratio of 1:2 to perform cell electrofusion. After cell fusion, HAT selective medium was used to mix the cells after fusion into a cell suspension, and then the cells were plated into 96-well cell culture plate by using a multichannel pipette. The fused 96-well cell culture plate was placed in a 5.5% CO₂ incubator, and cultured at a constant temperature of 37° C. After the hybridoma cell culture, the supernatant was taken for mother clone screening, and the cell culture supernatant was screened for antigen binding ability at protein and cell levels to obtain a mother clone cell line capable of binding to an antigen.

### 3.4 Hybridoma Subclone Screening

The mother clone cell line obtained by screening in step 3.3 was picked out, and the cells to be subcloned were pipetted gently and mixed well. 20 μl of cell suspension was taken and added to 20 μl of trypan blue solution for uniform mixing. 20 μl of the solution was taken for counting by using blood cell count plate. Using a limited dilution method, cells were plated in a 96-well cell culture plate with 1×HT replacement feeder cell culture medium, marked on the 96-well plate, and placed in CO₂ incubator for culture. The subcloned 96 well plate was incubated in a 37° C., 5.5% CO₂ incubator. After 7 days of culture, the supernatant was taken for monoclonal screening.

Through subclone screening, the obtained antibodies were selected for sequencing, and the CDR sequences of the antibodies were shown in Table 2 and Table 3 below. CDR sequences are determined by using AbM to define CDRs.

TABLE 2
Heavy Chain CDR Sequences of the Anti-CD112R Antibodies

| Antibody | SEQ | | SEQ | | SEQ ID | |
|---|---|---|---|---|---|---|
| No. | ID No: | HCDR1 | ID No: | HCDR2 | No: | HCDR3 |
| C1 | 1 | DYYLN | 64 | WIFPGSESTFYNEKFKG | 162 | DGGLRGPFDY |
| C3 | 2 | DYYIN | 65 | WIFPGSDYTYYNEKFKG | 162 | DGGLRGPFDY |
| C4 | 3 | DYFMN | 66 | DIIPNNDSPNYNQKFKG | 163 | GYFDV |
| C6 | 4 | AYYIN | 67 | WIFPGSDSTYYNEKFKG | 164 | DGELRGPFDY |
| C7, C7-2 | 5 | DYYMN | 68 | DINPNNGGTNFNQKFKG | 165 | LFFDY |
| C8 | 6 | DYYMF | 69 | TISDGGGYIYYPDSVKG | 166 | EDF |
| C9 | 7 | DIYMH | 70 | RIDPTNGNSEYDPRFRG | 167 | FGIYPYFDY |
| C10 | 8 | DSYMC | 71 | TISDGGGLTYYPDSVKG | 168 | EDL |
| C11 | 3 | DYFMN | 72 | DIHPNNGSPNYNQKFKG | 163 | GYFDV |
| C12 | 9 | DYYMY | 73 | TISDGGGYTYYPDSVKG | 168 | EDL |
| C13 | 3 | DYFMN | 74 | DINPNNGSPNYNQKFKG | 163 | GYFDV |
| C14 | 10 | SYWMH | 75 | VIDASDSNTKYNQKFKG | 169 | YYSGSSYGLDY |
| C16 | 10 | SYWMH | 75 | VIDASDSNTKYNQKFKG | 170 | YFSGSSYGLDY |
| C18 | 9 | DYYMY | 76 | TISDGGGFIYYPDSVKG | 168 | EDL |
| C19 | 9 | DYYMY | 77 | TISDAGGYTYYPDSVKG | 168 | EDL |
| C20 | 8 | DSYMC | 77 | TISDAGGYTYYPDSVKG | 168 | EDL |
| C21 | 7 | DIYMH | 78 | RVDPTNGNSEYDPRFQG | 167 | FGIYPYFDY |
| C22 | 11 | DYNIH | 79 | YIYPYNGGAGYNQKFKS | 171 | EELRRDYGMDY |
| C23 | 2 | DYYIN | 80 | WIFPGSGNTYYNEKFKG | 162 | DGGLRGPFDY |
| C24 | 12 | GYGIN | 81 | VIWSGGNTEYNAAFRS | 172 | DSTGLYAMDY |
| C25 | 10 | SYWMH | 82 | VIDASDSKTKYNQKFKG | 169 | YYSGSSYGLDY |
| C28 | 2 | DYYIN | 83 | WIYPGSGYTKYNEKLKG | 173 | DSFYAMDY |
| C31 | 13 | DYYIA | 84 | WIYLGSGHTMYNENFKG | 173 | DSFYAMDY |
| C33 | 2 | DYYIN | 85 | WIFPGSGSTYYNEKFKG | 174 | EDYFDRGDC |
| C36 | 14 | TYGMS | 86 | WINTYSGVPTYADDFKG | 175 | ELPFTTIGTKDY |
| C38 | 15 | GYWIE | 87 | EILPESGRTNYNEKFKG | 176 | LLFFDY |
| C39 | 16 | SYNIH | 88 | AIYPGNADISYNQKFRD | 177 | PLRIYAMDC |
| C41 | 2 | DYYIN | 89 | DIEPNNGGTNYNQKFKG | 178 | LYFDY |
| C43 | 17 | TYWMH | 75 | VIDASDSNTKYNQKFKG | 169 | YYSGSSYGLDY |
| C44 | 18 | DSYIN | 85 | WIFPGSGSTYYNEKFKG | 162 | DGGLRGPFDY |
| C45 | 2 | DYYIN | 90 | WIFPGSGTTYYNEKFKG | 162 | DGGLRGPFDY |
| C47-2 | 19 | SYWMQ | 91 | EIDPFDSYTNYNQKFKG | 179 | RVYYGYDRYFFDY |
| C48, C48-2 | 2 | DYYIN | 85 | WIFPGSGSTYYNEKFKG | 180 | DGSYDPPFAY |
| C51 | 20 | DYEMH | 92 | AIDPETGGTAYNQKFKG | 181 | WITTVVATLYYFDY |
| C52 | 20 | DYEMH | 92 | AIDPETGGTAYNQKFKG | 181 | WITTVVATLYYFDY |
| C55 | 21 | DYYMS | 93 | DVNPNNGDTSYNQKFKG | 182 | SGLPYVLMDY |
| C56 | 22 | GYALS | 94 | TIRDGGSYTYYSDNEKG | 183 | DQVNRGWFTY |
| C56-2 | 23 | DYALS | 94 | TIRDGGSYTYYSDNEKG | 183 | DQVNRGWFTY |
| C57 | 24 | DYYMH | 95 | HINPYNGDTSYNQKFKD | 184 | DSSNVGNAMDY |
| C58 | 21 | DYYMS | 93 | DVNPNNGDTSYNQKFKG | 182 | SGLPYVLMDY |
| C59 | 22 | GYALS | 94 | TIRDGGSYTYYSDNEKG | 183 | DQVNRGWFTY |
| C61 | 25 | SNYYWN | 96 | YISSDGINNYNPSLRN | 185 | GLRYGP |
| C62 | 26 | SYSMS | 97 | TIGGVGGNTYYPDSVKG | 186 | HEDYYYYAMDY |
| C63 | 27 | SYIMS | 98 | TIRGGGGNTYYLDSVKG | 187 | HEDYEWYAMDN |
| C64 | 28 | SYFMS | 99 | TISGGGDKTYYLDILKG | 188 | HEDYAWYAMDY |
| C66 | 22 | GYALS | 94 | TIRDGGSYTYYSDNEKG | 183 | DQVNRGWFTY |
| C67 | 5 | DYYMN | 100 | DINPDNGGSTYNQKFKG | 189 | SGPPYVLLDY |
| C69 | 24 | DYYMH | 101 | IINPYNGGTSYNQKFKG | 190 | DSSNVGNDMDY |
| C71 | 28 | SYFMS | 102 | TISGGGDNTYYPDSVKG | 191 | HEDYEWYAMDY |
| C72 | 28 | SYFMS | 103 | TIRGGGSYTYYLDSVKG | 192 | HEDYDYYAMDY |
| C75 | 29 | SYWIT | 104 | DIYPGNFSPNYNEKFKN | 163 | GYFDV |
| C76 | 30 | HYYKN | 105 | AINPNIGGPSYNQKFKG | 193 | SDDYGGYYFDY |
| C77 | 5 | DYYMN | 106 | DIYPNNGDTNYNQKFKG | 194 | GVGLGFPY |
| C78 | 31 | HYYVN | 107 | VIKPYNGAPSYNQKFKG | 195 | SDDFDGYAMDY |
| C79-2 | 31 | HYYVN | 107 | VIKPYNGAPSYNQKFKG | 195 | SDDFDGYAMDY |
| C79-3, C79-4 | 32 | NYGMS | 108 | TISRGGSFTYYPDSVKG | 196 | PIYYEYDGFAY |
| C80 | 29 | SYWIT | 109 | DIYPGNDSPNYNEKFKN | 197 | GYYDY |
| C81 | 33 | GYYVN | 110 | LIDPSNGGTNYNQKFKD | 198 | GTVY |
| C82 | 5 | DYYMN | 111 | TFNPNRGGPSYNLKFKG | 199 | ADDYDGYYFDY |
| C84, C84-2 | 34 | DYFIS | 112 | WIFPGSGSTYFNENFKG | 200 | YYGSNY |
| C85 | 35 | DYSMN | 113 | DINPNNGGTSYNQKFKG | 201 | VWFAY |
| C87 | 36 | ASAMH | 114 | SFTMYSDATEYSENFKG | 202 | GYSGNGFTY |
| C88 | 29 | SYWIT | 115 | DIHPGNTSPNYNEKFRT | 203 | GWGDY |
| C89 | 37 | DYGMY | 116 | YIRSDSSTIYYTDTVKG | 204 | RSPVVVNYAMDY |
| C90 | 5 | DYYMN | 117 | EINPKNGDTNYNQNFKG | 205 | EGVSSYYAMDY |
| C91 | 38 | GYYIN | 110 | LIDPSNGGTNYNQKFKD | 198 | GTVY |
| C92 | 39 | DYGMH | 118 | YIRSDSSTIYYADTVKG | 204 | RSPVVVNYAMDY |
| C93 | 5 | DYYMN | 119 | EINPKNGDTNYSQKFRD | 206 | EGVGSYYAMDY |
| C94 | 29 | SYWIT | 109 | DIYPGNDSPNYNEKFKN | 197 | GYYDY |
| C95 | 38 | GYYIN | 110 | LIDPSNGGTNYNQKFKD | 198 | GTVY |
| C96 | 5 | DYYMN | 120 | EINPNNGDTNYSQKFRD | 205 | EGVSSYYAMDY |
| C97 | 5 | DYYMN | 121 | EINPKNGDTNYNQNFKA | 207 | EGVSSYYGMDS |
| C98 | 35 | DYSMN | 122 | DINPNNGGTNYNQKFKG | 201 | VWFAY |
| C99 | 35 | DYSMN | 123 | DINPDNGGTSYNQKFKG | 208 | IWFAY |
| C100 | 5 | DYYMN | 124 | EINPNNGDTNFNQKFKG | 209 | EGGSSYYAMDY |
| C101 | 40 | DYNVD | 125 | AINPNDGGTSYNQKFEG | 210 | WVNNDWYFDV |
| C102 | 41 | NYGIS | 126 | EIYPGTINIYYSERFKG | 211 | DDGYYDAMDY |
| C103 | 10 | SYWMH | 127 | EIDPSDSYTNYNQKFKG | 212 | SIYYSNYVRYYFDY |
| C103-2 | 40 | DYNVD | 125 | AINPNDGGTSYNQKFEG | 213 | WVINDWYFDV |
| C104 | 42 | DYNMD | 128 | ANDPKNGGFSYNQRFKG | 214 | WDSNYWYFDV |
| C105 | 43 | DYSMD | 129 | AINPNNGGTSYNQKFKD | 215 | WDSDYWYFDV |
| C106 | 42 | DYNMD | 130 | AVNPNTGDISYTQKFKD | 216 | WSESWYFDV |
| C107 | 44 | DYNID | 131 | AIDPNNGGTNYNQKFKD | 217 | WDSNDWYFDV |
| C108, | 44 | DYNID | 132 | AIDPNNDSTNYNQKFKG | 218 | WDSDDWYFDV |
| C108-2 | | | | | | |
| C110 | 45 | DTYIH | 133 | GIDPAIGNTVSASKFQD | 219 | ERVHSNTLYYAMD |
| | | | | | | Y |
| C111, | 46 | SHGIS | 134 | EIYPGSESPYYSEKFKG | 220 | SDYSDYWAMDY |
| C111-2 | | | | | | |
| C112, | 47 | SSWMH | 135 | EINPSDGRTNYNEKFKS | 221 | NADRAYYTMDY |
| C112-2 | | | | | | |
| C113, | 48 | DYNLD | 136 | AMSPNNGATSYNQKFKG | 222 | WDSNSWYFDV |
| C113-2 | | | | | | |
| C114 | 49 | NSWIN | 137 | DIYPGNTFPNYNEKFKG | 163 | GYFDV |
| C115 | 45 | DTYIH | 138 | GIDPAIGNTLYASKFQD | 223 | ERVYSNTLYYAMD |
| | | | | | | Y |
| C116 | 50 | SYTMS | 139 | KISNSGQSTNYPDTVKG | 224 | YSWDVYAMDY |
| C117 | 51 | DYWMQ | 140 | AIFPGNGETRDTQKFKG | 225 | FNNYLDY |
| C118 | 47 | SSWMH | 141 | EINPSDGRTNYNEKFKN | 221 | NADRAYYTMDY |
| C119 | 52 | TYIMS | 142 | DISNSGGNTYYSDTVKG | 226 | PDNWDPFVY |
| C120 | 53 | DYFIN | 143 | DINPDNGAGNYNQKFKG | 178 | LYFDY |
| C121 | 53 | DYFIN | 144 | DINPDNGGTNYNQKFKG | 178 | LYFDY |
| C122 | 53 | DYFIN | 145 | DVNPDNGGTNYNQKFK | 227 | LFFDS |
| | | | | G | | |
| C123 | 54 | NFAMS | 146 | TIVNGGINTYYPDTVKG | 228 | HDDFLYYAMDY |
| C124 | 55 | SFAMS | 147 | TINNGGINTYYPDTVKG | 228 | HDDFLYYAMDY |
| C125 | 50 | SYTMS | 148 | ELSNSGGSFHYPDNVKG | 229 | HNWDVYAMDY |
| C129 | 56 | DYWIE | 149 | EILPGYISTNYNEKFKD | 230 | IVTTYYYFMDY |
| C132 | 57 | DYWIN | 150 | EILPGSVTTNYNEKFRG | 231 | GSDYSYYAMDY |
| C136 | 58 | ENYMY | 151 | TISDGASYTYYPDNVKG | 232 | DELIAPFAY |
| C138 | 59 | SYAMS | 152 | TITSGDIRTYYPDTVKG | 233 | HDDFHFYAMDY |
| C140 | 60 | NYWMN | 153 | AINPSNGYIGYNQKFKD | 234 | GEDFDGYAMDY |
| C143 | 54 | NFAMS | 146 | TIVNGGINTYYPDTVKG | 228 | HDDFLYYAMDY |
| C145 | 61 | DFFIN | 154 | DVNPDSNGANYNQKFK | 235 | LFFDN |
| | | | | G | | |
| C146 | 40 | DYNVD | 125 | AINPNDGGTSYNQKFEG | 210 | WVNNDWYFDV |
| C147 | 44 | DYNID | 155 | AINPNNGGTFYNQKPKG | 236 | WDSDSWYFDV |
| C148 | 44 | DYNID | 156 | AIDPNNGGTNYNQKFKG | 217 | WDSNDWYFDV |
| C149 | 62 | TFGLGIG | 157 | HIWWDDDKYYNPALKS | 237 | GTYWFTY |
| C150 | 24 | DYYMH | 158 | INPYNGDTSYNQKFKG | 184 | DSSNVGNAMDY |
| C151 | 3 | DYFMN | 159 | DINPNNGDTTYSQKFMG | 189 | SGPPYVLLDY |
| C152 | 63 | DYWIQ | 160 | AIFPENGDTRYTQKFKG | 238 | FINYVDY |
| C153 | 42 | DYNMD | 161 | AIDPKNGATSYNQKFKG | 214 | WDSNYWYFDV |

TABLE 3
Light Chain CDR Sequences of the Anti-CD112R Antibodies

| Antibody | SEQ ID | | SEQ ID | | SEQ ID | |
|---|---|---|---|---|---|---|
| No. | No: | LCDR1 | No: | LCDR2 | No: | LCDR3 |
| C1 | 239 | KASQDINKYIA | 307 | YTSTLHP | 357 | LQYDYLLT |
| C3 | 239 | KASQDINKYIA | 308 | YTSTLQP | 358 | LQYDNLHT |
| C4 | 240 | KSSQSLLNSGNQKNFLA | 309 | GTSTRES | 359 | QNDHSYPWT |
| C6 | 239 | KASQDINKYIA | 310 | YTSTLQA | 360 | LQYDNLLT |
| C7 | 241 | KSSQSLLNSGNQKNYLA | 311 | GASTRES | 361 | QNAHSSPYT |
| C7-2 | 242 | KASQNVGTAVA | 312 | SASNRYT | 362 | QQYSSYRT |
| C8 | 243 | RASQDIGRSLN | 313 | ATSRLDS | 363 | LQYASFPYT |
| C9 | 244 | KASQSVNKAVV | 314 | YASNRYT | 364 | QQDYSSPWT |
| C10 | 245 | RASQDIGSSLN | 313 | ATSRLDS | 365 | LQYASSPYT |
| C11 | 241 | KSSQSLLNSGNQKNYLA | 311 | GASTRES | 359 | QNDHSYPWT |
| C12 | 245 | RASQDIGSSLN | 313 | ATSRLDS | 363 | LQYASFPYT |
| C13 | 246 | KSSQSLLNSGTQKNYLA | 311 | GASTRES | 359 | QNDHSYPWT |
| C14 | 247 | RASQDISNYLN | 315 | YTSRLHS | 366 | QQGNMFPPT |
| C16 | 247 | RASQDISNYLN | 315 | YTSRLHS | 366 | QQGNMFPPT |
| C18 | 243 | RASQDIGRSLN | 313 | ATSRLDS | 363 | LQYASFPYT |
| C19 | 248 | RASQDIGYSLN | 313 | ATSRLDS | 363 | LQYASFPYT |
| C20 | 243 | RASQDIGRSLN | 313 | ATSRLDS | 365 | LQYASSPYT |
| C21 | 244 | KASQSVNKAVV | 314 | YASNRYT | 364 | QQDYSSPWT |
| C22 | 249 | RASENIYSYLA | 316 | NAKTLAE | 367 | QHHYGTPYT |
| C23 | 239 | KASQDINKYIA | 317 | FTSTLQS | 368 | LQYDYLYT |
| C24 | 250 | RTSQDISNFLN | 315 | YTSRLHS | 369 | QQGYMLPPT |
| C25 | 247 | RASQDISNYLN | 315 | YTSRLHS | 370 | QQGNTLPPT |
| C28 | 242 | KASQNVGTAVA | 312 | SASNRYT | 371 | LQYSSYLT |
| C31 | 242 | KASQNVGTAVA | 312 | SASNRYT | 372 | QQYSTYLT |
| C33 | 251 | SASSSIN YMH | 318 | DTSKLAS | 373 | HQRSSYPFT |
| C36 | 252 | RASQFISNNLH | 319 | YASQSIS | 374 | QQSNSWPHT |
| C38 | 253 | KSSQSLLDSDGKTYLN | 320 | LVSKLDS | 375 | WQGTHFPLT |
| C39 | 254 | KASQNVGTNVA | 321 | SASYRYS | 376 | QQYNSRPLT |
| C41 | 241 | KSSQSLLNSGNQKNYLA | 311 | GASTRES | 377 | QNDHSYPFT |
| C43 | 247 | RASQDISNYLN | 315 | YTSRLHS | 366 | QQGNMFPPT |
| C44 | 239 | KASQDINKYIA | 322 | YTSTLQS | 378 | LQYDHLLT |
| C45 | 255 | KANQDINKYIA | 308 | YTSTLQP | 368 | LQYDYLYT |
| C47-2 | 239 | KASQDINKYIA | 322 | YTSTLQS | 378 | LQYDHLLT |
| C48 | 256 | SASSSVSYMH | 323 | STSNLAS | 379 | QQRSSYPPT |
| C48-2 | 239 | KASQDINKYIA | 308 | YTSTLQP | 380 | LQYDNLYT |
| C51 | 257 | KASQNVRTAVA | 324 | LASNRHT | 381 | LQHWNYPLT |
| C52 | 257 | KASQNVRTAVA | 324 | LASNRHT | 381 | LQHWNYPLT |
| C55 | 258 | RASENINSYLA | 325 | NTKTLTE | 382 | QHHYGTPLT |
| C56, C56-2 | 259 | SASSSVSSSYFH | 323 | STSNLAS | 383 | HQHHRSPLT |
| C57 | 247 | RASQDISNYLN | 326 | YSSRLHS | 384 | QQGYTLPRT |
| C58 | 247 | RASQDISNYLN | 326 | YSSRLHS | 385 | QQGNTLPRT |
| C59 | 259 | SASSSVSSSYFH | 323 | STSNLAS | 383 | HQHHRSPLT |
| C61 | 260 | TLSSQHSTYTIE | 327 | GSHRTGD | 386 | GVGDINNYI |
| C62 | 261 | SANSTVSYIY | 328 | DTSNLAS | 387 | QQWSSFPFT |
| C63 | 262 | SARSSVSYMY | 328 | DTSNLAS | 388 | QQWSSYPLT |
| C64 | 263 | SASSSVNYIY | 328 | DTSNLAS | 388 | QQWSSYPLT |
| C66 | 239 | KASQDINKYIA | 308 | YTSTLQP | 389 | LQYDNLYM |
| C67 | 264 | QTSENIYSYLA | 329 | NTQTLAE | 390 | QHHYGVPLT |
| C69 | 247 | RASQDISNYLN | 326 | YSSRLHS | 385 | QQGNTLPRT |
| C71 | 265 | SASSSVSYIY | 328 | DTSNLAS | 391 | QQWTSYPLT |
| C72 | 266 | SASSSVSYMY | 328 | DTSNLAS | 388 | QQWSSYPLT |
| C75 | 241 | KSSQSLLNSGNQKNYLA | 311 | GASTRES | 377 | QNDHSYPFT |
| C76 | 267 | RANSSVNYMH | 330 | ATSNLAS | 392 | QQWTSNPLT |
| C77 | 268 | QASQVVSTAVA | 331 | SASFRYS | 393 | QQHYRTPYT |
| C78 | 256 | SASSSVSYMH | 318 | DTSKLAS | 392 | QQWTSNPLT |
| C79-3 | 256 | SASSSVSYMH | 318 | DTSKLAS | 392 | QQWTSNPLT |
| C79-2, | 269 | RSSQSIVHSNGNTYLE | 332 | KVSNRFS | 394 | FQGSHVPRT |
| C79-4 | | | | | | |
| C80 | 241 | KSSQSLLNSGNQKNYLA | 311 | GASTRES | 395 | QNDHSYPHT |
| C81 | 270 | RASQDIGNNLN | 333 | ATSSLDS | 396 | LQYASSPVT |
| C82 | 256 | SASSSVSYMH | 318 | DTSKLAS | 397 | QQWTNNPLT |
| C84 | 269 | RSSQSIVHSNGNTYLE | 334 | TVSNRLS | 398 | FQTSHVPPT |
| C84-2 | 271 | RASQSISSWLA | 335 | KASTLES | 399 | QQYNTYWT |
| C85 | 241 | KSSQSLLNSGNQKNYLA | 311 | GASTRES | 400 | QNAHSYPFT |
| C87 | 272 | RASESVDNYGISFMH | 336 | RASNLES | 401 | QQSNKDPMYT |
| C88 | 241 | KSSQSLLNSGNQKNYLA | 311 | GASTRES | 402 | QNDHGYPLT |
| C89 | 273 | RASENIYSHLV | 337 | NGKILAE | 403 | QHHYGSPIT |
| C90 | 274 | KSSQSLLNSFNQKNFLA | 338 | FASTRES | 404 | QQHHSTPPT |
| C91 | 270 | RASQDIGNNLN | 333 | ATSSLDS | 405 | LQYASSPPT |
| C92 | 275 | RASENIYSYLV | 337 | NGKILAE | 406 | QHHYGSPLT |
| C93 | 276 | KSSQSLLSSNNQKSFLA | 338 | FASTRES | 407 | QQHYSTPPT |
| C94 | 241 | KSSQSLLNSGNQKNYLA | 311 | GASTRES | 395 | QNDHSYPHT |
| C95 | 270 | RASQDIGNNLN | 333 | ATSSLDS | 405 | LQYASSPPT |
| C96 | 277 | KSSQSLLNSNNQKNFLA | 338 | FASTRES | 407 | QQHYSTPPT |
| C97 | 278 | KSSQSLLNGFNQKNFLA | 338 | FASTRES | 404 | QQHHSTPPT |
| C98 | 279 | KSSQSLLSSGNQKNFLA | 311 | GASTRES | 408 | QNAHSSPFT |
| C99 | 280 | KSSQSLLNSGIQKNFLA | 311 | GASTRES | 400 | QNAHSYPFT |
| C100 | 281 | KSSQSLLSNNNQKNFLA | 338 | FASTRES | 407 | QQHYSTPPT |
| C101 | 282 | KASQNVRSAVD | 339 | SASNRHT | 409 | LQHWNYPYT |
| C102 | 245 | RASQDIGSSLN | 333 | ATSSLDS | 410 | LQYTSSPWT |
| C103, | 239 | KASQDINKYIA | 308 | YTSTLQP | 411 | LQYDNLWT |
| C103-2 | | | | | | |
| C104 | 283 | KASQDVRSAVA | 324 | LASNRHT | 412 | LQHWTYPYT |
| C105 | 284 | KASQNVRTAVD | 339 | SASNRHT | 409 | LQHWNYPYT |
| C106 | 285 | KASQNVRAAVD | 340 | LASSRHT | 409 | LQHWNYPYT |
| C107 | 286 | KASQNVRIAVD | 341 | SSSNRHT | 413 | LQHWISPYT |
| C108 | 282 | KASQNVRSAVD | 341 | SSSNRHT | 414 | LQHWNSPYT |
| C108-2 | 287 | KASEDIYNRLA | 342 | GTTSLEI | 415 | QQYWSTPWT |
| C110 | 288 | RASKSVSISGYSYMH | 343 | LASNLES | 416 | QHSRELPPT |
| C111 | 289 | KASQNVRSAVA | 324 | LASNRHT | 417 | LQHWNYPWT |
| C111-2 | 290 | RASQSVSSNLA | 344 | GASTRAT | 418 | HQYNNWPRT |
| C112 | 291 | KSSQSLLNSFNQRNFLA | 345 | FASTRDS | 407 | QQHYSTPPT |
| C112-2 | 290 | RASQSVSSNLA | 344 | GASTRAT | 418 | HQYNNWPRT |
| C113 | 292 | KASQIVRTAVD | 346 | SASTRHT | 409 | LQHWNYPYT |
| C113-2 | 290 | RASQSVSSNLA | 344 | GASTRAT | 418 | HQYNNWPRT |
| C114 | 240 | KSSQSLLNSGNQKNFLA | 347 | GASTRQS | 419 | QNDHSYPYT |
| C115 | 288 | RASKSVSISGYSYMH | 343 | LASNLES | 416 | QHSRELPPT |
| C116 | 249 | RASENIYSYLA | 348 | NAETL VE | 420 | QHHYGDPT |
| C117 | 256 | SASSSVSYMH | 349 | DTSKLTS | 421 | QQWISNPYT |
| C118 | 293 | KSSQSLLNGFNQRNFLA | 345 | FASTRDS | 407 | QQHYSTPPT |
| C119 | 294 | KASEDIYARLA | 350 | GATSLET | 422 | QQHWSTPWT |
| C120 | 295 | RSSQSLLNSGNQKNYLA | 311 | GASTRES | 423 | QNAHTSPYT |
| C121 | 241 | KSSQSLLNSGNQKNYLA | 311 | GASTRES | 361 | QNAHSSPYT |
| C122 | 241 | KSSQSLLNSGNQKNYLA | 311 | GASTRES | 361 | QNAHSSPYT |
| C123 | 296 | SASSSVSYMF | 328 | DTSNLAS | 391 | QQWTSYPLT |
| C124 | 266 | SASSSVSYMY | 328 | DTSNLAS | 391 | QQWTSYPLT |
| C125 | 297 | RASENVNSYLA | 351 | NAQTLAE | 424 | QHHYGNPT |
| C129 | 298 | SASSSVTYMH | 323 | STSNLAS | 425 | QQRSSYPWT |
| C132 | 299 | KSSQSLLNIFNQKNFLA | 338 | FASTRES | 407 | QQHYSTPPT |
| C136 | 300 | KASQDVGTAVA | 352 | WASTRHT | 426 | QQYNSYPWT |
| C138 | 301 | SASSSVSYLY | 328 | DTSNLAS | 388 | QQWSSYPLT |
| C140 | 239 | KASQDINKYIA | 308 | YTSTLQP | 360 | LQYDNLLT |
| C143 | 302 | SASSSVNYMF | 328 | DTSNLAS | 391 | QQWTSYPLT |
| C145 | 241 | KSSQSLLNSGNQKNYLA | 311 | GASTRES | 361 | QNAHSSPYT |
| C146 | 282 | KASQNVRSAVD | 353 | SASSRHT | 409 | LQHWNYPYT |
| C147 | 303 | KASENVRAAVD | 324 | LASNRHT | 409 | LQHWNYPYT |
| C148 | 282 | KASQNVRSAVD | 341 | SSSNRHT | 414 | LQHWNSPYT |
| C149 | 304 | RSSQSIEHSNGNTYLQ | 332 | KVSNRFS | 427 | FQGSHVPWT |
| C150 | 247 | RASQDISNYLN | 326 | YSSRLHS | 428 | QQGHALPRT |
| C151 | 249 | RASENIYSYLA | 354 | NAKTLTE | 382 | QHHYGTPLT |
| C152 | 305 | SASSSVNNIH | 355 | DTTKLTS | 429 | QQWNINPYT |
| C153 | 306 | KASQNIRSAVA | 356 | LTSNRHT | 409 | LQHWNYPYT |

### Example 4: Antibody Construction. Expression and Purification

### 4.1 Plasmid Construction

The coding sequences for VHs in the Fab sequence of the screened monoclonal were linked to the coding sequences for the heavy chain constant region (SEQ ID NO: 659) of the human IgG1 to obtain the heavy chain coding sequence of human murine chimeric antibodies, and the coding sequences for VL in the Fab sequence were linked to the coding sequence of the Kappa type (SEQ ID NO: 660) of the human light chain constant region (CL) to obtain the light chain coding sequence of human murine chimeric antibodies. The coding sequences of the heavy and light chains of the antibody were respectively inserted into eukaryotic expression vector plasmids, transformed into E. coli DH5α (Shanghai Sangon), and cultured overnight at 37° C. Plasmid extraction was performed by using an endotoxin-free plasmid extraction kit (Qiagen) to obtain an antibody plasmid without endotoxin for eukaryotic expression.

### 4.2 Expression and Purification of Antibodies

The full-length sequences of the antibodies obtained were expressed by the Expi293F transient expression system (Thermo Fisher, A14635) or the ExpiCHO transient expression system (Thermo Fisher, A29133), and the specific methods can be referred to the operation guidelines of the respective kits by the manufacturers. The isolation and purification of the antibodies were referenced to the purification of the antibodies in Example 1.

### Example 5: Testing Physical and Chemical Properties of the Antibodies

### 5.1 SDS-PAGE Characterization of the Antibodies

Preparation of a non-reducing solution: 3 μg of each obtained antibody were added into 2×SDS loading buffer and 40 mM iodoacetamide, respectively, heated at 75° C. for 10 min, cooled to room temperature, and centrifuged at 12000 rpm for 5 min to obtain the supernatant.

Preparation of a reducing solution: 3 μg of each obtained antibody were added into 2×SDS loading buffer and 5 mM DTT, respectively, heated at 99° C. for 5 min, cooled to room temperature, and centrifuged immediately. The supernatant was added to Bis-tris 4-15% gradient gel (GenScript Biotech Corporation) for gel electrophoresis and the protein bands were developed by Coomassie brilliant blue staining.

The protein gel (decolorized with decolorization liquid to gel background) with the developed protein bands was photographed using an Odyssey 9140 two-color infrared laser imaging system, and the purity of the reduced and non-reduced bands was calculated by its own software.

The test results showed that the bands of respective antibodies of the non-reductive gel were at about 150 kD, and the bands of the reductive gel were at about 55 kD and about 25 kD, with expected sizes.

### Example 6: Detection of Antigen Binding Activity of the Antibodies

In this example, the binding of the expressed antibodies to the human CD112R antigen protein huCD112R-His was detected based on ELISA method, and the binding of the expressed antibodies to the human CD112R overexpressing cell huCD112R-CHO K1 was also detected based on FACS method.

6.1 Detection of the binding capacity of the antibodies to antigen protein huCD112R-His based on ELISA

A 96-well ELISA plate (30 μL/well) was coated with 0.5 μg/mL human CD112R antigen protein huCD112R-His (Acro Biosysterm, PVG-H52H4) overnight at 4° C. On the next day, the plate was washed with PBST for 3 times and then blocked with 5% skim milk for 2 h; after washing with PBST for 3 times, respective antibodies and positive control antibody Tab-1 with gradient dilution (1 μg/ml, 3 times gradient dilution, 9 concentration points) were added and incubated for 1 h. Then, after washing 3 times with PBST, secondary antibody goat-anti-human Fc-HRP (Abcam, ab97225) was added and incubated for 30 min. After the incubation, the plate was washed 6 times with PBST, dried and developed with TMB for 15 min at 25° C., 1M HCl was added to terminate the reaction, and the absorbance was read by a microplate reader at OD450.

The results showed (Table 4) that both the antibodies obtained in the present application and the antigen protein huCD112R had better binding ability, and some antibodies had better binding ability with the antigen protein than the positive control antibody Tab-1.

TABLE 4
EC50 values of the antibodies to the human antigen protein

| Antibody | EC50(nM) |
|---|---|
| C1 | 0.075 |
| C3 | 0.046 |
| C4 | 0.032 |
| C6 | 0.055 |
| C7 | 0.034 |
| C8 | 0.046 |
| C9 | 0.052 |
| C11 | 0.056 |
| C12 | 0.155 |
| C13 | 0.065 |
| C14 | 0.052 |
| C18 | 0.069 |
| C19 | 0.043 |
| C20 | 2.268 |
| C21 | 0.066 |
| C22 | 0.029 |
| C23 | 0.060 |
| C24 | 0.048 |
| C25 | 0.052 |
| C28 | 0.049 |
| C31 | 0.038 |
| C33 | 1.296 |
| C36 | 0.219 |
| C38 | 0.064 |
| C39 | 0.691 |
| C41 | 0.033 |
| C42 | 0.056 |
| C43 | 0.051 |
| C44 | 0.053 |
| C45 | 0.059 |
| C51 | 0.228 |
| C55 | 0.056 |
| C57 | 0.034 |
| C61 | 0.089 |
| C62 | 0.032 |
| C63 | 0.054 |
| C64 | 0.034 |
| C67 | 0.060 |
| C56-2 | 0.032 |
| C69 | 0.023 |
| C59 | 0.044 |
| C71 | 0.037 |
| C101 | 0.043 |
| C105 | 0.021 |
| C110 | 0.032 |
| C111 | 0.036 |
| C113 | 0.038 |
| C114 | 0.042 |
| C115 | 0.029 |
| C116 | 0.025 |
| C117 | 0.041 |
| C118 | 0.029 |
| C119 | 0.025 |
| C120 | 0.035 |
| C121 | 0.037 |
| C122 | 0.030 |
| C123 | 0.035 |
| C124 | 0.033 |
| C125 | 0.049 |
| C129 | 0.034 |
| C132 | 0.038 |
| C136 | 0.039 |
| C138 | 0.039 |
| C143 | 0.025 |
| C145 | 0.038 |
| C146 | 0.036 |
| C152 | 0.082 |
| Tab-1 | 0.034 |
| C112 | 0.028 |

6.2 Detection of the Binding Capacity of the Antibodies to huCD112R-CHO K1 Cells Based on FACS

In this example, the binding activity of human CD112R overexpressing cell huCD112R-CHO K1 to the antibody was evaluated.

The specific method was as follows: the logarithmic growth phase huCD112R-CHO K1 cells were prepared into a single cell suspension, the density was adjusted to 2×10⁵ cells/mL, 50 μL, per well was added to the 96-well round bottom plate, centrifuged at 4° C. and 300 g. and the supernatant was removed. The detection antibody and the positive control antibody Tab−1 were added to respective wells, and mixed well and incubated at 4° C. for 40 min. The incubated cell mixture was washed three times, 100 μL of diluted secondary Goat F(ab′)₂ Anti-Human IgG-Fc (Abcam, ab98596) was added, incubated at 4° C. in the dark for 30 min, washed three times and then detected by flow cytometry, and the fluorescence intensity value (MFI) was read.

Conclusion: on huCD112R-CHO K1 cells, the antibodies of the invention had binding capacity with CD112R overexpression cells.

### Example 7: Detection of Cross-Activity of Antibody Species and Cognates (Elisa Method)

In this example, the cross-reactivity of individual antibodies obtained in the present application on human and monkey species was detected. The binding capacity assay was performed with the antibody prepared in Example 4 and the monkey antigen protein (Uniprot ID: AOA2K5WVV8) and the monkey CD112R expressing cell strain cynoCD112R-CHO K1. For specific operations, refer to Example 6.1.

The results (Table 5) showed that all antibodies obtained in the present application can bind to the monkey antigen protein and the cell strain overexpressing the monkey protein, showing good cross-activity of human monkeys, so the antibodies obtained in the present application had a wide application in experiments and tests based on a monkey model.

The antibodies of the invention all had a better human-monkey species cross-activity.

TABLE 5
EC50 values of the antibodies to the monkey antigen protein

| Antibody | EC50(nM) |
|---|---|
| C1 | 0.116 |
| C3 | 0.118 |
| C4 | 0.059 |
| C6 | 0.058 |
| C7 | 0.080 |
| C8 | 0.047 |
| C9 | 0.070 |
| C11 | 0.079 |
| C12 | 0.768 |
| C13 | 0.059 |
| C14 | 0.067 |
| C18 | 0.069 |
| C19 | 0.034 |
| C20 | 8.363 |
| C21 | 0.068 |
| C22 | 0.125 |
| C23 | 0.125 |
| C24 | 0.169 |
| C25 | 3.803 |
| C28 | 0.054 |
| C31 | 0.120 |
| C36 | 0.101 |
| C39 | 0.141 |
| C41 | 0.052 |
| C42 | 0.078 |
| C43 | 0.044 |
| C44 | 0.076 |
| C45 | 0.114 |
| C51 | 0.224 |
| C55 | 1.291 |
| C57 | 0.077 |
| C62 | 0.043 |
| C63 | 0.090 |
| C64 | 0.054 |
| C67 | 0.080 |
| C56-2 | 0.044 |
| C69 | 0.051 |
| C59 | 0.049 |
| C71 | 0.050 |
| C101 | 0.085 |
| C105 | 0.065 |
| C110 | 0.034 |
| C111 | 0.074 |
| C112 | 0.055 |
| C113 | 0.104 |
| C114 | 0.077 |
| C115 | 0.034 |
| C116 | 0.061 |
| C117 | 0.045 |
| C118 | 0.057 |
| C119 | 0.080 |
| C120 | 0.062 |
| C121 | 0.055 |
| C122 | 0.031 |
| C123 | 0.056 |
| C124 | 0.059 |
| C125 | 0.079 |
| C132 | 0.094 |
| C136 | 0.060 |
| C138 | 0.052 |
| C143 | 0.040 |
| C145 | 0.042 |
| C146 | 0.074 |
| C152 | 0.074 |

### Example 8: Antibody Affinity Kinetics (Bincore) Analysis

In this example, the affinity between the obtained antibody or the positive control antibody Tab-1 and the antigen protein huCD112R-His was detected based on the Biacore device. The anti-CD112R antibody sample was diluted to 10 μg/mL using running buffer HBS-EP+(10 mM HEPES, 150 mM NaCl, 3 mM EDTA and 0.05% P20, pH 7.4) (BR100826, GE), respectively, and the human CD112R antigen protein huCD112R-His (Acro Biosysterm, PVG-H52H4) was diluted with HBS-EP+ buffer at 2×gradient to be between 0.03125 nM and 2 nM. The anti-CD112R antibody was captured with Protein A biosensor chip (BR100354, GE), with a capture time of 60s and a flow rate of 30 μL/min. Then, human CD112R samples diluted to different concentrations were loaded sequentially from zero to the highest concentration, with a binding time of 120s, a dissociation time of 360s, and a flow rate of 30 μL/min. Biacore X100 instrument was used to detect the reaction signal in real time to obtain binding and dissociation curves. After the dissociation of each experimental cycle was completed, the Protein A biosensor chip was cleaned and regenerated with 10 mM Glycine-HCl (pH 1.5) (29127558, GE), the regeneration time was set to 30 s, and the flow rate was 30 μL/min. Parameter fitting: the experiment was run in a single-cycle mode to analyze the time as the abscissa and the response value as the ordinate. After double reference deduction was performed on the obtained data, fitting was performed by using BIAcore X100 (GE) analysis software, and the fitting model used was a 1:1 Langmuir binding model, and affinity indicators such as binding dissociation constants were determined. The results showed that the affinity of the antibodies of the invention was superior or comparable to that of the positive control Tab-1.

TABLE 6
KD values of binding of the antibodies to antigen protein huCD112R-His

| Antibody | KD(M) |
|---|---|
| Tab-1 | 3.46E−10 |
| C1 | 9.05E−10 |
| C3 | 1.81E−10 |
| C4 | 1.41E−11 |
| C7 | 5.76E−11 |
| C8 | 3.97E−10 |
| C11 | 6.22E−11 |
| C12 | 2.90E−09 |
| C13 | 2.18E−11 |
| C14 | 6.87E−11 |
| C18 | 2.06E−10 |
| C19 | 5.67E−10 |
| C20 | 1.14E−10 |
| C24 | 4.67E−11 |
| C41 | 2.20E−11 |
| C55 | 5.06E−10 |
| C57 | 2.19E−10 |
| C62 | 5.71E−10 |
| C55 | 3.348−10 |
| C57 | 1.30E−10 |
| C62 | 7.04E−10 |
| C63 | 4.88E−10 |
| C64 | 8.00E−10 |
| C67 | 2.00E−11 |
| C71 | 1.58E−10 |
| C101 | 3.92E−11 |
| C105 | 7.16E−10 |
| C114 | 3.56E−11 |
| C115 | 2.22E−10 |
| C120 | 7.69E−11 |
| C124 | 2.43E−10 |
| C129 | 3.75E−10 |
| C132 | 1.56E−10 |
| C138 | 5.50E−10 |
| C152 | 2.74E−10 |
| C154 | 7.24E−10 |
| C155 | 6.03E−10 |
| C156 | 9.46E−10 |

### Example 9: Detection of Blocking the Signaling Pathway of Binding of CD112R to CD112 by the Antibodies (Reporter Gene Method)

The reporter gene method consists of two genetically engineered cell lines, one is GS-J2C/CD112R effector cell line expressing human CD112R and luciferase reporter gene driven by NFAT-RE, the other is GS-C2/CD112 target cell line expressing human CD112 in an engineered cell surface protein, intending to activate homologous TCR in an antigen-independent manner. When the GS-J2C/CD112R effector cell line was co-cultured with the GS-C2/CD112 target cell line, the activation of the TCR signaling pathway was inhibited due to the CD112R/CD112 interaction, thereby inhibiting the expression of the luciferase driven by the NFAT, so the corresponding fluorescence signal was relatively low; the addition of the CD112R therapeutic antibody blocked the CD112R/CD112 interaction, thereby restoring the TCR signaling pathway and increasing the fluorescence signal value.

First, GS-2/CD112 target cells were constructed. Human CD112 full-length DNA fragment (NP_001036189.1) was inserted onto a lentiviral expression vector (Blasticidin resistance), mixed with a helper plasmid and transfected into 293T cells, packaged into CD112-Blastcidin recombinant lentivirus. CHO-K1/APC cells were infected with the above recombinant lentivirus (designated as GS-C2), and a cell pool of successfully infected with CD112 was obtained by screening with 10 μg/m of Blasticidin. GS-C2/CD112 monoclonal cells were selected by a limited dilution method.

GS-J2C/CD112R effector cells construction. Human CD112R full-length DNA fragment (XP_011514877.1) was inserted onto a Puromycin resistant lentiviral expression vector, mixed with a helper plasmid and transfected into 293T cells, packaged into CD112-Puromycin recombinant lentivirus. Jurkat-Luc Reporter cell was infected with the above lentivirus (designated as GS-J2C), and a cell pool of successfully infected with CD112R was obtained by screening with 1 μg/ml of Puromycin. GS-J2C/PVRIG monoclonal cells were selected by a limited dilution method.

The target cells were collected by centrifugation and resuspended using respective complete media (10% FBS F-12K). The target cell density was adjusted, inoculated into the experimental plate, and the experimental plate was transferred to a cell incubator (37° C./5% CO₂) for incubation for about 16 hours). The effector cells were collected by centrifugation and then were resuspended using an assay buffer (1.25% FBS RPMI 1(40) and cell density was adjusted. The plate incubated with the target cells was taken out from the cell incubator and the medium was removed. The control and the test sample working solution and the effector cell suspension were sequentially transferred to the experiment board. The experimental plate was transferred to a cell incubator (37° C./5% CO₂) for incubation for 6 hours). The incubated experiment plate was taken out of the cell incubator, and the luciferase detection reagent working solution was transferred to the corresponding wells of the experiment plate, and incubated at room temperature for 3-5 minutes. The chemiluminescent values were read using PHERA star, and data was recorded.

The results showed that the antibodies of the invention effectively blocked the binding of CD112R to CD112.

TABLE 7
Blokcing effects of the antibodies

| Antibody | EC50(μg/ml) |
|---|---|
| C1 | 0.045 |
| C3 | 0.040 |
| C4 | 0.024 |
| C7 | 0.031 |
| C8 | 0.026 |
| C11 | 0.021 |
| C12 | 0.024 |
| C13 | 0.019 |
| C14 | 0.052 |
| C18 | 0.034 |
| C19 | 0.033 |
| C20 | 0.025 |
| C24 | 0.050 |
| C41 | 0.024 |
| C55 | 0.052 |
| C57 | 0.061 |
| C62 | 0.032 |
| C63 | 0.023 |
| C64 | 0.039 |
| C67 | 0.030 |
| C71 | 0.029 |
| C124 | 0.015 |
| C129 | 0.045 |
| C132 | 0.024 |
| C138 | 0.029 |
| C152 | 0.082 |
| Tab-1 | 0.065 |
| C154 | 0.071 |
| C155 | 0.082 |
| C156 | 0.096 |

### Example 10: Blocking the Binding of CD112R to CD112 by the Antibodies Enhancing Antigen-Specific CD8 T Cell Activation (Primary Immune Cell Method)

Assaying the activation of the antibodies on antigen-specific CD8⁺ T cells. PBMC cells of specific genotype (HLA-A*0201) were collected by centrifugation, resuspended using RPMI 1640 culture medium (Gibco) supplemented with 10% FBS (Gibco) and added with a volume of pp65(495-503) polypeptide (CEF20, Cytomegalovirus, CMV pp65(495-503), GenScript) and cytokines (IL-2 Human and IL-7 Human, GenScript), to induce the generation of antigen-specific CD8⁺ T cells. After 11 days of culture, the PBMC cells were collected by centrifugation and CD8⁺ T cells were obtained by using human CD8⁺ T cell isolation kit (17953, STEMCELL). 5×10⁴ targets cells (Ksaumi-1 and Colo-205) were added into a 96-well bottom plate, and the pp65(495-503) polypeptide working solution was added into respective wells, and then placing in a cell culture incubator for 1 hour. 25×10⁴ CD8⁺ T cells were added into respective 96-well plate. Then, the antibodies to be assayed were added into corresponding wells according to detection concentrations. The 96-well plate was placed in a cell culture incubator (37° C./5% CO₂) for 18-22 hours. After incubation, the 96-well plate was taken out and centrifuged at 2000 rpm for 5 min, the supernatant was transferred into a new 96-well plate. The cytokine interferon γ (IFN-γ) was assayed according to the instruction of the cytokine detection kit (62HIFNGPEH, Cisbio).

TABLE 8

| Antibody | | IFN-γ(pg/ml) | |
|---|---|---|---|
| Concentration | 0.5 μg/ml | 5 μg/ml | 50 μg/ml |
| PMA + Iono | | | 768.1802 |
| Human IgG1 | 623.9928 | 669.4832 | 680.8562 |
| C12 | 607.8522 | 677.7104 | 823.5244 |
| C13 | 623.9640 | 658.3758 | 767.2709 |
| C14 | 619.4987 | 674.0936 | 759.3625 |
| C19 | 570.8819 | 744.4827 | 828.833 |
| C24 | 610.7736 | 656.0283 | 733.1607 |
| C41 | 573.3831 | 750.7923 | 659.2455 |
| C1 | S42.6812 | 640.8722 | 755.0672 |
| C3 | 534.5355 | 633.3608 | 755.4967 |
| Tab-1 | 695.9589 | 717.9702 | 763.2948 |
| C4 | 662.69 | 740.1729 | 874.9431 |
| C8 | 722.1538 | 783.3482 | 818.8240 |
| C11 | 675.5745 | 747.3697 | 617.3214 |
| Antibody | IFN-γ(pg/ml) | Antibody | IFN-γ(pg/ml) |
| Concentration | 0.5 μg/ml | 5 μg/ml | Highest concentration |
| | | | Recorded individually |
| C7 | 817.85 | 837.39 | 1108.80 (concentration |
| | | | 38.5 μg/ml) |
| C18 | 748.21 | 801.89 | 1082.83 (concentration |
| | | | 22.75 μg/ml ) |
| C20 | 707.57 | 834.8126 | 1095.479 (concentration |
| | | | 31.5 μg/ml) |

The results showed that the antibodies of the invention effectively blocked the binding of CD112R and CDI 112, resulting in enhanced activation of antigen-specific CD8 Tcells and secretion of cytokine interferon γ (IFN-γ).

In addition to those described herein, modifications to the invention are intended to fall within the scope of the following claims in light of the foregoing description. Each reference cited in this application, including all patents, patent applications, journal articles, books, and any other publications, are incorporated herein by reference in entirety.

Sequence listing

| SEQ ID | Sequence | |
|---|---|---|
| NO: | Description | Sequence information |
| 657 | Tab 1 | QVQLVQSGAEVKKPGASVKVSCKASGYTFTDYNINWVRQAPGQGLEWMGYIYPYIGG |
| | VH | SGYAQKFQGRVTMTRDTSTSTVYMELSSLRSEDTAVYYCAREDKTARNAMDYWGQGT |
| | | LVTVSSASTKGPSVFPLAPCSRSTSESTAALGCLVKDYFPEPVTVSWNSGALTSGVHTFP |
| | | AVLQSSGLYSLSSVVTVPSSSLGTKTYTCNVDHKPSNTKVDKRVESKYGPPCPPCPAPEF |
| | | LGGPSVFLFPPKPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFFLYSRLT |
| | | VDKSRWQEGNVFSCSVMHEALHNHYTQKSLSLSPGK |
| 658 | Tab 1 | DIQMTQSPSSLSASVGDRVTITCRVSENIYSNLAWYQQKPGKAPKLLIYEATNLAEGVPSR |
| | VL | FSGSGSGTDFTLTISSLQPEDFATYYCQHFWGTPYTFGQGTKLEIKRTVAAPSVFIFPPSDE |
| | | QLKSGTASVVCLLNNFYPREAKVQWKVDNALQSGNSQESVTEQDSKDSTYSLSSTLTLS |
| | | KADYEKHKVYACEVTHQGLSSPVTKSFNRGEC |
| 659 | Human | ASTKGPSVFPLAPSSKSTSGGTAALGCLVKDYFPEPVTVSWNSGALTSGVHTFPAVLQSS |
| | IgG1 heavy | GLYSLSSVVTVPSSSLGTQTYICNVNHKPSNTKVDKKVEPKSCDKTHTCPPCPAPELLGG |
| | chain | PSVFLFPPKPKDTLMISRTPEVTCVVVDVSHEDPEVKFNWYVDGVEVHNAKTKPREEQY |
| | constant | NSTYRVVSVLTVLHQDWLNGKEYKCKVSNKALPAPIEKTISKAKGQPREPQVYTLPPSR |
| | region | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 660 | Human light | RTVAAPSVFIFPPSDEQLKSGTASVVCLLNNFYPREAKVQWKVDNALQSGNSQESVTEQ |
| | chain | DSKDSTYSLSSTLTLSKADYEKHKVYACEVTHQGLSSPVTKSFNRGEC |
| | constant | |
| | region (CL) | |
| | Kappa | |
| 661 | Human | EPKSCDKTHTCPPCPAPELLQGPSVFLFPPKPKDTLMISRTPEVTCVVVDVSHEDPEVKEN |
| | IgG1 Fo | WYVDQVEVHNAKTKPREEQYNSTYRVVSVLTVLHQDWLNGKEYKCKVSNKALPAPIE |
| | | KTISKAKGQPREPQVYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQKSLSLSPGK |
| 662 | PVRIG DNA | atgagaacagaggcccaagtgcctgctctgcagcctccggagcctggcct |
| | | ggaaggcgccatgggccaccggaccctggtgctgccttgggtcctgctga |
| | | ccctgtgtgtgaccgccggcacacctgaggtgtgggtgcaggttagaatg |
| | | gaggccacagagctgagctcttttaccatccggtgcggattcctgggatc |
| | | cggcagcatttctctggtcaccgtgtcttggggcggacctaatggcgctg |
| | | gcggcaccacactggccgtgctgcaccctgagcggggcatcagacagtgg |
| | | gcccctgccagacaggccagatgggaaacccagagcagcatctctctgat |
| | | cctggaaggaagcggcgccagcagcccctgcgccaacacaacattctgct |
| | | graagttcgcctccttcccagagggaagctgggaggcctgcggcagcctg |
| | | cctcctagcagtgatcccggcctcagcgcccctcctacccctgctccaat |
| | | cctgagagccgacctggccggaatcctgggcgtgtccggcgtgctgcttt |
| | | tcggctgcgtgtacctgctgcatctgctgaggcggcacaagcacagacca |
| | | gcccctagactgcaaccttctagaaccagccctcaggcccctcgcgcccg |
| | | ggcttgggcccctagccaggcctctcaggctgctctgcatgtgccctacg |
| | | caaccatcaacaccagctgtagacctgccaccctggacaccgcccacccc |
| | | cacggaggcccaagctggtcggccagcctgcccacccacgccgcccacag |
| | | accccagggccccgccgcttgggctagcacccctatccccgcaagaggca |
| | | gctttgtgtccgtggaaaacggcctgtacgcccaggccggcgagagacct |
| | | ccacacacaggccctggcctgacactgttccccgacccccggggccccag |
| | | agccatggaaggccctctgggcgtgcgg |
| 430 | C1 VH | QVHLQQSGPELVKPGASLKISCKASGYTFTDYYLNWVKQRPGQGLEWIGWIFPGSESTF |
| | | YNEKFKGKATLTVDKSSSTAYMLLNSLTSEDSAVYFCTRDGGLRGPFDYWGQGTTLTV |
| | | SS |
| 431 | C1 VL | DIQLTQSPSSLSASLGGKVTITCKASQDINKYIAWCQHKPGKGPRLLIHYTSTLHPGIPSRF |
| | | SGSGSGRDYSFSISNLEPEDSATYYCLQYDYLLTFGGGTKLEIK |
| 432 | C3 VH | QVQLQQSGPELVKPGASVKISCRSSGYTFTDYYINWVKQRPGQGLEWIGWIFPGSDYTY |
| | | YNEKFKGKATLTVDKSSTTAYMLLSSLTSEDSAVYFCARDGGLRGPFDYWGQGTTLTV |
| | | SS |
| 433 | C3 VL | DIQMTQSPSSLSASLGGKVTITCKASQDINKYIAWYQQKPGKGPRLLIHYTSTLQPGIPSR |
| | | FSGSGSGRNSSFSISNLEPEDIATYYCLQYDNLHTFGGGTKLEIK |
| 434 | C4 VH | EVQLQQSGPELVKPGASVKISCKASGYTFTDYFMNWVKQSHGKSLEWIGDIIPNNDSPN |
| | | YNQKFKGKATLTVDKSSNTAYMELRSLTSEDSAVYYCASGYFDVWGTGTTVTVSS |
| 435 | C4 VL | DIVMTQSPSSLSVSAGEKVTMSCKSSQSLLNSGNQKNFLAWYQQKPGQPPKLLIYGTST |
| | | RESGVPDRFTGSGSGTDFTLTISSVQAEDLAVYYCQNDHSYPWTFGGGTKLEIK |
| 436 | C6 VH | QVHLHQSGPELVKPGASVKISCKASGYTFTAYYINWVKQRPGQGLEWIGWIFPGSDSTY |
| | | YNEKFKGRATLTVDKSSSTAYMLLNSLTSEDSAVYFCARDGELRGPFDYWGQGTTLTV |
| | | SS |
| 437 | C6 VL | DIQMTQSPSSLSASLGGKVTITCKASQDINKYIAWYQHKPGKSPRLLIHYTSTLQAGIPSR |
| | | FSGSGSGRDYSFSISNLEPEDSATYYCLQYDNLLTFGGGTKLEIK |
| 438 | C7 VH and | EVQLQQSGPELVKPGASVKISCKASGYTLTDYYMNWVKQSHGKSLEWIGDINPNNGGT |
| | C7-2 VH | NFNQKFKGKATLTIDKSSSTTYMELRSLTSEDSAVYYCASLFFDYWGQGTTLTVSS |
| 439 | C7 VL | DIVMTQSPSSLSVSAGEKVTMSCKSSQSLLNSGNQKNYLAWYQQKPGQPPKLLIYGAST |
| | | RESGVPDRFTGSGSGTDFTLTISSVQAEDLALYYCQNAHSSPYTFGGGTKLEIK |
| 440 | C7-2 VL | DIVMTQSQKFMSTTVGDRVSITCKASQNVGTAVAWYQQKPGQSPELLIYSASNRYTGV |
| | | PDRFTGSGSGTDFTLTISNMQSEDLADYFCQQYSSYRTFGGGTKLEIK |
| 441 | C8 VH | EVQLVASGGGLVKPGGSLKLSCAASGFTFSDYYMFWVRQTPEKRLEWVATISDQGGYI |
| | | YYPDSVKGRFTISRDNDRNSLYLQMSSLKSEDTAIYYCGREDFWGQGTSVTVSS |
| 442 | C8 VL | DIQMTQSPSSLSASLGERVSLTCRASQDIGRSLNWLQQEPDGSVRRLIYATSRLDSGVPK |
| | | RFSGSRSGSDYSLTISSLESEDFVDYYCLQYASFPYTFGGGTKLEIK |
| 443 | C9 VH | EVQLQQSGAELVKPGASVKLSCTASGFNIKDIYMHWLKQRPEQGLEWIGRIDPTNGNSE |
| | | YDPRFRGKATLTADTSSNIAYLQLSSLTSEDTAVYYCARFGIYPYFDYWGQGTTLTVSS |
| 444 | C9 VL | GIVMTQTPKFLLVSAGDRVTITCKASQSVNKAVVWYQQKPGQSPKLLISYASNRYTGVP |
| | | DRFTGSGYGTDFTFSISTVQAEDLAVYFCQQDYSSPWTFGGGTKLEIK |
| 445 | C10 VH | EVELVESGGGLVKPGGSLKLSCAASGFTFSDSYMCWVRQTPEKRLEWVATISDGGGLT |
| | | YYPDSVKGRFTISRDNDRNNLYLQMDSLKSEDTAVYYCGREDLWGQGTSVIVSS |
| 446 | C10 VL | DIQMTQSPSSLSASLGERVSLTCRASQDIGSSLNWLQQEPDGTIKRLIYATSRLDSGVPKR |
| | | FSGSRSGSDYSLTISSLESEDFVDYYCLQYASSPYTFGGGTKLEIK |
| 447 | C11 VH | EVQLQQSGPELVKPGASVKISCKASGYTFTDYFMNWVRQSHGKSLEWIGDIHPNNGSPN |
| | | YNQKFKGKATLTVGKSSSTAYMELRSLTSEDSAVYYCATGYFDVWGTGTTVTVSS |
| 448 | C11 VL | DIVMTQSPSSLSVSAGEKVTMSCKSSQSLLNSGNQKNYLAWYQQKPGQPPKLLIYGAST |
| | | RESGVPDRFTGSGSGTDFTLTISSVQAEDLAVYYCQNDHSYPWTFGGGTKLEIK |
| 449 | C12 VH | EVQLVASGGGLVKPGGSLKLSCAASGFTFSDYYMYWVRQTPEKRLEWVATISDQGGYT |
| | | YYPDSVKGRFTISRDNDRNSLYLQMSSLKSEDTAMYYCGREDLWGQGTSVTVSS |
| 450 | C12 VL | DIQMTQSPSSLSASLGERVSLTCRASQDIGSSLNWLQQEPDGTVKRLIYATSRLDSGVPK |
| | | RFSGSRSGSDYSLTISSLESEDFVDYYCLQYASFPYTFGGGTKLEIK |
| 451 | C13 VH | EVQLQQSGPELVKPGASVKISCKASGYTFTDYFMNWVKQSHGKSLEWIGDINPNNGSPN |
| | | YNQKFKGRATLTVDKSSSTAYMELRSLTSEDSAVYYCASGYFDVWGTGTTVTVSS |
| 452 | C13 VL | DIVMTQSPSSLSVSAGEKVTMSCKSSQSLLNSGTQKNYLAWYQQKPGQPPKLLIYGAST |
| | | RESGVPDRFTGSGSGTDFTLTISSVQAEDLAVYYCQNDHSYPWTFGGGTKLEIK |
| 453 | C14 VH | QVQLQQPGAELVRPGTSVKLSCKASGNTFTSYWMHWVKQRPGQGHEWIGVIDASDSN |
| | | TKYNQKFKGKATLTVDTSSSTAYMQLSSLTSEDSAVYYCARYYSGSSYGLDYWGQGTT |
| | | LTVSS |
| 454 | C14 VL | DIQMTQTASSLSASLGDRVTISCRASQDISNYLNWYQQKPDGTVKLLIYYTSRLHSGVPS |
| | | RFSGSGSGTDFSLTITHLEQEDIASYFCQQGNMFPPTFGGGTKLEIK |
| 455 | C16 VH | QVQLQQPGAELVRPGTSVKLSCKASGNTFTSYWMHWVKQRPGQGHEWIGVIDASDSN |
| | | TKYNQKFKGKATLTVDTSSSTAYMQLSSLTSEDSAVYYCARYFSGSSYGLDYWGQGTT |
| | | LTVSS |
| 456 | C16 VL | DIQMTQTASSLSASLGDRVTISCRASQDISNYLNWYQQKPDGTVKLLIYYTSRLHSGVPS |
| | | RFSGSGSGTDYSLTITNLEQEDIASYFCQQGNMFPPTFGGGTKLEIK |
| 457 | C18 VH | EVQLVESGGGLVKPGGSLKLSCAASGFTFSDYYMYWVRQTPEKRLEWVATISDGGGFI |
| | | YYPDSVKGRFTISRDNDRNSLYLLMSSLKSEDTAMYFCGREDLWGQGTSVTVSS |
| 458 | C18 VL | DIQMTQSPSSLSASLGERVSLTCRASQDIGRSLNWLQQGPDGTVKRLIYATSRLDSGVPT |
| | | RFSGSRSGSDYSLTISSLESEDFVDYYCLQYASFPYTFGGGTKLEIK |
| 459 | C19 VH | EVQLVESGGGLVKPGGSLKLSCAASGFTFSDYYMYWVRQTPEKRLEWVATISDAGGYT |
| | | YYPDSVKGRFTISRDNARNSLYLQMSSLKSEDTAMYFCGREDLWGQGTSVTVSS |
| 460 | C19 VL | DIQMTQSPSSLSASLGERVSLTCRASQDIGYSLNWLQQEPDGSVRRLIYATSRLDSGVPK |
| | | RFSGSRSGSDYSLTISSLESEDFVDYYCLQYASFPYTFGGGTKLEIK |
| 461 | C20 VH | EVQLVESGGGLVKPGGSLKLSCAASGFTFSDSYMCWVRQTPEKRLEWVATISDAGGYT |
| | | YYPDSVKGRFTISRDNDRNNLYLQMSGLKSEDTAVYYCGREDLWGQGTSVTVSS |
| 462 | C20 VL | DIQMTQSPSSLSASLGERVSLTCRASQDIGRSLNWLQQEPDGTIKRLIYATSRLDSGVPKR |
| | | FSGSRSGSDYSLTISSLESEDFVDYYCLQYASSPYTFGGGTKLEIK |
| 463 | C21 VH | EVQLQQSGAELVKPGASVKLSCTASGFNIKDIYMHWLKQRPEQGLEWIGRVDPINGNS |
| | | EYDPRFQGKATLTADTSSNIAYLQLSSLTSEDTAVYYCARFGIYPYFDYWGQGTTLTVSS |
| 464 | C21 VL | SIVMTQTPKFLLVSAGDRVTITCKASQSVNKAVVWYQQKPGQSPKLLINYASNRYTGVP |
| | | DRFTGSGYGTDFTFSISTVQAEDLAVYFCQQDYSSPWTFGGGTKLEIK |
| 465 | C22 VH | EVQLQQSGPEPVKPGASVKISCKASGYTFTDYNIHWVRQSHGKSLEWIGYIYPYNGGAG |
| | | YNQKFKSKATLTVDSSSSTAYMELRSLTSEDSAVYYCAREELRRDYGMDYWGQGTSVT |
| | | VSS |
| 466 | C22 VL | DIQMTQSPASLSASVGETVTITCRASENIYSYLAWYQQKQGKSPQLLVYNAKTLAEGVP |
| | | SRFSGSGSGTQFSLKINSLQPEDFGSYYCQHHYGTPYTFGGGTKLETK |
| 467 | C23 VH | QVQLQQSGPELVKPGTSVKISCKASGYTFTDYYINWVKQRPGQGLEWIGWIFPGSGNTY |
| | | YNEKFKGRATLTVDKSSSTAYMLLSSLTSEDSAVYFCARDGGLRGPFDYWGQGTTLTV |
| | | SS |
| 468 | C23 VL | DIQMTQSPSSLSASLGGKVTITCKASQDINKYIAWFQHKPGKGPRLLIHFTSTLQSGIPSRF |
| | | SGSGSGGDYSFSISNLEPEDIATYYCLQYDYLYTFGGGTTLEIK |
| 469 | C24 VH | QVQLKQSGPGLVQPSQNLSITCTVSGFSLTGYGINWVRQSPGKGLEWLGVIWSGGNTEY |
| | | NAAFRSRLSISKDTSKSQVFLKMNSLQADDTAIYSCARDSTGLYAMDYWGPGTSVTVSS |
| 470 | C24 VL | DLQMTQTTSSLSASLGDRVTISCRTSQDISNFLNWYQQKPDGPLKLLIYYTSRLHSGVPS |
| | | RFSGSGSGTDYSLTISNLEQEDIATYFCQQGYMLPPTFGGGTKLEIK |
| 471 | C25 VH | QVQLQQPGAELVRPGTSVKLSCKASGNTFTSYWMHWVKQRPGQGHEWIGVIDASDSK |
| | | TKYNQKFKGKATLTVDTSSSTAYMQLSSLTSEDSAVYYCARYYSGSSYGLDYWGQGTT |
| | | LTVSS |
| 472 | C25 VL | DIQMTQTASSLSASLGDRVTISCRASQDISNYLNWYQQKPDGTVKLLIYYTSRLHSGVPS |
| | | RFSGSGSGTDYSLTISNLEQEDIATYFCQQGNTLPPTFGGGTKLEIK |
| 473 | C28 VH | QIQLQQSGPELVKPGPSVKISCKASGYTFTDYYINWVKQRPGQGLEWIGWIYPGSGYTK |
| | | YNEKLKGRATLTVDTSSSTAYMQLSSLTSEDSAVYFCARDSFYAMDYWGQGTSVTVSS |
| 474 | C28 VL | DIVMTQSQKFMSTTVGDRVSITCKASQNVGTAVAWYQQKPGQSPKLLIYSASNRYTGV |
| | | PDRFTGSGSGTDFTLTITNMQSEDLADYFCLQYSSYLTFGAGTKLELK |
| 475 | C31 VH | QIQLQQSGPELVKPGASVKISCKASGYTFTDYYIAWVKQRPGQGLEWIGWIYLGSGHTM |
| | | YNENEKGKATLTVDTSSSTAYMQLSSLTSEDSAVYFCARDSFYAMDYWGQGTSVTVSS |
| 476 | C31 VL | DIVMTQSQKFMSTTVGDRVSITCKASQNVGTAVAWYQQKPGQSPKLLIYSASNRYTGV |
| | | PDRFTGSGSGTDFTLTISNMQSEDLADYFCQQYSTYLTFGAGTKLELK |
| 477 | C33 VH | QVQLQQSGPELVKPGASVKISCKASGYTFTDYYINWVKQRPGQGLEWIGWIFPGSGSTY |
| | | YNEKFKGKATPTVDKYSSTAYMLLSSLTSEDSAVYFCAREDYFDRGDCWGQGTSVTVS |
| | | S |
| 478 | C33 VL | QIVLTQSPAIMSASPGEKVTMTCSASSSINYMHWYQQKPGTSPKRWIYDTSKLASGVPA |
| | | RFSGSGSGTSYSLTISSMEAEDAATYYCHQRSSYPFTFGSGTKLEIK |
| 479 | C36 VH | QIQLVQSGPELKKPGETVKISCKASGYTFTTYGMSWVKQAPGKGLKWMGWINTYSGVP |
| | | TYADDFKGRFAFSLETSASNAYLQINNLKNEDTATYFCARELPFTTIIGTKDYWGQGTTL |
| | | TVSS |
| 480 | C36 VL | DIVLIQSPATLSVTPGDSVSLSCRASQFISNNLHWYQQKSHESPRLLIKYASQSISGIPSRFS |
| | | GGGSGTDFTLTVNSVETEDFGMYFCQQSNSWPHTFGGGTKLEIK |
| 481 | C38 VH | QVQLQQSGAELMKPGASVKLSCKATGYTFTGYWIEWVKQRPGHGLEWIGEILPESGRT |
| | | NYNEKFKGKATFTADTSSNTAYMQLSSLTTEDSAIYYCASLLFFDYWGQGTTLTVSS |
| 482 | C38 VL | DVVLTQTPLTLTVTIGQPASISCKSSQSLLDSDGKTYLNWLLQRPGQSPKRLIYLVSKLDS |
| | | GVPDRFTGSGSGTDFTLKISRVEAEDLGVYYCWQGTHFPLTFGAGTKLELK |
| 483 | C39 VH | QAYLQQSGAELVRPGASVKMSCKASGYTFTSYNIHWVKQTPRQGLEWIGAIYPGNADIS |
| | | YNQKFRDKATLTVDKSSSTAYMQLSSLTSEDSAVYFCAKPLRIYAMDCWGQGTSVTVS |
| | | S |
| 484 | C39 VL | DIVMTQSQKFMSTSVGDRVSVTCKASQNVGTNVAWYQQKPGQSPKALIYSASYRYSGV |
| | | PDRFTGSGSGTDFTLTISNVQSEDLADYFCQQYNSRPLTFGGGTKLEIK |
| 485 | C41 VH | EVQLQQSGPELVKPGASVKISCKASGNTFTDYYINWVKQSHGKSLEWIGDIEPNNGGTN |
| | | YNQKFKGKATLTVDKSSSTAYMELRSLTSEDSAIYYCATLYFDYWGQGTTLTVSS |
| 486 | C41 VL | DIVMTQSPSSLSVSAGEKVTMSCESSQSLLNSGNQKNYLAWYQQKPGQPPKLLIYGAST |
| | | RESGVPDRFTGSGSGTDFTLTISSVQAEDLAVYYCQNDHSYPFTFGSGTKLEIK |
| 487 | C43 VH | QVQLQQPGAELVRPGTSVKLSCKASGNTFTTYWMHWVKQRPGQGHEWIGVIDASDSN |
| | | TKYNQKFKGKATLTVDTSSSTAYMQLSSLTSEDSAVYYCARYYSGSSYGLDYWGQGTT |
| | | LTVSS |
| 456 | C43 VL | DIQMTQTASSLSASLGDRVTISCRASQDISNYLNWYQQKPDGTVKLLIYYTSRLHSGVPS |
| | | RFSGSGSGTDYSLTITNLEQEDIASYFCQQGNMFPPTFGGGTKLEIK |
| 488 | C44 VH | QVQLQQSGPELMKPGASLKISCKASGYTFTDSYINWVKQRPGQGLEWIGWIFPGSGSTY |
| | | YNEKFKGTATLTVDKSSSTAYMLLSSLTSEDSAAYFCARDGGLRGPFDYWGQGTTLTV |
| | | SS |
| 489 | C44 VL | DIQMTQSPSSLSASLGGKVTITCKASQDINKYIAWYQHKPGKGPRLLIHYTSTLQSGIPSR |
| | | FSGSGSGRDYFFSISNLESEDIATYYCLQYDHLLTFGGGTKLEIK |
| 490 | C45 VH | QVHLQQSGPDLVKPGVSVKISCKASGYTFTDYYINWVKQRPGQGLEWIGWIFPGSGTTY |
| | | YNEKFKGKATLTVDKSSSTAYMLLSSLTSEDSAVYFCARDGGLRGPFDYWGQGTTLTV |
| | | SS |
| 491 | C45 VL | DIQMTQSPSSLSASLGGKVTITCKANQDINKYIAWYQYKPGKGPRLLIHYTSTLQPDIPSR |
| | | FSGSGSGRDFSFSISNLEPEDIATYYCLQYDYLYTFGGGTKLEIK |
| 492 | C47-2 VH | QVQLQQPGAELVKPGASVKLSCKASGYSFTSYWMQWIKQRPGQGLEWIGEIDPFDSYT |
| | | NYNQKFKGKATLTVDTSSSTAYMQLSSLTSEDSAVYYCASRVYYGYDRYFFDYWGQG |
| | | TTLTVSS |
| 489 | C47-2 VL | DIQMTQSPSSLSASLGGKVTITCKASQDINKYIAWYQHKPGKGPRLLIHYTSTLQSGIPSR |
| | | FSGSGSGRDYFFSISNLESEDIATYYCLQYDHLLTFGGGTKLEIK |
| 493 | C48 VH and | QVQLQQSGPELVKPGASVKISCKASGYTFTDYYINWVKQRPGQGLEWIGWIFPGSGSTY |
| | C48-2 VH | YNEKFKGKATLTVDKSSSTAYMLLSSLTSEDSAVYFCAPDGSYDPPFAYWGLGTLVTVS |
| | | A |
| 494 | C48 VL | QIVLTQSPAIMSASPGEKVTITCSASSSVSYMHWFQQKPGTSPKLWIYSTSNLASGVPAR |
| | | FSGSGSGTSYSLTISRMEAEDAATYYCQQRSSYPPTFGAGTKLELK |
| 495 | C48-2 VL | DIQMTQSPSSLSASLGGKVTITCKASQDINKYIAWYQHKPGKGPRLLIHYTSTLQPGIPSR |
| | | FSGSGSGRDYSFSISNLEPEDIATYYCLQYDNLYTFGGGTKLEIK |
| 496 | C51 VH | QVQLQQSGAELVRPGASVTLSCKASGYTFTDYEMHWVKQTPVHGLEWIGAIDPETGGT |
| | | AYNQKFKGKAILTADKSSSTAYMELRSLTSEDSAVYYCTRWITTVVATLYYFDYWGQG |
| | | TTLTVSS |
| 497 | C51 VL | DIVMTQSQKFMSTSVGDRVSITCKASQNVRTAVAWYQQKPGQSPKALIYLASNRHTGV |
| | | PDRFTGSGSGTDFTLTISNVQSEDLADYFCLQHWNYPLTFGAGTKLELK |
| 498 | C52 VH | RVQLQQSGAELVRPGASVTLSCKASGYTFTDYEMHWVKQTPVHGLEWIGAIDPETGGT |
| | | AYNQKFKGKAILTADKSSSTAYMELRSLTSEDSAVYYCTRWITTVVATLYYFDYWGQG |
| | | TTLTVSS |
| 497 | C52 VL | DIVMTQSQKFMSTSVGDRVSITCKASQNVRTAVAWYQQKPGQSPKALIYLASNRHTGV |
| | | PDRFTGSGSGTDFTLTISNVQSEDLADYFCLQHWNYPLTFGAGTKLELK |
| 499 | C55 VH | EVQLQQSGPELVKPGASVKISCKASGYTFTDYYMSWVKQSHGKSLEWIGDVNPNNGDT |
| | | SYNQKFKGKATLTVDKSSSTAYMELRSLTSEDSAVYYCAASGLPYVLMDYWGQGTSV |
| | | TVSS |
| 500 | C55 VL | DIQMTQSPASLSASVGETVTITCRASENINSYLAWYQQKQGKSPQLLVYNTKTLTEDVPS |
| | | RFSGSGSGTQFSLKINSLQPEDFGSYYCQHHYGTPLTFGGGTKLELK |
| 501 | C56 VH | EVQLVESGGGLVKPGGSLKLSCAASGFTFSGYALSWVRQTPEKRLEWVATIRDGGSYT |
| | | YYSDNEKGRFTISRDNAKNNLYLQMSHLKSEDTAIYYCTRDQVNRGWFTYWGQGTLV |
| | | TVSA |
| 502 | C56-2 VH | EVQLVESGGGLVKPGGSLKLSCAASGFTFSDYALSWVRQTPEKRLEWVATIRDGGSYT |
| | | YYSDNEKGRFTISRDNAKNNLYLQMSHLRSEDTAMYYCTRDQVNRGWFTYWGQGTLV |
| | | TVSA |
| 503 | C56 VL and | QVILTQSPAIMSASLGERVTMTCSASSSVSSSYFHWFQQKPGSSPKLWIYSTSNLASGVPP |
| | C56-2 VL | RFSGSGSGTSYSLTISSMEAEDAATYYCHQHHRSPLTFGAGTKLELK |
| 504 | C57 VH | EVQLQQSGPVLVKPGASVKMSCKASGYIFTDYYMHWVKQSHGKSLEWIGIINPYNGDT |
| | | SYNQKFKDKATLTVDKSSSTAYMELNSLTSEDSAVYYCARDSSNVGNAMDYWGQGTS |
| | | VTVSS |
| 505 | C57 VL | DIQMTQTTSSLSASLGDRVTISCRASQDISNYLNWYLQKPDGTIKLLIYYSSRLHSGVPSR |
| | | FSGSGSGTDYSLTISNLEQEDIATYFCQQGYTLPRTFGGGTKLEII |
| 499 | C58 VH | EVQLQQSGPELVKPGASVKISCKASGYTFTDYYMSWVKQSHGKSLEWIGDVNPNNGDT |
| | | SYNQKFKGKATLTVDKSSSTAYMELRSLTSEDSAVYYCAASGLPYVLMDYWGQGTSV |
| | | TVSS |
| 506 | C58 VL | DIQMTQTTSSLSASLGDRVTISCRASQDISNYLNWYLRKPDGTVKLLIYYSSRLHSGVPS |
| | | RFSGSGSGTDYSLTISNLEQEDIATYFCQQGNTLPRTFGGGTKLEIK |
| 507 | C59 VH | EVQLVESGGGLVKPGGSLKLSCAASGFTFSGYALSWVRQTPEKRLEWVATIRDGGSYT |
| | | YYSDNEKGRFTISRDNAKNNLYLQMSHLKSEDTAMYYCTRDQVNRGWFTYWGQGTL |
| | | VTVSA |
| 503 | C59 VL | QVILTQSPAIMSASLGERVTMTCSASSSVSSSYFHWFQQKPGSSPKLWIYSTSNLASGVPP |
| | | RFSGSGSGTSYSLTISSMEAEDAATYYCHQHHRSPLTFGAGTKLELK |
| 508 | C61 VH | DVQLQESGPGLVKPSQSLSLTCSVTGYSITSNYYWNWIRQFPGNNLEWMGYISSDGTNN |
| | | YNPSLRNRISITRDTSKNQFFLKLNSVTTEDTATYYCARGLRYGPWGQGTTLTVSS |
| 509 | C61 VL | QLILTQSSSASFSLGASAKLTCTLSSQHSTYTIEWYQQQPLKAPKYVMEVKNDGSHRTG |
| | | DGIPDRFSGSSSGADRYLSISNIQPEDEAIYICGVGDINNYIFGGGTKVTVL |
| 510 | C62 VH | EVMLVESGGGLVKPGGSLKLSCAASGFSFSSYSMSWVRQTPEKRLECVATIGGVQGNT |
| | | YYPDSVKGRFSISRDNAKNTLYLQMSSLRSEDTALYYCARHEDYYYYAMDYWGQGTS |
| | | VTVSS |
| 511 | C62 VL | QIVLTQSPAIMSASPGEKVTMTCSANSTVSYIYWYQQKPGSSPRLLIYDTSNLASGVPVR |
| | | FSGSGSGTSYYLTISRMEAEDAATYYCQQWSSFPFTFGSGTKLEIK |
| 512 | C63 VH | EVMLVESGGGLVKPGGSLKLSCAASGFIFSSYIMSWVRQTPEKRLEWVATIRGGGGNTY |
| | | YLDSVKGRFIISRDNAKNNLYLQMSSLRSEDTALYYCARHEDYEWYAMDNWGQGTSV |
| | | TVSS |
| 513 | C63 VL | QIVLTQSPAIMSASPGEKVTLTCSARSSVSYMYWYQQKPGSSPKLLIYDTSNLASGVPVR |
| | | FSGSGSGTSYSLTISRMEAEDAATYYCQQWSSYPLTFGAGTKLELK |
| 514 | C64 VH | EVMLVESGGDLVKPGGSLKLSCAASGFTFSSYFMSWVRQTPEKRLEWVATISGGGDKT |
| | | YYLDILKGRFTISRDNAKNTLYLQMSSLRSEDTALYYCARHEDYAWYAMDYWGQGTS |
| | | VTVSS |
| 515 | C64 VL | QIVLTQSPAIMSASPGEKVTMTCSASSSVNYIYWYQQKPGSSPRLLIYDTSNLASGVPVR |
| | | FSGSGSGTSYSLTISRMEAEDAATYYCQQWSSYPLTFGAGTKLELK |
| 501 | C66 VH | EVQLVESGGGLVKPGGSLKLSCAASGFTFSGYALSWVRQTPEKRLEWVATIRDGGSYT |
| | | YYSDNEKGRFTISRDNAKNNLYLQMSHLKSEDTAIYYCTRDQVNRGWFTYWGQGTLV |
| | | TVSA |
| 516 | C66 VL | DIQMTQSPSSLSASLGGKVTITCKASQDINKYIAWYQHKPGKGPRLLIHYTSTLQPGIPSR |
| | | FSGSGSGRDYSFNISNLEPEDIATYYCLQYDNLYMFGGGTKLEIK |
| 517 | C67 VH | EAQLQQSGPELVKPGASVKISCKASGYTFTDYYMNWVKQSHGKSLDWIGDINPDNGGS |
| | | TYNQKFKGKATLTVDKSSSTAYMELRSLTSEDSAVYYCSASGPPYVLLDYWGQGTSVI |
| | | VSS |
| 518 | C67 VL | DIQMTQSPASLSASVGETVTITCQTSENIYSYLAWYQQKQGKSPQLLVYNTQTLAEGVPS |
| | | RFSGSRSGAQFSLKINSLQPEDFGSYYCQHHYGVPLTFGAGTKLELK |
| 519 | C69 VH | EVQLQQSGPVLVKPGASVKMSCKASGYRLTDYYMHWVKQSHGKSLEWIGIINPYNGGT |
| | | SYNQKFKGKATLTVDKSSSTAYMELNSLTSDDSAVYYCARDSSNVGNDMDYWGQGTS |
| | | VSVSS |
| 520 | C69 VL | DIQMTQTTSSLSASLGDRVTISCRASQDISNYLNWYLQKPDGTVKLLIYYSSRLHSGVPS |
| | | RFSGSGSGTDYSLTINNLEQEDIATYFCQQGNTLPRTFGGGTKLEIR |
| 521 | C71 VH | EVILVESGGGLVKPGGSRKLSCAASGFTFSSYFMSWVRQTPEKRLEWVATISGGGDNTY |
| | | YPDSVKGRFTISRDNAKNTLYLQMSSLRSEDTALYYCARHEDYEWYAMDYWGQGTSV |
| | | TVSS |
| 522 | C71 VL | QIVLTQSPAIMSTSPGEKVTMTCSASSSVSYIYWYQQKPGSSPRLLIYDTSNLASGVPVRF |
| | | SGSGSGTSYSLTISRMEAEDAATYYCQQWTSYPLTFGAGTKLELK |
| 523 | C72 VH | EVQLVESGGDLVKPGGSLKLSCAASGFTFSSYFMSWVRQTPDKRLEWVATIRGGGSYT |
| | | YYLDSVKGRFTVSRDNAKNTLYLQMSSLKSEDTAMYYCARHEDYDYYAMDYWGQGT |
| | | SVTVSS |
| 524 | C72 VL | QIVLTQSPAIMSASPGEKVTMTCSASSSVSYMYWYQQKPGSSPRLLIYDTSNLASGVPVR |
| | | FSGSGSGTSYSLTISRMEAEDAATYYCQQWSSYPLTFGAGTKLELK |
| 525 | C75 VH | QAQLQQPGAELVKPGASVKMSCKASGYTFTSYWITWVKQRPGQGLEWIGDIYPGNFSP |
| | | NYNEKFKNKATLTVDTSSSTAYMQLSSLTSEDSAVYYCAKGYFDVWGTGTTVTVSS |
| 486 | C75 VL | DIVMTQSPSSLSVSAGEKVTMSCKSSQSLLNSGNQKNYLAWYQQKPGQPPKLLIYGAST |
| | | RESGVPDRFTGSGSGTDFTLTISSVQAEDLAVYYCQNDHSYPFTFGSGTKLEIK |
| 526 | C76 VH | EVQLQQSGPELVKPGASVKISCKASGYTFTHYYKNWVKQSHGKSLEWIGAINPNIGGPS |
| | | YNQKFKGKATLTVDKSSSTVYMELRSLTSEDSAVYYCARSDDYGGYYFDYWGQGTTL |
| | | TVSS |
| 527 | C76 VL | QIVLSQSPALLSASPGEKVTMTCRANSSVNYMHWYQQKPGSSPKPWIYATSNLASGVPA |
| | | RFSGSGSGTSFSLTISRVEAEDAATYYCQQWTSNPLTFGAGTKLELK |
| 528 | C77 VH | EVQLQQSGPELVKPGASVKISCKASGYTFTDYYMNWVRQSHGKSLEWIGDIYPNNGDT |
| | | NYNQKFKGKATLTVDKSSKTADMELRSLTSEDSAVYYCIRGVGLGFPYWGQGTLVTVS |
| | | A |
| 529 | C77 VL | DIVMTQSHKFISTSVGDGVSITCQASQVVSTAVAWYQQKPGQSPRLLLHSASFRYSGVP |
| | | DRFTGSGSGTDFTFTISTVQAEDLAVYYCQQHYRTPYTFGGGTKLEIK |
| 530 | C78 VH | EVQLQQSGPVLVKPGASVKMSCEPSGYTFTHYYVNWMRQSHGKSLEWIGVIKPYNGAP |
| | | SYNQKFKGKATLTVDKSSSKINMELNSLTSEDSAVYYCARSDDFDGYAMDYWGQGTS |
| | | VTVSS |
| 531 | C78 VL | QIVLTQSPAIMFASPGEKVTMTCSASSSVSYMHWYQQKSGTSPKRCISDTSKLASGVPAR |
| | | FSGSGSGTSYSLTISSMEAEDAATYYCQQWTSNPLTFGSGTKLELK |
| 530 | C79-2 VH | EVQLQQSGPVLVKPGASVKMSCEPSGYTFTHYYVNWMRQSHGKSLEWIGVIKPYNGAP |
| | | SYNQKFKGKATLTVDKSSSKINMELNSLTSEDSAVYYCARSDDFDGYAMDYWGQGTS |
| | | VTVSS |
| 532 | C79-3 VH | EVQLVESGGDLVKPGGSLKLSCAASGFTFNNYGMSWVRQTPDKRLEWVATISRGGSFT |
| | and C79-4 | YYPDSVKGRFTISRDNAKNTLYLQMSSLKSEDTAMYYCARPIYYEYDGFAYWGQGTLV |
| | VH | TVSA |
| 531 | C79-3 VL | QIVLTQSPAIMFASPGEKVTMTCSASSSVSYMHWYQQKSGTSPKRCISDTSKLASGVPAR |
| | | FSGSGSGTSYSLTISSMEAEDAATYYCQQWTSNPLTFGSGTKLELK |
| 533 | C79-2 VL | DVLMTQTPLSLPVSLGDQASISCRSSQSIVHSNGNTYLEWYLQKPGQSPKLLIYKVSNRF |
| | and C79-4 | SGVPDRFSGSGSGTDFTLKINRVEAEDLGVYSCFQGSHVPRTFGGGTKLEIK |
| | VL | |
| 534 | C80 VH | QVQLQQPGAELVKAGASVKMSCKASGYTFTSYWITWVKQRPGQGLEWIGDIYPGNDSP |
| | | NYNEKFKNKATLTVDTSSSTAYMQLSSLTSEDSALYYCARGYYDYWGQGTTLTVSS |
| 535 | C80 VL | DIVMTQSPSSLSVSAGEKVTMSCKSSQSLLNSGNQKNYLAWYQQKTGQPPKLLIYGAST |
| | | RESGVPDRFTGSGSGTDFTLTISSVQAEDLAVYYCQNDHSYPHTFGAGTKLELK |
| 536 | C81 VH | EVQLQQSGPVLVKPGASVKMSCKASGNTFNGYYVNWVKQSHGKSLEWIGLIDPSNGGT |
| | | NYNQKFKDKATLTVDKSSTTAYMEFNSLTSEDSSVYYCAGGTVYWGQGTTLTVSS |
| 537 | C81 VL | DIQMTQSPSSLSASLGERVSLTCRASQDIGNNLNWLQQEPDGTIKRLIYATSSLDSGVPKR |
| | | FSGSRSGSDYSLTISSLESEDFVDYYCLQYASSPVTFGGGTKLEIK |
| 538 | C82 VH | EVQLQQSGPELVKPGASVKISCKASGYMFTDYYMNWVKQSHGGSLEWIGTFNPNRGGP |
| | | SYNLKFKGKATLTVDKSSITAYMDLRSLTSEDSAVYYCARADDYDGYYFDYWGQGTT |
| | | LTVSS |
| 539 | C82 VL | QIVLTQSPAIMSASPGEKVTMTCSASSSVSYMHWYQQKSGTSPKRWIYDTSKLASGVPA |
| | | RFSGSGSGTSYSLTISSMEAEDAATYYCQQWINNPLTFGAGTKLELK |
| 540 | C84 VH and | QVQLQQSGPELVKPGASVRISCKASGYSFTDYFISWVKQRPGQGLEWIGWIFPGSGSTYF |
| | C84-2 VH | NENFKGEATLTADKSSSTAYLLLSSLTSEDSAVYFCTTYYGSNYWGQGTTLTVSS |
| 541 | C84 VL | DVLMTQAPLSLPVSLGDQASISCRSSQSIVHSNGNTYLEWYLQKPGQSPKLLIYTVSNRL |
| | | SGVPDRFSGSGSGTDFTLKISRVEAEDLGIYYCFQTSHVPPTFGGGTKLEIK |
| 542 | C84-2 VL | DIQMTQSPSTLSASVGDRVTITCRASQSISSWLAWYQQKPGKAPKVLIYKASTLESGVPS |
| | | RFSGSGSGTDFTLTISSLQPEDFATYNCQQYNTYWTFGQGTKVEIK |
| 543 | C85 VH | EVQLQQSGPELVKPGASVKISCKASGYTFTDYSMNWVKQSHGKSLEWIGDINPNNGGTS |
| | | YNQKFKGKATLTVDKSSSTAYMELRSLTSEDSAVYYCARVWFAYWGQGTLVTVSA |
| 544 | C85 VL | DIVMTQSPSSLSVSAGEKVTLSCKSSQSLLNSGNQKNYLAWYQQKPGQPPKLLIYGAST |
| | | RESGVPDRFTGSGSGTDFTLTISSVQAEDLALYYCQNAHSYPFTFGSGTKLDIK |
| 545 | C87 VH | QHELQQSGAELVRPGSSVKLSCKDSYFAFMASAMHWVKQRPGHGLEWIGSFTMYSDA |
| | | TEYSENFKGKATLTANTSSNTAYMELSSLISEDSAVYYCASGYSGNGFTYWGQGTLVTV |
| | | SA |
| 546 | C87 VL | DIVLTQSPASLAMSLGQRATISCRASESVDNYGISFMHWYQQKPGQPPKLLIYRASNLES |
| | | GIPARFSGSGSRTDFTLTINPVETDDVATYYCQQSNKDPMYTFGGGTKLEIR |
| 547 | C88 VH | QVQLQQPGAELVKPGASVNMSCKASGYTFSSYWITWVKQRPGQGLEWIGDIHPGNTSP |
| | | NYNEKFRTKATLTVDTSSSTAYMQLSSLTSEDSAVYYCARGWGDYWGQGTTLTVSS |
| 548 | C88 VL | DIVMTQSPSSLSVAAGEKVTMSCKSSQSLLNSGNQKNYLAWYQQKPGQPPKLLIYGAST |
| | | RESGVPDRFTGSGSGTDFTLTISSVQAEDLAVYYCQNDHGYPLTFGTGTKLELK |
| 549 | C89 VH | EVQLVESGGDLVKPGGSLKLSCVASGLTFSDYGMYWIRQAPEKGLEWVAYIRSDSSTIY |
| | | YTDTVKGRFTISRDNAKNTLFLQMTSLRSEDTAIYYCARRSPVVVNYAMDYWGQGTSV |
| | | TVSS |
| 550 | C89 VL | DIQMTQSPASLSASVGETVTITCRASENIYSHLVWYQQKQGKSPHLLVYNGKILAEGVPS |
| | | RFSGSGSGTQFSLKINSLQPEDFGSYYCQHHYGSPITFGAGTKLELN |
| 551 | C90 VH | EVQLQQSGPELVKPGTSVKISCKASGYTFTDYYMNWVRQSHGKSLEWIGEINPKNGDT |
| | | NYNQNFKGKATLTVDKSSNTAYMELRSLTSEDSAVFYCAREGVSSYYAMDYWGQGTS |
| | | VTVSS |
| 552 | C90 VL | DIVMTQSPSSLAMSVGQKVTMSCKSSQSLLNSFNQKNFLAWYQQKPGQSPKLLVYFAS |
| | | TRESGVPDRFIGSGSGTDFTLTITSVQAEDLADYFCQQHHSTPPTFGGGTKLEIN |
| 553 | C91 VH | EVQLQQSGPVLVKPGASVKMSCKASGNTFNGYYINWVKQSHGKSLEWIGLIDPSNGGT |
| | | NYNQKFKDKATLTVDKSSTTAYMEFNTLTSEDSSVYYCAGGTVYWGQGTTLTVSS |
| 554 | C91 VL | DIQMTQSPSSLSASLGERVSLTCRASQDIGNNLNWLQQEPDGTIKRLIYATSSLDSGVPKR |
| | | FSGSRSGSDYSLTISSLESEDFVDYYCLQYASSPPTFGGGTKLEIK |
| 555 | C92 VH | EVQLVESGGGLVKPGGSLKLSCAASGITFSDYGMHWVRQAPEKGLEWVAYIRSDSSTIY |
| | | YADTVKGRFTISRDNAKNTLFLQMTSLRSDDTAMYYCARRSPVVVNYAMDYWGQGTS |
| | | VTVSS |
| 556 | C92 VL | DIQMTQSPASLSASVGETVTITCRASENIYSYLVWYQQKQGKSPQLLVYNGKILAEGVPS |
| | | RFSGSGSGTQFSLKINSLQPEDFGSYYCQHHYGSPLTFGAGTKLELK |
| 557 | C93 VH | EVHLQQSGPELVKPGASVKISCKASGYTFTDYYMNWMKQSHGKGLEWIGEINPKNGDT |
| | | NYSQKFRDKATLTVDKSSNTAFMKLRSLTSEDSAVYFCAKEGVGSYYAMDYWGQGTS |
| | | VTVSS |
| 558 | C93 VL | DIVMTQSPSSLAMSVGQKVTMSCKSSQSLLSSNNQKSFLAWYQQKPGQSPKLLVSFAST |
| | | RESGVPDRFIGGGSGTDFTLTISSVQPEDLADYFCQQHYSTPPTFGEGTKLEIK |
| 559 | C94 VH | QVQLQQPGAELVKAGASVKMSCKASGYTFTSYWITWVKQRPGQGLEWIGDIYPGNDSP |
| | | NYNEKFKNKATLTVDTSSSTAYMQLSSLTSEDSAVYYCTRGYYDYWGQGTTLTVSS |
| 560 | C94 VL | DIVMTQSPSSLSVSAGEKVTMSCKSSQSLLNSGNQKNYLAWYQQKPGQPPKLLIYGAST |
| | | RESGVPDRFTGSGSGTDFTLTISSVQAEDLAVYYCQNDHSYPHTFGAGTKLELR |
| 561 | C95 VH | EVQLQQSGPVL.VKPGASVKMSCKASGNTFNGYYINWVKQRHGKSLEWIGLIDPSNGGT |
| | | NYNQKFKDKATLTVDKSSTTAYMEFNSLTSDDSSVYYCAGGTVYWGQGTTLTVSS |
| 554 | C95 VL | DIQMTQSPSSLSASLGERVSLTCRASQDIGNNLNWLQQEPDGTIKRLIYATSSLDSGVPKR |
| | | FSGSRSGSDYSLTISSLESEDFVDYYCLQYASSPPTFGGGTKLEIK |
| 562 | C96 VH | EVQLQQSGPELVKPGASVKISCKASGYMFTDYYMNWVKQSHGKSLEWIGEINPNNGDT |
| | | NYSQKFRDKATLTVDKSSNIAFMELRSLTSEDSAVYFCAKEGVSSYYAMDYWGQGTSV |
| | | TVSS |
| 563 | C96 VL | DIVMTQSPSSLAMSVGQKVTMSCKSSQSLLNSNNQKNFLAWYQQKPGQSPKLLVYFAS |
| | | TRESGVPDRFIGGGSGTDFTLTISNVQPEDLADYFCQQHYSTPPTFGGGTKLEIK |
| 564 | C97 VH | EVQLQQSGPELVKPGTSVKISCKASGYTFTDYYMNWVRQSHEKSLEWIGEINPKNGDTN |
| | | YNQNFKAKATLTVDKSSNTAYMELRSLTSEDSAVFYCAREGVSSYYGMDSWGQGTSV |
| | | TVSS |
| 565 | C97 VL | DIVMTQSPSSLAVSVGQKVTMSCKSSQSLLNGFNQKNFLAWYQQKPGQSPKLLVYFAS |
| | | TRESGVPDRFIGSGSGTDFTLTITSVQAEDLADYFCQQHHSTPPTFGGGTKLEIN |
| 566 | C98 VH | EVQLQQSGPELVKPGASVKMSCKASGYTFTDYSMNWVKQSHGKSLEWIGDINPNNGGT |
| | | NYNQKFKGKATLTVDRSSSTAYMELRSLTSEDSAVYYCARVWFAYWGQGTLVTVSA |
| 567 | C98 VL | DIVMTQSPSSLSVSEGEKVTMSCKSSQSLLSSGNQKNFLAWYQQKAGQPPKLLIYGAST |
| | | RESGVPDRFTGSGSGTDFTLTISSVQAEDLAVYYCQNAHSSPFTFGSGTKLEIK |
| 568 | C99 VH | EVQLQQSGPELVKPGASVKISCKASGYTFTDYSMNWVKQSHGKSLEWIGDINPDNGGTS |
| | | YNQKFKGKATLTVDQSSSTAYMELRSLTSEDSAVYYCARIWFAYWGQGTLVTVSA |
| 569 | C99 VL | DIVMTQSPSSLSVSAGEKVTMSCKSSQSLLNSGIQKNFLAWYQQKPGQPPKLLIYGASTR |
| | | ESGVPDRFTGSGSGTDFTLTISSVQAEDLAVYYCQNAHSYPFTFGSGTKLEIK |
| 570 | C100 VH | EVQLQQSGPELVKPGASVKISCKASGYTFTDYYMNWVRQSHEKSLEWIGEINPNNGDT |
| | | NFNQKFKGKATLTVDKSSSTAFMDLRSLTSKDSAVYYCAREGGSSYYAMDYWGQGTS |
| | | VTVSS |
| 571 | C100 VL | DIVMTQSPSSLAMSVGQKVTMSCKSSQSLLSNNNQKNFLAWYQQKPGQSPKLLVYFAS |
| | | TRESGVPDRFIGSGSGTDFTLTISSVQAEDLADYFCQQHYSTPPTFGGGTKVEIK |
| 572 | C101 VH | EVQLQQSGPELVKPGSSVKISCEASGFTFADYNVDWVKQSHGKSLEWIGAINPNDGGTS |
| | | YNQKFEGKATLTVDKSSTTAYMELRSLTSEDSAVYYCARWVNNDWYFDVWGTGTTVT |
| | | VSS |
| 573 | C101 VL | DIVMTQSQKFMSTSVGDRVSITCKASQNVRSAVDWYQQKPGQSPKTLIHSASNRHTGVP |
| | | DRFTGSGSGTDFTLTISNIQSEDLADYFCLQHWNYPYTFGGGTKLEVK |
| 574 | C102 VH | QVQLQQSGAELARPGASVKLSCKASGYTFTNYGISWVKQRTGQGLEWIGEIYPGTINIY |
| | | YSERFKGKATLTADKSSSTAYMQLTSLTSEDSAVYFCARDDGYYDAMDYWGQGTSVT |
| | | VSS |
| 575 | C102 VL | DIQMTQSPSSLSASLGQKVILTCRASQDIGSSLNWLQQEPDGTIKRLIYATSSLDSGVPKR |
| | | FSGSRSGSDYSLTISSLESEDFVVYYCLQYTSSPWTFGGGTKLEIK |
| 576 | C103 VH | QVQLQQPGAELVKPGASVKLSCKASGYTFTSYWMHWVKQRPGQGLEWIGEIDPSDSYT |
| | | NYNQKFKGKATLTVDKSSSTAYMQLSSLTSEDSAVYYCASSIYYSNYVRYYFDYWGQG |
| | | TTLTVSS |
| 577 | C103-2 VH | EVQLQQSGPELVKPGSSVKISCEASGYTFADYNVDWVKQSHGKSLEWIGAINPNDGGTS |
| | | YNQKFEGKATLTVDKSSTTAYMELRSLTSEDSAVYYCARWVINDWYFDVWGTGTTVT |
| | | VSS |
| 578 | C103 VL | DIQMTQSPSSLSASLGGKVTITCKASQDINKYIAWYQHKPGKGPRLLIHYTSTLQPGIPSR |
| | and C103-2 | FSGSGSGRDYSFSISNLEPEDIATYYCLQYDNLWTFGGGTKLEIK |
| | VL | |
| 579 | C104 VH | EVQLQQSGPELVRPGSSVKISCKASGYTFTDYNMDWVKQSHGKSLEWIGANDPKNGGF |
| | | SYNQRFKGKATLTVDKSSSTAYMELRSLTSEDSAVYYCARWDSNYWYFDVWGTGTTV |
| | | TVSS |
| 580 | C104 VL | DIVMTQSQKFMSTSIGDRVSITYKASQDVRSAVAWYQQKPGQSPKALIYLASNRHTGVP |
| | | DRFTGSGSGTDFTLTITNVQSEDLADYFCLQHWTYPYTFGGGTKLEIK |
| 581 | C105 VH | EVQLQQSGPELVKPGSSMKISCKASGYTFTDYSMDWVKQSHGKSLEWIGAINPNNGGTS |
| | | YNQKFKDKATLTVDRSSSTAYMELRSLTSEDSAVYYCARWDSDYWYFDVWATGTTVT |
| | | VSS |
| 582 | C105 VL | DIVMTQSQKFMSTSVGDRVSITCKASQNVRTAVDWYQQKPGQSPKALIYSASNRHTGV |
| | | PDRFTGSGSGTDFTLTISNVQSEDLADYFCLQHWNYPYTFGGGTKLEIK |
| 583 | C106 VH | EVQLQQSGPELVKPGSSVKISCKASGYTFTDYNMDWVKQSHGQSLEYIGAVNPNTGDIS |
| | | YTQKFKDKATLTVDKSSSTAYMELRSLTSEDSAVYYCARWSESWYFDVWGTGTTVTV |
| | | SS |
| 584 | C106 VL | DIVMTQSQKLMSTSVGDRVSITCKASQNVRAAVDWYQQKPRQSPKALIFLASSRHTGVP |
| | | DRFTGSGSGTDFTLTITNVQSEDLATYFCLQHWNYPYTFGGGTRVEIK |
| 585 | C107 VH | EVQLQQSGPELVKPGSSVKISCKASGYTFTDYNIDWVKQSHGKSLEWIGAIDPNNGGTN |
| | | YNQKFKDKATLTVDKSSSTAYMELRSLTSEDSAVYYCTRWDSNDWYFDVWATGTTVT |
| | | VSS |
| 586 | C107 VL | DIVMTQSQKFMSTSVGDRVSITCKASQNVRIAVDWYQQKPGQSPKALIFSSSNRHTGVP |
| | | DRFTGSGSGSDFTLTIRNVQSEDLADYFCLQHWISPYTFGGGTKLEIR |
| 587 | C108 VH | EVQLQQSGPELAKPGSSVKISCKASGYTFTDYNIDWVKQSHGKSLEWIGAIDPNNDSTN |
| | and C108-2 | YNQKFKGKATLTVDKSSSTAYMELRSLTSEDSAVYYCTRWDSDDWYFDVWATGTTVT |
| | VH | VSS |
| 588 | C108 VL | DIVMTQSQKFMSTSVGDRVSITCKASQNVRSAVDWYQQKPGQSPKALIFSSSNRHTGVP |
| | | DRFTGSGSGTDFTLTIRNVQSEDLADYFCLQHWNSPYTFGGGTKLEIR |
| 589 | C108-2 VL | DIQMTQSSSSFSVSRGDRVTITCKASEDIYNRLAWYQQKPGNAPRLLISGTTSLEIGFPSRF |
| | | SGSGSGKDYTLSISSLQTEDVATYYCQQYWSTPWTFGGGTKLEIK |
| 590 | C110 VH | EVQLQQSGAELAKPGASVKLSCTVSGFNIKDTYIHWVKQRPEQGLEWIGGIDPAIGNTVS |
| | | ASKFQDKATITADTSSSTAYMQLSSLTSGDTAVYYCARERVHSNTLYYAMDYWGQGTS |
| | | VTVSS |
| 591 | C110 VL | DIVLTQSPASLAVSLGQRATISCRASKSVSISGYSYMHWYQQKPGQPPKLLIYLASNLES |
| | | GVPARFSGSGSGTDFTLNIHPVEEEDAATYYCQHSRELPPTFGGGTKLEIK |
| 592 | C111 VH | QVQLQQSGPELARPGASVKLSCKASGYTLTSHGISWVKQRTGQGLEWIGEIYPGSESPY |
| | and C111-2 | YSEKFKGKATLTADKSSNIAYMQLSSLTSEDSAVYFCARSDYSDYWAMDYWGQGTSV |
| | VH | TVSS |
| 593 | C111 VL | DIVMTQSQKCMSTSVGDRVSITCKASQNVRSAVAWYQQKPGQSPKTLIYLASNRHTGV |
| | | PDRFTGSGSGTDFTLTITNVQSEDLADYFCLQHWNYPWTFGGGTELEIK |
| 594 | C111-2 VL | EIVMTQSPATLSVSPGERATLSCRASQSVSSNLAWYQQKPGQAPRLLIYGASTRATGIPA |
| | | RFSGSGSGTEFTLTISSLQSEDFAVYYCHQYNNWPRTFGQGTKVEIK |
| 595 | C112 VH | QVQLQQPGAELVKPGASVELSCKASGYTFTSSWMHWVKQRPGQGLEWIGEINPSDGRT |
| | and C112-2 | NYNEKFKSKATLTVDHSSSTAYMQLSSLTSEDSAVYYCASNADRAYYTMDYWGQGTS |
| | VH | VTVSS |
| 596 | C112 VL | DIVMTQSPSSLAMSVGQKVTMSCKSSQSLLNSFNQRNFLAWYQQKPGQSPKLLVYFAS |
| | | TRDSGVPDRFIGSGSGTDFTLTISSVQAEDLADYFCQQHYSTPPTFGGGTKLEIK |
| 594 | C112-2 VL | EIVMTQSPATLSVSPGERATLSCRASQSVSSNLAWYQQKPGQAPRLLIYGASTRATGIPA |
| | | RFSGSGSGTEFTLTISSLQSEDFAVYYCHQYNNWPRTFGQGTKVEIK |
| 597 | C113 VH | EVQLQQSGPELVKPGSSVKISCKASGYTFTDYNLDWMKQSHGSSLEWIGAMSPNNGAT |
| | and C113-2 | SYNQKFKGKATLTVDKSSSTAYMELRSLTSEDSAVYYCARWDSNSWYFDVWGTGTTV |
| | VH | TVSS |
| 598 | C113 VL | DIVMTQSQKFMSTSVGDRVSITCKASQIVRTAVDWYQQKSGQSPKALIYSASTRHTGVP |
| | | DRFTGSGSGTDFTLTIDNVQSEDLAEYFCLQHWNYPYTFGGGTKLEIK |
| 594 | C113-2 VL | EIVMTQSPATLSVSPGERATLSCRASQSVSSNLAWYQQKPGQAPRLLIYGASTRATGIPA |
| | | RFSGSGSGTEFTLTISSLQSEDFAVYYCHQYNNWPRTFGQGTKVEIK |
| 599 | C114 VH | QVQLQQSGAELVRPGTSVKMSCKAAGYTFTNSWINWVKQRPGHGLEWIGDIYPGNTFP |
| | | NYNEKFKGKATLTADTSSSTAYMQLSSLTSEDSAVYYCTGGYFDVWGTGTTVTVSS |
| 600 | C114 VL | DIVMTQSPSSLSVSAGEKVTMSCKSSQSLLNSGNQKNFLAWYQQKPGQPPKLLIFGAST |
| | | RQSGVPDRFTGSGSGTDFTLTISSVQAEDLAVYYCQNDHSYPYTFGGGTKLEMK |
| 601 | C115 VH | EVQLQQSGAELVKPGASVKLSCTASGFNIEDTYIHWVKRRPEQGLEWIGGIDPAIGNTLY |
| | | ASKFQDMATITADTSSSTAYMQLSSLTSGDTAVYYCARERVYSNTLYYAMDYWGQGT |
| | | SVTVSS |
| 602 | C115 VL | DIVLTQSPASLVVSLGQRATISCRASKSVSISGYSYMHWYQQKPGQPPKLLIYLASNLES |
| | | GVPARFSGSGSGTDFTLNIHPVEAEDAATYYCQHSRELPPTFGGGTNLEIK |
| 603 | C116 VH | EVKLVESGGGLVQPGGSLKLSCAVSGFTFSSYTMSWVRQTPEKRLELVAKISNSGGSTN |
| | | YPDTVKGRFTLSRDNVKNTLYLQMSSLKSEDTAIYYCTRYSWDVYAMDYWGQGTSVT |
| | | VSS |
| 604 | C116 VL | DIQMTQSPASLSASVGETVIITCRASENIYSYLAWYQQKQGKSPHLLVYNAETLVEGVPS |
| | | RESGSGSATQFSLKINSLQPEDFGTYYCQHHYGDPTFGGGTKLEIK |
| 605 | C117 VH | QVQLQQSGAELARPGASVKMSCKASGYTFTDYWMQWVKQRPGQGLEWIGAIFPGNGE |
| | | TRDTQKFKGRATLTADRSSSTAYIQLSSLTSEDSAVYYCATENNYLDYWGQGTTLTVAS |
| 606 | C117 VL | QIVLTQSPAIMSASPGEKVTMTCSASSSVSYMHWYQQKSGASPKRWIYDTSKLTSGVPA |
| | | RFSGSGSGTSYSLTISNMEAEDAATYYCQQWISNPYTFGGGTKLEIK |
| 607 | C118 VH | QVQLQQPGAELVKPGTSVKLSCKAAGYIFTSSWMHWVKQRPGQGLEWIGEINPSDGRT |
| | | NYNEKFKNKATLTVDHSSSTAYMQLSSLTSEDSAVYYCASNADRAYYTMDYWGQGTS |
| | | VTVSS |
| 608 | C118 VL | DIVMTQSPSSLAMSVGQKVTMSCKSSQSLLNGFNQRNFLAWYQQKPGQSPKLLVYFAS |
| | | TRDSGVPDRFIGSGSGTDFTLTISSVQAEDLADYFCQQHYSTPPTFGGGTKLEIK |
| 609 | C119 VH | EVKLVESGGGLVQPGGSLKLSCVASGFTFNTYIMSWVRQTPEKRLELVADISNSGGNTY |
| | | YSDTVKGRFTISRDNAKNTLYLQMSSLKSEDTAMYYCARPDNWDPFVYWGQGTLVTV |
| | | SA |
| 610 | C119 VL | DIQMTQSSSSFSVSLGDRVTITCKASEDIYARLAWYQQKPGNAPRLLISGATSLETGVPSR |
| | | FSGSGSGKNYTLSITSLQTEDVGTYYCQQHWSTPWTFGGGTNLEIK |
| 611 | C120 VH | EVQLQQSGPEVVKPGASVKISCKASGNTLTDYFINWVKQSHGKNLEWIGDINPDNGAG |
| | | NYNQKFKGKATLTVDKSSGTAFMGLRSLTSEDSAVYYCATLYFDYWGQGTTLTVSS |
| 612 | C120 VL | DIVMTQSPSSLSVSTGEKVTMSCRSSQSLLNSGNQKNYLAWYQQKPGQPPKLLIYGAST |
| | | RESGVPDRFTGSGSGTDFTLTISSVQAEDLAIYYCQNAHTSPYTFGGGTRLEIK |
| 613 | C121 VH | EVQLQQSGPEVVKPGASVKISCKASGNTFTDYFINWVKQSHGKSLEWIGDINPDNGGTN |
| | | YNQKFKGKATLTVDKSSGTAFMGLRSLTSEDSAVYYCATLYFDYWGQGTSLTVSS |
| 614 | C121 VL | DIVMTQSPSSLSVSAGEKVTMSCKSSQSLLNSGNQKNYLAWYQQKPGQPPKLLIYGAST |
| | | RESGVPDRFTGSGSGTDFTLTISSVQAEDLAVYYCQNAHSSPYTFGGGTRLEIK |
| 615 | C122 VH | EVQLQQSGPEVVKPGASVKISCKASGNTFTDYFINWVKQRHGKSLEWIGDVNPDNGGT |
| | | NYNQKFKGKATLTVDKSSGTAFMGLRSLTSEDSAVYYCATLFFDSWGQGTTLTVSS |
| 616 | C122 VL | DIVMTQSPSSLNVSAGEKVTMSCKSSQSLLNSGNQKNYLAWYQQKPGQPPKLLIYGAST |
| | | RESGVPDRFTGSGSGTDFTLTISSVQAEDLALYYCQNAHSSPYTFGGGTKLEIK |
| 617 | C123 VH | EVQLVESGGGLVKPGGSLKLSCAASGFTFSNFAMSWVRQTPAMRLESVATIVNGGINTY |
| | | YPDTVKGRFTVSRDNAKNTLYLQMSSLRSEDTAIYYCARHDDFLYYAMDYWGQGTSV |
| | | TVSS |
| 618 | C123 VL | QIVLTQSPAIMSASPGEKVTMTCSASSSVSYMFWYQQKPGSSPRLLIYDTSNLASGVPVR |
| | | FSGSGSGTSYSLTISRLEAEDAATYYCQQWTSYPLTFGAGTKLDLK |
| 619 | C124 VH | EVQLVESGGGLVKPGGSLKLSCAASGFTFSSFAMSWVRQTPAKRLESVATINNGGINTY |
| | | YPDTVKGRFTVSRDNAKNTLYLQMSSLRSEDTAMYYCARHDDFLYYAMDYWGQGTS |
| | | VTVSS |
| 620 | C124 VL | QIVLTQSPAIMSASPGEKVTMTCSASSSVSYMYWYQQKPGSSPRLLIYDTSNLASGVPVR |
| | | FSGSGSGTSYSLTISRMEAEDAATYYCQQWTSYPLTFGAGTKLELK |
| 621 | C125 VH | EVKLVESGGGLVQPGGSLKLSCAASGFTFSSYTMSWVRQTPEKRLELVAELSNSGGSFH |
| | | YPDNVKGRFTISRDNAKNTLYLQMSSLKSEDTAMYYCARHNWDVYAMDYWGQGTSV |
| | | TVSS |
| 622 | C125 VL | DIQMSQSPASLSASVGETVTITCRASENVNSYLAWYQQKQGRSPQFLVYNAQTLAEGVP |
| | | SRFSGGGSGTQFSLKINSLQPEDFGSYYCQHHYGNPTFGGGTKLEIK |
| 623 | C129 VH | QVQLQQSGAELMTPGSSVKLSCKATGYTFTDYWIEWVKKRPGHGLEWIGEILPGYISTN |
| | | YNEKFKDKATFTADTSSNTTYMQLSSLTSEDSAVYYCVLIVTTYYYFMDYWGQGTSVT |
| | | VSS |
| 624 | C129 VL | QIVLTQSPAIMSASPGEKVTITCSASSSVTYMHWFQQKPGTSPKLWIYSTSNLASGVPVR |
| | | FSGSGSGTSYSLTISRMEAEDAATYYCQQRSSYPWTFGGGTKLEIK |
| 625 | C132 VH | QVQLQQSGAELMKPGSSVNLSCKATDYTFTDYWINWVKQRPGHGLEWIGEILPGSVTT |
| | | NYNEKFRGKATFTADTSSNTAYMQLSSLTSEDSAVYFCARGSDYSYYAMDYWGQGTS |
| | | VTVSS |
| 626 | C132 VL | DIVMTQSPSSLAMSVGQKVTMSCKSSQSLLNIFNQKNFLAWYQQKPGQSPKLLVYFAST |
| | | RESGVPDRFIGSGSGTDFTLTISNVQAEDLADYFCQQHYSTPPTFGGGTKLEIK |
| 627 | C136 VH | DVQLVESGGGLVKPGGSLKLSCAASGFSFSENYMYWVRQTPEKRLEWVATISDGASYT |
| | | YYPDNVKGRFTISRDTAKNNLYLQMSHLKSEDTAMYYCTRDELIAPFAYWGQGTLVTV |
| | | SA |
| 628 | C136 VL | DIVMTQSHKFMSTSVGDRVSITCKASQDVGTAVAWFQQKPGQSPKLLIYWASTRHTGV |
| | | PDRFTGSGSGTDFTLTISNVQSEDLADYFCQQYNSYPWTFGGGTKLEIK |
| 629 | C138 VH | EVQLVESGGGLVKPGGSLKLSCAASGFTFSSYAMSWVRQTPAKRLEWVATITSGDIRTY |
| | | YPDTVKGRFTISRDNAKNTLYLQMRSLRSEDTAMYYCVGHDDFHFYAMDYWGQGTSV |
| | | TVSS |
| 630 | C138 VL | QIVLTQSPAIMSASPGEKVTMTCSASSSVSYLYWYQQKPGSSPRLLIYDTSNLASGVPVR |
| | | FSGSGSGTSYSLTLSRMEAEDAATYYCQQWSSYPLTFGAGTKLELK |
| 631 | C140 VH | QVQLQQSGAELARPGTSVKMSCKASGYIFTNYWMNWVQQRPGQGLEWIGAINPSNGYI |
| | | GYNQKFKDKATLTADKSSNTAYMQLRSLTSEDSAIYYCTRGEDFDGYAMDYWGQGTS |
| | | VTVSS |
| 632 | C140 VL | DIQMTQSPSSLSASLGGKVTITCKASQDINKYIAWYQHKPGKGPRLLIHYTSTLQPGIPSR |
| | | FSGSGSGRDYSFSISNLEPEDIATYYCLQYDNLLTFGAGTKLELK |
| 617 | C143 VH | EVQLVESGGGLVKPGGSLKLSCAASGFTFSNFAMSWVRQTPAMRLESVATIVNGGINTY |
| | | YPDTVKGRFTVSRDNAKNTLYLQMSSLRSEDTAIYYCARHDDFLYYAMDYWGQGTSV |
| | | TVSS |
| 633 | C143 VL | QIVLTQSPAIMSASPGEKVTMTCSASSSVNYMFWYQQKPGSSPRLLIYDTSNLASGVPVR |
| | | FSGSGSGTSYSLTISRLEAEDAATYYCQQWTSYPLTFGAGTKLDLK |
| 634 | C145 VH | EVQLQQSGPEVVKPGASVRISCKASGNTFTDFFINWVKQSHGKSLEWIGDVNPDSNGAN |
| | | YNQKFKGKATLTVDKSSGTAFMGLRSLTSEDSAVYYCATLFFDNWGQGTTLTVSS |
| 635 | C145 VL | DIVMTQSPSSLNVSAGEKVTMSCKSSQSLLNSGNQKNYLAWYQQKPGQPPKLLIYGAST |
| | | RESGVPDRFTGSGSGTDFILTISSVQAEDLALYYCQNAHSSPYTFGGGTKLEIK |
| 636 | C146 VH | EVQLQQSGPELVKPGSSVKISCEASGYTFADYNVDWVKQSHGKSLEWIGAINPNDGGTS |
| | | YNQKFEGKATLTVDKSSTTVYMELRSLTSEDSAVYYCARWVNNDWYFDVWGTGTTVT |
| | | VSS |
| 637 | C146 VL | DIVMTQSQKFMSTSVGDRVSITCKASQNVRSAVDWYQQKPGQSPKTLIHSASSRHTGVP |
| | | DRFTGSGSGTDFTLTISNIQSEDLADYFCLQHWNYPYTFGGGTKLEVK |
| 638 | C147 VH | EVQLQQSGPELLKPGSSVNISCKASGYTFTDYNIDWVKQSRGKSLEWIGAINPNNGGTFY |
| | | NQKFKGQATLTVYKSSSTAYMELRSLTSEDSAVYYCVRWDSDSWYFDVWGTGTAVTV |
| | | SS |
| 639 | C147 VL | DIVMTQSQKFMSTSVGERVSITCKASENVRAAVDWYQQKPGQSPKALIYLASNRHTGIP |
| | | DRFTGSGSGTDFTLIISNVQSEDLADYFCLQHWNYPYTFGGGTKLEIT |
| 640 | C148 VH | EVQLQQSGPELAKPGSSVKISCKASGYTFTDYNIDWVKESHGKSLEWIGAIDPNNGGTN |
| | | YNQKFKGKATLTVDKSSSTAYMELRSLTSEDSAVYYCTRWDSNDWYFDVWATGTTVT |
| | | VSS |
| 641 | C148 VL | DIVMTQSQKFMSTSVGDRVSITCKASQNVRSAVDWYQQKPGQSPKALIFSSSNRHTGVP |
| | | DRFAGSGSGTDFNLTIRNVQSEDLADYFCLQHWNSPYTFGGGTKLEIR |
| 642 | C149 VH | QVTLKESGPGILQPSQTLSLTCSFSGFSLSTFGLGIGWIRQPSGKGLEWLAHIWWDDDKY |
| | | YNPALKSRLTISKDISKNQVFLKIANVDTADTATYYCATGTYWFTYWGQGTLVTVSA |
| 643 | C149 VL | DVLMTQTPLSLPVSLGDQASISCRSSQSIEHSNGNTYLQWYLQKPGQSPKLLIYKVSNRF |
| | | SGVPDRFSGSGSGTDFTLKISRVEAEDLGVYYCFQGSHVPWTFGGGTKLEIK |
| 644 | C150 VH | EVQLQQSGPVLVKPGASVKMSCKASGYRLTDYYMHWVKQSHGKSLEWIGIINPYNGDT |
| | | SYNQKFKGKATLTVDKSSSTAYMELDGLTSDDSAVYYCARDSSNVGNAMDYWGQGTS |
| | | VTVSS |
| 645 | C150 VL | DIQMTQTTSSLSASLGDRITISCRASQDISNYLNWYLQKPDGTIKLLIYYSSRLHSGVPSRF |
| | | SGSGSGTDYSLTITNLEQEDIATYFCQQGHALPRTFGGGTKLEIK |
| 646 | C151 VH | EVQLQQSGPELVKPGASVKISCKGSGYTFTDYFMNWVKQSHGKSLEWVGDINPNNGDT |
| | | TYSQKFMGKATLTVDKSSSTAYMELRSLTSEDSAVYHCAASGPPYVLLDYWGQGTSVT |
| | | VSS |
| 647 | C151 VL | DIQMTQSPASLSASVGETVTITCRASENIYSYLAWYQQKQGKSPQLLVYNAKTLTEGVP |
| | | TRESGSGSGTQFSLKIDSLKPEDFGTYYCQHHYGTPLTFGAGTKLELN |
| 648 | C152 VH | QVQLQQSGTILARPGASVKMSCRASDYTFTDYWIQWIKQRPGQGLEWIGAIFPENGDTR |
| | | YTQKFKGRATLTADKSSSTAYMQVNSLTSEDSAVYYCATFINYVDYWGQGTTLTVSS |
| 649 | C152 VL | QIVLIQSPAIMSASPGEKVTMTCSASSSVNNIHWYQQKSGTSPKRWIYDTTKLTSGVPAR |
| | | FSGSGSGTSYSLTISSMEAEDAATYYCQQWNINPYTFGGGTKLEIK |
| 650 | C153 VH | EVQLQQSGPELVKPGSLVKISCKTSGYTFTDYNMDWVKQSHGKSLEWIGAIDPKNGATS |
| | | YNQKFKGKATLTVDKSSSTAYMELRSLTSEDSAVYYCARWDSNYWYFDVWGTGTTVT |
| | | VSS |
| 651 | C153 VL | DIVMTQSQKIMSTSVGDRVSMTCKASQNIRSAVAWYQQKPGQSPKALIYLTSNRHTGVP |
| | | DRFTGSGSGTDFTLAISDLQSEDLADYFCLQHWNYPYTFGGGTELEIK |
| 652 | C154VH | EVQLVESGGGLVKPGGSLRLSCAASGFTFSDYYMYWVRQAPGKGLEWVSTISDAGGYT |
| | | YYPDSVKGRFTISRDNAKNSLYLQMNSLRAEDTAVYYCAREDLWGQGTLVTVSS |
| 653 | C154VL | DIQMTQSPSSLSASVGDRVTITCRASQDIGYSLNWFQQKPGKAPKRLIYATSRLDSGVPK |
| | | RFSGSGSGTDFTLTISSLQPEDFATYYCLQYASFPYTFGGGTKLEIK |
| 654 | C155VH | EVQLVESGGGLVKPGGSLRLSCAASGFTFSDYYMYWVRQAPGKGLEWVATISDAGGYT |
| | | YYPDSVKGRFTISRDNAKNSLYLQMNSLRAEDTAVYYCGREDLWGQGTLVTVSS |
| 655 | C155VL | DIQMTQSPSSLSASVGDRVTITCRASQDIGYSLNWFQQKPGKAPRRLIYATSRLDSGVPKR |
| | | FSGSGSGTDFTLTISSLQPEDFATYYCLQYASFPYTFGGGTKLEIK |
| 656 | C156VH | EVQLVESGGGLVKPGGSLRLSCAASGFTFSDYYMYWVRQAPGKGLEWVATISDAGGYT |
| | | YYPDSVKGRFTISRDNAKNSLYLQMNSLRAEDTAVYFCGREDLWGQGTLVTVSS |
| 653 | C156VL | DIQMTQSPSSLSASVGDRVTITCRASQDIGYSLNWFQQKPGKAPKRLIYATSRLDSGVPK |
| | | RFSGSGSGTDFTLTISSLQPEDFATYYCLQYASFPYTFGGGTKLEIK |

1-12. (canceled)

13. An anti-CD112R antibody or antigen-binding fragment thereof that specifically binds to CD112R, comprising:
1) a variable heavy chain polypeptide which comprises the HCDR1, HCDR2 and HCDR3 polypeptides comprised in the variable heavy chain polypeptide of SEQ ID NO: 459 and a variable light chain polypeptide which comprises the LCDR1, LCDR2 and LCDR3 polypeptides comprised in the variable light chain polypeptide of SEQ ID NO: 460;
2) a variable heavy chain polypeptide which comprises the HCDR1, HCDR2 and HCDR3 polypeptides comprised in the variable heavy chain polypeptide of SEQ ID NO: 430 and a variable light chain polypeptide which comprises the LCDR1, LCDR2 and LCDR3 polypeptides comprised in the variable light chain polypeptide of SEQ ID NO: 431;
3) a variable heavy chain polypeptide which comprises the HCDR1, HCDR2 and HCDR3 polypeptides comprised in the variable heavy chain polypeptide of SEQ ID NO: 432 and a variable light chain polypeptide which comprises the LCDR1, LCDR2 and LCDR3 polypeptides comprised in the variable light chain polypeptide of SEQ ID NO: 433;
4) a variable heavy chain polypeptide which comprises the HCDR1, HCDR2 and HCDR3 polypeptides comprised in the variable heavy chain polypeptide of SEQ ID NO: 434 and a variable light chain polypeptide which comprises the LCDR1, LCDR2 and LCDR3 polypeptides comprised in the variable light chain polypeptide of SEQ ID NO: 435;
5) a variable heavy chain polypeptide which comprises the HCDR1, HCDR2 and HCDR3 polypeptides comprised in the variable heavy chain polypeptide of SEQ ID NO: 438 and a variable light chain polypeptide which comprises the LCDR1, LCDR2 and LCDR3 polypeptides comprised in the variable light chain polypeptide of SEQ ID NO: 439;
6) a variable heavy chain polypeptide which comprises the HCDR1, HCDR2 and HCDR3 polypeptides comprised in the variable heavy chain polypeptide of SEQ ID NO: 441 and a variable light chain polypeptide which comprises the LCDR1, LCDR2 and LCDR3 polypeptides comprised in the variable light chain polypeptide of SEQ ID NO: 442;
7) a variable heavy chain polypeptide which comprises the HCDR1, HCDR2 and HCDR3 polypeptides comprised in the variable heavy chain polypeptide of SEQ ID NO: 447 and a variable light chain polypeptide which comprises the LCDR1, LCDR2 and LCDR3 polypeptides comprised in the variable light chain polypeptide of SEQ ID NO: 448;
8) a variable heavy chain polypeptide which comprises the HCDR1, HCDR2 and HCDR3 polypeptides comprised in the variable heavy chain polypeptide of SEQ ID NO: 449 and a variable light chain polypeptide which comprises the LCDR1, LCDR2 and LCDR3 polypeptides comprised in the variable light chain polypeptide of SEQ ID NO: 450;
9) a variable heavy chain polypeptide which comprises the HCDR1, HCDR2 and HCDR3 polypeptides comprised in the variable heavy chain polypeptide of SEQ ID NO: 451 and a variable light chain polypeptide which comprises the LCDR1, LCDR2 and LCDR3 polypeptides comprised in the variable light chain polypeptide of SEQ ID NO: 452;
10) a variable heavy chain polypeptide which comprises the HCDR1, HCDR2 and HCDR3 polypeptides comprised in the variable heavy chain polypeptide of SEQ ID NO: 453 and a variable light chain polypeptide which comprises the LCDR1, LCDR2 and LCDR3 polypeptides comprised in the variable light chain polypeptide of SEQ ID NO: 454;
11) a variable heavy chain polypeptide which comprises the HCDR1, HCDR2 and HCDR3 polypeptides comprised in the variable heavy chain polypeptide of SEQ ID NO: 457 and a variable light chain polypeptide which comprises the LCDR1, LCDR2 and LCDR3 polypeptides comprised in the variable light chain polypeptide of SEQ ID NO: 458;
12) a variable heavy chain polypeptide which comprises the HCDR1, HCDR2 and HCDR3 polypeptides comprised in the variable heavy chain polypeptide of SEQ ID NO: 461 and a variable light chain polypeptide which comprises the LCDR1, LCDR2 and LCDR3 polypeptides comprised in the variable light chain polypeptide of SEQ ID NO: 462;
13) a variable heavy chain polypeptide which comprises the HCDR1, HCDR2 and HCDR3 polypeptides comprised in the variable heavy chain polypeptide of SEQ ID NO: 469 and a variable light chain polypeptide which comprises the LCDR1, LCDR2 and LCDR3 polypeptides comprised in the variable light chain polypeptide of SEQ ID NO: 470; or
14) a variable heavy chain polypeptide which comprises the HCDR1, HCDR2 and HCDR3 polypeptides comprised in the variable heavy chain polypeptide of SEQ ID NO: 485 and a variable light chain polypeptide which comprises the LCDR1, LCDR2 and LCDR3 polypeptides comprised in the variable light chain polypeptide of SEQ ID NO: 486; and
wherein the numbering of said CDR polypeptides is according to Kabat.

14. The anti-CD112R antibody or antigen-binding fragment thereof of claim 13, which is a monoclonal antibody.

15. The anti-CD112R antibody or antigen-binding fragment thereof of claim 13, which is humanized.

16. The anti-CD112R antibody or antigen-binding fragment thereof of claim 13, wherein the antigen-binding fragment is selected from a Fab, Fab′-SH, Fv, scFv or (Fab′)₂ fragment.

17. The anti-CD112R antibody or antigen-binding fragment thereof of claim 13, which comprises a constant region, wherein at least a portion of the constant region comprises a human consensus constant region sequence.

18. The anti-CD112R antibody or antigen-binding fragment thereof of claim 13, which comprises a human constant region, optionally a human IgG1, IgG2, IgG3 or IgG4 constant region.

19. The antibody or antigen-binding fragment thereof of claim 13, which comprises a heavy chain constant region which comprises the amino acid sequence of SEQ ID NO; 659, and a light chain constant region which comprises the amino acid sequence of SEQ ID NO: 660.

20. An isolated polynucleotide encoding the anti-CD112R antibody or antigen-binding fragment thereof of claim 13, or an expression vector comprising said polynucleotide.

21. An isolated or recombinant host cell comprising the polynucleotide or expression vector of claim 20.

22. A method of producing an anti-CD112R antibody or antigen-binding fragment thereof according to claim 13 comprising culturing a recombinant cell comprising a polynucleotide encoding the anti-CD112R antibody or antigen-binding fragment thereof or an expression vector comprising said polynucleotide under conditions which permit expression of the anti-CD112R antibody or antigen-binding fragment thereof encoded by the polynucleotide and optionally isolating the anti-CD112R antibody or antigen-binding fragment thereof from the culture medium comprising said recombinant cell.

23. A pharmaceutical composition comprising an anti-CD112R antibody or antigen-binding fragment thereof according to claim 13, and a pharmaceutically acceptable carrier or excipient.

24. A method for preventing or treating a CD112R-mediated disease in a subject in need thereof, comprising administering to the subject a prophylactically or therapeutically effective amount of
(i) an antibody or antigen-binding fragment thereof according to claim 13,
(ii) a polynucleotide encoding said antibody or antigen-binding fragment thereof according to claim 13 or an expression vector comprising said polynucleotide, or
(iii) a recombinant cell which comprises a polynucleotide encoding said antibody or antigen-binding fragment thereof according to claim 13 or an expression vector comprising said polynucleotide.

25. The method of claim 24, wherein the CD112R-mediated disease or disorder is a CD112R-associated cancer, optionally selected from melanoma, renal cancer, prostate cancer, breast cancer, colon cancer, lung cancer, bone cancer, pancreatic cancer, skin cancer, head and neck cancer, uterine cancer, ovarian cancer and rectal cancer.

26. An anti-CD112R antibody or antigen-binding fragment thereof which comprises
1) a variable heavy chain polypeptide which comprises the amino acid sequence of SEQ ID NO: 459 and a variable light chain polypeptide which comprises the amino acid sequence of SEQ ID NO: 460;
2) a variable heavy chain polypeptide which comprises the amino acid sequence of SEQ ID NO: 430 and a variable light chain polypeptide which comprises the amino acid sequence of SEQ ID NO: 431;
3) a variable heavy chain polypeptide which comprises the amino acid sequence of SEQ ID NO: 432 and a variable light chain polypeptide which comprises the amino acid sequence of SEQ ID NO: 433;
4) a variable heavy chain polypeptide which comprises the amino acid sequence of SEQ ID NO: 434 and a variable light chain polypeptide which comprises the amino acid sequence of SEQ ID NO: 435;
5) a variable heavy chain polypeptide which comprises the amino acid sequence of SEQ ID NO: 438 and a variable light chain polypeptide which comprises the amino acid sequence of SEQ ID NO: 439;
6) a variable heavy chain polypeptide which comprises the amino acid sequence of SEQ ID NO: 441 and a variable light chain polypeptide which comprises the amino acid sequence of SEQ ID NO: 442;
7) a variable heavy chain polypeptide which comprises the amino acid sequence of SEQ ID NO: 447 and a variable light chain polypeptide which comprises the amino acid sequence of SEQ ID NO: 448;
8) a variable heavy chain polypeptide which comprises the amino acid sequence of SEQ ID NO: 449 and a variable light chain polypeptide which comprises the amino acid sequence of SEQ ID NO: 450;
9) a variable heavy chain polypeptide which comprises the amino acid sequence of SEQ ID NO: 451 and a variable light chain polypeptide which comprises the amino acid sequence of SEQ ID NO: 452;
10) a variable heavy chain polypeptide which comprises the amino acid sequence of SEQ ID NO: 453 and a variable light chain polypeptide which comprises the amino acid sequence of SEQ ID NO: 454;
11) a variable heavy chain polypeptide which comprises the amino acid sequence of SEQ ID NO: 457 and a variable light chain polypeptide which comprises the amino acid sequence of SEQ ID NO: 458;
12) a variable heavy chain polypeptide which comprises the amino acid sequence of SEQ ID NO: 461 and a variable light chain polypeptide which comprises the amino acid sequence of SEQ ID NO: 462;
13) a variable heavy chain polypeptide which comprises the amino acid sequence of SEQ ID NO: 469 and a variable light chain polypeptide which comprises the amino acid sequence of SEQ ID NO: 470; or
14) a variable heavy chain polypeptide which comprises the amino acid sequence of SEQ ID NO: 485 and a variable light polypeptide which comprises the amino acid sequence of SEQ ID NO: 486.

27. An isolated polynucleotide encoding the anti-CD112R antibody or antigen-binding fragment thereof of claim 26, or an expression vector comprising said polynucleotide.

28. An isolated or recombinant host cell comprising the polynucleotide or expression vector of claim 27.

29. A method of producing an anti-CD112R antibody or antigen-binding fragment thereof according to claim 26 comprising culturing a recombinant cell comprising a polynucleotide encoding the anti-CD112R antibody or antigen-binding fragment thereof under conditions which permit expression of the anti-CD112R antibody or antigen-binding fragment thereof encoded by the polynucleotide and optionally isolating the anti-CD112R antibody or antigen-binding fragment thereof from the culture medium comprising said recombinant cell.

30. A pharmaceutical composition comprising an anti-CD112R antibody or antigen-binding fragment thereof according to claim 26, a polynucleotide or expression vector according to claim 27, or an isolated or recombinant cell according to claim 28, and a pharmaceutically acceptable carrier or excipient.

31. A method for preventing or treating a CD112R-mediated disease in a subject in need thereof, comprising administering to the subject a prophylactically or therapeutically effective amount of
(i) an anti-CD112R antibody or antigen-binding fragment thereof according to claim 26,
(ii) a polynucleotide encoding said anti-CD112R antibody or antigen-binding fragment thereof or an expression vector comprising said polynucleotide, or
(iii) an isolated or recombinant cell comprising a polynucleotide encoding said anti-CD112R antibody or antigen-binding fragment thereof or an expression vector comprising said polynucleotide, and
optionally a pharmaceutically acceptable carrier or excipient.

32. The method of claim 31, wherein the CD112R-mediated disease or disorder is a CD112R-associated cancer, optionally selected from melanoma, renal cancer, prostate cancer, breast cancer, colon cancer, lung cancer, bone cancer, pancreatic cancer, skin cancer, head and neck cancer, uterine cancer, ovarian cancer and rectal cancer.


SEQUENCE LISTING (662 sequences)

SEQ ID NO: 1 - protein, 5 aa, Synthetic construct
DYYLN

SEQ ID NO: 2 - protein, 5 aa, Synthetic construct
DYYIN

SEQ ID NO: 3 - protein, 5 aa, Synthetic construct
DYFMN

SEQ ID NO: 4 - protein, 5 aa, Synthetic construct
AYYIN

SEQ ID NO: 5 - protein, 5 aa, Synthetic construct
DYYMN

SEQ ID NO: 6 - protein, 5 aa, Synthetic construct
DYYMF

SEQ ID NO: 7 - protein, 5 aa, Synthetic construct
DIYMH

SEQ ID NO: 8 - protein, 5 aa, Synthetic construct
DSYMC

SEQ ID NO: 9 - protein, 5 aa, Synthetic construct
DYYMY

SEQ ID NO: 10 - protein, 5 aa, Synthetic construct
SYWMH

SEQ ID NO: 11 - protein, 5 aa, Synthetic construct
DYNIH

SEQ ID NO: 12 - protein, 5 aa, Synthetic construct
GYGIN

SEQ ID NO: 13 - protein, 5 aa, Synthetic construct
DYYIA

SEQ ID NO: 14 - protein, 5 aa, Synthetic construct
TYGMS

SEQ ID NO: 15 - protein, 5 aa, Synthetic construct
GYWIE

SEQ ID NO: 16 - protein, 5 aa, Synthetic construct
SYNIH

SEQ ID NO: 17 - protein, 5 aa, Synthetic construct
TYWMH

SEQ ID NO: 18 - protein, 5 aa, Synthetic construct
DSYIN

SEQ ID NO: 19 - protein, 5 aa, Synthetic construct
SYWMQ

SEQ ID NO: 20 - protein, 5 aa, Synthetic construct
DYEMH

SEQ ID NO: 21 - protein, 5 aa, Synthetic construct
DYYMS

SEQ ID NO: 22 - protein, 5 aa, Synthetic construct
GYALS

SEQ ID NO: 23 - protein, 5 aa, Synthetic construct
DYALS

SEQ ID NO: 24 - protein, 5 aa, Synthetic construct
DYYMH

SEQ ID NO: 25 - protein, 6 aa, Synthetic construct
SNYYWN

SEQ ID NO: 26 - protein, 5 aa, Synthetic construct
SYSMS

SEQ ID NO: 27 - protein, 5 aa, Synthetic construct
SYIMS

SEQ ID NO: 28 - protein, 5 aa, Synthetic construct
SYFMS

SEQ ID NO: 29 - protein, 5 aa, Synthetic construct
SYWIT

SEQ ID NO: 30 - protein, 5 aa, Synthetic construct
HYYKN

SEQ ID NO: 31 - protein, 5 aa, Synthetic construct
HYYVN

SEQ ID NO: 32 - protein, 5 aa, Synthetic construct
NYGMS

SEQ ID NO: 33 - protein, 5 aa, Synthetic construct
GYYVN

SEQ ID NO: 34 - protein, 5 aa, Synthetic construct
DYFIS

SEQ ID NO: 35 - protein, 5 aa, Synthetic construct
DYSMN

SEQ ID NO: 36 - protein, 5 aa, Synthetic construct
ASAMH

SEQ ID NO: 37 - protein, 5 aa, Synthetic construct
DYGMY

SEQ ID NO: 38 - protein, 5 aa, Synthetic construct
GYYIN

SEQ ID NO: 39 - protein, 5 aa, Synthetic construct
DYGMH

SEQ ID NO: 40 - protein, 5 aa, Synthetic construct
DYNVD

SEQ ID NO: 41 - protein, 5 aa, Synthetic construct
NYGIS

SEQ ID NO: 42 - protein, 5 aa, Synthetic construct
DYNMD

SEQ ID NO: 43 - protein, 5 aa, Synthetic construct
DYSMD

SEQ ID NO: 44 - protein, 5 aa, Synthetic construct
DYNID

SEQ ID NO: 45 - protein, 5 aa, Synthetic construct
DTYIH

SEQ ID NO: 46 - protein, 5 aa, Synthetic construct
SHGIS

SEQ ID NO: 47 - protein, 5 aa, Synthetic construct
SSWMH

SEQ ID NO: 48 - protein, 5 aa, Synthetic construct
DYNLD

SEQ ID NO: 49 - protein, 5 aa, Synthetic construct
NSWIN

SEQ ID NO: 50 - protein, 5 aa, Synthetic construct
SYTMS

SEQ ID NO: 51 - protein, 5 aa, Synthetic construct
DYWMQ

SEQ ID NO: 52 - protein, 5 aa, Synthetic construct
TYIMS

SEQ ID NO: 53 - protein, 5 aa, Synthetic construct
DYFIN

SEQ ID NO: 54 - protein, 5 aa, Synthetic construct
NFAMS

SEQ ID NO: 55 - protein, 5 aa, Synthetic construct
SFAMS

SEQ ID NO: 56 - protein, 5 aa, Synthetic construct
DYWIE

SEQ ID NO: 57 - protein, 5 aa, Synthetic construct
DYWIN

SEQ ID NO: 58 - protein, 5 aa, Synthetic construct
ENYMY

SEQ ID NO: 59 - protein, 5 aa, Synthetic construct
SYAMS

SEQ ID NO: 60 - protein, 5 aa, Synthetic construct
NYWMN

SEQ ID NO: 61 - protein, 5 aa, Synthetic construct
DFFIN

SEQ ID NO: 62 - protein, 7 aa, Synthetic construct
TFGLGIG

SEQ ID NO: 63 - protein, 5 aa, Synthetic construct
DYWIQ

SEQ ID NO: 64 - protein, 17 aa, Synthetic construct
WIFPGSESTFYNEKFKG

SEQ ID NO: 65 - protein, 17 aa, Synthetic construct
WIFPGSDYTYYNEKFKG

SEQ ID NO: 66 - protein, 17 aa, Synthetic construct
DIIPNNDSPNYNQKFKG

SEQ ID NO: 67 - protein, 17 aa, Synthetic construct
WIFPGSDSTYYNEKFKG

SEQ ID NO: 68 - protein, 17 aa, Synthetic construct
DINPNNGGTNFNQKFKG

SEQ ID NO: 69 - protein, 17 aa, Synthetic construct
TISDGGGYIYYPDSVKG

SEQ ID NO: 70 - protein, 17 aa, Synthetic construct
RIDPTNGNSEYDPRFRG

SEQ ID NO: 71 - protein, 17 aa, Synthetic construct
TISDGGGLTYYPDSVKG

SEQ ID NO: 72 - protein, 17 aa, Synthetic construct
DIHPNNGSPNYNQKFKG

SEQ ID NO: 73 - protein, 17 aa, Synthetic construct
TISDGGGYTYYPDSVKG

SEQ ID NO: 74 - protein, 17 aa, Synthetic construct
DINPNNGSPNYNQKFKG

SEQ ID NO: 75 - protein, 17 aa, Synthetic construct
VIDASDSNTKYNQKFKG

SEQ ID NO: 76 - protein, 17 aa, Synthetic construct
TISDGGGFIYYPDSVKG

SEQ ID NO: 77 - protein, 17 aa, Synthetic construct
TISDAGGYTYYPDSVKG

SEQ ID NO: 78 - protein, 17 aa, Synthetic construct
RVDPTNGNSEYDPRFQG

SEQ ID NO: 79 - protein, 17 aa, Synthetic construct
YIYPYNGGAGYNQKFKS

SEQ ID NO: 80 - protein, 17 aa, Synthetic construct
WIFPGSGNTYYNEKFKG

SEQ ID NO: 81 - protein, 16 aa, Synthetic construct
VIWSGGNTEYNAAFRS

SEQ ID NO: 82 - protein, 17 aa, Synthetic construct
VIDASDSKTKYNQKFKG

SEQ ID NO: 83 - protein, 17 aa, Synthetic construct
WIYPGSGYTKYNEKLKG

SEQ ID NO: 84 - protein, 17 aa, Synthetic construct
WIYLGSGHTMYNENFKG

SEQ ID NO: 85 - protein, 17 aa, Synthetic construct
WIFPGSGSTYYNEKFKG

SEQ ID NO: 86 - protein, 17 aa, Synthetic construct
WINTYSGVPTYADDFKG

SEQ ID NO: 87 - protein, 17 aa, Synthetic construct
EILPESGRTNYNEKFKG

SEQ ID NO: 88 - protein, 17 aa, Synthetic construct
AIYPGNADISYNQKFRD

SEQ ID NO: 89 - protein, 17 aa, Synthetic construct
DIEPNNGGTNYNQKFKG

SEQ ID NO: 90 - protein, 17 aa, Synthetic construct
WIFPGSGTTYYNEKFKG

SEQ ID NO: 91 - protein, 17 aa, Synthetic construct
EIDPFDSYTNYNQKFKG

SEQ ID NO: 92 - protein, 17 aa, Synthetic construct
AIDPETGGTAYNQKFKG

SEQ ID NO: 93 - protein, 17 aa, Synthetic construct
DVNPNNGDTSYNQKFKG

SEQ ID NO: 94 - protein, 17 aa, Synthetic construct
TIRDGGSYTYYSDNEKG

SEQ ID NO: 95 - protein, 17 aa, Synthetic construct
IINPYNGDTSYNQKFKD

SEQ ID NO: 96 - protein, 16 aa, Synthetic construct
YISSDGTNNYNPSLRN

SEQ ID NO: 97 - protein, 17 aa, Synthetic construct
TIGGVGGNTYYPDSVKG

SEQ ID NO: 98 - protein, 17 aa, Synthetic construct
TIRGGGGNTYYLDSVKG

SEQ ID NO: 99 - protein, 17 aa, Synthetic construct
TISGGGDKTYYLDILKG

SEQ ID NO: 100 - protein, 17 aa, Synthetic construct
DINPDNGGSTYNQKFKG

SEQ ID NO: 101 - protein, 17 aa, Synthetic construct
IINPYNGGTSYNQKFKG

SEQ ID NO: 102 - protein, 17 aa, Synthetic construct
TISGGGDNTYYPDSVKG

SEQ ID NO: 103 - protein, 17 aa, Synthetic construct
TIRGGGSYTYYLDSVKG

SEQ ID NO: 104 - protein, 17 aa, Synthetic construct
DIYPGNFSPNYNEKFKN

SEQ ID NO: 105 - protein, 17 aa, Synthetic construct
AINPNIGGPSYNQKFKG

SEQ ID NO: 106 - protein, 17 aa, Synthetic construct
DIYPNNGDTNYNQKFKG

SEQ ID NO: 107 - protein, 17 aa, Synthetic construct
VIKPYNGAPSYNQKFKG

SEQ ID NO: 108 - protein, 17 aa, Synthetic construct
TISRGGSFTYYPDSVKG

SEQ ID NO: 109 - protein, 17 aa, Synthetic construct
DIYPGNDSPNYNEKFKN

SEQ ID NO: 110 - protein, 17 aa, Synthetic construct
LIDPSNGGTNYNQKFKD

SEQ ID NO: 111 - protein, 17 aa, Synthetic construct
TFNPNRGGPSYNLKFKG

SEQ ID NO: 112 - protein, 17 aa, Synthetic construct
WIFPGSGSTYFNENFKG

SEQ ID NO: 113 - protein, 17 aa, Synthetic construct
DINPNNGGTSYNQKFKG

SEQ ID NO: 114 - protein, 17 aa, Synthetic construct
SFTMYSDATEYSENFKG

SEQ ID NO: 115 - protein, 17 aa, Synthetic construct
DIHPGNTSPNYNEKFRT

SEQ ID NO: 116 - protein, 17 aa, Synthetic construct
YIRSDSSTIYYTDTVKG

SEQ ID NO: 117 - protein, 17 aa, Synthetic construct
EINPKNGDTNYNQNFKG

SEQ ID NO: 118 - protein, 17 aa, Synthetic construct
YIRSDSSTIYYADTVKG

SEQ ID NO: 119 - protein, 17 aa, Synthetic construct
EINPKNGDTNYSQKFRD

SEQ ID NO: 120 - protein, 17 aa, Synthetic construct
EINPNNGDTNYSQKFRD

SEQ ID NO: 121 - protein, 17 aa, Synthetic construct
EINPKNGDTNYNQNFKA

SEQ ID NO: 122 - protein, 17 aa, Synthetic construct
DINPNNGGTNYNQKFKG

SEQ ID NO: 123 - protein, 17 aa, Synthetic construct
DINPDNGGTSYNQKFKG

SEQ ID NO: 124 - protein, 17 aa, Synthetic construct
EINPNNGDTNFNQKFKG

SEQ ID NO: 125 - protein, 17 aa, Synthetic construct
AINPNDGGTSYNQKFEG

SEQ ID NO: 126 - protein, 17 aa, Synthetic construct
EIYPGTINIYYSERFKG

SEQ ID NO: 127 - protein, 17 aa, Synthetic construct
EIDPSDSYTNYNQKFKG

SEQ ID NO: 128 - protein, 17 aa, Synthetic construct
ANDPKNGGFSYNQRFKG

SEQ ID NO: 129 - protein, 17 aa, Synthetic construct
AINPNNGGTSYNQKFKD

SEQ ID NO: 130 - protein, 17 aa, Synthetic construct
AVNPNTGDISYTQKFKD

SEQ ID NO: 131 - protein, 17 aa, Synthetic construct
AIDPNNGGTNYNQKFKD

SEQ ID NO: 132 - protein, 17 aa, Synthetic construct
AIDPNNDSTNYNQKFKG

SEQ ID NO: 133 - protein, 17 aa, Synthetic construct
GIDPAIGNTVSASKFQD

SEQ ID NO: 134 - protein, 17 aa, Synthetic construct
EIYPGSESPYYSEKFKG

SEQ ID NO: 135 - protein, 17 aa, Synthetic construct
EINPSDGRTNYNEKFKS

SEQ ID NO: 136 - protein, 17 aa, Synthetic construct
AMSPNNGATSYNQKFKG

SEQ ID NO: 137 - protein, 17 aa, Synthetic construct
DIYPGNTFPNYNEKFKG

SEQ ID NO: 138 - protein, 17 aa, Synthetic construct
GIDPAIGNTLYASKFQD

SEQ ID NO: 139 - protein, 17 aa, Synthetic construct
KISNSGGSTNYPDTVKG

SEQ ID NO: 140 - protein, 17 aa, Synthetic construct
AIFPGNGETRDTQKFKG

SEQ ID NO: 141 - protein, 17 aa, Synthetic construct
EINPSDGRTNYNEKFKN

SEQ ID NO: 142 - protein, 17 aa, Synthetic construct
DISNSGGNTYYSDTVKG

SEQ ID NO: 143 - protein, 17 aa, Synthetic construct
DINPDNGAGNYNQKFKG

SEQ ID NO: 144 - protein, 17 aa, Synthetic construct
DINPDNGGTNYNQKFKG

SEQ ID NO: 145 - protein, 17 aa, Synthetic construct
DVNPDNGGTNYNQKFKG

SEQ ID NO: 146 - protein, 17 aa, Synthetic construct
TIVNGGINTYYPDTVKG

SEQ ID NO: 147 - protein, 17 aa, Synthetic construct
TINNGGINTYYPDTVKG

SEQ ID NO: 148 - protein, 17 aa, Synthetic construct
ELSNSGGSFHYPDNVKG

SEQ ID NO: 149 - protein, 17 aa, Synthetic construct
EILPGYISTNYNEKFKD

SEQ ID NO: 150 - protein, 17 aa, Synthetic construct
EILPGSVTTNYNEKFRG

SEQ ID NO: 151 - protein, 17 aa, Synthetic construct
TISDGASYTYYPDNVKG

SEQ ID NO: 152 - protein, 17 aa, Synthetic construct
TITSGDIRTYYPDTVKG

SEQ ID NO: 153 - protein, 17 aa, Synthetic construct
AINPSNGYIGYNQKFKD

SEQ ID NO: 154 - protein, 17 aa, Synthetic construct
DVNPDSNGANYNQKFKG

SEQ ID NO: 155 - protein, 17 aa, Synthetic construct
AINPNNGGTFYNQKFKG

SEQ ID NO: 156 - protein, 17 aa, Synthetic construct
AIDPNNGGTNYNQKFKG

SEQ ID NO: 157 - protein, 16 aa, Synthetic construct
HIWWDDDKYYNPALKS

SEQ ID NO: 158 - protein, 17 aa, Synthetic construct
IINPYNGDTSYNQKFKG

SEQ ID NO: 159 - protein, 17 aa, Synthetic construct
DINPNNGDTTYSQKFMG

SEQ ID NO: 160 - protein, 17 aa, Synthetic construct
AIFPENGDTRYTQKFKG

SEQ ID NO: 161 - protein, 17 aa, Synthetic construct
AIDPKNGATSYNQKFKG

SEQ ID NO: 162 - protein, 10 aa, Synthetic construct
DGGLRGPFDY

SEQ ID NO: 163 - protein, 5 aa, Synthetic construct
GYFDV

SEQ ID NO: 164 - protein, 10 aa, Synthetic construct
DGELRGPFDY

SEQ ID NO: 165 - protein, 5 aa, Synthetic construct
LFFDY

SEQ ID NO: 166 - skipped

SEQ ID NO: 167 - protein, 9 aa, Synthetic construct
FGIYPYFDY

SEQ ID NO: 168 - skipped

SEQ ID NO: 169 - protein, 11 aa, Synthetic construct
YYSGSSYGLDY

SEQ ID NO: 170 - protein, 11 aa, Synthetic construct
YFSGSSYGLDY

SEQ ID NO: 171 - protein, 11 aa, Synthetic construct
EELRRDYGMDY

SEQ ID NO: 172 - protein, 10 aa, Synthetic construct
DSTGLYAMDY

SEQ ID NO: 173 - protein, 8 aa, Synthetic construct
DSFYAMDY

SEQ ID NO: 174 - protein, 9 aa, Synthetic construct
EDYFDRGDC

SEQ ID NO: 175 - protein, 13 aa, Synthetic construct
ELPFTTIIGTKDY

SEQ ID NO: 176 - protein, 6 aa, Synthetic construct
LLFFDY

SEQ ID NO: 177 - protein, 9 aa, Synthetic construct
PLRIYAMDC

SEQ ID NO: 178 - protein, 5 aa, Synthetic construct
LYFDY

SEQ ID NO: 179 - protein, 13 aa, Synthetic construct
RVYYGYDRYFFDY

SEQ ID NO: 180 - protein, 10 aa, Synthetic construct
DGSYDPPFAY

SEQ ID NO: 181 - protein, 14 aa, Synthetic construct
WITTVVATLYYFDY

SEQ ID NO: 182 - protein, 10 aa, Synthetic construct
SGLPYVLMDY

SEQ ID NO: 183 - protein, 10 aa, Synthetic construct
DQVNRGWFTY

SEQ ID NO: 184 - protein, 11 aa, Synthetic construct
DSSNVGNAMDY

SEQ ID NO: 185 - protein, 6 aa, Synthetic construct
GLRYGP

SEQ ID NO: 186 - protein, 11 aa, Synthetic construct
HEDYYYYAMDY

SEQ ID NO: 187 - protein, 11 aa, Synthetic construct
HEDYEWYAMDN

SEQ ID NO: 188 - protein, 11 aa, Synthetic construct
HEDYAWYAMDY

SEQ ID NO: 189 - protein, 10 aa, Synthetic construct
SGPPYVLLDY

SEQ ID NO: 190 - protein, 11 aa, Synthetic construct
DSSNVGNDMDY

SEQ ID NO: 191 - protein, 11 aa, Synthetic construct
HEDYEWYAMDY

SEQ ID NO: 192 - protein, 11 aa, Synthetic construct
HEDYDYYAMDY

SEQ ID NO: 193 - protein, 11 aa, Synthetic construct
SDDYGGYYFDY

SEQ ID NO: 194 - protein, 8 aa, Synthetic construct
GVGLGFPY

SEQ ID NO: 195 - protein, 11 aa, Synthetic construct
SDDFDGYAMDY

SEQ ID NO: 196 - protein, 11 aa, Synthetic construct
PIYYEYDGFAY

SEQ ID NO: 197 - protein, 5 aa, Synthetic construct
GYYDY

SEQ ID NO: 198 - protein, 4 aa, Synthetic construct
GTVY

SEQ ID NO: 199 - protein, 11 aa, Synthetic construct
ADDYDGYYFDY

SEQ ID NO: 200 - protein, 6 aa, Synthetic construct
YYGSNY

SEQ ID NO: 201 - protein, 5 aa, Synthetic construct
VWFAY

SEQ ID NO: 202 - protein, 9 aa, Synthetic construct
GYSGNGFTY

SEQ ID NO: 203 - protein, 5 aa, Synthetic construct
GWGDY

SEQ ID NO: 204 - protein, 12 aa, Synthetic construct
RSPVVVNYAMDY

SEQ ID NO: 205 - protein, 11 aa, Synthetic construct
EGVSSYYAMDY

SEQ ID NO: 206 - protein, 11 aa, Synthetic construct
EGVGSYYAMDY

SEQ ID NO: 207 - protein, 11 aa, Synthetic construct
EGVSSYYGMDS

SEQ ID NO: 208 - protein, 5 aa, Synthetic construct
IWFAY

SEQ ID NO: 209 - protein, 11 aa, Synthetic construct
EGGSSYYAMDY

SEQ ID NO: 210 - protein, 10 aa, Synthetic construct
WVNNDWYFDV

SEQ ID NO: 211 - protein, 10 aa, Synthetic construct
DDGYYDAMDY

SEQ ID NO: 212 - protein, 14 aa, Synthetic construct
SIYYSNYVRYYFDY

SEQ ID NO: 213 - protein, 10 aa, Synthetic construct
WVINDWYFDV

SEQ ID NO: 214 - protein, 10 aa, Synthetic construct
WDSNYWYFDV

SEQ ID NO: 215 - protein, 10 aa, Synthetic construct
WDSDYWYFDV

SEQ ID NO: 216 - protein, 9 aa, Synthetic construct
WSESWYFDV

SEQ ID NO: 217 - protein, 10 aa, Synthetic construct
WDSNDWYFDV

SEQ ID NO: 218 - protein, 10 aa, Synthetic construct
WDSDDWYFDV

SEQ ID NO: 219 - protein, 14 aa, Synthetic construct
ERVHSNTLYYAMDY

SEQ ID NO: 220 - protein, 11 aa, Synthetic construct
SDYSDYWAMDY

SEQ ID NO: 221 - protein, 11 aa, Synthetic construct
NADRAYYTMDY

SEQ ID NO: 222 - protein, 10 aa, Synthetic construct
WDSNSWYFDV

SEQ ID NO: 223 - protein, 14 aa, Synthetic construct
ERVYSNTLYYAMDY

SEQ ID NO: 224 - protein, 10 aa, Synthetic construct
YSWDVYAMDY

SEQ ID NO: 225 - protein, 7 aa, Synthetic construct
FNNYLDY

SEQ ID NO: 226 - protein, 9 aa, Synthetic construct
PDNWDPFVY

SEQ ID NO: 227 - protein, 5 aa, Synthetic construct
LFFDS

SEQ ID NO: 228 - protein, 11 aa, Synthetic construct
HDDFLYYAMDY

SEQ ID NO: 229 - protein, 10 aa, Synthetic construct
HNWDVYAMDY

SEQ ID NO: 230 - protein, 11 aa, Synthetic construct
IVTTYYYFMDY

SEQ ID NO: 231 - protein, 11 aa, Synthetic construct
GSDYSYYAMDY

SEQ ID NO: 232 - protein, 9 aa, Synthetic construct
DELIAPFAY

SEQ ID NO: 233 - protein, 11 aa, Synthetic construct
HDDFHFYAMDY

SEQ ID NO: 234 - protein, 11 aa, Synthetic construct
GEDFDGYAMDY

SEQ ID NO: 235 - protein, 5 aa, Synthetic construct
LFFDN

SEQ ID NO: 236 - protein, 10 aa, Synthetic construct
WDSDSWYFDV

SEQ ID NO: 237 - protein, 7 aa, Synthetic construct
GTYWFTY

SEQ ID NO: 238 - protein, 7 aa, Synthetic construct
FINYVDY

SEQ ID NO: 239 - protein, 11 aa, Synthetic construct
KASQDINKYIA

SEQ ID NO: 240 - protein, 17 aa, Synthetic construct
KSSQSLLNSGNQKNFLA

SEQ ID NO: 241 - protein, 17 aa, Synthetic construct
KSSQSLLNSGNQKNYLA

SEQ ID NO: 242 - protein, 11 aa, Synthetic construct
KASQNVGTAVA

SEQ ID NO: 243 - protein, 11 aa, Synthetic construct
RASQDIGRSLN

SEQ ID NO: 244 - protein, 11 aa, Synthetic construct
KASQSVNKAVV

SEQ ID NO: 245 - protein, 11 aa, Synthetic construct
RASQDIGSSLN

SEQ ID NO: 246 - protein, 17 aa, Synthetic construct
KSSQSLLNSGTQKNYLA

SEQ ID NO: 247 - protein, 11 aa, Synthetic construct
RASQDISNYLN

SEQ ID NO: 248 - protein, 11 aa, Synthetic construct
RASQDIGYSLN

SEQ ID NO: 249 - protein, 11 aa, Synthetic construct
RASENIYSYLA

SEQ ID NO: 250 - protein, 11 aa, Synthetic construct
RTSQDISNFLN

SEQ ID NO: 251 - protein, 10 aa, Synthetic construct
SASSSINYMH

SEQ ID NO: 252 - protein, 11 aa, Synthetic construct
RASQFISNNLH

SEQ ID NO: 253 - protein, 16 aa, Synthetic construct
KSSQSLLDSDGKTYLN

SEQ ID NO: 254 - protein, 11 aa, Synthetic construct
KASQNVGTNVA

SEQ ID NO: 255 - protein, 11 aa, Synthetic construct
KANQDINKYIA

SEQ ID NO: 256 - protein, 10 aa, Synthetic construct
SASSSVSYMH

SEQ ID NO: 257 - protein, 11 aa, Synthetic construct
KASQNVRTAVA

SEQ ID NO: 258 - protein, 11 aa, Synthetic construct
RASENINSYLA

SEQ ID NO: 259 - protein, 12 aa, Synthetic construct
SASSSVSSSYFH

SEQ ID NO: 260 - protein, 12 aa, Synthetic construct
TLSSQHSTYTIE

SEQ ID NO: 261 - protein, 10 aa, Synthetic construct
SANSTVSYIY

SEQ ID NO: 262 - protein, 10 aa, Synthetic construct
SARSSVSYMY

SEQ ID NO: 263 - protein, 10 aa, Synthetic construct
SASSSVNYIY

SEQ ID NO: 264 - protein, 11 aa, Synthetic construct
QTSENIYSYLA

SEQ ID NO: 265 - protein, 10 aa, Synthetic construct
SASSSVSYIY

SEQ ID NO: 266 - protein, 10 aa, Synthetic construct
SASSSVSYMY

SEQ ID NO: 267 - protein, 10 aa, Synthetic construct
RANSSVNYMH

SEQ ID NO: 268 - protein, 11 aa, Synthetic construct
QASQVVSTAVA

SEQ ID NO: 269 - protein, 16 aa, Synthetic construct
RSSQSIVHSNGNTYLE

SEQ ID NO: 270 - protein, 11 aa, Synthetic construct
RASQDIGNNLN

SEQ ID NO: 271 - protein, 11 aa, Synthetic construct
RASQSISSWLA

SEQ ID NO: 272 - protein, 15 aa, Synthetic construct
RASESVDNYGISFMH

SEQ ID NO: 273 - protein, 11 aa, Synthetic construct
RASENIYSHLV

SEQ ID NO: 274 - protein, 17 aa, Synthetic construct
KSSQSLLNSFNQKNFLA

SEQ ID NO: 275 - protein, 11 aa, Synthetic construct
RASENIYSYLV

SEQ ID NO: 276 - protein, 17 aa, Synthetic construct
KSSQSLLSSNNQKSFLA

SEQ ID NO: 277 - protein, 17 aa, Synthetic construct
KSSQSLLNSNNQKNFLA

SEQ ID NO: 278 - protein, 17 aa, Synthetic construct
KSSQSLLNGFNQKNFLA

SEQ ID NO: 279 - protein, 17 aa, Synthetic construct
KSSQSLLSSGNQKNFLA

SEQ ID NO: 280 - protein, 17 aa, Synthetic construct
KSSQSLLNSGIQKNFLA

SEQ ID NO: 281 - protein, 17 aa, Synthetic construct
KSSQSLLSNNNQKNFLA

SEQ ID NO: 282 - protein, 11 aa, Synthetic construct
KASQNVRSAVD

SEQ ID NO: 283 - protein, 11 aa, Synthetic construct
KASQDVRSAVA

SEQ ID NO: 284 - protein, 11 aa, Synthetic construct
KASQNVRTAVD

SEQ ID NO: 285 - protein, 11 aa, Synthetic construct
KASQNVRAAVD

SEQ ID NO: 286 - protein, 11 aa, Synthetic construct
KASQNVRIAVD

SEQ ID NO: 287 - protein, 11 aa, Synthetic construct
KASEDIYNRLA

SEQ ID NO: 288 - protein, 15 aa, Synthetic construct
RASKSVSISGYSYMH

SEQ ID NO: 289 - protein, 11 aa, Synthetic construct
KASQNVRSAVA

SEQ ID NO: 290 - protein, 11 aa, Synthetic construct
RASQSVSSNLA

SEQ ID NO: 291 - protein, 17 aa, Synthetic construct
KSSQSLLNSFNQRNFLA

SEQ ID NO: 292 - protein, 11 aa, Synthetic construct
KASQIVRTAVD

SEQ ID NO: 293 - protein, 17 aa, Synthetic construct
KSSQSLLNGFNQRNFLA

SEQ ID NO: 294 - protein, 11 aa, Synthetic construct
KASEDIYARLA

SEQ ID NO: 295 - protein, 17 aa, Synthetic construct
RSSQSLLNSGNQKNYLA

SEQ ID NO: 296 - protein, 10 aa, Synthetic construct
SASSSVSYMF

SEQ ID NO: 297 - protein, 11 aa, Synthetic construct
RASENVNSYLA

SEQ ID NO: 298 - protein, 10 aa, Synthetic construct
SASSSVTYMH

SEQ ID NO: 299 - protein, 17 aa, Synthetic construct
KSSQSLLNIFNQKNFLA

SEQ ID NO: 300 - protein, 11 aa, Synthetic construct
KASQDVGTAVA

SEQ ID NO: 301 - protein, 10 aa, Synthetic construct
SASSSVSYLY

SEQ ID NO: 302 - protein, 10 aa, Synthetic construct
SASSSVNYMF

SEQ ID NO: 303 - protein, 11 aa, Synthetic construct
KASENVRAAVD

SEQ ID NO: 304 - protein, 16 aa, Synthetic construct
RSSQSIEHSNGNTYLQ

SEQ ID NO: 305 - protein, 10 aa, Synthetic construct
SASSSVNNIH

SEQ ID NO: 306 - protein, 11 aa, Synthetic construct
KASQNIRSAVA

SEQ ID NO: 307 - protein, 7 aa, Synthetic construct
YTSTLHP

SEQ ID NO: 308 - protein, 7 aa, Synthetic construct
YTSTLQP

SEQ ID NO: 309 - protein, 7 aa, Synthetic construct
GTSTRES

SEQ ID NO: 310 - protein, 7 aa, Synthetic construct
YTSTLQA

SEQ ID NO: 311 - protein, 7 aa, Synthetic construct
GASTRES

SEQ ID NO: 312 - protein, 7 aa, Synthetic construct
SASNRYT

SEQ ID NO: 313 - protein, 7 aa, Synthetic construct
ATSRLDS

SEQ ID NO: 314 - protein, 7 aa, Synthetic construct
YASNRYT

SEQ ID NO: 315 - protein, 7 aa, Synthetic construct
YTSRLHS

SEQ ID NO: 316 - protein, 7 aa, Synthetic construct
NAKTLAE

SEQ ID NO: 317 - protein, 7 aa, Synthetic construct
FTSTLQS

SEQ ID NO: 318 - protein, 7 aa, Synthetic construct
DTSKLAS

SEQ ID NO: 319 - protein, 7 aa, Synthetic construct
YASQSIS

SEQ ID NO: 320 - protein, 7 aa, Synthetic construct
LVSKLDS

SEQ ID NO: 321 - protein, 7 aa, Synthetic construct
SASYRYS

SEQ ID NO: 322 - protein, 7 aa, Synthetic construct
YTSTLQS

SEQ ID NO: 323 - protein, 7 aa, Synthetic construct
STSNLAS

SEQ ID NO: 324 - protein, 7 aa, Synthetic construct
LASNRHT

SEQ ID NO: 325 - protein, 7 aa, Synthetic construct
NTKTLTE

SEQ ID NO: 326 - protein, 7 aa, Synthetic construct
YSSRLHS

SEQ ID NO: 327 - protein, 7 aa, Synthetic construct
GSHRTGD

SEQ ID NO: 328 - protein, 7 aa, Synthetic construct
DTSNLAS

SEQ ID NO: 329 - protein, 7 aa, Synthetic construct
NTQTLAE

SEQ ID NO: 330 - protein, 7 aa, Synthetic construct
ATSNLAS

SEQ ID NO: 331 - protein, 7 aa, Synthetic construct
SASFRYS

SEQ ID NO: 332 - protein, 7 aa, Synthetic construct
KVSNRFS

SEQ ID NO: 333 - protein, 7 aa, Synthetic construct
ATSSLDS

SEQ ID NO: 334 - protein, 7 aa, Synthetic construct
TVSNRLS

SEQ ID NO: 335 - protein, 7 aa, Synthetic construct
KASTLES

SEQ ID NO: 336 - protein, 7 aa, Synthetic construct
RASNLES

SEQ ID NO: 337 - protein, 7 aa, Synthetic construct
NGKILAE

SEQ ID NO: 338 - protein, 7 aa, Synthetic construct
FASTRES

SEQ ID NO: 339 - protein, 7 aa, Synthetic construct
SASNRHT

SEQ ID NO: 340 - protein, 7 aa, Synthetic construct
LASSRHT

SEQ ID NO: 341 - protein, 7 aa, Synthetic construct
SSSNRHT

SEQ ID NO: 342 - protein, 7 aa, Synthetic construct
GTTSLEI

SEQ ID NO: 343 - protein, 7 aa, Synthetic construct
LASNLES

SEQ ID NO: 344 - protein, 7 aa, Synthetic construct
GASTRAT

SEQ ID NO: 345 - protein, 7 aa, Synthetic construct
FASTRDS

SEQ ID NO: 346 - protein, 7 aa, Synthetic construct
SASTRHT

SEQ ID NO: 347 - protein, 7 aa, Synthetic construct
GASTRQS

SEQ ID NO: 348 - protein, 7 aa, Synthetic construct
NAETLVE

SEQ ID NO: 349 - protein, 7 aa, Synthetic construct
DTSKLTS

SEQ ID NO: 350 - protein, 7 aa, Synthetic construct
GATSLET

SEQ ID NO: 351 - protein, 7 aa, Synthetic construct
NAQTLAE

SEQ ID NO: 352 - protein, 7 aa, Synthetic construct
WASTRHT

SEQ ID NO: 353 - protein, 7 aa, Synthetic construct
SASSRHT

SEQ ID NO: 354 - protein, 7 aa, Synthetic construct
NAKTLTE

SEQ ID NO: 355 - protein, 7 aa, Synthetic construct
DTTKLTS

SEQ ID NO: 356 - protein, 7 aa, Synthetic construct
LTSNRHT

SEQ ID NO: 357 - protein, 8 aa, Synthetic construct
LQYDYLLT

SEQ ID NO: 358 - protein, 8 aa, Synthetic construct
LQYDNLHT

SEQ ID NO: 359 - protein, 9 aa, Synthetic construct
QNDHSYPWT

SEQ ID NO: 360 - protein, 8 aa, Synthetic construct
LQYDNLLT

SEQ ID NO: 361 - protein, 9 aa, Synthetic construct
QNAHSSPYT

SEQ ID NO: 362 - protein, 8 aa, Synthetic construct
QQYSSYRT

SEQ ID NO: 363 - protein, 9 aa, Synthetic construct
LQYASFPYT

SEQ ID NO: 364 - protein, 9 aa, Synthetic construct
QQDYSSPWT

SEQ ID NO: 365 - protein, 9 aa, Synthetic construct
LQYASSPYT

SEQ ID NO: 366 - protein, 9 aa, Synthetic construct
QQGNMFPPT

SEQ ID NO: 367 - protein, 9 aa, Synthetic construct
QHHYGTPYT

SEQ ID NO: 368 - protein, 8 aa, Synthetic construct
LQYDYLYT

SEQ ID NO: 369 - protein, 9 aa, Synthetic construct
QQGYMLPPT

SEQ ID NO: 370 - protein, 9 aa, Synthetic construct
QQGNTLPPT

SEQ ID NO: 371 - protein, 8 aa, Synthetic construct
LQYSSYLT

SEQ ID NO: 372 - protein, 8 aa, Synthetic construct
QQYSTYLT

SEQ ID NO: 373 - protein, 9 aa, Synthetic construct
HQRSSYPFT

SEQ ID NO: 374 - protein, 9 aa, Synthetic construct
QQSNSWPHT

SEQ ID NO: 375 - protein, 9 aa, Synthetic construct
WQGTHFPLT

SEQ ID NO: 376 - protein, 9 aa, Synthetic construct
QQYNSRPLT

SEQ ID NO: 377 - protein, 9 aa, Synthetic construct
QNDHSYPFT

SEQ ID NO: 378 - protein, 8 aa, Synthetic construct
LQYDHLLT

SEQ ID NO: 379 - protein, 9 aa, Synthetic construct
QQRSSYPPT

SEQ ID NO: 380 - protein, 8 aa, Synthetic construct
LQYDNLYT

SEQ ID NO: 381 - protein, 9 aa, Synthetic construct
LQHWNYPLT

SEQ ID NO: 382 - protein, 9 aa, Synthetic construct
QHHYGTPLT

SEQ ID NO: 383 - protein, 9 aa, Synthetic construct
HQHHRSPLT

SEQ ID NO: 384 - protein, 9 aa, Synthetic construct
QQGYTLPRT

SEQ ID NO: 385 - protein, 9 aa, Synthetic construct
QQGNTLPRT

SEQ ID NO: 386 - protein, 9 aa, Synthetic construct
GVGDINNYI

SEQ ID NO: 387 - protein, 9 aa, Synthetic construct
QQWSSFPFT

SEQ ID NO: 388 - protein, 9 aa, Synthetic construct
QQWSSYPLT

SEQ ID NO: 389 - protein, 8 aa, Synthetic construct
LQYDNLYM

SEQ ID NO: 390 - protein, 9 aa, Synthetic construct
QHHYGVPLT

SEQ ID NO: 391 - protein, 9 aa, Synthetic construct
QQWTSYPLT

SEQ ID NO: 392 - protein, 9 aa, Synthetic construct
QQWTSNPLT

SEQ ID NO: 393 - protein, 9 aa, Synthetic construct
QQHYRTPYT

SEQ ID NO: 394 - protein, 9 aa, Synthetic construct
FQGSHVPRT

SEQ ID NO: 395 - protein, 9 aa, Synthetic construct
QNDHSYPHT

SEQ ID NO: 396 - protein, 9 aa, Synthetic construct
LQYASSPVT

SEQ ID NO: 397 - protein, 9 aa, Synthetic construct
QQWTNNPLT

SEQ ID NO: 398 - protein, 9 aa, Synthetic construct
FQTSHVPPT

SEQ ID NO: 399 - protein, 8 aa, Synthetic construct
QQYNTYWT

SEQ ID NO: 400 - protein, 9 aa, Synthetic construct
QNAHSYPFT

SEQ ID NO: 401 - protein, 10 aa, Synthetic construct
QQSNKDPMYT

SEQ ID NO: 402 - protein, 9 aa, Synthetic construct
QNDHGYPLT

SEQ ID NO: 403 - protein, 9 aa, Synthetic construct
QHHYGSPIT

SEQ ID NO: 404 - protein, 9 aa, Synthetic construct
QQHHSTPPT

SEQ ID NO: 405 - protein, 9 aa, Synthetic construct
LQYASSPPT

SEQ ID NO: 406 - protein, 9 aa, Synthetic construct
QHHYGSPLT

SEQ ID NO: 407 - protein, 9 aa, Synthetic construct
QQHYSTPPT

SEQ ID NO: 408 - protein, 9 aa, Synthetic construct
QNAHSSPFT

SEQ ID NO: 409 - protein, 9 aa, Synthetic construct
LQHWNYPYT

SEQ ID NO: 410 - protein, 9 aa, Synthetic construct
LQYTSSPWT

SEQ ID NO: 411 - protein, 8 aa, Synthetic construct
LQYDNLWT

SEQ ID NO: 412 - protein, 9 aa, Synthetic construct
LQHWTYPYT

SEQ ID NO: 413 - protein, 9 aa, Synthetic construct
LQHWISPYT

SEQ ID NO: 414 - protein, 9 aa, Synthetic construct
LQHWNSPYT

SEQ ID NO: 415 - protein, 9 aa, Synthetic construct
QQYWSTPWT

SEQ ID NO: 416 - protein, 9 aa, Synthetic construct
QHSRELPPT

SEQ ID NO: 417 - protein, 9 aa, Synthetic construct
LQHWNYPWT

SEQ ID NO: 418 - protein, 9 aa, Synthetic construct
HQYNNWPRT

SEQ ID NO: 419 - protein, 9 aa, Synthetic construct
QNDHSYPYT

SEQ ID NO: 420 - protein, 8 aa, Synthetic construct
QHHYGDPT

SEQ ID NO: 421 - protein, 9 aa, Synthetic construct
QQWISNPYT

SEQ ID NO: 422 - protein, 9 aa, Synthetic construct
QQHWSTPWT

SEQ ID NO: 423 - protein, 9 aa, Synthetic construct
QNAHTSPYT

SEQ ID NO: 424 - protein, 8 aa, Synthetic construct
QHHYGNPT

SEQ ID NO: 425 - protein, 9 aa, Synthetic construct
QQRSSYPWT

SEQ ID NO: 426 - protein, 9 aa, Synthetic construct
QQYNSYPWT

SEQ ID NO: 427 - protein, 9 aa, Synthetic construct
FQGSHVPWT

SEQ ID NO: 428 - protein, 9 aa, Synthetic construct
QQGHALPRT

SEQ ID NO: 429 - protein, 9 aa, Synthetic construct
QQWNINPYT

SEQ ID NO: 430 - protein, 119 aa, Synthetic construct
QVHLQQSGPELVKPGASLKISCKASGYTFTDYYLNWVKQRPGQGLEWIGWIFPGSESTFYNEKFKGKATLTVDKSSSTAYMLLNSLTSEDSAVYFCTRDGGLRGPFDYWGQGTTLTVSS

SEQ ID NO: 431 - protein, 106 aa, Synthetic construct
DIQLTQSPSSLSASLGGKVTITCKASQDINKYIAWCQHKPGKGPRLLIHYTSTLHPGIPSRFSGSGSGRDYSFSISNLEPEDSATYYCLQYDYLLTFGGGTKLEIK

SEQ ID NO: 432 - protein, 119 aa, Synthetic construct
QVQLQQSGPELVKPGASVKISCRSSGYTFTDYYINWVKQRPGQGLEWIGWIFPGSDYTYYNEKFKGKATLTVDKSSTTAYMLLSSLTSEDSAVYFCARDGGLRGPFDYWGQGTTLTVSS

SEQ ID NO: 433 - protein, 106 aa, Synthetic construct
DIQMTQSPSSLSASLGGKVTITCKASQDINKYIAWYQQKPGKGPRLLIHYTSTLQPGIPSRFSGSGSGRNSSFSISNLEPEDIATYYCLQYDNLHTFGGGTKLEIK

SEQ ID NO: 434 - protein, 114 aa, Synthetic construct
EVQLQQSGPELVKPGASVKISCKASGYTFTDYFMNWVKQSHGKSLEWIGDIIPNNDSPNYNQKFKGKATLTVDKSSNTAYMELRSLTSEDSAVYYCASGYFDVWGTGTTVTVSS

SEQ ID NO: 435 - protein, 113 aa, Synthetic construct
DIVMTQSPSSLSVSAGEKVTMSCKSSQSLLNSGNQKNFLAWYQQKPGQPPKLLIYGTSTRESGVPDRFTGSGSGTDFTLTISSVQAEDLAVYYCQNDHSYPWTFGGGTKLEIK

SEQ ID NO: 436 - protein, 119 aa, Synthetic construct
QVHLHQSGPELVKPGASVKISCKASGYTFTAYYINWVKQRPGQGLEWIGWIFPGSDSTYYNEKFKGRATLTVDKSSSTAYMLLNSLTSEDSAVYFCARDGELRGPFDYWGQGTTLTVSS

SEQ ID NO: 437 - protein, 106 aa, Synthetic construct
DIQMTQSPSSLSASLGGKVTITCKASQDINKYIAWYQHKPGKSPRLLIHYTSTLQAGIPSRFSGSGSGRDYSFSISNLEPEDSATYYCLQYDNLLTFGGGTKLEIK

SEQ ID NO: 438 - protein, 114 aa, Synthetic construct
EVQLQQSGPELVKPGASVKISCKASGYTLTDYYMNWVKQSHGKSLEWIGDINPNNGGTNFNQKFKGKATLTIDKSSSTTYMELRSLTSEDSAVYYCASLFFDYWGQGTTLTVSS

SEQ ID NO: 439 - protein, 113 aa, Synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLALYYCQNAHSSPYTFGGGTKLEIK

SEQ ID NO: 440 - protein, 106 aa, Synthetic construct
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYRTFGGGTKLEIK

SEQ ID NO: 441 - protein, 112 aa, Synthetic construct
EVQLVASGGGLVKPGGSLKLSCAASGFTFSDYYMFWVRQTPEKRLEWVATISDGGGYIYYPDSVKGRFTISRDNDRNSLYLQMSSLKSEDTAIYYCGREDFWGQGTSVTVSS

SEQ ID NO: 442 - protein, 107 aa, Synthetic construct
DIQMTQSPSSLSASLGERVSLTCRASQDIGRSLNWLQQEPDGSVRRLIYATSRLDSGVPKRFSGSRSGSDYSLTISSLESEDFVDYYCLQYASFPYTFGGGTKLEIK

SEQ ID NO: 443 - protein, 118 aa, Synthetic construct
EVQLQQSGAELVKPGASVKLSCTASGFNIKDIYMHWLKQRPEQGLEWIGRIDPTNGNSEYDPRFRGKATLTADTSSNIAYLQLSSLTSEDTAVYYCARFGIYPYFDYWGQGTTLTVSS

SEQ ID NO: 444 - protein, 107 aa, Synthetic construct
GIVMTQTPKFLLVSAGDRVTITCKASQSVNKAVVWYQQKPGQSPKLLISYASNRYTGVPDRFTGSGYGTDFTFSISTVQAEDLAVYFCQQDYSSPWTFGGGTKLEIK

SEQ ID NO: 445 - protein, 112 aa, Synthetic construct
EVELVESGGGLVKPGGSLKLSCAASGFTFSDSYMCWVRQTPEKRLEWVATISDGGGLTYYPDSVKGRFTISRDNDRNNLYLQMDSLKSEDTAVYYCGREDLWGQGTSVIVSS

SEQ ID NO: 446 - protein, 107 aa, Synthetic construct
DIQMTQSPSSLSASLGERVSLTCRASQDIGSSLNWLQQEPDGTIKRLIYATSRLDSGVPKRFSGSRSGSDYSLTISSLESEDFVDYYCLQYASSPYTFGGGTKLEIK

SEQ ID NO: 447 - protein, 114 aa, Synthetic construct
EVQLQQSGPELVKPGASVKISCKASGYTFTDYFMNWVRQSHGKSLEWIGDIHPNNGSPNYNQKFKGKATLTVGKSSSTAYMELRSLTSEDSAVYYCATGYFDVWGTGTTVTVSS

SEQ ID NO: 448 - protein, 113 aa, Synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPWTFGGGTKLEIK

SEQ ID NO: 449 - protein, 112 aa, Synthetic construct
EVQLVASGGGLVKPGGSLKLSCAASGFTFSDYYMYWVRQTPEKRLEWVATISDGGGYTYYPDSVKGRFTISRDNDRNSLYLQMSSLKSEDTAMYYCGREDLWGQGTSVTVSS

SEQ ID NO: 450 - protein, 107 aa, Synthetic construct
DIQMTQSPSSLSASLGERVSLTCRASQDIGSSLNWLQQEPDGTVKRLIYATSRLDSGVPKRFSGSRSGSDYSLTISSLESEDFVDYYCLQYASFPYTFGGGTKLEIK

SEQ ID NO: 451 - protein, 114 aa, Synthetic construct
EVQLQQSGPELVKPGASVKISCKASGYTFTDYFMNWVKQSHGKSLEWIGDINPNNGSPNYNQKFKGRATLTVDKSSSTAYMELRSLTSEDSAVYYCASGYFDVWGTGTTVTVSS

SEQ ID NO: 452 - protein, 113 aa, Synthetic construct
DIVMTQSPSSLSVSAGEKVTMSCKSSQSLLNSGTQKNYLAWYQQKPGQPPKLLIYGASTRESGVPDRFTGSGSGTDFTLTISSVQAEDLAVYYCQNDHSYPWTFGGGTKLEIK

SEQ ID NO: 453 - protein, 120 aa, Synthetic construct
QVQLQQPGAELVRPGTSVKLSCKASGNTFTSYWMHWVKQRPGQGHEWIGVIDASDSNTKYNQKFKGKATLTVDTSSSTAYMQLSSLTSEDSAVYYCARYYSGSSYGLDYWGQGTTLTVSS

SEQ ID NO: 454 - protein, 107 aa, Synthetic construct
DIQMTQTASSLSASLGDRVTISCRASQDISNYLNWYQQKPDGTVKLLIYYTSRLHSGVPSRFSGSGSGTDFSLTITHLEQEDIASYFCQQGNMFPPTFGGGTKLEIK

SEQ ID NO: 455 - protein, 120 aa, Synthetic construct
QVQLQQPGAELVRPGTSVKLSCKASGNTFTSYWMHWVKQRPGQGHEWIGVIDASDSNTKYNQKFKGKATLTVDTSSSTAYMQLSSLTSEDSAVYYCARYFSGSSYGLDYWGQGTTLTVSS

SEQ ID NO: 456 - protein, 107 aa, Synthetic construct
DIQMTQTASSLSASLGDRVTISCRASQDISNYLNWYQQKPDGTVKLLIYYTSRLHSGVPSRFSGSGSGTDYSLTITNLEQEDIASYFCQQGNMFPPTFGGGTKLEIK

SEQ ID NO: 457 - protein, 112 aa, Synthetic construct
EVQLVESGGGLVKPGGSLKLSCAASGFTFSDYYMYWVRQTPEKRLEWVATISDGGGFIYYPDSVKGRFTISRDNDRNSLYLLMSSLKSEDTAMYFCGREDLWGQGTSVTVSS

SEQ ID NO: 458 - protein, 107 aa, Synthetic construct
DIQMTQSPSSLSASLGERVSLTCRASQDIGRSLNWLQQGPDGTVKRLIYATSRLDSGVPTRFSGSRSGSDYSLTISSLESEDFVDYYCLQYASFPYTFGGGTKLEIK

SEQ ID NO: 459 - protein, 112 aa, Synthetic construct
EVQLVESGGGLVKPGGSLKLSCAASGFTFSDYYMYWVRQTPEKRLEWVATISDAGGYTYYPDSVKGRFTISRDNARNSLYLQMSSLKSEDTAMYFCGREDLWGQGTSVTVSS

SEQ ID NO: 460 - protein, 107 aa, Synthetic construct
DIQMTQSPSSLSASLGERVSLTCRASQDIGYSLNWLQQEPDGSVRRLIYATSRLDSGVPKRFSGSRSGSDYSLTISSLESEDFVDYYCLQYASFPYTFGGGTKLEIK

SEQ ID NO: 461 - protein, 112 aa, Synthetic construct
EVQLVESGGGLVKPGGSLKLSCAASGFTFSDSYMCWVRQTPEKRLEWVATISDAGGYTYYPDSVKGRFTISRDNDRNNLYLQMSGLKSEDTAVYYCGREDLWGQGTSVTVSS

SEQ ID NO: 462 - protein, 107 aa, Synthetic construct
DIQMTQSPSSLSASLGERVSLTCRASQDIGRSLNWLQQEPDGTIKRLIYATSRLDSGVPKRFSGSRSGSDYSLTISSLESEDFVDYYCLQYASSPYTFGGGTKLEIK

SEQ ID NO: 463 - protein, 118 aa, Synthetic construct
EVQLQQSGAELVKPGASVKLSCTASGFNIKDIYMHWLKQRPEQGLEWIGRVDPTNGNSEYDPRFQGKATLTADTSSNIAYLQLSSLTSEDTAVYYCARFGIYPYFDYWGQGTTLTVSS

SEQ ID NO: 464 - protein, 107 aa, Synthetic construct
SIVMTQTPKFLLVSAGDRVTITCKASQSVNKAVVWYQQKPGQSPKLLINYASNRYTGVPDRFTGSGYGTDFTFSISTVQAEDLAVYFCQQDYSSPWTFGGGTKLEIK

SEQ ID NO: 465 - protein, 120 aa, Synthetic construct
EVQLQQSGPEPVKPGASVKISCKASGYTFTDYNIHWVRQSHGKSLEWIGYIYPYNGGAGYNQKFKSKATLTVDSSSSTAYMELRSLTSEDSAVYYCAREELRRDYGMDYWGQGTSVTVSS

SEQ ID NO: 466 - protein, 107 aa, Synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLETK

SEQ ID NO: 467 - protein, 119 aa, Synthetic construct
QVQLQQSGPELVKPGTSVKISCKASGYTFTDYYINWVKQRPGQGLEWIGWIFPGSGNTYYNEKFKGRATLTVDKSSSTAYMLLSSLTSEDSAVYFCARDGGLRGPFDYWGQGTTLTVSS

SEQ ID NO: 468 - protein, 106 aa, Synthetic construct
DIQMTQSPSSLSASLGGKVTITCKASQDINKYIAWFQHKPGKGPRLLIHFTSTLQSGIPSRFSGSGSGGDYSFSISNLEPEDIATYYCLQYDYLYTFGGGTTLEIK

SEQ ID NO: 469 - protein, 118 aa, Synthetic construct
QVQLKQSGPGLVQPSQNLSITCTVSGFSLTGYGINWVRQSPGKGLEWLGVIWSGGNTEYNAAFRSRLSISKDTSKSQVFLKMNSLQADDTAIYSCARDSTGLYAMDYWGPGTSVTVSS

SEQ ID NO: 470 - protein, 107 aa, Synthetic construct
DLQMTQTTSSLSASLGDRVTISCRTSQDISNFLNWYQQKPDGPLKLLIYYTSRLHSGVPSRFSGSGSGTDYSLTISNLEQEDIATYFCQQGYMLPPTFGGGTKLEIK

SEQ ID NO: 471 - protein, 120 aa, Synthetic construct
QVQLQQPGAELVRPGTSVKLSCKASGNTFTSYWMHWVKQRPGQGHEWIGVIDASDSKTKYNQKFKGKATLTVDTSSSTAYMQLSSLTSEDSAVYYCARYYSGSSYGLDYWGQGTTLTVSS

SEQ ID NO: 472 - protein, 107 aa, Synthetic construct
DIQMTQTASSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEIK

SEQ ID NO: 473 - protein, 117 aa, Synthetic construct
QIQLQQSGPELVKPGPSVKISCKASGYTFTDYYINWVKQRPGQGLEWIGWIYPGSGYTKYNEKLKGRATLTVDTSSSTAYMQLSSLTSEDSAVYFCARDSFYAMDYWGQGTSVTVSS

SEQ ID NO: 474 - protein, 106 aa, Synthetic construct
DIVMTQSQKFMSTTVGDRVSITCKASQNVGTAVAWYQQKPGQSPKLLIYSASNRYTGVPDRFTGSGSGTDFTLTITNMQSEDLADYFCLQYSSYLTFGAGTKLELK

SEQ ID NO: 475 - protein, 117 aa, Synthetic construct
QIQLQQSGPELVKPGASVKISCKASGYTFTDYYIAWVKQRPGQGLEWIGWIYLGSGHTMYNENFKGKATLTVDTSSSTAYMQLSSLTSEDSAVYFCARDSFYAMDYWGQGTSVTVSS

SEQ ID NO: 476 - protein, 106 aa, Synthetic construct
DIVMTQSQKFMSTTVGDRVSITCKASQNVGTAVAWYQQKPGQSPKLLIYSASNRYTGVPDRFTGSGSGTDFTLTISNMQSEDLADYFCQQYSTYLTFGAGTKLELK

SEQ ID NO: 477 - protein, 118 aa, Synthetic construct
QVQLQQSGPELVKPGASVKISCKASGYTFTDYYINWVKQRPGQGLEWIGWIFPGSGSTYYNEKFKGKATPTVDKYSSTAYMLLSSLTSEDSAVYFCAREDYFDRGDCWGQGTSVTVSS

SEQ ID NO: 478 - protein, 106 aa, Synthetic construct
QIVLTQSPAIMSASPGEKVTMTCSASSSINYMHWYQQKPGTSPKRWIYDTSKLASGVPARFSGSGSGTSYSLTISSMEAEDAATYYCHQRSSYPFTFGSGTKLEIK

SEQ ID NO: 479 - protein, 122 aa, Synthetic construct
QIQLVQSGPELKKPGETVKISCKASGYTFTTYGMSWVKQAPGKGLKWMGWINTYSGVPTYADDFKGRFAFSLETSASNAYLQINNLKNEDTATYFCARELPFTTIIGTKDYWGQGTTLTVSS

SEQ ID NO: 480 - protein, 107 aa, Synthetic construct
DIVLIQSPATLSVTPGDSVSLSCRASQFISNNLHWYQQKSHESPRLLIKYASQSISGIPSRFSGGGSGTDFTLTVNSVETEDFGMYFCQQSNSWPHTFGGGTKLEIK

SEQ ID NO: 481 - protein, 115 aa, Synthetic construct
QVQLQQSGAELMKPGASVKLSCKATGYTFTGYWIEWVKQRPGHGLEWIGEILPESGRTNYNEKFKGKATFTADTSSNTAYMQLSSLTTEDSAIYYCASLLFFDYWGQGTTLTVSS

SEQ ID NO: 482 - protein, 112 aa, Synthetic construct
DVVLTQTPLTLTVTIGQPASISCKSSQSLLDSDGKTYLNWLLQRPGQSPKRLIYLVSKLDSGVPDRFTGSGSGTDFTLKISRVEAEDLGVYYCWQGTHFPLTFGAGTKLELK

SEQ ID NO: 483 - protein, 118 aa, Synthetic construct
QAYLQQSGAELVRPGASVKMSCKASGYTFTSYNIHWVKQTPRQGLEWIGAIYPGNADISYNQKFRDKATLTVDKSSSTAYMQLSSLTSEDSAVYFCAKPLRIYAMDCWGQGTSVTVSS

SEQ ID NO: 484 - protein, 107 aa, Synthetic construct
DIVMTQSQKFMSTSVGDRVSVTCKASQNVGTNVAWYQQKPGQSPKALIYSASYRYSGVPDRFTGSGSGTDFTLTISNVQSEDLADYFCQQYNSRPLTFGGGTKLEIK

SEQ ID NO: 485 - protein, 114 aa, Synthetic construct
EVQLQQSGPELVKPGASVKISCKASGNTFTDYYINWVKQSHGKSLEWIGDIEPNNGGTNYNQKFKGKATLTVDKSSSTAYMELRSLTSEDSAIYYCATLYFDYWGQGTTLTVSS

SEQ ID NO: 486 - protein, 113 aa, Synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPFTFGSGTKLEIK

SEQ ID NO: 487 - protein, 120 aa, Synthetic construct
QVQLQQPGAELVRPGTSVKLSCKASGNTFTTYWMHWVKQRPGQGHEWIGVIDASDSNTKYNQKFKGKATLTVDTSSSTAYMQLSSLTSEDSAVYYCARYYSGSSYGLDYWGQGTTLTVSS

SEQ ID NO: 488 - protein, 119 aa, Synthetic construct
QVQLQQSGPELMKPGASLKISCKASGYTFTDSYINWVKQRPGQGLEWIGWIFPGSGSTYYNEKFKGTATLTVDKSSSTAYMLLSSLTSEDSAAYFCARDGGLRGPFDYWGQGTTLTVSS

SEQ ID NO: 489 - protein, 106 aa, Synthetic construct
DIQMTQSPSSLSASLGGKVTITCKASQDINKYIAWYQHKPGKGPRLLIHYTSTLQSGIPSRFSGSGSGRDYFFSISNLESEDIATYYCLQYDHLLTFGGGTKLEIK

SEQ ID NO: 490 - protein, 119 aa, Synthetic construct
QVHLQQSGPDLVKPGVSVKISCKASGYTFTDYYINWVKQRPGQGLEWIGWIFPGSGTTYYNEKFKGKATLTVDKSSSTAYMLLSSLTSEDSAVYFCARDGGLRGPFDYWGQGTTLTVSS

SEQ ID NO: 491 - protein, 106 aa, Synthetic construct
DIQMTQSPSSLSASLGGKVTITCKANQDINKYIAWYQYKPGKGPRLLIHYTSTLQPDIPSRFSGSGSGRDFSFSISNLEPEDIATYYCLQYDYLYTFGGGTKLEIK

SEQ ID NO: 492 - protein, 122 aa, Synthetic construct
QVQLQQPGAELVKPGASVKLSCKASGYSFTSYWMQWIKQRPGQGLEWIGEIDPFDSYTNYNQKFKGKATLTVDTSSSTAYMQLSSLTSEDSAVYYCASRVYYGYDRYFFDYWGQGTTLTVSS

SEQ ID NO: 493 - protein, 119 aa, Synthetic construct
QVQLQQSGPELVKPGASVKISCKASGYTFTDYYINWVKQRPGQGLEWIGWIFPGSGSTYYNEKFKGKATLTVDKSSSTAYMLLSSLTSEDSAVYFCAPDGSYDPPFAYWGLGTLVTVSA

SEQ ID NO: 494 - protein, 106 aa, Synthetic construct
QIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGAGTKLELK

SEQ ID NO: 495 - protein, 106 aa, Synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLYTFGGGTKLEIK

SEQ ID NO: 496 - protein, 123 aa, Synthetic construct
QVQLQQSGAELVRPGASVTLSCKASGYTFTDYEMHWVKQTPVHGLEWIGAIDPETGGTAYNQKFKGKAILTADKSSSTAYMELRSLTSEDSAVYYCTRWITTVVATLYYFDYWGQGTTLTVSS

SEQ ID NO: 497 - protein, 107 aa, Synthetic construct
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGAGTKLELK

SEQ ID NO: 498 - protein, 123 aa, Synthetic construct
RVQLQQSGAELVRPGASVTLSCKASGYTFTDYEMHWVKQTPVHGLEWIGAIDPETGGTAYNQKFKGKAILTADKSSSTAYMELRSLTSEDSAVYYCTRWITTVVATLYYFDYWGQGTTLTVSS

SEQ ID NO: 499 - protein, 119 aa, Synthetic construct
EVQLQQSGPELVKPGASVKISCKASGYTFTDYYMSWVKQSHGKSLEWIGDVNPNNGDTSYNQKFKGKATLTVDKSSSTAYMELRSLTSEDSAVYYCAASGLPYVLMDYWGQGTSVTVSS

SEQ ID NO: 500 - protein, 107 aa, Synthetic construct
DIQMTQSPASLSASVGETVTITCRASENINSYLAWYQQKQGKSPQLLVYNTKTLTEDVPSRFSGSGSGTQFSLKINSLQPEDFGSYYCQHHYGTPLTFGGGTKLELK

SEQ ID NO: 501 - protein, 119 aa, Synthetic construct
EVQLVESGGGLVKPGGSLKLSCAASGFTFSGYALSWVRQTPEKRLEWVATIRDGGSYTYYSDNEKGRFTISRDNAKNNLYLQMSHLKSEDTAIYYCTRDQVNRGWFTYWGQGTLVTVSA

SEQ ID NO: 502 - protein, 119 aa, Synthetic construct
EVQLVESGGGLVKPGGSLKLSCAASGFTFSDYALSWVRQTPEKRLEWVATIRDGGSYTYYSDNEKGRFTISRDNAKNNLYLQMSHLRSEDTAMYYCTRDQVNRGWFTYWGQGTLVTVSA

SEQ ID NO: 503 - protein, 108 aa, Synthetic construct
QVILTQSPAIMSASLGERVTMTCSASSSVSSSYFHWFQQKPGSSPKLWIYSTSNLASGVPPRFSGSGSGTSYSLTISSMEAEDAATYYCHQHHRSPLTFGAGTKLELK

SEQ ID NO: 504 - protein, 120 aa, Synthetic construct
EVQLQQSGPVLVKPGASVKMSCKASGYIFTDYYMHWVKQSHGKSLEWIGIINPYNGDTSYNQKFKDKATLTVDKSSSTAYMELNSLTSEDSAVYYCARDSSNVGNAMDYWGQGTSVTVSS

SEQ ID NO: 505 - protein, 107 aa, Synthetic construct
DIQMTQTTSSLSASLGDRVTISCRASQDISNYLNWYLQKPDGTIKLLIYYSSRLHSGVPSRFSGSGSGTDYSLTISNLEQEDIATYFCQQGYTLPRTFGGGTKLEII

SEQ ID NO: 506 - protein, 107 aa, Synthetic construct
DIQMTQTTSSLSASLGDRVTISCRASQDISNYLNWYLRKPDGTVKLLIYYSSRLHSGVPSRFSGSGSGTDYSLTISNLEQEDIATYFCQQGNTLPRTFGGGTKLEIK

SEQ ID NO: 507 - protein, 119 aa, Synthetic construct
EVQLVESGGGLVKPGGSLKLSCAASGFTFSGYALSWVRQTPEKRLEWVATIRDGGSYTYYSDNEKGRFTISRDNAKNNLYLQMSHLKSEDTAMYYCTRDQVNRGWFTYWGQGTLVTVSA

SEQ ID NO: 508 - protein, 115 aa, Synthetic construct
DVQLQESGPGLVKPSQSLSLTCSVTGYSITSNYYWNWIRQFPGNNLEWMGYISSDGTNNYNPSLRNRISITRDTSKNQFFLKLNSVTTEDTATYYCARGLRYGPWGQGTTLTVSS

SEQ ID NO: 509 - protein, 111 aa, Synthetic construct
QLILTQSSSASFSLGASAKLTCTLSSQHSTYTIEWYQQQPLKAPKYVMEVKNDGSHRTGDGIPDRFSGSSSGADRYLSISNIQPEDEAIYICGVGDINNYIFGGGTKVTVL

SEQ ID NO: 510 - protein, 120 aa, Synthetic construct
EVMLVESGGGLVKPGGSLKLSCAASGFSFSSYSMSWVRQTPEKRLECVATIGGVGGNTYYPDSVKGRFSISRDNAKNTLYLQMSSLRSEDTALYYCARHEDYYYYAMDYWGQGTSVTVSS

SEQ ID NO: 511 - protein, 106 aa, Synthetic construct
QIVLTQSPAIMSASPGEKVTMTCSANSTVSYIYWYQQKPGSSPRLLIYDTSNLASGVPVRFSGSGSGTSYYLTISRMEAEDAATYYCQQWSSFPFTFGSGTKLEIK

SEQ ID NO: 512 - protein, 120 aa, Synthetic construct
EVMLVESGGGLVKPGGSLKLSCAASGFIFSSYIMSWVRQTPEKRLEWVATIRGGGGNTYYLDSVKGRFIISRDNAKNNLYLQMSSLRSEDTALYYCARHEDYEWYAMDNWGQGTSVTVSS

SEQ ID NO: 513 - protein, 106 aa, Synthetic construct
QIVLTQSPAIMSASPGEKVTLTCSARSSVSYMYWYQQKPGSSPKLLIYDTSNLASGVPVRFSGSGSGTSYSLTISRMEAEDAATYYCQQWSSYPLTFGAGTKLELK

SEQ ID NO: 514 - protein, 120 aa, Synthetic construct
EVMLVESGGDLVKPGGSLKLSCAASGFTFSSYFMSWVRQTPEKRLEWVATISGGGDKTYYLDILKGRFTISRDNAKNTLYLQMSSLRSEDTALYYCARHEDYAWYAMDYWGQGTSVTVSS

SEQ ID NO: 515 - protein, 106 aa, Synthetic construct
QIVLTQSPAIMSASPGEKVTMTCSASSSVNYIYWYQQKPGSSPRLLIYDTSNLASGVPVRFSGSGSGTSYSLTISRMEAEDAATYYCQQWSSYPLTFGAGTKLELK

SEQ ID NO: 516 - protein, 106 aa, Synthetic construct
DIQMTQSPSSLSASLGGKVTITCKASQDINKYIAWYQHKPGKGPRLLIHYTSTLQPGIPSRFSGSGSGRDYSFNISNLEPEDIATYYCLQYDNLYMFGGGTKLEIK

SEQ ID NO: 517 - protein, 119 aa, Synthetic construct
EAQLQQSGPELVKPGASVKISCKASGYTFTDYYMNWVKQSHGKSLDWIGDINPDNGGSTYNQKFKGKATLTVDKSSSTAYMELRSLTSEDSAVYYCSASGPPYVLLDYWGQGTSVIVSS

SEQ ID NO: 518 - protein, 107 aa, Synthetic construct
DIQMTQSPASLSASVGETVTITCQTSENIYSYLAWYQQKQGKSPQLLVYNTQTLAEGVPSRFSGSRSGAQFSLKINSLQPEDFGSYYCQHHYGVPLTFGAGTKLELK

SEQ ID NO: 519 - protein, 120 aa, Synthetic construct
EVQLQQSGPVLVKPGASVKMSCKASGYRLTDYYMHWVKQSHGKSLEWIGIINPYNGGTSYNQKFKGKATLTVDKSSSTAYMELNSLTSDDSAVYYCARDSSNVGNDMDYWGQGTSVSVSS

SEQ ID NO: 520 - protein, 107 aa, Synthetic construct
DIQMTQTTSSLSASLGDRVTISCRASQDISNYLNWYLQKPDGTVKLLIYYSSRLHSGVPSRFSGSGSGTDYSLTINNLEQEDIATYFCQQGNTLPRTFGGGTKLEIR

SEQ ID NO: 521 - protein, 120 aa, Synthetic construct
EVILVESGGGLVKPGGSRKLSCAASGFTFSSYFMSWVRQTPEKRLEWVATISGGGDNTYYPDSVKGRFTISRDNAKNTLYLQMSSLRSEDTALYYCARHEDYEWYAMDYWGQGTSVTVSS

SEQ ID NO: 522 - protein, 106 aa, Synthetic construct
QIVLTQSPAIMSTSPGEKVTMTCSASSSVSYIYWYQQKPGSSPRLLIYDTSNLASGVPVRFSGSGSGTSYSLTISRMEAEDAATYYCQQWTSYPLTFGAGTKLELK

SEQ ID NO: 523 - protein, 120 aa, Synthetic construct
EVQLVESGGDLVKPGGSLKLSCAASGFTFSSYFMSWVRQTPDKRLEWVATIRGGGSYTYYLDSVKGRFTVSRDNAKNTLYLQMSSLKSEDTAMYYCARHEDYDYYAMDYWGQGTSVTVSS

SEQ ID NO: 524 - protein, 106 aa, Synthetic construct
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 525 - protein, 114 aa, Synthetic construct
QAQLQQPGAELVKPGASVKMSCKASGYTFTSYWITWVKQRPGQGLEWIGDIYPGNFSPNYNEKFKNKATLTVDTSSSTAYMQLSSLTSEDSAVYYCAKGYFDVWGTGTTVTVSS

SEQ ID NO: 526 - protein, 120 aa, Synthetic construct
EVQLQQSGPELVKPGASVKISCKASGYTFTHYYKNWVKQSHGKSLEWIGAINPNIGGPSYNQKFKGKATLTVDKSSSTVYMELRSLTSEDSAVYYCARSDDYGGYYFDYWGQGTTLTVSS

SEQ ID NO: 527 - protein, 106 aa, Synthetic construct
QIVLSQSPALLSASPGEKVTMTCRANSSVNYMHWYQQKPGSSPKPWIYATSNLASGVPARFSGSGSGTSFSLTISRVEAEDAATYYCQQWTSNPLTFGAGTKLELK

SEQ ID NO: 528 - protein, 117 aa, Synthetic construct
EVQLQQSGPELVKPGASVKISCKASGYTFTDYYMNWVRQSHGKSLEWIGDIYPNNGDTNYNQKFKGKATLTVDKSSKTADMELRSLTSEDSAVYYCIRGVGLGFPYWGQGTLVTVSA

SEQ ID NO: 529 - protein, 107 aa, Synthetic construct
DIVMTQSHKFISTSVGDGVSITCQASQVVSTAVAWYQQKPGQSPRLLLHSASFRYSGVPDRFTGSGSGTDFTFTISTVQAEDLAVYYCQQHYRTPYTFGGGTKLEIK

SEQ ID NO: 530 - protein, 120 aa, Synthetic construct
EVQLQQSGPVLVKPGASVKMSCEPSGYTFTHYYVNWMRQSHGKSLEWIGVIKPYNGAPSYNQKFKGKATLTVDKSSSKINMELNSLTSEDSAVYYCARSDDFDGYAMDYWGQGTSVTVSS

SEQ ID NO: 531 - protein, 106 aa, Synthetic construct
QIVLTQSPAIMFASPGEKVTMTCSASSSVSYMHWYQQKSGTSPKRCISDTSKLASGVPARFSGSGSGTSYSLTISSMEAEDAATYYCQQWTSNPLTFGSGTKLELK

SEQ ID NO: 532 - protein, 120 aa, Synthetic construct
EVQLVESGGDLVKPGGSLKLSCAASGFTFNNYGMSWVRQTPDKRLEWVATISRGGSFTYYPDSVKGRFTISRDNAKNTLYLQMSSLKSEDTAMYYCARPIYYEYDGFAYWGQGTLVTVSA

SEQ ID NO: 533 - protein, 112 aa, Synthetic construct
DVLMTQTPLSLPVSLGDQASISCRSSQSIVHSNGNTYLEWYLQKPGQSPKLLIYKVSNRFSGVPDRFSGSGSGTDFTLKINRVEAEDLGVYSCFQGSHVPRTFGGGTKLEIK

SEQ ID NO: 534 - protein, 114 aa, Synthetic construct
QVQLQQPGAELVKAGASVKMSCKASGYTFTSYWITWVKQRPGQGLEWIGDIYPGNDSPNYNEKFKNKATLTVDTSSSTAYMQLSSLTSEDSALYYCARGYYDYWGQGTTLTVSS

SEQ ID NO: 535 - protein, 113 aa, Synthetic construct
DIVMTQSPSSLSVSAGEKVTMSCKSSQSLLNSGNQKNYLAWYQQKTGQPPKLLIYGASTRESGVPDRFTGSGSGTDFTLTISSVQAEDLAVYYCQNDHSYPHTFGAGTKLELK

SEQ ID NO: 536 - protein, 113 aa, Synthetic construct
EVQLQQSGPVLVKPGASVKMSCKASGNTFNGYYVNWVKQSHGKSLEWIGLIDPSNGGTNYNQKFKDKATLTVDKSSTTAYMEFNSLTSEDSSVYYCAGGTVYWGQGTTLTVSS

SEQ ID NO: 537 - protein, 107 aa, Synthetic construct
DIQMTQSPSSLSASLGERVSLTCRASQDIGNNLNWLQQEPDGTIKRLIYATSSLDSGVPKRFSGSRSGSDYSLTISSLESEDFVDYYCLQYASSPVTFGGGTKLEIK

SEQ ID NO: 538 - protein, 120 aa, Synthetic construct
EVQLQQSGPELVKPGASVKISCKASGYMFTDYYMNWVKQSHGGSLEWIGTFNPNRGGPSYNLKFKGKATLTVDKSSITAYMDLRSLTSEDSAVYYCARADDYDGYYFDYWGQGTTLTVSS

SEQ ID NO: 539 - protein, 106 aa, Synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWTNNPLTFGAGTKLELK

SEQ ID NO: 540 - protein, 115 aa, Synthetic construct
QVQLQQSGPELVKPGASVRISCKASGYSFTDYFISWVKQRPGQGLEWIGWIFPGSGSTYFNENFKGEATLTADKSSSTAYLLLSSLTSEDSAVYFCTTYYGSNYWGQGTTLTVSS

SEQ ID NO: 541 - protein, 112 aa, Synthetic construct
DVLMTQAPLSLPVSLGDQASISCRSSQSIVHSNGNTYLEWYLQKPGQSPKLLIYTVSNRLSGVPDRFSGSGSGTDFTLKISRVEAEDLGIYYCFQTSHVPPTFGGGTKLEIK

SEQ ID NO: 542 - protein, 106 aa, Synthetic construct
DIQMTQSPSTLSASVGDRVTITCRASQSISSWLAWYQQKPGKAPKVLIYKASTLESGVPSRFSGSGSGTDFTLTISSLQPEDFATYNCQQYNTYWTFGQGTKVEIK

SEQ ID NO: 543 - protein, 114 aa, Synthetic construct
EVQLQQSGPELVKPGASVKISCKASGYTFTDYSMNWVKQSHGKSLEWIGDINPNNGGTSYNQKFKGKATLTVDKSSSTAYMELRSLTSEDSAVYYCARVWFAYWGQGTLVTVSA

SEQ ID NO: 544 - protein, 113 aa, Synthetic construct
DIVMTQSPSSLSVSAGEKVTLSCKSSQSLLNSGNQKNYLAWYQQKPGQPPKLLIYGASTRESGVPDRFTGSGSGTDFTLTISSVQAEDLALYYCQNAHSYPFTFGSGTKLDIK

SEQ ID NO: 545 - protein, 118 aa, Synthetic construct
QHELQQSGAELVRPGSSVKLSCKDSYFAFMASAMHWVKQRPGHGLEWIGSFTMYSDATEYSENFKGKATLTANTSSNTAYMELSSLISEDSAVYYCASGYSGNGFTYWGQGTLVTVSA

SEQ ID NO: 546 - protein, 112 aa, Synthetic construct
DIVLTQSPASLAMSLGQRATISCRASESVDNYGISFMHWYQQKPGQPPKLLIYRASNLESGIPARFSGSGSRTDFTLTINPVETDDVATYYCQQSNKDPMYTFGGGTKLEIR

SEQ ID NO: 547 - protein, 114 aa, Synthetic construct
QVQLQQPGAELVKPGASVNMSCKASGYTFSSYWITWVKQRPGQGLEWIGDIHPGNTSPNYNEKFRTKATLTVDTSSSTAYMQLSSLTSEDSAVYYCARGWGDYWGQGTTLTVSS

SEQ ID NO: 548 - protein, 113 aa, Synthetic construct
DIVMTQSPSSLSVAAGEKVTMSCKSSQSLLNSGNQKNYLAWYQQKPGQPPKLLIYGASTRESGVPDRFTGSGSGTDFTLTISSVQAEDLAVYYCQNDHGYPLTFGTGTKLELK

SEQ ID NO: 549 - protein, 121 aa, Synthetic construct
EVQLVESGGDLVKPGGSLKLSCVASGLTFSDYGMYWIRQAPEKGLEWVAYIRSDSSTIYYTDTVKGRFTISRDNAKNTLFLQMTSLRSEDTAIYYCARRSPVVVNYAMDYWGQGTSVTVSS

SEQ ID NO: 550 - protein, 107 aa, Synthetic construct
DIQMTQSPASLSASVGETVTITCRASENIYSHLVWYQQKQGKSPHLLVYNGKILAEGVPSRFSGSGSGTQFSLKINSLQPEDFGSYYCQHHYGSPITFGAGTKLELN

SEQ ID NO: 551 - protein, 120 aa, Synthetic construct
EVQLQQSGPELVKPGTSVKISCKASGYTFTDYYMNWVRQSHGKSLEWIGEINPKNGDTNYNQNFKGKATLTVDKSSNTAYMELRSLTSEDSAVFYCAREGVSSYYAMDYWGQGTSVTVSS

SEQ ID NO: 552 - protein, 113 aa, Synthetic construct
DIVMTQSPSSLAMSVGQKVTMSCKSSQSLLNSFNQKNFLAWYQQKPGQSPKLLVYFASTRESGVPDRFIGSGSGTDFTLTITSVQAEDLADYFCQQHHSTPPTFGGGTKLEIN

SEQ ID NO: 553 - protein, 113 aa, Synthetic construct
EVQLQQSGPVLVKPGASVKMSCKASGNTFNGYYINWVKQSHGKSLEWIGLIDPSNGGTNYNQKFKDKATLTVDKSSTTAYMEFNTLTSEDSSVYYCAGGTVYWGQGTTLTVSS

SEQ ID NO: 554 - protein, 107 aa, Synthetic construct
DIQMTQSPSSLSASLGERVSLTCRASQDIGNNLNWLQQEPDGTIKRLIYATSSLDSGVPKRFSGSRSGSDYSLTISSLESEDFVDYYCLQYASSPPTFGGGTKLEIK

SEQ ID NO: 555 - protein, 121 aa, Synthetic construct
EVQLVESGGGLVKPGGSLKLSCAASGITFSDYGMHWVRQAPEKGLEWVAYIRSDSSTIYYADTVKGRFTISRDNAKNTLFLQMTSLRSDDTAMYYCARRSPVVVNYAMDYWGQGTSVTVSS

SEQ ID NO: 556 - protein, 107 aa, Synthetic construct
DIQMTQSPASLSASVGETVTITCRASENIYSYLVWYQQKQGKSPQLLVYNGKILAEGVPSRFSGSGSGTQFSLKINSLQPEDFGSYYCQHHYGSPLTFGAGTKLELK

SEQ ID NO: 557 - protein, 120 aa, Synthetic construct
EVHLQQSGPELVKPGASVKISCKASGYTFTDYYMNWMKQSHGKGLEWIGEINPKNGDTNYSQKFRDKATLTVDKSSNTAFMKLRSLTSEDSAVYFCAKEGVGSYYAMDYWGQGTSVTVSS

SEQ ID NO: 558 - protein, 113 aa, Synthetic construct
DIVMTQSPSSLAMSVGQKVTMSCKSSQSLLSSNNQKSFLAWYQQKPGQSPKLLVSFASTRESGVPDRFIGGGSGTDFTLTISSVQPEDLADYFCQQHYSTPPTFGEGTKLEIK

SEQ ID NO: 559 - protein, 114 aa, Synthetic construct
QVQLQQPGAELVKAGASVKMSCKASGYTFTSYWITWVKQRPGQGLEWIGDIYPGNDSPNYNEKFKNKATLTVDTSSSTAYMQLSSLTSEDSAVYYCTRGYYDYWGQGTTLTVSS

SEQ ID NO: 560 - protein, 113 aa, Synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPHTFGAGTKLELR

SEQ ID NO: 561 - protein, 113 aa, Synthetic construct
EVQLQQSGPVLVKPGASVKMSCKASGNTFNGYYINWVKQRHGKSLEWIGLIDPSNGGTNYNQKFKDKATLTVDKSSTTAYMEFNSLTSDDSSVYYCAGGTVYWGQGTTLTVSS

SEQ ID NO: 562 - protein, 120 aa, Synthetic construct
EVQLQQSGPELVKPGASVKISCKASGYMFTDYYMNWVKQSHGKSLEWIGEINPNNGDTNYSQKFRDKATLTVDKSSNIAFMELRSLTSEDSAVYFCAKEGVSSYYAMDYWGQGTSVTVSS

SEQ ID NO: 563 - protein, 113 aa, Synthetic construct
DIVMTQSPSSLAMSVGQKVTMSCKSSQSLLNSNNQKNFLAWYQQKPGQSPKLLVYFASTRESGVPDRFIGGGSGTDFTLTISNVQPEDLADYFCQQHYSTPPTFGGGTKLEIK

SEQ ID NO: 564 - protein, 120 aa, Synthetic construct
EVQLQQSGPELVKPGTSVKISCKASGYTFTDYYMNWVRQSHEKSLEWIGEINPKNGDTNYNQNFKAKATLTVDKSSNTAYMELRSLTSEDSAVFYCAREGVSSYYGMDSWGQGTSVTVSS

SEQ ID NO: 565 - protein, 113 aa, Synthetic construct
DIVMTQSPSSLAVSVGQKVTMSCKSSQSLLNGFNQKNFLAWYQQKPGQSPKLLVYFASTRESGVPDRFIGSGSGTDFTLTITSVQAEDLADYFCQQHHSTPPTFGGGTKLEIN

SEQ ID NO: 566 - protein, 114 aa, Synthetic construct
EVQLQQSGPELVKPGASVKMSCKASGYTFTDYSMNWVKQSHGKSLEWIGDINPNNGGTNYNQKFKGKATLTVDRSSSTAYMELRSLTSEDSAVYYCARVWFAYWGQGTLVTVSA

SEQ ID NO: 567 - protein, 113 aa, Synthetic construct
DIVMTQSPSSLSVSEGEKVTMSCKSSQSLLSSGNQKNFLAWYQQKAGQPPKLLIYGASTRESGVPDRFTGSGSGTDFTLTISSVQAEDLAVYYCQNAHSSPFTFGSGTKLEIK

SEQ ID NO: 568 - protein, 114 aa, Synthetic construct
EVQLQQSGPELVKPGASVKISCKASGYTFTDYSMNWVKQSHGKSLEWIGDINPDNGGTSYNQKFKGKATLTVDQSSSTAYMELRSLTSEDSAVYYCARIWFAYWGQGTLVTVSA

SEQ ID NO: 569 - protein, 113 aa, Synthetic construct
DIVMTQSPSSLSVSAGEKVTMSCKSSQSLLNSGIQKNFLAWYQQKPGQPPKLLIYGASTRESGVPDRFTGSGSGTDFTLTISSVQAEDLAVYYCQNAHSYPFTFGSGTKLEIK

SEQ ID NO: 570 - protein, 120 aa, Synthetic construct
EVQLQQSGPELVKPGASVKISCKASGYTFTDYYMNWVRQSHEKSLEWIGEINPNNGDTNFNQKFKGKATLTVDKSSSTAFMDLRSLTSKDSAVYYCAREGGSSYYAMDYWGQGTSVTVSS

SEQ ID NO: 571 - protein, 113 aa, Synthetic construct
DIVMTQSPSSLAMSVGQKVTMSCKSSQSLLSNNNQKNFLAWYQQKPGQSPKLLVYFASTRESGVPDRFIGSGSGTDFTLTISSVQAEDLADYFCQQHYSTPPTFGGGTKVEIK

SEQ ID NO: 572 - protein, 119 aa, Synthetic construct
EVQLQQSGPELVKPGSSVKISCEASGFTFADYNVDWVKQSHGKSLEWIGAINPNDGGTSYNQKFEGKATLTVDKSSTTAYMELRSLTSEDSAVYYCARWVNNDWYFDVWGTGTTVTVSS

SEQ ID NO: 573 - protein, 107 aa, Synthetic construct
DIVMTQSQKFMSTSVGDRVSITCKASQNVRSAVDWYQQKPGQSPKTLIHSASNRHTGVPDRFTGSGSGTDFTLTISNIQSEDLADYFCLQHWNYPYTFGGGTKLEVK

SEQ ID NO: 574 - protein, 119 aa, Synthetic construct
QVQLQQSGAELARPGASVKLSCKASGYTFTNYGISWVKQRTGQGLEWIGEIYPGTINIYYSERFKGKATLTADKSSSTAYMQLTSLTSEDSAVYFCARDDGYYDAMDYWGQGTSVTVSS

SEQ ID NO: 575 - protein, 107 aa, Synthetic construct
DIQMTQSPSSLSASLGQKVILTCRASQDIGSSLNWLQQEPDGTIKRLIYATSSLDSGVPKRFSGSRSGSDYSLTISSLESEDFVVYYCLQYTSSPWTFGGGTKLEIK

SEQ ID NO: 576 - protein, 123 aa, Synthetic construct
QVQLQQPGAELVKPGASVKLSCKASGYTFTSYWMHWVKQRPGQGLEWIGEIDPSDSYTNYNQKFKGKATLTVDKSSSTAYMQLSSLTSEDSAVYYCASSIYYSNYVRYYFDYWGQGTTLTVSS

SEQ ID NO: 577 - protein, 119 aa, Synthetic construct
EVQLQQSGPELVKPGSSVKISCEASGYTFADYNVDWVKQSHGKSLEWIGAINPNDGGTSYNQKFEGKATLTVDKSSTTAYMELRSLTSEDSAVYYCARWVINDWYFDVWGTGTTVTVSS

SEQ ID NO: 578 - protein, 106 aa, Synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLWTFGGGTKLEIK

SEQ ID NO: 579 - protein, 119 aa, Synthetic construct
EVQLQQSGPELVRPGSSVKISCKASGYTFTDYNMDWVKQSHGKSLEWIGANDPKNGGFSYNQRFKGKATLTVDKSSSTAYMELRSLTSEDSAVYYCARWDSNYWYFDVWGTGTTVTVSS

SEQ ID NO: 580 - protein, 107 aa, Synthetic construct
DIVMTQSQKFMSTSIGDRVSITYKASQDVRSAVAWYQQKPGQSPKALIYLASNRHTGVPDRFTGSGSGTDFTLTITNVQSEDLADYFCLQHWTYPYTFGGGTKLEIK

SEQ ID NO: 581 - protein, 119 aa, Synthetic construct
EVQLQQSGPELVKPGSSMKISCKASGYTFTDYSMDWVKQSHGKSLEWIGAINPNNGGTSYNQKFKDKATLTVDRSSSTAYMELRSLTSEDSAVYYCARWDSDYWYFDVWATGTTVTVSS

SEQ ID NO: 582 - protein, 107 aa, Synthetic construct
DIVMTQSQKFMSTSVGDRVSITCKASQNVRTAVDWYQQKPGQSPKALIYSASNRHTGVPDRFTGSGSGTDFTLTISNVQSEDLADYFCLQHWNYPYTFGGGTKLEIK

SEQ ID NO: 583 - protein, 118 aa, Synthetic construct
EVQLQQSGPELVKPGSSVKISCKASGYTFTDYNMDWVKQSHGQSLEYIGAVNPNTGDISYTQKFKDKATLTVDKSSSTAYMELRSLTSEDSAVYYCARWSESWYFDVWGTGTTVTVSS

SEQ ID NO: 584 - protein, 107 aa, Synthetic construct
DIVMTQSQKLMSTSVGDRVSITCKASQNVRAAVDWYQQKPRQSPKALIFLASSRHTGVPDRFTGSGSGTDFTLTITNVQSEDLATYFCLQHWNYPYTFGGGTRVEIK

SEQ ID NO: 585 - protein, 119 aa, Synthetic construct
EVQLQQSGPELVKPGSSVKISCKASGYTFTDYNIDWVKQSHGKSLEWIGAIDPNNGGTNYNQKFKDKATLTVDKSSSTAYMELRSLTSEDSAVYYCTRWDSNDWYFDVWATGTTVTVSS

SEQ ID NO: 586 - protein, 107 aa, Synthetic construct
DIVMTQSQKFMSTSVGDRVSITCKASQNVRIAVDWYQQKPGQSPKALIFSSSNRHTGVPDRFTGSGSGSDFTLTIRNVQSEDLADYFCLQHWISPYTFGGGTKLEIR

SEQ ID NO: 587 - protein, 119 aa, Synthetic construct
EVQLQQSGPELAKPGSSVKISCKASGYTFTDYNIDWVKQSHGKSLEWIGAIDPNNDSTNYNQKFKGKATLTVDKSSSTAYMELRSLTSEDSAVYYCTRWDSDDWYFDVWATGTTVTVSS

SEQ ID NO: 588 - protein, 107 aa, Synthetic construct
DIVMTQSQKFMSTSVGDRVSITCKASQNVRSAVDWYQQKPGQSPKALIFSSSNRHTGVPDRFTGSGSGTDFTLTIRNVQSEDLADYFCLQHWNSPYTFGGGTKLEIR

SEQ ID NO: 589 - protein, 107 aa, Synthetic construct
DIQMTQSSSSFSVSRGDRVTITCKASEDIYNRLAWYQQKPGNAPRLLISGTTSLEIGFPSRFSGSGSGKDYTLSISSLQTEDVATYYCQQYWSTPWTFGGGTKLEIK

SEQ ID NO: 590 - protein, 123 aa, Synthetic construct
EVQLQQSGAELAKPGASVKLSCTVSGFNIKDTYIHWVKQRPEQGLEWIGGIDPAIGNTVSASKFQDKATITADTSSSTAYMQLSSLTSGDTAVYYCARERVHSNTLYYAMDYWGQGTSVTVSS

SEQ ID NO: 591 - protein, 111 aa, Synthetic construct
DIVLTQSPASLAVSLGQRATISCRASKSVSISGYSYMHWYQQKPGQPPKLLIYLASNLESGVPARFSGSGSGTDFTLNIHPVEEEDAATYYCQHSRELPPTFGGGTKLEIK

SEQ ID NO: 592 - protein, 120 aa, Synthetic construct
QVQLQQSGPELARPGASVKLSCKASGYTLTSHGISWVKQRTGQGLEWIGEIYPGSESPYYSEKFKGKATLTADKSSNIAYMQLSSLTSEDSAVYFCARSDYSDYWAMDYWGQGTSVTVSS

SEQ ID NO: 593 - protein, 107 aa, Synthetic construct
DIVMTQSQKCMSTSVGDRVSITCKASQNVRSAVAWYQQKPGQSPKTLIYLASNRHTGVPDRFTGSGSGTDFTLTITNVQSEDLADYFCLQHWNYPWTFGGGTELEIK

SEQ ID NO: 594 - protein, 107 aa, Synthetic construct
EIVMTQSPATLSVSPGERATLSCRASQSVSSNLAWYQQKPGQAPRLLIYGASTRATGIPARFSGSGSGTEFTLTISSLQSEDFAVYYCHQYNNWPRTFGQGTKVEIK

SEQ ID NO: 595 - protein, 120 aa, Synthetic construct
QVQLQQPGAELVKPGASVKLSCKASGYTFTSSWMHWVKQRPGQGLEWIGEINPSDGRTNYNEKFKSKATLTVDHSSSTAYMQLSSLTSEDSAVYYCASNADRAYYTMDYWGQGTSVTVSS

SEQ ID NO: 596 - protein, 113 aa, Synthetic construct
DIVMTQSPSSLAMSVGQKVTMSCKSSQSLLNSFNQRNFLAWYQQKPGQSPKLLVYFASTRDSGVPDRFIGSGSGTDFTLTISSVQAEDLADYFCQQHYSTPPTFGGGTKLEIK

SEQ ID NO: 597 - protein, 119 aa, Synthetic construct
EVQLQQSGPELVKPGSSVKISCKASGYTFTDYNLDWMKQSHGSSLEWIGAMSPNNGATSYNQKFKGKATLTVDKSSSTAYMELRSLTSEDSAVYYCARWDSNSWYFDVWGTGTTVTVSS

SEQ ID NO: 598 - protein, 107 aa, Synthetic construct
DIVMTQSQKFMSTSVGDRVSITCKASQIVRTAVDWYQQKSGQSPKALIYSASTRHTGVPDRFTGSGSGTDFTLTIDNVQSEDLAEYFCLQHWNYPYTFGGGTKLEIK

SEQ ID NO: 599 - protein, 114 aa, Synthetic construct
QVQLQQSGAELVRPGTSVKMSCKAAGYTFTNSWINWVKQRPGHGLEWIGDIYPGNTFPNYNEKFKGKATLTADTSSSTAYMQLSSLTSEDSAVYYCTGGYFDVWGTGTTVTVSS

SEQ ID NO: 600 - protein, 113 aa, Synthetic construct
DIVMTQSPSSLSVSAGEKVTMSCKSSQSLLNSGNQKNFLAWYQQKPGQPPKLLIFGASTRQSGVPDRFTGSGSGTDFTLTISSVQAEDLAVYYCQNDHSYPYTFGGGTKLEMK

SEQ ID NO: 601 - protein, 123 aa, Synthetic construct
EVQLQQSGAELVKPGASVKLSCTASGFNIEDTYIHWVKRRPEQGLEWIGGIDPAIGNTLYASKFQDMATITADTSSSTAYMQLSSLTSGDTAVYYCARERVYSNTLYYAMDYWGQGTSVTVSS

SEQ ID NO: 602 - protein, 111 aa, Synthetic construct
DIVLTQSPASLVVSLGQRATISCRASKSVSISGYSYMHWYQQKPGQPPKLLIYLASNLESGVPARFSGSGSGTDFTLNIHPVEAEDAATYYCQHSRELPPTFGGGTNLEIK

SEQ ID NO: 603 - protein, 119 aa, Synthetic construct
EVKLVESGGGLVQPGGSLKLSCAVSGFTFSSYTMSWVRQTPEKRLELVAKISNSGGSTNYPDTVKGRFTLSRDNVKNTLYLQMSSLKSEDTAIYYCTRYSWDVYAMDYWGQGTSVTVSS

SEQ ID NO: 604 - protein, 106 aa, Synthetic construct
DIQMTQSPASLSASVGETVIITCRASENIYSYLAWYQQKQGKSPHLLVYNAETLVEGVPSRFSGSGSATQFSLKINSLQPEDFGTYYCQHHYGDPTFGGGTKLEIK

SEQ ID NO: 605 - protein, 116 aa, Synthetic construct
QVQLQQSGAELARPGASVKMSCKASGYTFTDYWMQWVKQRPGQGLEWIGAIFPGNGETRDTQKFKGRATLTADRSSSTAYIQLSSLTSEDSAVYYCATFNNYLDYWGQGTTLTVAS

SEQ ID NO: 606 - protein, 106 aa, Synthetic construct
QIVLTQSPAIMSASPGEKVTMTCSASSSVSYMHWYQQKSGASPKRWIYDTSKLTSGVPARFSGSGSGTSYSLTISNMEAEDAATYYCQQWISNPYTFGGGTKLEIK

SEQ ID NO: 607 - protein, 120 aa, Synthetic construct
QVQLQQPGAELVKPGTSVKLSCKAAGYIFTSSWMHWVKQRPGQGLEWIGEINPSDGRTNYNEKFKNKATLTVDHSSSTAYMQLSSLTSEDSAVYYCASNADRAYYTMDYWGQGTSVTVSS

SEQ ID NO: 608 - protein, 113 aa, Synthetic construct
DIVMTQSPSSLAMSVGQKVTMSCKSSQSLLNGFNQRNFLAWYQQKPGQSPKLLVYFASTRDSGVPDRFIGSGSGTDFTLTISSVQAEDLADYFCQQHYSTPPTFGGGTKLEIK

SEQ ID NO: 609 - protein, 118 aa, Synthetic construct
EVKLVESGGGLVQPGGSLKLSCVASGFTFNTYIMSWVRQTPEKRLELVADISNSGGNTYYSDTVKGRFTISRDNAKNTLYLQMSSLKSEDTAMYYCARPDNWDPFVYWGQGTLVTVSA

SEQ ID NO: 610 - protein, 107 aa, Synthetic construct
DIQMTQSSSSFSVSLGDRVTITCKASEDIYARLAWYQQKPGNAPRLLISGATSLETGVPSRFSGSGSGKNYTLSITSLQTEDVGTYYCQQHWSTPWTFGGGTNLEIK

SEQ ID NO: 611 - protein, 114 aa, Synthetic construct
EVQLQQSGPEVVKPGASVKISCKASGNTLTDYFINWVKQSHGKNLEWIGDINPDNGAGNYNQKFKGKATLTVDKSSGTAFMGLRSLTSEDSAVYYCATLYFDYWGQGTTLTVSS

SEQ ID NO: 612 - protein, 113 aa, Synthetic construct
DIVMTQSPSSLSVSTGEKVTMSCRSSQSLLNSGNQKNYLAWYQQKPGQPPKLLIYGASTRESGVPDRFTGSGSGTDFTLTISSVQAEDLAIYYCQNAHTSPYTFGGGTRLEIK

SEQ ID NO: 613 - protein, 114 aa, Synthetic construct
EVQLQQSGPEVVKPGASVKISCKASGNTFTDYFINWVKQSHGKSLEWIGDINPDNGGTNYNQKFKGKATLTVDKSSGTAFMGLRSLTSEDSAVYYCATLYFDYWGQGTSLTVSS

SEQ ID NO: 614 - protein, 113 aa, Synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNAHSSPYTFGGGTRLEIK

SEQ ID NO: 615 - protein, 114 aa, Synthetic construct
EVQLQQSGPEVVKPGASVKISCKASGNTFTDYFINWVKQRHGKSLEWIGDVNPDNGGTNYNQKFKGKATLTVDKSSGTAFMGLRSLTSEDSAVYYCATLFFDSWGQGTTLTVSS

SEQ ID NO: 616 - protein, 113 aa, Synthetic construct
DIVMTQSPSSLNVSAGEKVTMSCKSSQSLLNSGNQKNYLAWYQQKPGQPPKLLIYGASTRESGVPDRFTGSGSGTDFTLTISSVQAEDLALYYCQNAHSSPYTFGGGTKLEIK

SEQ ID NO: 617 - protein, 120 aa, Synthetic construct
EVQLVESGGGLVKPGGSLKLSCAASGFTFSNFAMSWVRQTPAMRLESVATIVNGGINTYYPDTVKGRFTVSRDNAKNTLYLQMSSLRSEDTAIYYCARHDDFLYYAMDYWGQGTSVTVSS

SEQ ID NO: 618 - protein, 106 aa, Synthetic construct
QIVLTQSPAIMSASPGEKVTMTCSASSSVSYMFWYQQKPGSSPRLLIYDTSNLASGVPVRFSGSGSGTSYSLTISRLEAEDAATYYCQQWTSYPLTFGAGTKLDLK

SEQ ID NO: 619 - protein, 120 aa, Synthetic construct
EVQLVESGGGLVKPGGSLKLSCAASGFTFSSFAMSWVRQTPAKRLESVATINNGGINTYYPDTVKGRFTVSRDNAKNTLYLQMSSLRSEDTAMYYCARHDDFLYYAMDYWGQGTSVTVSS

SEQ ID NO: 620 - protein, 106 aa, Synthetic construct
QIVLTQSPAIMSASPGEKVTMTCSASSSVSYMYWYQQKPGSSPRLLIYDTSNLASGVPVRFSGSGSGTSYSLTISRMEAEDAATYYCQQWTSYPLTFGAGTKLELK

SEQ ID NO: 621 - protein, 119 aa, Synthetic construct
EVKLVESGGGLVQPGGSLKLSCAASGFTFSSYTMSWVRQTPEKRLELVAELSNSGGSFHYPDNVKGRFTISRDNAKNTLYLQMSSLKSEDTAMYYCARHNWDVYAMDYWGQGTSVTVSS

SEQ ID NO: 622 - protein, 106 aa, Synthetic construct
DIQMSQSPASLSASVGETVTITCRASENVNSYLAWYQQKQGRSPQFLVYNAQTLAEGVPSRFSGGGSGTQFSLKINSLQPEDFGSYYCQHHYGNPTFGGGTKLEIK

SEQ ID NO: 623 - protein, 120 aa, Synthetic construct
QVQLQQSGAELMTPGSSVKLSCKATGYTFTDYWIEWVKKRPGHGLEWIGEILPGYISTNYNEKFKDKATFTADTSSNTTYMQLSSLTSEDSAVYYCVLIVTTYYYFMDYWGQGTSVTVSS

SEQ ID NO: 624 - protein, 106 aa, Synthetic construct
QIVLTQSPAIMSASPGEKVTITCSASSSVTYMHWFQQKPGTSPKLWIYSTSNLASGVPVRFSGSGSGTSYSLTISRMEAEDAATYYCQQRSSYPWTFGGGTKLEIK

SEQ ID NO: 625 - protein, 120 aa, Synthetic construct
QVQLQQSGAELMKPGSSVNLSCKATDYTFTDYWINWVKQRPGHGLEWIGEILPGSVTTNYNEKFRGKATFTADTSSNTAYMQLSSLTSEDSAVYFCARGSDYSYYAMDYWGQGTSVTVSS

SEQ ID NO: 626 - protein, 113 aa, Synthetic construct
DIVMTQSPSSLAMSVGQKVTMSCKSSQSLLNIFNQKNFLAWYQQKPGQSPKLLVYFASTRESGVPDRFIGSGSGTDFTLTISNVQAEDLADYFCQQHYSTPPTFGGGTKLEIK

SEQ ID NO: 627 - protein, 118 aa, Synthetic construct
DVQLVESGGGLVKPGGSLKLSCAASGFSFSENYMYWVRQTPEKRLEWVATISDGASYTYYPDNVKGRFTISRDTAKNNLYLQMSHLKSEDTAMYYCTRDELIAPFAYWGQGTLVTVSA

SEQ ID NO: 628 - protein, 107 aa, Synthetic construct
DIVMTQSHKFMSTSVGDRVSITCKASQDVGTAVAWFQQKPGQSPKLLIYWASTRHTGVPDRFTGSGSGTDFTLTISNVQSEDLADYFCQQYNSYPWTFGGGTKLEIK

SEQ ID NO: 629 - protein, 120 aa, Synthetic construct
EVQLVESGGGLVKPGGSLKLSCAASGFTFSSYAMSWVRQTPAKRLEWVATITSGDIRTYYPDTVKGRFTISRDNAKNTLYLQMRSLRSEDTAMYYCVGHDDFHFYAMDYWGQGTSVTVSS

SEQ ID NO: 630 - protein, 106 aa, Synthetic construct
QIVLTQSPAIMSASPGEKVTMTCSASSSVSYLYWYQQKPGSSPRLLIYDTSNLASGVPVRFSGSGSGTSYSLTLSRMEAEDAATYYCQQWSSYPLTFGAGTKLELK

SEQ ID NO: 631 - protein, 120 aa, Synthetic construct
QVQLQQSGAELARPGTSVKMSCKASGYIFTNYWMNWVQQRPGQGLEWIGAINPSNGYIGYNQKFKDKATLTADKSSNTAYMQLRSLTSEDSAIYYCTRGEDFDGYAMDYWGQGTSVTVSS

SEQ ID NO: 632 - protein, 106 aa, Synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLTFGAGTKLELK

SEQ ID NO: 633 - protein, 106 aa, Synthetic construct
QIVLTQSPAIMSASPGEKVTMTCSASSSVNYMFWYQQKPGSSPRLLIYDTSNLASGVPVRFSGSGSGTSYSLTISRLEAEDAATYYCQQWTSYPLTFGAGTKLDLK

SEQ ID NO: 634 - protein, 114 aa, Synthetic construct
EVQLQQSGPEVVKPGASVRISCKASGNTFTDFFINWVKQSHGKSLEWIGDVNPDSNGANYNQKFKGKATLTVDKSSGTAFMGLRSLTSEDSAVYYCATLFFDNWGQGTTLTVSS

SEQ ID NO: 635 - protein, 113 aa, Synthetic construct
DIVMTQSPSSLNVSAGEKVTMSCKSSQSLLNSGNQKNYLAWYQQKPGQPPKLLIYGASTRESGVPDRFTGSGSGTDFILTISSVQAEDLALYYCQNAHSSPYTFGGGTKLEIK

SEQ ID NO: 636 - protein, 119 aa, Synthetic construct
EVQLQQSGPELVKPGSSVKISCEASGYTFADYNVDWVKQSHGKSLEWIGAINPNDGGTSYNQKFEGKATLTVDKSSTTVYMELRSLTSEDSAVYYCARWVNNDWYFDVWGTGTTVTVSS

SEQ ID NO: 637 - protein, 107 aa, Synthetic construct
DIVMTQSQKFMSTSVGDRVSITCKASQNVRSAVDWYQQKPGQSPKTLIHSASSRHTGVPDRFTGSGSGTDFTLTISNIQSEDLADYFCLQHWNYPYTFGGGTKLEVK

SEQ ID NO: 638 - protein, 119 aa, Synthetic construct
EVQLQQSGPELLKPGSSVNISCKASGYTFTDYNIDWVKQSRGKSLEWIGAINPNNGGTFYNQKFKGQATLTVYKSSSTAYMELRSLTSEDSAVYYCVRWDSDSWYFDVWGTGTAVTVSS

SEQ ID NO: 639 - protein, 107 aa, Synthetic construct
DIVMTQSQKFMSTSVGERVSITCKASENVRAAVDWYQQKPGQSPKALIYLASNRHTGIPDRFTGSGSGTDFTLIISNVQSEDLADYFCLQHWNYPYTFGGGTKLEIT

SEQ ID NO: 640 - protein, 119 aa, Synthetic construct
EVQLQQSGPELAKPGSSVKISCKASGYTFTDYNIDWVKESHGKSLEWIGAIDPNNGGTNYNQKFKGKATLTVDKSSSTAYMELRSLTSEDSAVYYCTRWDSNDWYFDVWATGTTVTVSS

SEQ ID NO: 641 - protein, 107 aa, Synthetic construct
DIVMTQSQKFMSTSVGDRVSITCKASQNVRSAVDWYQQKPGQSPKALIFSSSNRHTGVPDRFAGSGSGTDFNLTIRNVQSEDLADYFCLQHWNSPYTFGGGTKLEIR

SEQ ID NO: 642 - protein, 117 aa, Synthetic construct
QVTLKESGPGILQPSQTLSLTCSFSGFSLSTFGLGIGWIRQPSGKGLEWLAHIWWDDDKYYNPALKSRLTISKDTSKNQVFLKIANVDTADTATYYCATGTYWFTYWGQGTLVTVSA

SEQ ID NO: 643 - protein, 112 aa, Synthetic construct
DVLMTQTPLSLPVSLGDQASISCRSSQSIEHSNGNTYLQWYLQKPGQSPKLLIYKVSNRFSGVPDRFSGSGSGTDFTLKISRVEAEDLGVYYCFQGSHVPWTFGGGTKLEIK

SEQ ID NO: 644 - protein, 120 aa, Synthetic construct
EVQLQQSGPVLVKPGASVKMSCKASGYRLTDYYMHWVKQSHGKSLEWIGIINPYNGDTSYNQKFKGKATLTVDKSSSTAYMELDGLTSDDSAVYYCARDSSNVGNAMDYWGQGTSVTVSS

SEQ ID NO: 645 - protein, 107 aa, Synthetic construct
DIQMTQTTSSLSASLGDRITISCRASQDISNYLNWYLQKPDGTIKLLIYYSSRLHSGVPSRFSGSGSGTDYSLTITNLEQEDIATYFCQQGHALPRTFGGGTKLEIK

SEQ ID NO: 646 - protein, 119 aa, Synthetic construct
EVQLQQSGPELVKPGASVKISCKGSGYTFTDYFMNWVKQSHGKSLEWVGDINPNNGDTTYSQKFMGKATLTVDKSSSTAYMELRSLTSEDSAVYHCAASGPPYVLLDYWGQGTSVTVSS

SEQ ID NO: 647 - protein, 107 aa, Synthetic construct
DIQMTQSPASLSASVGETVTITCRASENIYSYLAWYQQKQGKSPQLLVYNAKTLTEGVPTRFSGSGSGTQFSLKIDSLKPEDFGTYYCQHHYGTPLTFGAGTKLELN

SEQ ID NO: 648 - protein, 116 aa, Synthetic construct
QVQLQQSGTILARPGASVKMSCRASDYTFTDYWIQWIKQRPGQGLEWIGAIFPENGDTRYTQKFKGRATLTADKSSSTAYMQVNSLTSEDSAVYYCATFINYVDYWGQGTTLTVSS

SEQ ID NO: 649 - protein, 106 aa, Synthetic construct
QIVLIQSPAIMSASPGEKVTMTCSASSSVNNIHWYQQKSGTSPKRWIYDTTKLTSGVPARFSGSGSGTSYSLTISSMEAEDAATYYCQQWNINPYTFGGGTKLEIK

SEQ ID NO: 650 - protein, 119 aa, Synthetic construct
EVQLQQSGPELVKPGSLVKISCKTSGYTFTDYNMDWVKQSHGKSLEWIGAIDPKNGATSYNQKFKGKATLTVDKSSSTAYMELRSLTSEDSAVYYCARWDSNYWYFDVWGTGTTVTVSS

SEQ ID NO: 651 - protein, 107 aa, Synthetic construct
DIVMTQSQKIMSTSVGDRVSMTCKASQNIRSAVAWYQQKPGQSPKALIYLTSNRHTGVPDRFTGSGSGTDFTLAISDLQSEDLADYFCLQHWNYPYTFGGGTELEIK

SEQ ID NO: 652 - protein, 112 aa, Synthetic construct
EVQLVESGGGLVKPGGSLRLSCAASGFTFSDYYMYWVRQAPGKGLEWVSTISDAGGYTYYPDSVKGRFTISRDNAKNSLYLQMNSLRAEDTAVYYCAREDLWGQGTLVTVSS

SEQ ID NO: 653 - protein, 107 aa, Synthetic construct
DIQMTQSPSSLSASVGDRVTITCRASQDIGYSLNWFQQKPGKAPKRLIYATSRLDSGVPKRFSGSGSGTDFTLTISSLQPEDFATYYCLQYASFPYTFGGGTKLEIK

SEQ ID NO: 654 - protein, 112 aa, Synthetic construct
EVQLVESGGGLVKPGGSLRLSCAASGFTFSDYYMYWVRQAPGKGLEWVATISDAGGYTYYPDSVKGRFTISRDNAKNSLYLQMNSLRAEDTAVYYCGREDLWGQGTLVTVSS

SEQ ID NO: 655 - protein, 107 aa, Synthetic construct
DIQMTQSPSSLSASVGDRVTITCRASQDIGYSLNWFQQKPGKAPRRLIYATSRLDSGVPKRFSGSGSGTDFTLTISSLQPEDFATYYCLQYASFPYTFGGGTKLEIK

SEQ ID NO: 656 - protein, 112 aa, Synthetic construct
EVQLVESGGGLVKPGGSLRLSCAASGFTFSDYYMYWVRQAPGKGLEWVATISDAGGYTYYPDSVKGRFTISRDNAKNSLYLQMNSLRAEDTAVYFCGREDLWGQGTLVTVSS

SEQ ID NO: 657 - protein, 447 aa, synthetic construct
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 658 - protein, 214 aa, synthetic construct
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 659 - protein, 330 aa, synthetic construct [sequence omitted]

SEQ ID NO: 660 - protein, 107 aa, synthetic construct [sequence omitted]

SEQ ID NO: 661 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 662 - DNA, 978 bp, synthetic construct
  source 1..978: mol_type = other DNA
atgagaacagaggcccaagtgcctgctctgcagcctccggagcctggcctggaaggcgccatgggccaccggaccctggtgctgccttgggtcctgctgaccctgtgtgtgaccgccggcacacctgaggtgtgggtgcaggttagaatggaggccacagagctgagctcttttaccatccggtgcggattcctgggatccggcagcatttctctggtcaccgtgtcttggggcggacctaatggcgctggcggcaccacactggccgtgctgcaccctgagcggggcatcagacagtgggcccctgccagacaggccagatgggaaacccagagcagcatctctctgatcctggaaggaagcggcgccagcagcccctgcgccaacacaacattctgctgcaagttcgcctccttcccagagggaagctgggaggcctgcggcagcctgcctcctagcagtgatcccggcctcagcgcccctcctacccctgctccaatcctgagagccgacctggccggaatcctgggcgtgtccggcgtgctgcttttcggctgcgtgtacctgctgcatctgctgaggcggcacaagcacagaccagcccctagactgcaaccttctagaaccagccctcaggcccctcgcgcccgggcttgggcccctagccaggcctctcaggctgctctgcatgtgccctacgcaaccatcaacaccagctgtagacctgccaccctggacaccgcccacccccacggaggcccaagctggtgggccagcctgcccacccacgccgcccacagaccccagggccccgccgcttgggctagcacccctatccccgcaagaggcagctttgtgtccgtggaaaacggcctgtacgcccaggccggcgagagacctccacacacaggccctggcctgacactgttccccgacccccggggccccagagccatggaaggccctctgggcgtgcgg